SEQUENCE LISTING (545 sequences)

SEQ ID NO: 1 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 2 - protein, 586 aa, synthetic construct
MAPKKKRKVGGGLVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 3 - protein, 566 aa, synthetic construct
MAPKKKRKVGGGSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 4 - protein, 546 aa, synthetic construct
MAPKKKRKVGGGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 5 - protein, 526 aa, synthetic construct
MAPKKKRKVGGGRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 6 - protein, 506 aa, synthetic construct
MAPKKKRKVGGGSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 7 - protein, 491 aa, synthetic construct
MAPKKKRKVGGGRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 8 - protein, 1223 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFARLAKLGGGAPAVGGGPKAADKGLPGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 9 - protein, 1203 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFARLAKLGGGAPAVGGGPKAADKGLPVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 10 - protein, 1183 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFARLAKLGGGAPAVGGGPKAADKGLPDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 11 - protein, 1163 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFARLAKLGGGAPAVGGGPKAADKGLPEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 12 - protein, 1150 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFARLAKLGGGAPAVGGGPKAADKGLPILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFSEQIDNO

SEQ ID NO: 13 - protein, 1123 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFARLAKLGGGAPAVGGGPKAADKGLPKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 14 - protein, 1108 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFARLAKLGGGAPAVGGGPKAADKGLPSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 15 - protein, 7 aa, synthetic construct
PKKKRKV

SEQ ID NO: 16 - protein, 25 aa, synthetic construct
ARLAKLGGGAPAVGGGPKAADKGLP

SEQ ID NO: 17 - DNA, 20 bp, synthetic construct
  source 1..20: mol_type = other DNA
gtgggcgaagatagcgacag

SEQ ID NO: 18 - DNA, 21 bp, synthetic construct
  source 1..21: mol_type = other DNA
gataccgaggaagccttcatc

SEQ ID NO: 19 - DNA, 18 bp, synthetic construct
  source 1..18: mol_type = other DNA
gagatcctggacgagcag

SEQ ID NO: 20 - DNA, 18 bp, synthetic construct
  source 1..18: mol_type = other DNA
atcctgacactgccccag

SEQ ID NO: 21 - DNA, 20 bp, synthetic construct
  source 1..20: mol_type = other DNA
aagagcaccagacggtctag

SEQ ID NO: 22 - DNA, 17 bp, synthetic construct
  source 1..17: mol_type = other DNA
agccagaggggccctac

SEQ ID NO: 23 - DNA, 17 bp, synthetic construct
  source 1..17: mol_type = other DNA
tccgccgccaactttcc

SEQ ID NO: 24 - DNA, 5419 bp, synthetic construct
  source 1..5419: mol_type = other DNA
ggcctaactggccagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtttagtcctgcaggccaccatggctcccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttctgatctagaaccggtcatggccgcaataaaatatctttattttcattacatctgtgtgttggttttttgtgtgttcgaactagatgctgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 25 - DNA, 20 bp, synthetic construct
  source 1..20: mol_type = other DNA
tctagaaccggtcatggccg

SEQ ID NO: 26 - DNA, 20 bp, synthetic construct
  source 1..20: mol_type = other DNA
ggtggcctgcaggactaaac

SEQ ID NO: 27 - DNA, 6044 bp, synthetic construct
  source 1..6044: mol_type = other DNA
taatacgactcactataaggagacaagcttgccaccatggctcccaagaagaagagaaaagttggcggcggaggcagcagcctggacgatgaacatattctgagcgccctgctgcagagcgacgatgagcttgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagcggacctactgcacctactgtcccagcaagatccggcggaaggccaacgccagctgcaaaaagtgcaagaaagtgatctgccgcgagcacaacatcgacatgtgccagagctgcttcgccagactggccaaacttggaggcggagcaccagctgttggcggaggacctaaagccgctgataagggactgcctggatccatggacgagaaaaccaccggctggcgcggaggacatgtggttgaaggacttgccggcgagctggaacagctgagagccagactggaacaccatcctcagggccagagagagcctggcggcagctatccttacgacgtgccagattatgccggaagctccctggatgacgagcacatcctgtctgcactgctgcagtctgacgacgaactcgtcggcgaggacagcgattccgaagtgtccgaccatgtctctgaggacgacgtccagagcgacaccgaagaggcctttattgatgaagtgcatgaggtgcagcccaccagctccggatctgagattctcgatgaacagaacgtcatcgaacagcccggcagctctctggcctccaacaggattcttaccctgcctcagcggaccatcagagggaaaaacaaacactgttggagcacctccaagtccaccaggcgcagtagagtcagcgccctgaatattgtgcgctcccagcggggaccaaccaggatgtgtagaaatatctatgatcccctgctctgttttaagctgttttttacggatgagattatcagcgagattgtcaaatggacgaatgccgaaatctccctgaagcgccgcgagagcatgacctctgccaccttccgggatacaaatgaagatgagatctatgctttctttgggattctcgtgatgacggctgtccgcaaggataatcacatgtccaccgatgatctgtttgatcggagcctgagcatggtctatgtgtctgtgatgtcccgcgatcgcttcgattttctgattcgctgcctgagaatggatgataagagcatccggcctacactgcgggaaaacgatgtctttacccctgtcagaaagatttgggatctctttattcaccagtgtattcagaattatacacccggcgcacacctcacaatcgatgagcagctcctcggctttcggggcagatgtccttttcgggtgtacattcctaacaaacccagcaaatatgggatcaaaattctcatgatgtgtgactctgggacgaagtatatgatcaatgggatgccgtatcttggacggggcacccagactaacggtgtcccactgggagaatattatgtcaaagaactttccaagccggtccacggctcctgccgcaacatcacatgcgataattggttcacctctataccgctcgccaaaaatctcctgcaagagccttataagctcaccattgtcggcacagtgcgctccaacaaacgcgagatccctgaagtgctcaagaacagcagatcccggccagtgggcacctccatgttttgttttgatggcccactgacactcgtcagctataagcctaagccagcaaaaatggtctacctcctgtcctcctgcgacgaggatgcctctatcaacgagtccacagggaaacctcaaatggttatgtattacaatcaaacgaaaggcggagtcgatacgctcgaccagatgtgttcagtgatgacatgctcccgcaagaccaaccggtggcctatggcactcctctatgggatgatcaacattgcttgtatcaattcctttatcatctactcccataatgtgtcctctaagggcgaaaaagtccagtctcggaaaaagttcatgaggaatctctatatgtccctgacctccagctttatgcggaagagactggaagctccaactctgaaacgctacctccgcgacaatatctccaacattctccccaaagaagtgcctggaacctccgacgactccaccgaagaaccagtcatgaagaaacgcacgtattgcacatactgcccctctaagatccgcagaaaggccaatgcctcttgtaaaaagtgtaaaaaagtcatttgtcgggaacacaatattgatatgtgccagtcctgcttttgatgagcggccgcggatccccgggtaccgaattcgatatctctatagtgtcacctaaatttaattaaaaaaaaaaaaaaaaaaaaaaaaaaaaaaaaaaaaaaaaaaaaaaaaaaaaaaaaaaaaaaaaaaaaaaaaaaaaaaaactagtagatctctcgaggctagccttaagggcgcctattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgagattatcaaaaaggatcttcacctagatccttttaaattaaaaatgaagttttaaatcaatctaaagtatatatgagtaaacttggtctgacagtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggttcgtctcgtcccggtctcctcccaatgcatgtcaatattggccattagccatattattcattggttatatagcataaatcaatattggctattggccattgcatacgttgtatctatatcataatatgtacatt

SEQ ID NO: 28 - DNA, 37 bp, synthetic construct
  source 1..37: mol_type = other DNA
gtgaggtttagtcctgcaggccaccatggctcccaag

SEQ ID NO: 29 - DNA, 49 bp, synthetic construct
  source 1..49: mol_type = other DNA
tattgcggccatgaccggttctagatcaaaagcaggactggcacatatc

SEQ ID NO: 30 - DNA, 7429 bp, synthetic construct
  source 1..7429: mol_type = other DNA
ggcctaactggccagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtttagtcctgcaggccaccatggctcccaagaagaagagaaaagttggcggcggaggcagcagcctggacgatgaacatattctgagcgccctgctgcagagcgacgatgagcttgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagcggacctactgcacctactgtcccagcaagatccggcggaaggccaacgccagctgcaaaaagtgcaagaaagtgatctgccgcgagcacaacatcgacatgtgccagagctgcttcgccagactggccaaacttggaggcggagcaccagctgttggcggaggacctaaagccgctgataagggactgcctggatccatggacgagaaaaccaccggctggcgcggaggacatgtggttgaaggacttgccggcgagctggaacagctgagagccagactggaacaccatcctcagggccagagagagcctggcggcagctatccttacgacgtgccagattatgccggaagctccctggatgacgagcacatcctgtctgcactgctgcagtctgacgacgaactcgtcggcgaggacagcgattccgaagtgtccgaccatgtctctgaggacgacgtccagagcgacaccgaagaggcctttattgatgaagtgcatgaggtgcagcccaccagctccggatctgagattctcgatgaacagaacgtcatcgaacagcccggcagctctctggcctccaacaggattcttaccctgcctcagcggaccatcagagggaaaaacaaacactgttggagcacctccaagtccaccaggcgcagtagagtcagcgccctgaatattgtgcgctcccagcggggaccaaccaggatgtgtagaaatatctatgatcccctgctctgttttaagctgttttttacggatgagattatcagcgagattgtcaaatggacgaatgccgaaatctccctgaagcgccgcgagagcatgacctctgccaccttccgggatacaaatgaagatgagatctatgctttctttgggattctcgtgatgacggctgtccgcaaggataatcacatgtccaccgatgatctgtttgatcggagcctgagcatggtctatgtgtctgtgatgtcccgcgatcgcttcgattttctgattcgctgcctgagaatggatgataagagcatccggcctacactgcgggaaaacgatgtctttacccctgtcagaaagatttgggatctctttattcaccagtgtattcagaattatacacccggcgcacacctcacaatcgatgagcagctcctcggctttcggggcagatgtccttttcgggtgtacattcctaacaaacccagcaaatatgggatcaaaattctcatgatgtgtgactctgggacgaagtatatgatcaatgggatgccgtatcttggacggggcacccagactaacggtgtcccactgggagaatattatgtcaaagaactttccaagccggtccacggctcctgccgcaacatcacatgcgataattggttcacctctataccgctcgccaaaaatctcctgcaagagccttataagctcaccattgtcggcacagtgcgctccaacaaacgcgagatccctgaagtgctcaagaacagcagatcccggccagtgggcacctccatgttttgttttgatggcccactgacactcgtcagctataagcctaagccagcaaaaatggtctacctcctgtcctcctgcgacgaggatgcctctatcaacgagtccacagggaaacctcaaatggttatgtattacaatcaaacgaaaggcggagtcgatacgctcgaccagatgtgttcagtgatgacatgctcccgcaagaccaaccggtggcctatggcactcctctatgggatgatcaacattgcttgtatcaattcctttatcatctactcccataatgtgtcctctaagggcgaaaaagtccagtctcggaaaaagttcatgaggaatctctatatgtccctgacctccagctttatgcggaagagactggaagctccaactctgaaacgctacctccgcgacaatatctccaacattctccccaaagaagtgcctggaacctccgacgactccaccgaagaaccagtcatgaagaaacgcacgtattgcacatactgcccctctaagatccgcagaaaggccaatgcctcttgtaaaaagtgtaaaaaagtcatttgtcgggaacacaatattgatatgtgccagtcctgcttttgatctagaaccggtcatggccgcaataaaatatctttattttcattacatctgtgtgttggttttttgtgtgttcgaactagatgctgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 31 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVRTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 32 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTKDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 33 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDKLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 34 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFRRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 35 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVRTAVRKDNHMSTDKLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 36 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVRTAVRKDNHMSTDDLFRRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 37 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTKDLFRRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 38 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDKLFRRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 39 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVRTAVRKDNHMSTDKLFRRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 40 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVKTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 41 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTRDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 42 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDRLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 43 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFKRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 44 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSESMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 45 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRDKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 46 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMDNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 47 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSESMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRDKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 48 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSESMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMDNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 49 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRDKFMDNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 50 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSESMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRDKFMDNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 51 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSDSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 52 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSREKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 53 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMENLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 54 - DNA, 5419 bp, synthetic construct
  source 1..5419: mol_type = other DNA
agctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtttagtcctgcaggccaccatggctcccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttctgatctagaaccggtcatggccgcaataaaatatctttattttcattacatctgtgtgttggttttttgtgtgttcgaactagatgctgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggcc

SEQ ID NO: 55 - protein, 598 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 56 - protein, 598 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 57 - protein, 99 aa, synthetic construct
GGGGSERPYACPVESCDRRFSRSDELTRHIRIHTGQKPFQCRICMRNFSRSDHLTTHIRTHTGEKPFACDICGRKFARSDERKRHTKIHLRQKDGGGGS

SEQ ID NO: 58 - protein, 609 aa, synthetic construct
MAPKKKRKVGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 59 - protein, 609 aa, synthetic construct
MAPKKKRKVGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 60 - skipped

SEQ ID NO: 61 - DNA, 40 bp, synthetic construct
  source 1..40: mol_type = other DNA
acgcccacgcttacatctttaaagatgtaagcgtgggcgt

SEQ ID NO: 62 - DNA, 38 bp, synthetic construct
  source 1..38: mol_type = other DNA
acgcccacgctacatctttaaagatgtagcgtgggcgt

SEQ ID NO: 63 - DNA, 36 bp, synthetic construct
  source 1..36: mol_type = other DNA
acgcccacgctcatctttaaagatgagcgtgggcgt

SEQ ID NO: 64 - DNA, 34 bp, synthetic construct
  source 1..34: mol_type = other DNA
acgcccacgctctctttaaagagagcgtgggcgt

SEQ ID NO: 65 - protein, 501 aa, synthetic construct
NKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 66 - protein, 501 aa, synthetic construct
NKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 67 - protein, 703 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 68 - protein, 91 aa, synthetic construct
GSSLDDEHILSALLQSDDELVGEDSDSEVSDHVSEDDVQSDTEEAFIDEVHEVQPTSSGSEILDEQNVIEQPGSSLASNRILTLPQRTIRG

SEQ ID NO: 69 - protein, 703 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 70 - protein, 23 aa, synthetic construct
MDYKDHDGDYKDHDIDYKDDDDK

SEQ ID NO: 71 - protein, 9 aa, synthetic construct
MAPKKKRKV

SEQ ID NO: 72 - DNA, 24 bp, synthetic construct
  source 1..24: mol_type = other DNA
ggcatccacagaggcgtgcccatg

SEQ ID NO: 73 - protein, 136 aa, synthetic construct
VDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 74 - protein, 136 aa, synthetic construct
VDLRTLGYSQQQQEKIKPKVRSTVAQHHEALVGHGFTHAHIVALSQHPAALGTVAVKYQDMIAALPEATHEAIVGVGKSRSGARALEALLTVAGELRGPPLQLDTGQLLKIAKRGGVTAVEAVHAWRNALTGAPLN

SEQ ID NO: 75 - protein, 135 aa, synthetic construct
VDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLN

SEQ ID NO: 76 - protein, 63 aa, synthetic construct
SIVAQLSRPDPALAALTNDHLVALACLGGRPALDAVKKGLPHAPALIKRTNRRIPERTSHRVA

SEQ ID NO: 77 - protein, 278 aa, synthetic construct
SIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPTHEGDQRRASSRKRSRSDRAVTGPSAQQSFEVRAPEQRDALHLPLSWRVKRPRTSIGGGLPDPGTPTAADLAASSTVMREQDEDPFAGAADDFPAFNEEELAWLMELLPQ

SEQ ID NO: 78 - protein, 5 aa, synthetic construct
GGGGS

SEQ ID NO: 79 - protein, 196 aa, synthetic construct
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 80 - protein, 594 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 81 - protein, 594 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 82 - protein, 594 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNAREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 83 - protein, 594 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 84 - protein, 594 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 85 - protein, 594 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 86 - protein, 511 aa, synthetic construct
TLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 87 - protein, 510 aa, synthetic construct
LPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 88 - protein, 509 aa, synthetic construct
PQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 89 - protein, 508 aa, synthetic construct
QRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 90 - protein, 507 aa, synthetic construct
RTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 91 - protein, 506 aa, synthetic construct
TIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 92 - protein, 505 aa, synthetic construct
IRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 93 - protein, 504 aa, synthetic construct
RGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 94 - protein, 503 aa, synthetic construct
GKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 95 - protein, 502 aa, synthetic construct
KNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 96 - protein, 501 aa, synthetic construct; identical to SEQ ID NO: 66

SEQ ID NO: 97 - protein, 500 aa, synthetic construct
KHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 98 - protein, 499 aa, synthetic construct
HCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 99 - protein, 498 aa, synthetic construct
CWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 100 - protein, 497 aa, synthetic construct
WSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 101 - protein, 496 aa, synthetic construct
STSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 102 - protein, 495 aa, synthetic construct
TSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 103 - protein, 494 aa, synthetic construct
SKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 104 - protein, 493 aa, synthetic construct
KSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 105 - protein, 492 aa, synthetic construct
STRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 106 - protein, 491 aa, synthetic construct
TRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 107 - protein, 34 aa, synthetic construct
  VARIANT 11..13
LTPDQVVAIAXXXGGKQALETVQRLLPVLCQDHG

SEQ ID NO: 108 - protein, 34 aa, synthetic construct
  VARIANT 11..13
LTPEQVVAIAXXXGGKQALETVQRLLPVLCQAHG

SEQ ID NO: 109 - protein, 34 aa, synthetic construct
  VARIANT 11..13
LTPDQVVAIAXXXGGKQALETVQRLLPVLCQAHG

SEQ ID NO: 110 - protein, 34 aa, synthetic construct
  VARIANT 11..13
LTPAQVVAIAXXXGGKQALETVQRLLPVLCQDHG

SEQ ID NO: 111 - protein, 20 aa, synthetic construct
  VARIANT 11..13
LTPEQVVAIAXXXGGRPALE

SEQ ID NO: 112 - protein, 326 aa, synthetic construct
LTPDQVVAIASNIGGKQALETVQRLLPVLCQDHGLTPEQVVAIASHDGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIANNNGGKQALETVQRLLPVLCQDHGLTPEQVVAIASHDGGRPALE

SEQ ID NO: 113 - protein, 326 aa, synthetic construct
LTPDQVVAIANNNGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIANNNGGRPALE

SEQ ID NO: 114 - protein, 326 aa, synthetic construct
LTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALE

SEQ ID NO: 115 - protein, 326 aa, synthetic construct
LTPDQVVAIANNNGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASNGGGRPALE

SEQ ID NO: 116 - protein, 326 aa, synthetic construct
LTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIANNNGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNIGGRPALE

SEQ ID NO: 117 - protein, 326 aa, synthetic construct
LTPDQVVAIANNNGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASNGGGRPALE

SEQ ID NO: 118 - protein, 326 aa, synthetic construct
LTPDQVVAIASHDGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIANNNGGRPALE

SEQ ID NO: 119 - protein, 326 aa, synthetic construct
LTPDQVVAIASNGGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNGGGRPALE

SEQ ID NO: 120 - protein, 326 aa, synthetic construct
LTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIANNNGGRPALE

SEQ ID NO: 121 - protein, 326 aa, synthetic construct
LTPDQVVAIANNNGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIANNNGGRPALE

SEQ ID NO: 122 - protein, 326 aa, synthetic construct
LTPDQVVAIASNGGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNIGGRPALE

SEQ ID NO: 123 - protein, 326 aa, synthetic construct
LTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASNIGGRPALE

SEQ ID NO: 124 - protein, 326 aa, synthetic construct
LTPDQVVAIANNNGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIANNNGGRPALE

SEQ ID NO: 125 - protein, 326 aa, synthetic construct
LTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALE

SEQ ID NO: 126 - protein, 326 aa, synthetic construct
LTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASNGGGRPALE

SEQ ID NO: 127 - protein, 326 aa, synthetic construct
LTPDQVVAIASNGGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIASHDGGRPALE

SEQ ID NO: 128 - protein, 326 aa, synthetic construct
LTPDQVVAIANNNGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASNIGGRPALE

SEQ ID NO: 129 - protein, 326 aa, synthetic construct
LTPDQVVAIASNIGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASHDGGRPALE

SEQ ID NO: 130 - protein, 326 aa, synthetic construct
LTPDQVVAIASHDGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASHDGGRPALE

SEQ ID NO: 131 - protein, 326 aa, synthetic construct
LTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALE

SEQ ID NO: 132 - protein, 326 aa, synthetic construct
LTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIANNNGGRPALE

SEQ ID NO: 133 - protein, 326 aa, synthetic construct
LTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALE

SEQ ID NO: 134 - protein, 326 aa, synthetic construct
LTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALE

SEQ ID NO: 135 - protein, 326 aa, synthetic construct
LTPDQVVAIASNGGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASNGGGRPALE

SEQ ID NO: 136 - protein, 326 aa, synthetic construct
LTPDQVVAIASNGGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASNGGGRPALE

SEQ ID NO: 137 - protein, 326 aa, synthetic construct
LTPDQVVAIASHDGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIANNNGGRPALE

SEQ ID NO: 138 - protein, 326 aa, synthetic construct
LTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIASNGGGRPALE

SEQ ID NO: 139 - protein, 326 aa, synthetic construct
LTPDQVVAIASNIGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNIGGRPALE

SEQ ID NO: 140 - protein, 326 aa, synthetic construct
LTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNIGGRPALE

SEQ ID NO: 141 - protein, 326 aa, synthetic construct
LTPDQVVAIASHDGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNIGGRPALE

SEQ ID NO: 142 - protein, 326 aa, synthetic construct
LTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALE

SEQ ID NO: 143 - protein, 326 aa, synthetic construct
LTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNGGGRPALE

SEQ ID NO: 144 - protein, 326 aa, synthetic construct
LTPDQVVAIANNNGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASHDGGRPALE

SEQ ID NO: 145 - protein, 326 aa, synthetic construct
LTPDQVVAIANNNGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGRPALE

SEQ ID NO: 146 - protein, 326 aa, synthetic construct
LTPDQVVAIANNNGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASNIGGRPALE

SEQ ID NO: 147 - protein, 326 aa, synthetic construct
LTPDQVVAIASNGGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASNGGGRPALE

SEQ ID NO: 148 - protein, 326 aa, synthetic construct
LTPDQVVAIANNNGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASNGGGRPALE

SEQ ID NO: 149 - protein, 326 aa, synthetic construct
LTPDQVVAIASHDGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIANNNGGRPALE

SEQ ID NO: 150 - protein, 326 aa, synthetic construct
LTPDQVVAIASHDGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIANNNGGRPALE

SEQ ID NO: 151 - protein, 326 aa, synthetic construct
LTPDQVVAIANNNGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASNIGGRPALE

SEQ ID NO: 152 - protein, 326 aa, synthetic construct
LTPDQVVAIASNGGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASNGGGRPALE

SEQ ID NO: 153 - protein, 326 aa, synthetic construct
LTPDQVVAIASNIGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIANNNGGRPALE

SEQ ID NO: 154 - protein, 326 aa, synthetic construct
LTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIASNGGGRPALE

SEQ ID NO: 155 - protein, 326 aa, synthetic construct
LTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNIGGRPALE

SEQ ID NO: 156 - protein, 326 aa, synthetic construct
LTPDQVVAIASNGGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGRPALE

SEQ ID NO: 157 - protein, 326 aa, synthetic construct
LTPDQVVAIASHDGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGRPALE

SEQ ID NO: 158 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASHDGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIANNNGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 159 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 160 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 161 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 162 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIANNNGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 163 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 164 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 165 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 166 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 167 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 168 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 169 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 170 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 171 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 172 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 173 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 174 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 175 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 176 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 177 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 178 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 179 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 180 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 181 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 182 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 183 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 184 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 185 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 186 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 187 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 188 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 189 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 190 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASHDGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIANNNGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 191 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 192 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 193 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 194 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIANNNGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 195 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 196 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 197 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 198 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 199 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 200 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 201 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 202 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 203 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 204 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 205 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 206 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 207 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 208 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 209 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 210 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 211 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 212 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 213 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 214 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 215 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 216 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 217 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 218 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 219 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 220 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 221 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 222 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 223 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 224 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 225 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 226 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 227 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 228 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 229 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 230 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 231 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 232 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 233 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 234 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 235 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 236 - protein, 550 aa, synthetic construct
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 237 - DNA, 558 bp, synthetic construct
  source 1..558: mol_type = other DNA
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgaataatga

SEQ ID NO: 238 - DNA, 1417 bp, synthetic construct
  source 1..1417: mol_type = other DNA
ggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 239 - DNA, 11 bp, synthetic construct
  source 1..11: mol_type = other DNA
tacgcccacgc

SEQ ID NO: 240 - protein, 11 aa, synthetic construct
TGCCACCTACG

SEQ ID NO: 241 - DNA, 11 bp, synthetic construct
  source 1..11: mol_type = other DNA
tgcagatgaac

SEQ ID NO: 242 - DNA, 11 bp, synthetic construct
  source 1..11: mol_type = other DNA
tggcccaccct

SEQ ID NO: 243 - DNA, 11 bp, synthetic construct
  source 1..11: mol_type = other DNA
tgcacgccgta

SEQ ID NO: 244 - DNA, 11 bp, synthetic construct
  source 1..11: mol_type = other DNA
tgagatgatgt

SEQ ID NO: 245 - DNA, 11 bp, synthetic construct
  source 1..11: mol_type = other DNA
tctcttgtaag

SEQ ID NO: 246 - DNA, 11 bp, synthetic construct
  source 1..11: mol_type = other DNA
ttcagtttgtt

SEQ ID NO: 247 - DNA, 11 bp, synthetic construct
  source 1..11: mol_type = other DNA
tcttttaggag

SEQ ID NO: 248 - DNA, 11 bp, synthetic construct
  source 1..11: mol_type = other DNA
tgcttcatagg

SEQ ID NO: 249 - DNA, 11 bp, synthetic construct
  source 1..11: mol_type = other DNA
tttagatcaca

SEQ ID NO: 250 - DNA, 11 bp, synthetic construct
  source 1..11: mol_type = other DNA
tatgatcctaa

SEQ ID NO: 251 - DNA, 11 bp, synthetic construct
  source 1..11: mol_type = other DNA
tgattgctaag

SEQ ID NO: 252 - DNA, 11 bp, synthetic construct
  source 1..11: mol_type = other DNA
ttctaggaaac

SEQ ID NO: 253 - DNA, 11 bp, synthetic construct
  source 1..11: mol_type = other DNA
ttttgtttcct

SEQ ID NO: 254 - DNA, 11 bp, synthetic construct
  source 1..11: mol_type = other DNA
ttggcagccac

SEQ ID NO: 255 - DNA, 11 bp, synthetic construct
  source 1..11: mol_type = other DNA
tgccactataa

SEQ ID NO: 256 - DNA, 11 bp, synthetic construct
  source 1..11: mol_type = other DNA
tataaatggac

SEQ ID NO: 257 - DNA, 11 bp, synthetic construct
  source 1..11: mol_type = other DNA
tccaacttgcc

SEQ ID NO: 258 - DNA, 11 bp, synthetic construct
  source 1..11: mol_type = other DNA
tcctagtctct

SEQ ID NO: 259 - DNA, 11 bp, synthetic construct
  source 1..11: mol_type = other DNA
tgtctcttttg

SEQ ID NO: 260 - DNA, 11 bp, synthetic construct
  source 1..11: mol_type = other DNA
ttgtctctttt

SEQ ID NO: 261 - DNA, 11 bp, synthetic construct
  source 1..11: mol_type = other DNA
tgcaatcaaac

SEQ ID NO: 262 - DNA, 11 bp, synthetic construct
  source 1..11: mol_type = other DNA
ttgagcggctt

SEQ ID NO: 263 - DNA, 11 bp, synthetic construct
  source 1..11: mol_type = other DNA
ttcacagaatt

SEQ ID NO: 264 - DNA, 11 bp, synthetic construct
  source 1..11: mol_type = other DNA
tcacagaattg

SEQ ID NO: 265 - DNA, 11 bp, synthetic construct
  source 1..11: mol_type = other DNA
tcttttttggt

SEQ ID NO: 266 - DNA, 11 bp, synthetic construct
  source 1..11: mol_type = other DNA
tacaaaaatca

SEQ ID NO: 267 - DNA, 11 bp, synthetic construct
  source 1..11: mol_type = other DNA
ttttaggttta

SEQ ID NO: 268 - DNA, 11 bp, synthetic construct
  source 1..11: mol_type = other DNA
tcaattcaaga

SEQ ID NO: 269 - DNA, 11 bp, synthetic construct
  source 1..11: mol_type = other DNA
ttttatggttt

SEQ ID NO: 270 - DNA, 11 bp, synthetic construct
  source 1..11: mol_type = other DNA
tttttatggtt

SEQ ID NO: 271 - DNA, 11 bp, synthetic construct
  source 1..11: mol_type = other DNA
tgatacaaagc

SEQ ID NO: 272 - DNA, 11 bp, synthetic construct
  source 1..11: mol_type = other DNA
tgacatgtgat

SEQ ID NO: 273 - DNA, 11 bp, synthetic construct
  source 1..11: mol_type = other DNA
tgaagaaacta

SEQ ID NO: 274 - DNA, 11 bp, synthetic construct
  source 1..11: mol_type = other DNA
ttatcccctgt

SEQ ID NO: 275 - DNA, 11 bp, synthetic construct
  source 1..11: mol_type = other DNA
tggctgtaatt

SEQ ID NO: 276 - DNA, 11 bp, synthetic construct
  source 1..11: mol_type = other DNA
tcacgcagaag

SEQ ID NO: 277 - DNA, 11 bp, synthetic construct
  source 1..11: mol_type = other DNA
tctgtgctctg

SEQ ID NO: 278 - DNA, 11 bp, synthetic construct
  source 1..11: mol_type = other DNA
tgagcttctaa

SEQ ID NO: 279 - DNA, 11 bp, synthetic construct
  source 1..11: mol_type = other DNA
ttgatggggct

SEQ ID NO: 280 - DNA, 11 bp, synthetic construct
  source 1..11: mol_type = other DNA
tatctctctag

SEQ ID NO: 281 - DNA, 11 bp, synthetic construct
  source 1..11: mol_type = other DNA
ttttatcgggt

SEQ ID NO: 282 - DNA, 11 bp, synthetic construct
  source 1..11: mol_type = other DNA
tgcatacaaga

SEQ ID NO: 283 - DNA, 11 bp, synthetic construct
  source 1..11: mol_type = other DNA
ttgagagcctc

SEQ ID NO: 284 - DNA, 11 bp, synthetic construct
  source 1..11: mol_type = other DNA
tcactggagat

SEQ ID NO: 285 - DNA, 42 bp, synthetic construct
  source 1..42: mol_type = other DNA
ggtacgcccacgctcaagctgaccctgaagcgtgggcgtatt

SEQ ID NO: 286 - DNA, 42 bp, synthetic construct
  source 1..42: mol_type = other DNA
ggtgccacctacggcaagctgaccctgaagttcatctgcatt

SEQ ID NO: 287 - DNA, 42 bp, synthetic construct
  source 1..42: mol_type = other DNA
ggtgccacctacggcaagctgaccctgaacgtaggtggcatt

SEQ ID NO: 288 - DNA, 42 bp, synthetic construct
  source 1..42: mol_type = other DNA
ggtgcagatgaacgcaagctgaccctgaagttcatctgcatt

SEQ ID NO: 289 - DNA, 42 bp, synthetic construct
  source 1..42: mol_type = other DNA
ggtggcccaccctcgtgaccaccctgacctacggcgtgcatt

SEQ ID NO: 290 - DNA, 42 bp, synthetic construct
  source 1..42: mol_type = other DNA
ggtggcccaccctcgtgaccaccctgaccagggtgggccatt

SEQ ID NO: 291 - DNA, 42 bp, synthetic construct
  source 1..42: mol_type = other DNA
ggtgcacgccgtacgtgaccaccctgacctacggcgtgcatt

SEQ ID NO: 292 - DNA, 42 bp, synthetic construct
  source 1..42: mol_type = other DNA
ggtgagatgatgtacaagctgaccctgatacatcatctcatt

SEQ ID NO: 293 - DNA, 42 bp, synthetic construct
  source 1..42: mol_type = other DNA
ggtctcttgtaagccaagctgaccctgagcttacaagagatt

SEQ ID NO: 294 - DNA, 42 bp, synthetic construct
  source 1..42: mol_type = other DNA
ggttcagtttgttacaagctgaccctgataacaaactgaatt

SEQ ID NO: 295 - DNA, 42 bp, synthetic construct
  source 1..42: mol_type = other DNA
ggtcttttaggagccaagctgaccctgagctcctaaaagatt

SEQ ID NO: 296 - DNA, 42 bp, synthetic construct
  source 1..42: mol_type = other DNA
ggtgcttcatagggcaagctgaccctgaccctatgaagcatt

SEQ ID NO: 297 - DNA, 42 bp, synthetic construct
  source 1..42: mol_type = other DNA
ggtttagatcacatcaagctgaccctgaatgtgatctaaatt

SEQ ID NO: 298 - DNA, 42 bp, synthetic construct
  source 1..42: mol_type = other DNA
ggtatgatcctaatcaagctgaccctgaattaggatcatatt

SEQ ID NO: 299 - DNA, 42 bp, synthetic construct
  source 1..42: mol_type = other DNA
ggtgattgctaagacaagctgaccctgatcttagcaatcatt

SEQ ID NO: 300 - DNA, 42 bp, synthetic construct
  source 1..42: mol_type = other DNA
ggttctaggaaactcaagctgaccctgaagtttcctagaatt

SEQ ID NO: 301 - DNA, 42 bp, synthetic construct
  source 1..42: mol_type = other DNA
ggttttgtttccttcaagctgaccctgaaaggaaacaaaatt

SEQ ID NO: 302 - DNA, 42 bp, synthetic construct
  source 1..42: mol_type = other DNA
ggttggcagccactcaagctgaccctgaagtggctgccaatt

SEQ ID NO: 303 - DNA, 42 bp, synthetic construct
  source 1..42: mol_type = other DNA
ggtgccactataagcaagctgaccctgacttatagtggcatt

SEQ ID NO: 304 - DNA, 42 bp, synthetic construct
  source 1..42: mol_type = other DNA
ggtataaatggacacaagctgaccctgatgtccatttatatt

SEQ ID NO: 305 - DNA, 42 bp, synthetic construct
  source 1..42: mol_type = other DNA
ggtccaacttgccacaagctgaccctgatggcaagttggatt

SEQ ID NO: 306 - DNA, 42 bp, synthetic construct
  source 1..42: mol_type = other DNA
ggtcctagtctctacaagctgaccctgatagagactaggatt

SEQ ID NO: 307 - DNA, 42 bp, synthetic construct
  source 1..42: mol_type = other DNA
ggtgtctcttttgacaagctgaccctgatcaaaagagacatt

SEQ ID NO: 308 - DNA, 42 bp, synthetic construct
  source 1..42: mol_type = other DNA
ggttgtctcttttacaagctgaccctgataaaagagacaatt

SEQ ID NO: 309 - DNA, 42 bp, synthetic construct
  source 1..42: mol_type = other DNA
ggtgcaatcaaacacaagctgaccctgatgtttgattgcatt

SEQ ID NO: 310 - DNA, 42 bp, synthetic construct
  source 1..42: mol_type = other DNA
ggttgagcggcttacaagctgaccctgataagccgctcaatt

SEQ ID NO: 311 - DNA, 42 bp, synthetic construct
  source 1..42: mol_type = other DNA
ggttcacagaattacaagctgaccctgataattctgtgaatt

SEQ ID NO: 312 - DNA, 42 bp, synthetic construct
  source 1..42: mol_type = other DNA
ggtcacagaattgacaagctgaccctgatcaattctgtgatt

SEQ ID NO: 313 - DNA, 42 bp, synthetic construct
  source 1..42: mol_type = other DNA
ggtcttttttggtacaagctgaccctgataccaaaaaagatt

SEQ ID NO: 314 - DNA, 42 bp, synthetic construct
  source 1..42: mol_type = other DNA
ggtacaaaaatcaacaagctgaccctgattgatttttgtatt

SEQ ID NO: 315 - DNA, 42 bp, synthetic construct
  source 1..42: mol_type = other DNA
ggttttaggtttaacaagctgaccctgattaaacctaaaatt

SEQ ID NO: 316 - DNA, 42 bp, synthetic construct
  source 1..42: mol_type = other DNA
ggtcaattcaagaacaagctgaccctgattcttgaattgatt

SEQ ID NO: 317 - DNA, 42 bp, synthetic construct
  source 1..42: mol_type = other DNA
ggttttatggtttacaagctgaccctgataaaccataaaatt

SEQ ID NO: 318 - DNA, 42 bp, synthetic construct
  source 1..42: mol_type = other DNA
ggtttttatggttacaagctgaccctgataaccataaaaatt

SEQ ID NO: 319 - DNA, 35 bp, synthetic construct
  source 1..35: mol_type = other DNA
ccctagaaagatagtctgcgtaaaattgacgcatg

SEQ ID NO: 320 - DNA, 63 bp, synthetic construct
  source 1..63: mol_type = other DNA
ccctagaaagataatcatattgtgacgtacgttaaagataatcatgcgtaaaattgacgcatg

SEQ ID NO: 321 - DNA, 88 bp, synthetic construct
  source 1..88: mol_type = other DNA
agagtagtccagggtttccgagggtttccgtcgactcagctcgtctcgagggcgtactaactgggccctttcccttttttttcctcag

SEQ ID NO: 322 - DNA, 612 bp, synthetic construct
  source 1..612: mol_type = other DNA
ggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 323 - DNA, 49 bp, synthetic construct
  source 1..49: mol_type = other DNA
aataaaatatctttattttcattacatctgtgtgttggttttttgtgtg

SEQ ID NO: 324 - DNA, 1474 bp, synthetic construct
  source 1..1474: mol_type = other DNA
tacctaatgatttttatcaaaatcattaagttaaggtagatacacatcttgtcatatgatcaaatggtttcgccaaaaatcaataatcagacaacaagatgtgcgaactcgatattttacacgactctctttaccaattctaccacaacgtggatcagcagttcaacctgttgatagtacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccaaaaaaaagcccgctcattaggcgggctgcgcccttaaggctagcggtctctttaaccctagaaagatagtctgcgtaaaattgacgcatgagagtagtccagggtttccgagggtttccgtcgactcagctcgtctcgagggcgtactaactgggccctttcccttttttttcctcagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgatctagaaccggtcatggccgcaataaaatatctttattttcattacatctgtgtgttggttttttgtgtgttcgaactagatgctgtcgaccgatgcccttgagagccttcaacccagtcagctccttccggtgggcgcggggcatgactatcgtcgccgcacttatcatgcgtcaattttacgcatgattatctttaacgtacgtcacaatatgattatctttctagggttaaagagacctgtaca

SEQ ID NO: 325 - DNA, 1264 bp, synthetic construct
  source 1..1264: mol_type = other DNA
agctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 326 - DNA, 105 bp, synthetic construct
  source 1..105: mol_type = other DNA
atggtgagcaagggcgaggagctgttcaccggggtggtgcccatcctggtcgagctggacggcgacgtaaacggccacaagttcagcgtgtccggcgagggcgag

SEQ ID NO: 327 - DNA, 37 bp, synthetic construct
  source 1..37: mol_type = other DNA
gtgagtatggatccctctaaaagcgggcatgacttct

SEQ ID NO: 328 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 329 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 330 - DNA, 38 bp, synthetic construct
  source 1..38: mol_type = other DNA
tgcagatgaacgcatgcttaactctaagttcatctgca

SEQ ID NO: 331 - DNA, 40 bp, synthetic construct
  source 1..40: mol_type = other DNA
tgcagatgaacgcatcgcttaactactaagttcatctgca

SEQ ID NO: 332 - DNA, 42 bp, synthetic construct
  source 1..42: mol_type = other DNA
tgcagatgaacgcattcgcttaactacctaagttcatctgca

SEQ ID NO: 333 - DNA, 44 bp, synthetic construct
  source 1..44: mol_type = other DNA
tgcagatgaacgcatctcgcttaactacgctaagttcatctgca

SEQ ID NO: 334 - DNA, 46 bp, synthetic construct
  source 1..46: mol_type = other DNA
tgcagatgaacgcaatctcgcttaactacgctgaagttcatctgca

SEQ ID NO: 335 - DNA, 48 bp, synthetic construct
  source 1..48: mol_type = other DNA
tgcagatgaacgcaatctcggcttaactactgctgaagttcatctgca

SEQ ID NO: 336 - DNA, 50 bp, synthetic construct
  source 1..50: mol_type = other DNA
tgcagatgaacgcaatctccggcttaactactgactgaagttcatctgca

SEQ ID NO: 337 - DNA, 52 bp, synthetic construct
  source 1..52: mol_type = other DNA
tgcagatgaacgcaatctcacggcttaactactgacctgaagttcatctgca

SEQ ID NO: 338 - DNA, 54 bp, synthetic construct
  source 1..54: mol_type = other DNA
tgcagatgaacgcaatgctcacggcttaactactgacactgaagttcatctgca

SEQ ID NO: 339 - DNA, 56 bp, synthetic construct
  source 1..56: mol_type = other DNA
tgcagatgaacgcaatgctgcacggcttaactactgacacctgaagttcatctgca

SEQ ID NO: 340 - DNA, 48 bp, synthetic construct
  source 1..48: mol_type = other DNA
tacgcccacgcgcaatctcggcttaactactgctgaagcgtgggcgta

SEQ ID NO: 341 - DNA, 50 bp, synthetic construct
  source 1..50: mol_type = other DNA
tacgcccacgcgcaatctccggcttaactactgactgaagcgtgggcgta

SEQ ID NO: 342 - DNA, 52 bp, synthetic construct
  source 1..52: mol_type = other DNA
tacgcccacgcgcaatctcacggcttaactactgacctgaagcgtgggcgta

SEQ ID NO: 343 - DNA, 54 bp, synthetic construct
  source 1..54: mol_type = other DNA
tacgcccacgcgcaatgctcacggcttaactactgacactgaagcgtgggcgta

SEQ ID NO: 344 - DNA, 56 bp, synthetic construct
  source 1..56: mol_type = other DNA
tacgcccacgcgcaatgctgcacggcttaactactgacacctgaagcgtgggcgta

SEQ ID NO: 345 - DNA, 48 bp, synthetic construct
  source 1..48: mol_type = other DNA
tgccacctacggcaatctcggcttaactactgctgaacgtaggtggca

SEQ ID NO: 346 - DNA, 50 bp, synthetic construct
  source 1..50: mol_type = other DNA
tgccacctacggcaatctccggcttaactactgactgaacgtaggtggca

SEQ ID NO: 347 - DNA, 52 bp, synthetic construct
  source 1..52: mol_type = other DNA
tgccacctacggcaatctcacggcttaactactgacctgaacgtaggtggca

SEQ ID NO: 348 - DNA, 54 bp, synthetic construct
  source 1..54: mol_type = other DNA
tgccacctacggcaatgctcacggcttaactactgacactgaacgtaggtggca

SEQ ID NO: 349 - DNA, 56 bp, synthetic construct
  source 1..56: mol_type = other DNA
tgccacctacggcaatgctgcacggcttaactactgacacctgaacgtaggtggca

SEQ ID NO: 350 - DNA, 48 bp, synthetic construct
  source 1..48: mol_type = other DNA
tggcccaccctgcaatctcggcttaactactgctgaaagggtgggcca

SEQ ID NO: 351 - DNA, 50 bp, synthetic construct
  source 1..50: mol_type = other DNA
tggcccaccctgcaatctccggcttaactactgactgaaagggtgggcca

SEQ ID NO: 352 - DNA, 52 bp, synthetic construct
  source 1..52: mol_type = other DNA
tggcccaccctgcaatctcacggcttaactactgacctgaaagggtgggcca

SEQ ID NO: 353 - DNA, 54 bp, synthetic construct
  source 1..54: mol_type = other DNA
tggcccaccctgcaatgctcacggcttaactactgacactgaaagggtgggcca

SEQ ID NO: 354 - DNA, 56 bp, synthetic construct
  source 1..56: mol_type = other DNA
tggcccaccctgcaatgctgcacggcttaactactgacacctgaaagggtgggcca

SEQ ID NO: 355 - DNA, 48 bp, synthetic construct
  source 1..48: mol_type = other DNA
tgcacgccgtagcaatctcggcttaactactgctgaatacggcgtgca

SEQ ID NO: 356 - DNA, 50 bp, synthetic construct
  source 1..50: mol_type = other DNA
tgcacgccgtagcaatctccggcttaactactgactgaatacggcgtgca

SEQ ID NO: 357 - DNA, 52 bp, synthetic construct
  source 1..52: mol_type = other DNA
tgcacgccgtagcaatctcacggcttaactactgacctgaatacggcgtgca

SEQ ID NO: 358 - DNA, 54 bp, synthetic construct
  source 1..54: mol_type = other DNA
tgcacgccgtagcaatgctcacggcttaactactgacactgaatacggcgtgca

SEQ ID NO: 359 - DNA, 56 bp, synthetic construct
  source 1..56: mol_type = other DNA
tgcacgccgtagcaatgctgcacggcttaactactgacacctgaatacggcgtgca

SEQ ID NO: 360 - DNA, 52 bp, synthetic construct
  source 1..52: mol_type = other DNA
tgagatgatgtaacatctattccattaatttcattaacttgcttacaagaga

SEQ ID NO: 361 - DNA, 52 bp, synthetic construct
  source 1..52: mol_type = other DNA
ttcagtttgttatatatgtgtctattaagttagattgtgagctcctaaaaga

SEQ ID NO: 362 - DNA, 52 bp, synthetic construct
  source 1..52: mol_type = other DNA
tgcttcatagggtagttctaaagattaaatcacttaatgcatgtgatctaaa

SEQ ID NO: 363 - DNA, 52 bp, synthetic construct
  source 1..52: mol_type = other DNA
tatgatcctaatcaggcttgtaaattaacttttgaggctctcttagcaatca

SEQ ID NO: 364 - DNA, 52 bp, synthetic construct
  source 1..52: mol_type = other DNA
ttctaggaaacttgaactatgattttaaaaaaagagaaagaaggaaacaaaa

SEQ ID NO: 365 - DNA, 52 bp, synthetic construct
  source 1..52: mol_type = other DNA
ttggcagccactgtcctaaatattttaaccaaatcaaaatcttatagtggca

SEQ ID NO: 366 - DNA, 50 bp, synthetic construct
  source 1..50: mol_type = other DNA
tataaatggactaaattctgcaattaaaagacacagactggcaagttgga

SEQ ID NO: 367 - DNA, 51 bp, synthetic construct
  source 1..51: mol_type = other DNA
tcctagtctctgataaaacagactttaaaccaacaaagatcaaaagagaca

SEQ ID NO: 368 - DNA, 52 bp, synthetic construct
  source 1..52: mol_type = other DNA
tcctagtctctgataaaacagactttaaaccaacaaagatcaaaagagacaa

SEQ ID NO: 369 - DNA, 50 bp, synthetic construct
  source 1..50: mol_type = other DNA
tgcaatcaaactagaactcaggattaagaatctcactcaaagccgctcaa

SEQ ID NO: 370 - DNA, 51 bp, synthetic construct
  source 1..51: mol_type = other DNA
ttcacagaattggaaaaaactactttaaagttcatatggaaccaaaaaaga

SEQ ID NO: 371 - DNA, 50 bp, synthetic construct
  source 1..50: mol_type = other DNA
tcacagaattggaaaaaactactttaaagttcatatggaaccaaaaaaga

SEQ ID NO: 372 - DNA, 50 bp, synthetic construct
  source 1..50: mol_type = other DNA
tacaaaaatcaattcaagatggattaaagatttaaacgttaaacctaaaa

SEQ ID NO: 373 - DNA, 50 bp, synthetic construct
  source 1..50: mol_type = other DNA
tcaattcaagatggattaaagatttaaacgttaaacctaaaaccataaaa

SEQ ID NO: 374 - DNA, 51 bp, synthetic construct
  source 1..51: mol_type = other DNA
tcaattcaagatggattaaagatttaaacgttaaacctaaaaccataaaaa

SEQ ID NO: 375 - DNA, 51 bp, synthetic construct
  source 1..51: mol_type = other DNA
tgatacaaagcggtttcgaataattaacttatttgttcccatcacatgtca

SEQ ID NO: 376 - DNA, 52 bp, synthetic construct
  source 1..52: mol_type = other DNA
tgaagaaactaaggcaccgagattttaagaaacttaattacacaggggataa

SEQ ID NO: 377 - DNA, 52 bp, synthetic construct
  source 1..52: mol_type = other DNA
tggctgtaattcgtgcatttttttttaagaaaaacgcctgccttctgcgtga

SEQ ID NO: 378 - DNA, 52 bp, synthetic construct
  source 1..52: mol_type = other DNA
tctgtgctctgatccctgaggcatttaatatgttcttattattagaagctca

SEQ ID NO: 379 - DNA, 51 bp, synthetic construct
  source 1..51: mol_type = other DNA
ttgatggggctagtagcctttccttaatgatagggtgtttctagagagata

SEQ ID NO: 380 - DNA, 52 bp, synthetic construct
  source 1..52: mol_type = other DNA
ttttatcgggtccaactcaaccatttaaggtatttgccagctcttgtatgca

SEQ ID NO: 381 - DNA, 51 bp, synthetic construct
  source 1..51: mol_type = other DNA
ttgagagcctccagagaaaggctcttaaaaatgcagcgcaatctccagtga

SEQ ID NO: 382 - DNA, 52 bp, synthetic construct
  source 1..52: mol_type = other DNA
tgcagatgaacgcaatctcacggtttaaatactgacctgaagttcatctgca

SEQ ID NO: 383 - DNA, 52 bp, synthetic construct
  source 1..52: mol_type = other DNA
tgccacctacggcaatctcacggtttaaatactgacctgaacgtaggtggca

SEQ ID NO: 384 - DNA, 52 bp, synthetic construct
  source 1..52: mol_type = other DNA
tgcacgccgtagcaatctcacggtttaaatactgacctgaatacggcgtgca

SEQ ID NO: 385 - DNA, 52 bp, synthetic construct
  source 1..52: mol_type = other DNA
tggcccaccctgcaatctcacggtttaaatactgacctgaaagggtgggcca

SEQ ID NO: 386 - DNA, 52 bp, synthetic construct
  source 1..52: mol_type = other DNA
tacgcccacgcgcaatctcacggtttaaatactgacctgaagcgtgggcgta

SEQ ID NO: 387 - protein, 703 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 388 - protein, 703 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 389 - protein, 703 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 390 - protein, 703 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 391 - protein, 703 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 392 - protein, 703 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 393 - protein, 703 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 394 - protein, 703 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 395 - protein, 703 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 396 - protein, 703 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 397 - protein, 703 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 398 - protein, 703 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 399 - protein, 703 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 400 - DNA, 4401 bp, synthetic construct
  source 1..4401: mol_type = other DNA
ctagcggtctctttaaccctagaaagatagtctgcgtaaaattgacgcatgcattcttgaaatattgctctctctttctaaatgacgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcggcctccaaggcctgcaggagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtttagtcctgcaggtcggccgccacgaccggtgccaccatgactgaatacaaaccaactgttcgcctggcaactcgtgatgatgttccacgtgcagttcgcaccctggctgctgcatttgctgactaccctgcaacccgtcacactgtggacccagaccgccacattgaacgtgtgactgaactgcaggagctgttcctgacccgtgtgggcctggacattggcaaagtgtgggtggcagatgatggtgctgctgtggcagtgtggaccacccctgaatctgttgaagctggtgcagtgtttgctgagattggcccacgcatggcagaactgtctggcagccgcctggcagcacaacagcagatggaaggtctgctggcaccacaccgcccaaaagaacctgcttggttcctggcaactgtgggtgtgagccctgaccaccagggtaagggcctgggctctgcagtggtgctgcctggtgtggaagcagctgaacgtgcaggtgtgcctgctttcctggagacctcagctccacgcaacctgcctttctatgaacgcctgggcttcactgtgactgctgatgtggaagtgccagaaggcccacgcacttggtgcatgactcgcaaaccaggtgctagccttggctccggcgagggcaggggaagtcttctaacatgcggggacgtggaggaaaatcccggcccactcggctacccctacgacgtgcccgactacgccagcggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttcgaactagatgctgtcgaccgatgcccttgagagccttcaacccagtcagctccttccggtgggcgcggggcaatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgactatcgtcgccgcacttattaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaagagacctgtacatacctaatgatttttatcaaaatcattaagttaaggtagatacacatcttgtcatatgatcaaatggtttcgccaaaaatcaataatcagacaacaagatgtgcgaactcgatattttacacgactctctttaccaattctaccacaacgtggatcagcagttcaacctgttgatagtacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccaaaaaaaagcccgctcattaggcgggctgcgcccttaagg

SEQ ID NO: 401 - protein, 763 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKSRSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 402 - protein, 762 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 403 - DNA, 42 bp, synthetic construct
  source 1..42: mol_type = other DNA
ggagcagatgaacgcaagctgaccctgaagttcatctgcttt

SEQ ID NO: 404 - DNA, 42 bp, synthetic construct
  source 1..42: mol_type = other DNA
ggcgcagatgaacgcaagctgaccctgaagttcatctgcgtt

SEQ ID NO: 405 - DNA, 42 bp, synthetic construct
  source 1..42: mol_type = other DNA
ggggcagatgaacgcaagctgaccctgaagttcatctgcctt

SEQ ID NO: 406 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKSRSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 407 - protein, 1070 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 408 - DNA, 52 bp, synthetic construct
  source 1..52: mol_type = other DNA
agcagatgaacgcaatctcacggcttaactactgacctgaagttcatctgct

SEQ ID NO: 409 - DNA, 52 bp, synthetic construct
  source 1..52: mol_type = other DNA
cgcagatgaacgcaatctcacggcttaactactgacctgaagttcatctgcg

SEQ ID NO: 410 - DNA, 52 bp, synthetic construct
  source 1..52: mol_type = other DNA
ggcagatgaacgcaatctcacggcttaactactgacctgaagttcatctgcc

SEQ ID NO: 411 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVCSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 412 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVDSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 413 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVESNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 414 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVFSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 415 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVGSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 416 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVHSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 417 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVISNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 418 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVKSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 419 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVLSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 420 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVMSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 421 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVNSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 422 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVPSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 423 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVQSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 424 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 425 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVSSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 426 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVTSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 427 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVVSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 428 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVWSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 429 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVYSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 430 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNCREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 431 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNDREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 432 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNEREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 433 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNFREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 434 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNGREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 435 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNHREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 436 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNIREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 437 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNKREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 438 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNLREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 439 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNMREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 440 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNNREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 441 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNPREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 442 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNQREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 443 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNRREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 444 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNSREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 445 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNTREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 446 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNVREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 447 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNWREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 448 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNYREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 449 - DNA, 6212 bp, synthetic construct
  source 1..6212: mol_type = other DNA
cgaaccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgtcatcgagctcgagatctggcgaagcgatgggggtcttgaaggcgtgctggtactccacgatgcccagctcggtgttgctgtgcagctcctccacgcggcggaaggcgaacatggggcccccgttctgcaggatgctggggtggatggcgctcttgaagtgcatgtggctgtccaccacgaagctgtagtagccgccgtcgcgcaggctgaaggtgcgggcgaagctgcccaccagcacgttatcgcccatggggtgcaggtgctccacggtggcgttgctgcggatgatcttgtcggtgaagatcacgctgtcctcggggaagccggtgcccaccaccttgaagtcgccgatcacgcggccggcctcgtagcggtagctgaagctcacgtgcagcacgccgccgtcctcgtacttctcgatgcgggtgttggtgtagccgccgttgttaaccctagaaagatagtctgcgtaaaattgacgcatgcaatctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgcagaaacatgcgtcaattttacgcatgattatctttaacgtacgtcacaatatgattatctttctagggttaatggcgtgcaggaaggggttctcgtagccgctggggtaggtgccgaagtggtagaagccgtagcccatcacgtggctcagcaggtaggggctgaaggtcagggcgcctttggtgctcttcatcttgttggtcatgcggccctgctcgggggtgccctctccgccgcccaccagctcgaactccacgccgttcagggtgccggtgatgcggcactcgatcttcatggcgggcatggtggcctgcaggactaaacctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggacagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaatttcaaatggctagagacttatcgaaagcagcgagacaggcgcgaaggtgccaccagattcgcacgcggcggccccagcgcccaggccaggcctcaactcaagcacgaggcgaaggggctcctaaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccaagcttggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtaccttcttgcagatgaacgcaatctcacggcttaaatactgacctgaagttcatctgcagactgccaccatggtttctaagggcgaagccgtgatcaaagaattcatgcggttcaaggtgcacatggaaggcagcatgaacggccacgagttcgagatcgaaggcgaaggcgagggcagaccttatgagggaacacagaccgccaagctgaaagtgaccaaaggcggccctctgcctttcagctgggacattctgagccctcagtttatgtacggcagccgggccttcacaaagcaccctgccgatattcccgactactacaagcagagcttccccgagggcttcaagtgggagagagtgatgaacttcgaggacggcggagccgtgaccgtgacacaggatacaagcctggaagatggcaccctgatctacaaagtgaagctgcggggcaccaactttccacctgatggccccgtgatgcagaaaaagaccatgggctgggaagccagcaccgagagactgtatcctgaggatggcgtgctgaagggcgacatcaagatggccctgagactgaaggatggcggcagatacctggccgacttcaagaccacctacaaggccaagaaacccgtgcagatgcctggcgcctacaacgtggacagaaagctggacatcaccagccacaacgaggactacaccgtggtggaacagtacgagcggagcgaaggcagacactctacaggcggaatggacgagctgtacaagagccatggcttcccgccggaggtggaggagcaggatgatggcacgctgcccatgtcttgtgcccaggagagcgggatggaccgtcaccctgcagcctgtgcttctgctaggatcaatgtgtgattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaagctagcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggtt

SEQ ID NO: 450 - protein, 1081 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 451 - protein, 1080 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 452 - protein, 1079 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 453 - protein, 1078 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 454 - protein, 1077 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 455 - protein, 1076 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 456 - protein, 1075 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 457 - protein, 1074 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 458 - protein, 1073 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 459 - protein, 1072 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 460 - protein, 1070 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 461 - protein, 1069 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 462 - protein, 1068 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 463 - protein, 1067 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 464 - protein, 1066 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 465 - protein, 1065 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 466 - protein, 1064 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 467 - protein, 1063 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 468 - protein, 1062 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 469 - protein, 1061 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 470 - DNA, 52 bp, synthetic construct
  source 1..52: mol_type = other DNA
tgcagatgaacgcaatctcacggcttaaatactgacctgaagttcatctgca

SEQ ID NO: 471 - protein, 13 aa, synthetic construct
SIVAQLSRPDPAL

SEQ ID NO: 472 - protein, 23 aa, synthetic construct
SIVAQLSRPDPALAALTNDHLVA

SEQ ID NO: 473 - protein, 33 aa, synthetic construct
SIVAQLSRPDPALAALTNDHLVALACLGGRPAL

SEQ ID NO: 474 - protein, 43 aa, synthetic construct
SIVAQLSRPDPALAALTNDHLVALACLGGRPALDAVKKGLPHA

SEQ ID NO: 475 - protein, 53 aa, synthetic construct
SIVAQLSRPDPALAALTNDHLVALACLGGRPALDAVKKGLPHAPALIKRTNRR

SEQ ID NO: 476 - protein, 73 aa, synthetic construct
SIVAQLSRPDPALAALTNDHLVALACLGGRPALDAVKKGLPHAPALIKRTNRRIPERTSHRVADHAQVVRVLG

SEQ ID NO: 477 - protein, 1021 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 478 - protein, 1031 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 479 - protein, 1041 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 480 - protein, 1051 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 481 - protein, 1061 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 482 - protein, 1081 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVADHAQVVRVLGGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 483 - protein, 1016 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 484 - protein, 1026 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVANKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 485 - protein, 1036 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 486 - protein, 1046 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHANKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 487 - protein, 1056 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 488 - protein, 1066 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVANKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 489 - protein, 1076 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVADHAQVVRVLGNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 490 - protein, 1029 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 491 - protein, 1039 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVAGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 492 - protein, 1049 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 493 - protein, 1059 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 494 - protein, 1069 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 495 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 496 - protein, 1026 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALGGGGSTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 497 - protein, 1036 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVAGGGGSTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 498 - protein, 1046 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALGGGGSTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 499 - protein, 1056 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAGGGGSTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 500 - protein, 1066 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRGGGGSTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 501 - protein, 1086 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVADHAQVVRVLGGGGGSTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 502 - protein, 1015 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALGGGGSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 503 - protein, 1025 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVAGGGGSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 504 - protein, 1035 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALGGGGSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 505 - protein, 1045 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAGGGGSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 506 - protein, 1055 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRGGGGSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 507 - protein, 1075 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVADHAQVVRVLGGGGGSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 508 - protein, 1011 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALGGGGSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 509 - protein, 1021 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVAGGGGSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 510 - protein, 1031 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALGGGGSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 511 - protein, 1041 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAGGGGSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 512 - protein, 1051 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRGGGGSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 513 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVADHAQVVRVLGGGGGSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 514 - DNA, 11 bp, synthetic construct
  source 1..11: mol_type = other DNA
tttgttcccat

SEQ ID NO: 515 - DNA, 11 bp, synthetic construct
  source 1..11: mol_type = other DNA
taacgggtagt

SEQ ID NO: 516 - DNA, 11 bp, synthetic construct
  source 1..11: mol_type = other DNA
ttgctggttat

SEQ ID NO: 517 - DNA, 11 bp, synthetic construct
  source 1..11: mol_type = other DNA
tttaaatatca

SEQ ID NO: 518 - protein, 326 aa, synthetic construct
LTPDQVVAIASNGGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIASNGGGRPALE

SEQ ID NO: 519 - protein, 326 aa, synthetic construct
LTPDQVVAIASNIGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGRPALE

SEQ ID NO: 520 - protein, 326 aa, synthetic construct
LTPDQVVAIASNGGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASNGGGRPALE

SEQ ID NO: 521 - protein, 326 aa, synthetic construct
LTPDQVVAIASNGGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNIGGRPALE

SEQ ID NO: 522 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 523 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 524 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 525 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 526 - DNA, 36 bp, synthetic construct
  source 1..36: mol_type = other DNA
ctattcacattaaaatttaattagagagggggtgga

SEQ ID NO: 527 - DNA, 39 bp, synthetic construct
  source 1..39: mol_type = other DNA
cggccagcctcggctcatttaaaggcaccagccgccgtt

SEQ ID NO: 528 - DNA, 38 bp, synthetic construct
  source 1..38: mol_type = other DNA
ggctacccagctagtttttaaaattttttgttgttgtt

SEQ ID NO: 529 - protein, 701 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPGGGGSERPFQCRICMRNFSTKPVLKIHTRTHTGEKPFQCRICMRNFSHKSSLTRHLRTHTGEKPFQCRICMRNFSIRTSLKRHLKTHLRGSQGGGGSRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 530 - protein, 701 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPGGGGSERPFQCRICMRNFSDGSTLRRHTRTHTGEKPFQCRICMRNFSQRSSLVRHLRTHTGEKPFQCRICMRNFSRSDHLSLHLKTHLRGSQGGGGSRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 531 - protein, 701 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGGGGGSERPFQCRICMRNFSTHAHLTRHTRTHTGEKPFQCRICMRNFSEAHHLSRHLRTHTGEKPFQCRICMRNFSRTEHLARHLKTHLRGSQGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 532 - protein, 701 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGGGGGSERPFQCRICMRNFSRRRNLQIHTRTHTGEKPFQCRICMRNFSQQTNLTRHLRTHTGEKPFQCRICMRNFSQPHGLTAHLKTHLRGSQGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 533 - protein, 701 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPGGGGSERPFQCRICMRNFSTTTVLARHTRTHTGEKPFQCRICMRNFSDSSVLRRHLRTHTGEKPFQCRICMRNFSERRGLARHLKTHLRGSQGGGGSRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 534 - protein, 701 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGGGGGSERPFQCRICMRNFSAPSKLARHTRTHTGEKPFQCRICMRNFSQRSDLTRHLRTHTGEKPFQCRICMRNFSRQDNLQRHLKTHLRGSQGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 535 - protein, 1311 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFARLAKLGGGAPAVGGGPKAADKGLPSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 536 - protein, 1679 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFARLAKLGGGAPAVGGGPKAADKGLPSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 537 - protein, 1679 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFARLAKLGGGAPAVGGGPKAADKGLPSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 538 - protein, 1679 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFARLAKLGGGAPAVGGGPKAADKGLPSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 539 - protein, 1679 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFARLAKLGGGAPAVGGGPKAADKGLPSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 540 - DNA, 11 bp, synthetic construct
  source 1..11: mol_type = other DNA
gaactccgggt

SEQ ID NO: 541 - DNA, 12 bp, synthetic construct
  source 1..12: mol_type = other DNA
tggcagtcgagt

SEQ ID NO: 542 - protein, 1070 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 543 - protein, 1120 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPDQVVAIANNNGGKQALETVQRLLPVLCQAHGLTPAQVVAIASHDGGKQALETVQRLLPVLCQDHGLTPDQVVAIASNIGGKQALETVQRLLPVLCQDHGLTPDQVVAIANNNGGKQALETVQRLLPVLCQDHGLTPDQVVAIASNGGGKQALETVQRLLPVLCQAHGLTPAQVVAIASHDGGKQALETVQRLLPVLCQDHGLTPDQVVAIANNNGGKQALETVQRLLPVLCQDHGLTPDQVVAIASNIGGKQALETVQRLLPVLCQDHGLTPDQVVAIANNNGGKQALETVQRLLPVLCQAHGLTPDQVVAIASNGGGKQALETVQRLLPVLCQDHGTSIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 544 - protein, 594 aa, synthetic construct
GGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 545 - DNA, 41 bp, synthetic construct
  source 1..41: mol_type = other DNA
acgcccacgctacatctttaaagatgtagatgaggatgacc

(19) United States
(12) Patent Application Publication
(10) Pub. No.: US 2024/0392262 A1
(43) Pub. Date: Nov. 28, 2024

(54) TRANSPOSASE AND USES THEREOF

(71) Applicant: Poseida Therapeutics, Inc., San Diego, CA (US)

(72) Inventors: Dongyang ZHANG, San Diego, CA (US); Blair B. MADISON, San Diego, CA (US); Joseph S. LUCAS, San Diego, CA (US); Olga BATALOV, San Diego, CA (US); Andres J. VALDERRAMA, San Diego, CA (US)

(21) Appl. No.: 18/696,129

(22) PCT Filed: Oct. 4, 2022
(86) PCT No.: PCT/US2022/077549

§ 371 Date: Mar. 27, 2024

Related U.S. Application Data
(60) Provisional application No. 63/369,863, filed on Jul. 29, 2022.
(60) Provisional application No. 63/312,928, filed on Feb. 23, 2022.
(60) Provisional application No. 63/252,028, filed on Oct. 4, 2021.

(51) Int. Cl. C12N 9/12; C12N 15/90

(52) U.S. Cl. CPC C12N 9/1241; C12N 15/907; C07K 2319/81; C12N 2800/90

(57) ABSTRACT

This disclosure generally relates to transposase domains, in particular, transposase domains comprising amino terminal deletions, as well as transposase domains forming obligate heterodimers and transposase domains comprising DNA targeting domains.

### CROSS-REFERENCE TO RELATED APPLICATIONS

The present application claims the benefit of U.S. Provisional Patent Applications No. 63/252,028 filed Oct. 4, 2021, U.S. Pat. No. 63,312,928 filed Feb. 23, 2022, and No. 63/369,863 filed Jul. 29, 2022, each of which is incorporated herein by reference in its entirety.

### REFERENCE TO SEQUENCE LISTING SUBMITTED ELECTRONICALLY

The instant application contains a Sequence Listing which has been submitted in XML format via Patent Center and is hereby incorporated by reference in its entirety. Said XML copy, created on Oct. 3, 2022 is named “POTH-069-001WO-SeqList_ST26” and is 787,153 bytes in size.

### FIELD

This disclosure generally relates to transposase domains, in particular, transposase domains comprising N-terminal deletions, as well as transposase domains forming obligate heterodimers and fusion proteins comprising the transposes domains and DNA targeting domains. Also provided are methods of use of the fusion proteins for site-specific transposition.

### BACKGROUND

Transposases may be used to introduce non-endogenous DNA sequences into genomic DNA, and are in many ways advantageous to other methods gene editing. However, there remains an unmet need for site-specific transposases for use in e.g., gene editing.

### SUMMARY

In one aspect, provided herein is a fusion protein comprising a first transposase domain; a linker; and a second transposase domain; wherein (a) the first and second transposase domain are the same; or (b) the first and second transposase domain are the same, except that the second transposase domain comprises an N-terminal deletion. In some embodiments, the first transposase domain is a piggyBac transposase domain. In some embodiments, the piggyBac transposase domain is a hyperactive piggyBac transposase domain. In some embodiments, the first transposase domain is a Super PiggyBac (SPB) transposase domain. In some embodiments, the second transposase domain is a piggyBac transposase domain. In some embodiments, the piggyBac transposase domain is a hyperactive piggyBac transposase domain. In some embodiments, the second transposase domain is a Super PiggyBac transposase domain. In some embodiments, the first transposase domain and the second transposase domain are piggyBac transposase domains. In some embodiments, the first piggyBac transposase domain and the second piggyBac transposase domains are hyperactive piggyBac transposase domains. In some embodiments, the first transposase domain is a SPB transposase domain. In some embodiments, the first transposase domain and the second transposase domain are SPB transposase domains.

In some embodiments, the N-terminal deletion of the second transposase domain comprises amino acids 1-20. In some embodiments, the amino terminal deletion of the second transposase domain comprises amino acids 1-40. In some embodiments, the amino terminal deletion of the second transposase domain comprises amino acids 1-60. In some embodiments, the amino terminal deletion of the second transposase domain comprises amino acids 1-80. In some embodiments, the amino terminal deletion of the second transposase domain comprises amino acids 1-100. In some embodiments, the amino terminal of the second transposase domain comprises amino acids 1-115. In some embodiments, the first transposase domain further comprises an in-frame nuclear localization signal (NLS).

In some embodiments, the linker is juxtaposed between the C-terminus of the first transposase domain and the N-terminus of the second transposase domain. In some embodiments, the linker comprises the sequence set forth in SEQ ID NO: 16.

In some embodiments, the fusion protein comprises the amino acid sequence of any one of SEQ ID NOs: 8-14. In some embodiments, the fusion protein further comprises a mutation in one or both transposase domains. In some embodiments, the mutation is (a) selected from the group consisting of M185R, M185K, D197K, D197R, D198K, D198R, D201K, and D201R or (b) selected from the group consisting of L204D, L204E, K500D, K500E, R504E, and R504D. In some embodiments, the fusion protein comprises two or three of the mutations selected from the group consisting of M185R, D198K and D201R in one or both transposase domains. In some embodiments, the fusion protein comprises two or three of the mutations selected from the group consisting of: L204E, K500D, and R504D in one or both transposase domains.

In another aspect, provided herein is a transposase domain comprising the sequence selected from any one of SEQ ID NOs: 31-53. In some embodiments, the transposase domain comprising the sequence selected from any one of SEQ ID NOs: 31-53 and further comprises one or more conservative amino acid sequences.

In another aspect, provided herein is a fusion protein comprising a first transposase domain, a linker; and a second transposase domain; wherein the first transposase domain and/or the second transposase domain comprise the same sequence selected from any one of SEQ ID NOs: 31-43. In another aspect, provided herein is a fusion protein comprising a first transposase domain, a linker; and a second transposase domain; wherein the first transposase domain and/or the second transposase domain comprise the same sequence selected from any one of SEQ ID NOs: 44-53.

In some embodiments, a fusion protein provided herein further comprises a DNA targeting domain. In some embodiments, the DNA targeting domain is attached to the N-terminus of the fusion protein. In some embodiments, the DNA targeting domain is attached to the C-terminus of the fusion protein. In some embodiments, the DNA targeting domain is selected from the group consisting of CRISPR, Zinc Finger, TALE, and transcription factors.

In another aspect, provided herein is a transposase domain comprising an N-terminal deletion as compared to the sequence set forth in SEQ ID NO: 1 or SEQ ID NO: 55 (with numbering beginning at residue 12 of SEQ ID NO: 1 and residue 5 of SEQ ID NO: 55). In some embodiments, the transposase domain is a piggyBac transposase domain. In some embodiments, the piggyBac transposase domain is a hyperactive piggyBac transposase domain. In some embodiments, the transposase domain is a SPB transposase domain. In some embodiments, the N-terminal deletion comprises amino acids 1-20. In some embodiments, the N-terminal deletion comprises amino acids 1-40. In some embodiments, the N-terminal deletion comprises amino acids 1-60. In some embodiments, the N-terminal deletion comprises amino acids 1-80. In some embodiments, the N-terminal deletion comprises amino acids 1-100. In some embodiments, N-terminal deletion comprises amino acids 1-115.

In some embodiments, the transposase domain further comprises an in-frame nuclear localization signal (NLS). In some embodiments, the in-frame NLS is fused to the amino terminus of the transposase domain. In some embodiments, the transposase domain comprises the amino acid sequence of any one of SEQ ID NOs: 2-7.

In another aspect, provided herein is a nucleic acid molecule, comprising a nucleotide sequence encoding a fusion protein described herein. In some embodiments, the nucleic acid molecule further comprises a promoter operably linked to the nucleotide sequence encoding the fusion protein. In some embodiments, the nucleic acid molecule further comprises a polyA sequence located downstream of the nucleotide sequence encoding the second transposase domain.

In another aspect, provided herein is a nucleic acid molecule, comprising a nucleotide sequence encoding a transposase domain described herein. In some embodiments, the nucleic acid molecule further comprises a promoter operably linked to the nucleotide sequence encoding the transposase domain. In some embodiments, the nucleic acid molecule further comprises a polyA sequence located downstream of the nucleotide sequence encoding the transposase domain.

In another aspect, provided herein is a cell comprising a nucleic acid molecule described herein. In some embodiments, the cell is derived from a patient. In some embodiments, the cell further comprises a chimeric antigen receptor (CAR). In some embodiments, the cell is an immune cell. In some embodiments, the cell is a T cell.

In another aspect, provided herein is a method of treating a disease or disorder in a patient, the method comprising administering a cell described herein to the patient. In some embodiments, the cell is autologous. In some embodiments, the cell is allogeneic. In some embodiments, the disease or disorder is cancer.

In another aspect, provided herein is a complex comprising (a) a first fusion protein comprising a first transposase domain, a linker, a second transposase domain, and a first DNA targeting domain, wherein (i) the first and second transposase domain are the same; or (ii) the first and second transposase domain are the same, except that the second transposase domain comprises an N-terminal deletion; and (b) a second fusion protein comprising a first transposase domain, a linker, a second transposase domain, and a second DNA targeting domain, wherein (i) the first and second transposase domain are the same; or (ii) the first and second transposase domain are the same, except that the second transposase domain comprises an N-terminal deletion; wherein the first DNA targeting domain and the second DNA targeting domain are different; wherein the transposase domains of the first fusion protein and the transpose domains of the second fusion protein have opposing charge that permits the two fusion proteins to form a complex.

In some embodiments, the transposase domains of the first fusion protein comprises at least one mutation and the transpose domains of the second fusion protein comprise at least one mutation that provides the opposing charge. In some embodiments, the first and second transposase domain of the first fusion protein and the first and second transposase domain of the second fusion protein are SPB transposase domains. In some embodiments, at least one mutation is selected from the group consisting of M185R, M185K, D197K, D197R, D198K, D198R, D201K, and D201R. In some embodiments, the at least one mutation is selected from the group consisting of L204D, L204E, K500D, K500E, R504E, and R504D.

In some embodiments, the N-terminal deletion comprises amino acids 1-20. In some embodiments, the N-terminal deletion comprises amino acids 1-40. In some embodiments, the N-terminal deletion comprises amino acids 1-60. In some embodiments, the N-terminal deletion comprises amino acids 1-80. In some embodiments, the N-terminal deletion comprises amino acids 1-100. In some embodiments, the N-terminal deletion comprises amino acids 1-115.

In some embodiments, the first DNA targeting domain is attached to the C-terminus of the first fusion protein and the second DNA targeting domain is attached to the C-terminus of the second fusion protein. In some embodiments, the first DNA targeting domain is attached to the N-terminus of the first fusion protein and the second DNA targeting domain is attached to the N-terminus of the second fusion protein. In some embodiments, the DNA targeting domains are selected from the group consisting of CRISPR, Zinc Finger, TALE, and transcription factors.

In another aspect, provided herein is a complex comprising (a) a first fusion protein comprising a first transposase domain, a linker, a second transposase domain, and a first DNA targeting domain, wherein the first and/or the second transposase domain of the first fusion protein comprise the same amino acid sequence set forth in any one of SEQ ID NOs: 31-43.; and (b) a second fusion protein comprising a first transposase domain, a linker, a second transposase domain, and a second DNA targeting domain, wherein the first and/or the second transposase domain of the second fusion protein comprise the same amino acid sequence set forth in any one of SEQ ID NOs: 44-53. In some embodiments, the DNA targeting domains are selected from the group consisting of CRISPR, Zinc Finger, TALE, and transcription factors.

In another aspect, provided herein is a fusion protein comprising, in N-terminal to C-terminal order: a nuclear localization signal (NLS), a DNA targeting domain, and a first transposase domain comprising the sequence of SEQ ID NO: 65 or 55. In some embodiments, the fusion protein further comprises a protein stabilization domain (PSD). In some embodiments, the PSD comprises SEQ ID NO: 68. In some embodiments, the DNA targeting domain comprises three Zinc Finger Motifs. In some embodiments, the DNA targeting domain comprises the sequence of SEQ ID NO: 57. In some embodiments, the DNA targeting domain comprises one or more TAL domains. In some embodiments, the DNA targeting domain binds to a nucleic acid sequence encoding GFP, ZFM268, phenylalanine hydroxylase (PAH), beta-2-microglobulin (B2M) or a LINE1 repeat element.

In some embodiments, the transposase domain comprises (a) at least one mutation selected from the group consisting of M92R, M92K, D104K, D104R, D105K, D105R, D108K, and D108R; or (b) at least one mutation selected from the group consisting of L111D, L111E, K407D, K407E, R411E, and R411D. In some embodiments, the fusion protein comprises the sequence of SEQ ID NO: 67 or 69.

In some embodiments, the fusion protein further comprises a second transposase domain. In some embodiments, the second transposase domain comprises the sequence of SEQ ID NO: 55 or 56. In some embodiments, the second transposase domain is connected to the C-terminus of the first transposase domain via a linker.

In another aspect, provided herein is a fusion protein, comprising: (a) a TAL Array; and (b) a Super piggyBac transposase (“SPB”) comprising a N-terminal deletion; wherein the TAL Array and the polynucleotide encoding the N-terminal deleted SPB are fused in-frame to encode a TAL Array—N-terminal deleted SPB fusion protein. In some embodiments, the fusion protein further comprises an in-frame GS or GGGGS linker positioned between the TAL Array and the N-terminal deleted SPB. In some embodiments, the SPB comprises a N-terminal deletion comprising a deletion of amino acids 1-83, 1-84, 1-85, 186, 1-87, 1-88, 1-89, 1-90, 1-91, 1-92, 1-93, 1-94, 1-95, 1-96, 1-97, 1-98, 1-99, 1-100, 1-101, 1-102 or 1-103. In some embodiments, the fusion protein further comprising one or more mutations in the SPB at amino acids R372A, K375A, or D450N. In some embodiments, the SPB comprises the sequence set forth in SEQ ID Nos. 81-106. In some embodiments, the SPB is an integration deficient SPB (PBx).

In another aspect, provided herein is a complex comprising: (a) a first fusion protein comprising, in N-terminal to C-terminal order: a first NLS, a first DNA targeting domain, a first transposase domain comprising the sequence of SEQ ID NO: 65 or 66, a linker, and a second transposase domain; and (b) a second fusion protein comprising in N-terminal to C-terminal order: a second NLS, a second DNA targeting domain, a third transposase domain comprising the sequence of SEQ ID NO: 65 or 66, a linker, and a fourth transposase domain; wherein the transposase domains of the first fusion protein and the transpose domains of the second fusion protein have opposing charge that permits the two fusion proteins to form a complex.

In some embodiments, the second and/or fourth transposase domains are SPB domains. In some embodiments, the second and/or fourth transposase domains are PBx transposase domains. In some embodiments, the second and/or fourth transposase domain comprises the sequence of SEQ ID NO: 55. In some embodiments, the second and/or fourth transposase domain comprises the sequence of SEQ ID NO: 56.

In some embodiments, the first transposase domain comprises at least one mutation selected from the group consisting of M92R, M92K, D104K, D104R, D105K, D105R, D108K, and D108R. In some embodiments, the second transposase domain comprises at least one mutation selected from the group consisting of M185R, M185K, D197K, D197R, D198K, D198R, D201K, and D201R. In some embodiments, the third transposase domain comprises at least one mutation selected from the group consisting of L111D, L111E, K407D, K407E, R411E, and R411D. In some embodiments, the fourth transposase domain comprises at least one mutation selected from the group consisting of L204D, L204E, K500D, K500E, and R504E, R504D.

In some embodiments, the first fusion protein further comprises a first PSD between the first NLS and the first DNA targeting domain and/or the second fusion protein further comprises a second PSD between the second NLS and the second DNA targeting domain. In some embodiments, the first and/or second PSD comprises the sequence of SEQ ID NO: 68.

In some embodiments, the first and/or second DNA targeting domain comprises three Zinc Fingers Motifs. In some embodiments, the first and/or second DNA targeting domain comprises the sequence of SEQ ID NO: 57.

In another aspect, provided herein is a polynucleotide comprising a nucleic acid sequence encoding a fusion protein provided herein. In another aspect, provided herein is a vector comprising a polynucleotide provided herein.

In another aspect, provided herein is a cell comprising a polynucleotide or a vector provided herein. In some embodiments, the cell further comprises a chimeric antigen receptor (CAR). In some embodiments, the cell is an immune cell.

In another aspect, provided herein is a pharmaceutical composition comprising a cell provided herein and a pharmaceutically acceptable carrier.

In another aspect, provided herein is a method of treating a disease or disorder in a patient, the method comprising administering to the patient a cell or a pharmaceutical composition provided herein. In some embodiments, the cell is allogeneic. In some embodiments, the disease or disorder is cancer.

In another aspect, provided herein is a method of modifying the genome of a cell, the method comprising: providing the cell with a fusion protein comprising in N-terminal to C-terminal order: an NLS, a PSD, a DNA targeting domain, and a transposase domain comprising the sequence of SEQ ID NO: 65 or 66; wherein the cell comprises a modified binding site comprising, in 5′ to 3′ order, the reverse of the sequence of a target site for the DNA targeting domain, a first spacer, a TTAA target integration site for SPB, a second spacer, and the complement of the sequence of the target site for the DNA targeting domain. In some embodiments, the DNA targeting domain comprises the sequence of SEQ ID NO: 57. In some embodiments, the fusion protein comprises the sequence of SEQ ID NO: 67. In some embodiments, the first spacer and the second spacer are each 7 bp in length. In some embodiments, the modified binding site comprises the sequence of any one of SEQ ID NOs: 61-64.

In another aspect, provided herein is an integration cassette for site-specific transposition of a DNA molecule into the genome of a cell. In one embodiment, the integration cassette for site-specific transposition of a nucleic acid into the genome of a cell comprises a nucleic acid comprising or consisting of a central transposon ITR integration site TTAA sequence flanked by at least one upstream Zinc Finger Motif DNA-binding domain binding site (“ZFM-DBD”) and at least one downstream ZFM-DBD, wherein each of the upstream and the downstream ZFM-DBD is separated from the TTAA sequence by 7 base pairs. In one embodiment, each of the at least one upstream and downstream ZFM-DBD sites is a ZFM268 binding site. In one embodiment, each of the ZFM268 binding sites comprises SEQ ID NO: 60. In one embodiment, the integration cassette comprises or consists of SEQ ID NO: 62.

In another aspect, provided herein is an integration cassette for site-specific transposition of a nucleic acid into the genome of a cell comprising or consisting of a nucleic acid comprising or consisting of a central transposon ITR integration site TTAA sequence flanked by an upstream TAL array target sequence and a downstream TAL array target sequence, wherein each of the upstream and the downstream TAL array target sequences is separated from the TTAA sequence by 12-14 base pairs.

In another aspect, provided herein is an integration cassette for site-specific transposition of a nucleic acid into the genome of a cell comprising a nucleic acid comprising a central transposon ITR integration site TTTAAA sequence flanked by an upstream TAL array target sequence and a downstream TAL array target sequence, wherein each of the upstream and the downstream TAL array target sequences is separated from the TTTAAA sequence by 12 base pairs.

In one embodiment, each of the at least one upstream and downstream TAL array target site sequences are the same. In one embodiment, each of the at least one upstream and downstream TAL array target site sequences are different. In one embodiment, each of the at least one upstream and downstream TAL Array target sites target a 7-30 bp (e.g., 10 bp) sequence of beta-2-microglobulin gene (“B2M”), phenylalanine hydroxylase gene (“PAH”) or a LINE1 repeat element. In one embodiment, the at least one upstream TAL array target sequence and the at least one downstream TAL array target sequence bind to a nucleic acid comprising the sequence GCGTGGGCG. In one embodiment, the integration cassette comprises SEQ ID NO: 62.

In certain aspects, provided is a cell comprising an integration cassette for site-specific transposition of a DNA molecule provided herein stably integrated into the genome of the cell.

In certain aspects, provided is a method for site-specific transposition of a DNA molecule into the genome of a cell comprising a stably integrated integration cassette, comprising introducing into the cell: a) a nucleic acid encoding a fusion protein comprising a DNA binding domain and a transposase; wherein the fusion protein is expressed in the cell, and b) a DNA molecule comprising a transposon; wherein the expressed fusion protein integrates the transposon by site-specific transposition into the TTAA sequence of the stably integrated integration cassette.

In certain aspects, provided is a method for generating an engineered cell by site-specific transposition comprising: introducing into a cell comprising a stably integrated integration cassette: a) a nucleic acid encoding a fusion protein comprising a DNA binding domain and a transposase; wherein the fusion protein is expressed in the cell, and b) a DNA molecule comprising a transposon; wherein the expressed fusion protein integrates the transposon by site-specific transposition into the TTAA sequence of the stably integrated integration cassette thereby generating the engineered cell.

In another aspect, provided herein is a fusion protein comprising, in N-terminal to C-terminal order: a DNA targeting domain and a first transposase domain comprising the sequence set forth in SEQ ID NO: 544, wherein the first transposase domain comprises a deletion of the 83-103 most N-terminal amino acids of SEQ ID NO: 544.

In some embodiments, the DNA targeting domain comprises three Zinc Finger Motifs. In some embodiments, the DNA targeting domain comprises one or more TAL domains. In some embodiments, the TAL domain comprises the sequence set forth in any one of SEQ ID NOs: 107-110. In some embodiments, the DNA targeting domain binds to a nucleic acid sequence encoding GFP, zinc finger 268 (ZFM268), phenylalanine hydroxylase (PAH), beta-2-microglobulin (B2M) or a LINE1 repeat element.

In some embodiments, the first transposase domain and the DNA targeting domain are connected by a linker. In some embodiments, the linker comprises the sequence GGGGS.

In some embodiments, the first transposase domain comprises an N-terminal deletion of amino acids 1-83, 1-84, 1-85, 186, 1-87, 1-88, 1-89, 1-90, 1-91, 1-92, 1-93, 1-94, 1-95, 1-96, 1-97, 1-98, 1-99, 1-100, 1-101, 1-102 or 1-103. In some embodiments, the transposase domain comprises the sequence set forth in any one of SEQ ID NOs: 86-106.

In some embodiments, the first transposase domain comprises (a) at least one mutation selected from the group consisting of M185R, M185K, D197K, D197R, D198K, D198R, D201K, and D201R or (b) at least one mutation selected from the group consisting of L204D, L204E, K500D, K500E, R504E, and R504D

In some embodiments, the fusion protein further comprises a second transposase domain C-terminal to the first transposase domain, wherein the second transposase domain comprises the sequence set forth in SEQ ID NO: 544. In some embodiments, the second transposase domain comprises a deletion of N-terminal amino acids 1-83, 1-84, 1-85, 186, 1-87, 1-88, 1-89, 1-90, 1-91, 1-92, 1-93, 1-94, 1-95, 1-96, 1-97, 1-98, 1-99, 1-100, 1-101, 1-102 or 1-103 of SEQ ID NO: 544. In some embodiments, the second transposase domain comprises (a) at least one mutation selected from the group consisting of M185R, M185K, D197K, D197R, D198K, D198R, D201K, and D201R; or (b) at least one mutation selected from the group consisting of L204D, L204E, K500D, K500E, R504E, and R504D.

In another aspect, provided herein is a polynucleotide comprising a nucleic acid sequence encoding a fusion protein provided herein. Also provided herein is a vector comprising a polynucleotide provided herein.

In another aspect, provided herein is a method of integrating a transgene into a genomic target site of a cell, the method comprising introducing into the cell a fusion protein provided herein and a transposon, wherein the transposon comprises, in 5′ to 3′ order: a 5′ITR, the transgene, and a 3′ ITR. In some embodiments, the transposon further comprises an exogenous promoter between the 5′ ITR and the transgene. In some embodiments, the transgene encodes a detectable marker. In some embodiments, the detectable marker is GFP. In some embodiments, the transgene is a gene that is not expressed by the cell prior to the introduction of the fusion protein and the transposon.

In some embodiments, the genomic target site is located on chromosome 17 or 21. In some embodiments, the genomic target site is located in the B2M gene. In some embodiments, the genomic target site is located in a repetitive element. In some embodiments, the repetitive element is a LINE element. In some embodiments, the genomic target site is located in an intron of a gene. In some embodiments, the genomic target site is located in the intron of the PAH gene. In some embodiments, the cell is in vivo.

In another aspect, provided herein is a method of modifying the genome of a cell, the method comprising: providing the cell with a fusion protein provided herein, wherein the cell comprises a modified binding site comprising, in 5′ to 3′ order, the reverse of the sequence of a target site for the DNA targeting domain, a first spacer, a TTAA target integration site for SPB, a second spacer, and the complement of the sequence of the target site for the DNA targeting domain.

In another aspect, provided herein is an integration cassette for site-specific transposition of a nucleic acid into the genome of a cell comprising a nucleic acid comprising or consisting of a central transposon ITR integration site TTAA sequence flanked by at least one upstream Zinc Finger Motif DNA-binding domain binding site (“ZFM-DBD”) and at least one downstream ZFM-DBD, wherein each of the upstream and the downstream ZFM-DBD is separated from the TTAA sequence by 7 base pairs.

In another aspect, provided herein is an integration cassette for site-specific transposition of a nucleic acid into the genome of a cell comprising or consisting of a nucleic acid comprising or consisting of a central transposon ITR integration site TTAA sequence flanked by an upstream TAL array target sequence and a downstream TAL array target sequence, wherein each of the upstream and the downstream TAL array target sequences is separated from the TTAA sequence by 12-14 base pairs.

In another aspect, provided herein is an integration cassette for site-specific transposition of a nucleic acid into the genome of a cell comprising a nucleic acid comprising a central transposon ITR integration site TTTAAA sequence flanked by an upstream TAL array target sequence and a downstream TAL array target sequence, wherein each of the upstream and the downstream TAL array target sequences is separated from the TTTAAA sequence by 12 base pairs. In some embodiments, the at least one upstream and downstream TAL array target site sequences are the same. In some embodiments, each of the at least one upstream and downstream TAL array target site sequences are different. In some embodiments, each of the at least one upstream and downstream TAL Array target sites target a 10 bp sequence of beta-2-microglobulin gene (“B2M”), phenylalanine hydroxylase gene (“PAH”) or a LINE1 repeat element. In some embodiments, the at least one upstream TAL array target sequence and the at least one downstream TAL array target sequence bind to a nucleic acid comprising the sequence GCGTGGGCG.

In another aspect, provided herein is a cell, comprising an integration cassette provided herein stably integrated into the genome of the cell. In another aspect, provided herein is a method for site-specific transposition of a DNA molecule into the genome of a cell, comprising introducing into a cell provided herein: a nucleic acid encoding a fusion protein comprising a DNA binding domain and a transposase; wherein the fusion protein is expressed in the cell; and a DNA molecule comprising a transposon; wherein the expressed fusion protein integrates the transposon by site-specific transposition into the TTAA sequence of the stably integrated integration cassette.

In another aspect, provided herein is a method for generating an engineered cell by site-specific transposition, comprising introducing into a cell provided herein a nucleic acid encoding a fusion protein comprising a DNA binding domain and a transposase; wherein the fusion protein is expressed in the cell; and a DNA molecule comprising a transposon; wherein the expressed fusion protein integrates the transposon by site-specific transposition into the TTAA sequence of the stably integrated integration cassette thereby generating the engineered cell.

### BRIEF DESCRIPTION OF DRAWINGS

FIG. 1A shows a schematic illustrating SPB constructs with N-terminal deletions described herein. FIG. 1B shows a schematic illustrating an SPB construct with an inserted DNA binding domain.

FIGS. 2A-2D illustrate the introduction of DNA binding domains into a transposase using obligate heterodimers.

FIG. 3 shows results of an excision reporter assay showing activity of wildtype transposase domains and transposase domains comprising N-terminal deletions. “−20aa” etc. indicate N-terminal deletions of 20, 40, 60, 80, or 115 amino acids.

FIGS. 4A and 4B shows results of an excision reporter assays and an integration reporter assays, respectively, showing excision or integration activity of a wildtype SPB domain and fusion proteins (“tdSPB”) comprising either two wildtype SPB transposase domains or one wildtype SPB transposase domain and one transposase domain comprising an N-terminal deletion. “−20aa” etc. indicate N-terminal deletions of 20, 40, 60, 80, or 115 amino acids in the second transposase domain.

FIGS. 5A-5H are a series of graphs showing results of excision activity and integration activity for various SPB transposase homodimers and heterodimers. K562 cells were nucleofected with dual luciferase reporter and a SPB-expressing plasmid. One day post transfection, luciferase signal was measured as a proxy for excision activity or integration activity.

FIG. 6A shows is a schematic depiction of the dual reporter plasmid design used to confirm the rates of excision and integration using each mutant transposon. Using an H-2kk GFP transposon reporter (Reporter 1), an increase in H2kk expression is observed if there is an increase in excision of the transposon. Using Reporter 2, an increase in GFP expression is observed if there is an increase in the integration of the transposon. In an alternative design of Reporter 2, an increase in Firefly luciferase expression is observed if there is an increase in excision of the transposon and an increase in NanoLuc is observed if there is an increase in the integration of the transposon. FIG. 6B is a schematic depiction of an H-2kk GFP transposon reporter (Reporter 1). Structural features of the transposon are shown both in a circular map and a linear map. An increase in H2kk expression is observed if there is an increase in excision of the transposon and an increase in GFP is observed if there is an increase in integration of the transposon. FIG. 6C is a schematic depiction of a Firefly luciferase NanoLuc transposon reporter. Structural features of the transposon are shown both in a circular map and a linear map. Firefly luciferase expression is observed if there is an increase in excision of the transposon and an increase in NanoLuc is observed if there is an increase in the integration of the transposon.

FIG. 7 us a schematic showing the Split GFP Splicing Site Specific Reporter.

FIG. 8 shows the integration and excision activity with wildtype SPB, SPB comprising an N-terminal deletion of 93 amino acids and a DNA targeting domain comprising three Zinc Finger Motifs (ZFM-SPB), and integration deficient SPB (PBx) comprising an N-terminal deletion of 93 amino acids and a DNA targeting domain comprising three Zinc Finger Motifs (ZFM-PBx) at modified target sites with varying lengths of spacers between the SPB target site and the ZFM target site.

FIGS. 9A, 9B, and 9C show off target genomic integration activity, on-target episomal integration activity, and the ratio of on target to off target activity, respectively, with SPB, ZFM-SPB, and ZFM-PBx.

FIGS. 10A-10C show excision activity and integration activity of ZFM-PBx and ZFM-PBx-NTD.

FIG. 11 shows a schematic of the GFP Excision Only Reporter.

FIG. 12 shows sequence-specificity of GFP TALENs using a single strand annealing (SSA) assay. L and R indicate left and right TAL arrays, respectively.

FIG. 13 shows sequence-specificity of PAH TALENs using a single strand annealing (SSA) assay. L and R indicate left and right TAL arrays, respectively.

FIG. 14 shows sequence-specificity of PAH TALENs using an episomal Split GFP Splicing Site-Specific Reporter assay.

FIG. 15 shows sequence-specificity of PAH TALENs with on-target and off-target array pairs using an episomal Split GFP Splicing Site-Specific Reporter assay.

FIG. 16 shows the rate of site-specific transposition into genomic DNA at six TTAA target sites in LINE1 repeat elements as detected by ddPCR. Transposon integration was measured with respect to a reference gene and is reported as % site specific transposition per haploid genome.

FIG. 17 shows ddPCR data demonstrating site-specific transposition into genomic DNA for four TTAA sites within the B2M gene. Droplets with high amplitude along the Y-axis contain an edited genomic DNA template.

FIG. 18 shows the integration activity of various PBx-ZFN fusion constructs determined by Split GFP assay.

FIG. 19 shows the integration activity of TAL-PBx fusion constructs harboring various truncations of the PBx N-terminal domain as determined by Split GFP assay. Reporters in which the TAL binding site was separated from the TTAA integration site by 11 bp, 12 bp, 13 bp, or 14 bp spacers were used.

FIG. 20 shows an illustration of various TAL-PBx fusion constructs. A set of TAL C-terminal domain truncations retaining 13, 23, 33, 43, 54, 63, or 73 amino acids were fused in combination with PBx N-terminally truncated by 85, 88, 93, 99, or 103 amino acids.

FIG. 21 shows the integration activity of the various TAL-PBx fusion constructs illustrated in FIG. 20 as determined by Split GFP assay. The TAL-PBx fusions were tested using target sites in which the TAL binding site was separated from the TTAA integration site by 11 bp, 12 bp, 13 bp, or 14 bp spacers.

FIG. 22 is a schematic of an “all-in-one site-specific excision/integration episomal reporter.” This episomal reporter system comprises a plasmid containing a transposon donor along with a transposon integration site all on the same plasmid. The transposon contains a CMV promoter. The transposon in this plasmid disrupts the open reading frame of a GFP preceded by an EF1a promoter and followed by poly adenylation signal sequence. The vector also contains, in the opposite orientation, a polyA and transcription pause site, a TTAA integration site adjacent to a target sequences and spacers, followed by a PEST destabilized mScarlet reporter and a poly adenylation signal sequence. This “all-in-one site-specific excision/integration episomal reporter” when transfected into cells alone, should express no GFP and no or little mScarlet. Upon transposon excision catalyzed by SPB, PBx, or ssSPB, GFP should be expressed. Upon site-specific integration of the CMV promoter containing transposon into its target site upstream of mScarlet resulting in its expression.

FIG. 23 shows the excision and site-specific integration activity of various TAL-PBx constructs containing mutations at positions 372 or 375.

FIG. 24 shows sequence-specificity of ZF-PBx designed to recognize ZF268, chr17, and chr21 target sites with on-target and off-target array pairs using an episomal Split GFP Splicing Site Specific Reporter assay.

FIG. 25A shows site-specific integration activity of ZF268-PBx and ZF268-tdPBx at target site with ZF268 binding sites on both sides of TTAA or on one side of TTAA as measured using an episomal Split GFP Splicing Site Specific Reporter assay.

FIG. 25B-C shows excision and site-specific integration activity of PAH2 or PAH3 TAL-PBx and TAL-tdPBX tested as pairs or as individual left or right fusion proteins as measured using an episomal Split GFP Splicing Site Specific Reporter assay.

FIG. 26A shows site-specific integration activity of TAL-PBx at a chr17 target site cloned into the episomal Split GFP splicing site specific reporter.

FIG. 26B-C show site-specific integration activity of TAL-PBx at a chr17 target in genomic DNA as measured by ddPCR. Droplets with high amplitude along the Y-axis contain an edited genomic DNA template. Droplets with high amplitude along the x-axis contain an genomic DNA reference gene template on the bottom plot.

### DETAILED DESCRIPTION

Provided herein are transposase domains and fusion proteins comprising the same, in particular, transposase domains comprising N-terminal deletions. The fusion proteins comprising said transposase domains may be further mutated so that they form obligate heterodimers. Also provided are methods of making the transposase domains and fusion proteins, cells that are modified using the fusion proteins provided herein and methods of treatment using such cells.

Transposase domains provided herein may be, for example, wildtype transposase domains or integration deficient (excision only) transposase domains.

Also provided herein are fusion proteins comprising one or more transposase domains and a DNA targeting domain. In some embodiment, the fusion protein further comprises a protein stabilization domain.

### Transposase Domains and Fusion Proteins Comprising Transposase Domains

In one aspect, provided herein are transposase domains and fusion proteins comprising the same (e.g., comprising a first and a second transposase domain). In some embodiments, the transposase domain is a piggyBac transposase domain. In some embodiments, the piggyBac transposase domain is a hyperactive piggyBac transposase domain. In preferred embodiments, the transposase domain is a Super piggyBac™ transposase domains (SPB). Non-limiting examples of SPB transposases are described in detail in U.S. Pat. Nos. 6,218,182; 6,962,810; 8,399,643 and PCT Publication No. WO 2010/099296.

In some embodiments, the transposase domain is a Super PiggyBac transposase (SPB) domain. An exemplary wildtype SPB sequence comprising a nuclear localization sequence (NLS) is shown in SEQ ID NO: 1 with the NLS shown in italics, hyperactive mutations shown in bold, and the Cysteine Rich Domain (CRD) underlined. The numbering of sequence of the SPB transposase domain for the purpose of describing deletions and mutations begins at residue 12 of SEQ ID NO: 1.

(SEQ ID NO: 1)
MAPKKKRKVGGGGSSLDDEHILSALLQSDDELVGEDSDSEVSDHVS
EDDVQSDTEEAFIDEVHEVQPTSSGSEILDEQNVIEQPGSSLASN
RILTLPQRTIRGKNKHCWSTSKSTRRSRVSALNIVRSQRGPTRMC
RNIYDPLLCFKLFFTDEIISEIVKWTNAEISLKRRESMTSATFRD
TNEDEIYAFFGILVMTAVRKDNHMSTDDLFDRSLSMVYVSVMSRD
RFDFLIRCLRMDDKSIRPTLRENDVFTPVRKIWDLFIHQCIQNYT
PGAHLTIDEQLLGFRGRCPFRVYIPNKPSKYGIKILMMCDSGTKY
MINGMPYLGRGTQTNGVPLGEYYVKELSKPVHGSCRNITCDNWFT
SIPLAKNLLQEPYKLTIVGTVRSNKREIPEVLKNSRSRPVGTSMF
CFDGPLTLVSYKPKPAKMVYLLSSCDEDASINESTGKPQMVMYYN
QTKGGVDTLDQMCSVMTCSRKTNRWPMALLYGMINIACINSFIIY
SHNVSSKGEKVQSRKKFMRNLYMSLTSSFMRKRLEAPTLKRYLRD
NISNILPKEVPGTSDDSTEEPVMKKRTYCTYCPSKIRRKANASCK
KCKKVICREHNIDMCQSCF

An exemplary sequence of wildtype SPB transposase which is lacking the NLS domain is set forth in SEQ ID NO: 55. The numbering of sequence of the SPB transposase domain for the purpose of describing deletions and mutations begins at residue 5 of SEQ ID NO: 55.

The transposase domains used in the fusion proteins described herein can be isolated or derived from an insect, vertebrate, crustacean or urochordate as described in more detail in PCT Publication No. WO 2019/173636 and PCT/US2019/049816. In preferred aspects, the SPB transposase domain is isolated or derived from the insect Trichoplusia ni (GenBank Accession No. AAA87375) or Bombyx mori (GenBank Accession No. BAD11135).

In some embodiments, the transposase domain is integration deficient. An integration deficient transposase domain is a transposase that can excise its corresponding transposon, but that integrates the excised transposon at a lower frequency than a corresponding wild type transposase. Examples of integration deficient transposases are disclosed in U.S. Pat. Nos. 6,218,185; 6,962,810, 8,399,643 and WO 2019/173636. A list of integration deficient amino acid substitutions is disclosed in U.S. Pat. No. 10,041,077. A wildtype SPB may be rendered integration deficient by introducing mutations, for example, K93A, R372A, K375A, R376A and/or D450N (relative to SEQ ID NO: 55, with numbering beginning at residue 5). It is believed that the introduction of mutations R372A, K375A, R376A and D450N renders the transposase integration deficient, but retains the excision function. An exemplary sequence of an integration-deficient transposase domain is PBx comprising an NLS is set forth in SEQ ID NO: 56. The sequence of an integration deficient PBx transpose domain not comprising an NLS is set forth in SEQ ID NO: 544:

(SEQ ID NO: 544)
GGSSLDDEHILSALLQSDDELVGEDSDSEVSDHVSEDDVQSDTEEAFID
EVHEVQPTSSGSEILDEQNVIEQPGSSLASNRILTLPQRTIRGKNKHCW
STSKSTRRSRVSALNIVRSQRGPTRMCRNIYDPLLCFKLFFTDEIISEI
VKWTNAEISLKRRESMTSATFRDTNEDEIYAFFGILVMTAVRKDNHMST
DDLFDRSLSMVYVSVMSRDRFDFLIRCLRMDDKSIRPTLRENDVFTPVR
KIWDLFIHQCIQNYTPGAHLTIDEQLLGFRGRCPFRVYIPNKPSKYGIK
ILMMCDSGTKYMINGMPYLGRGTQTNGVPLGEYYVKELSKPVHGSCRNI
TCDNWFTSIPLAKNLLQEPYKLTIVGTVASNAREIPEVLKNSRSRPVGT
SMFCFDGPLTLVSYKPKPAKMVYLLSSCDEDASINESTGKPQMVMYYNQ
TKGGVDTLNQMCSVMTCSRKTNRWPMALLYGMINIACINSFIIYSHNVS
SKGEKVQSRKKFMRNLYMSLTSSFMRKRLEAPTLKRYLRDNISNILPKE
VPGTSDDSTEEPVMKKRTYCTYCPSKIRRKANASCKKCKKVICREHNID
MCQSCF.

### Transposase Domains Comprising N-Terminal Deletions

In some embodiments, provided herein are transposase domains (e.g., SPB transposase domains or PBx transposase domains) comprising a deletion of a portion of the amino terminus (also referred to as the “N-terminus” or the “N-terminal Domain,” or “NTD) of the transposase domain. Without wishing to be bound by theory, it is believed that, in the context of a tandem dimer transposase (or a dimer comprising two fusion proteins described herein) the N-terminal domain of a transposase (e.g., SPB) may introduce steric hindrance between the two dimers of a tandem dimer, or between a dimer and the DNA.

In some embodiments, the deleted portion of the N-terminus is about 20 amino acids, about 40 amino acids, about 60 amino acids, about 80 amino acids, about 100 amino acids or about 115 amino acids. In some embodiments, the deleted portion of the N-terminus is about 15-25 amino acids, about 25-35 amino acids, about 35-45 amino acids, about 45-55 amino acids, about 55-65 amino acids, about 65-75 amino acids, about 75-85 amino acids, about 85-95 amino acids, about 95-105 amino acids, or about 105-120 amino acids.

In some embodiments, the transposase domain comprises a deletion of amino acids 1-20 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-40 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-60 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-80 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-83 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-84 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-85 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-86 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-87 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-88 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-89 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-90 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-91 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-92 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-93 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-94 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-95 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-96 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-97 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-98 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-99 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-100 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-101 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-102 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-103 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544. In some embodiments, the transposase domain comprises a deletion of amino acids 1-115 of the N-terminus relative to SEQ ID NO: 1, 55, or 56, with numbering beginning at residue 12 of SEQ ID NO: 1 and residue 5 of SEQ ID NO: 55 and 56, or relative to SEQ ID NO: 544.

Illustrative sequences of an SPB transposase domain with a deletion of amino acids 1-93 of the N-terminus and of a PBx transposase domain with a deletion of amino acids 1-93 of the N-terminus are shown in SEQ ID NOs: 65 and 66, respectively:

(SEQ ID NO: 65)
NKHCWSTSKSTRRSRVSALNIVRSQRGPTRMCRNIYDPLLCFKLFFTDE
IISEIVKWTNAEISLKRRESMTSATFRDTNEDEIYAFFGILVMTAVRKD
NHMSTDDLFDRSLSMVYVSVMSRDRFDFLIRCLRMDDKSIRPTLRENDV
FTPVRKIWDLFIHQCIQNYTPGAHLTIDEQLLGFRGRCPFRVYIPNKPS
KYGIKILMMCDSGTKYMINGMPYLGRGTQTNGVPLGEYYVKELSKPVHG
SCRNITCDNWFTSIPLAKNLLQEPYKLTIVGTVRSNKREIPEVLKNSRS
RPVGTSMFCFDGPLTLVSYKPKPAKMVYLLSSCDEDASINESTGKPQMV
MYYNQTKGGVDTLDQMCSVMTCSRKTNRWPMALLYGMINIACINSFIIY
SHNVSSKGEKVQSRKKFMRNLYMSLTSSFMRKRLEAPTLKRYLRDNISN
ILPKEVPGTSDDSTEEPVMKKRTYCTYCPSKIRRKANASCKKCKKVICR
EHNIDMCQSCF
(SEQ ID NO: 66)
NKHCWSTSKSTRRSRVSALNIVRSQRGPTRMCRNIYDPLLCFKLFFTDE
IISEIVKWTNAEISLKRRESMTSATFRDTNEDEIYAFFGILVMTAVRKD
NHMSTDDLFDRSLSMVYVSVMSRDRFDFLIRCLRMDDKSIRPTLRENDV
FTPVRKIWDLFIHQCIQNYTPGAHLTIDEQLLGFRGRCPFRVYIPNKPS
KYGIKILMMCDSGTKYMINGMPYLGRGTQTNGVPLGEYYVKELSKPVHG
SCRNITCDNWFTSIPLAKNLLQEPYKLTIVGTVASNAREIPEVLKNSRS
RPVGTSMFCFDGPLTLVSYKPKPAKMVYLLSSCDEDASINESTGKPQMV
MYYNQTKGGVDTLNQMCSVMTCSRKTNRWPMALLYGMINIACINSFIIY
SHNVSSKGEKVQSRKKFMRNLYMSLTSSFMRKRLEAPTLKRYLRDNISN
ILPKEVPGTSDDSTEEPVMKKRTYCTYCPSKIRRKANASCKKCKKVICR
EHNIDMCQSCF

Other illustrative sequences of SPB transpose domains comprising N-terminal deletions are set forth in SEQ ID NOs: 2-7. Illustrative sequences of PBx transposase domains comprising N-terminal deletions are set forth in SEQ ID NOs: 86-106 in Table 1.

TABLE 1
Illustrative sequences of N-terminally deleted PBx Domains

| Deletion | Sequence |
|---|---|
| PBx Delta | TLPQRTIRGKNKHCWSTSKSTRRSRVSALNIVRSQRGPTRMCRNIYDPLLCFKLF |
| 83 N- | FTDEIISEIVKWTNAEISLKRRESMTSATFRDTNEDEIYAFFGILVMTAVRKDNH |
| Terminal | MSTDDLFDRSLSMVYVSVMSRDRFDFLIRCLRMDDKSIRPTLRENDVFTPVRKI |
| | WDLFIHQCIQNYTPGAHLTIDEQLLGFRGRCPFRVYIPNKPSKYGIKILMMCDSG |
| | TKYMINGMPYLGRGTQTNGVPLGEYYVKELSKPVHGSCRNITCDNWFTSIPLA |
| | KNLLQEPYKLTIVGTVASNAREIPEVLKNSRSRPVGTSMFCFDGPLTLVSYKPKP |
| | AKMVYLLSSCDEDASINESTGKPQMVMYYNQTKGGVDTLNQMCSVMTCSRK |
| | TNRWPMALLYGMINIACINSFIIYSHNVSSKGEKVQSRKKFMRNLYMSLTSSFM |
| | RKRLEAPTLKRYLRDNISNILPKEVPGTSDDSTEEPVMKKRTYCTYCPSKIRRKA |
| | NASCKKCKKVICREHNIDMCQSCF (SEQ ID NO: 86) |
| PBx Delta | LPQRTIRGKNKHCWSTSKSTRRSRVSALNIVRSQRGPTRMCRNIYDPLLCFKLFF |
| 84 N- | TDEIISEIVKWTNAEISLKRRESMTSATFRDTNEDEIYAFFGILVMTAVRKDNHM |
| Terminal | STDDLFDRSLSMVYVSVMSRDRFDFLIRCLRMDDKSIRPTLRENDVFTPVRKIW |
| | DLFIHQCIQNYTPGAHLTIDEQLLGFRGRCPFRVYIPNKPSKYGIKILMMCDSGT |
| | KYMINGMPYLGRGTQTNGVPLGEYYVKELSKPVHGSCRNITCDNWFTSIPLAK |
| | NLLQEPYKLTIVGTVASNAREIPEVLKNSRSRPVGTSMFCFDGPLTLVSYKPKPA |
| | KMVYLLSSCDEDASINESTGKPQMVMYYNQTKGGVDTLNQMCSVMTCSRKT |
| | NRWPMALLYGMINIACINSFIIYSHNVSSKGEKVQSRKKFMRNLYMSLTSSFMR |
| | KRLEAPTLKRYLRDNISNILPKEVPGTSDDSTEEPVMKKRTYCTYCPSKIRRKAN |
| | ASCKKCKKVICREHNIDMCQSCF (SEQ ID NO: 87) |
| PBx Delta | PQRTIRGKNKHCWSTSKSTRRSRVSALNIVRSQRGPTRMCRNIYDPLLCFKLFFT |
| 85 N- | DEIISEIVKWTNAEISLKRRESMTSATFRDTNEDEIYAFFGILVMTAVRKDNHMS |
| Terminal | TDDLFDRSLSMVYVSVMSRDRFDFLIRCLRMDDKSIRPTLRENDVFTPVRKIWD |
| | LFIHQCIQNYTPGAHLTIDEQLLGFRGRCPFRVYIPNKPSKYGIKILMMCDSGTK |
| | YMINGMPYLGRGTQTNGVPLGEYYVKELSKPVHGSCRNITCDNWFTSIPLAKN |
| | LLQEPYKLTIVGTVASNAREIPEVLKNSRSRPVGTSMFCFDGPLTLVSYKPKPAK |
| | MVYLLSSCDEDASINESTGKPQMVMYYNQTKGGVDTLNQMCSVMTCSRKTN |
| | RWPMALLYGMINIACINSFIIYSHNVSSKGEKVQSRKKFMRNLYMSLTSSFMRK |
| | RLEAPTLKRYLRDNISNILPKEVPGTSDDSTEEPVMKKRTYCTYCPSKIRRKANA |
| | SCKKCKKVICREHNIDMCQSCF (SEQ ID NO: 88) |
| PBx Delta | QRTIRGKNKHCWSTSKSTRRSRVSALNIVRSQRGPTRMCRNIYDPLLCFKLFFT |
| 86 N- | DEIISEIVKWTNAEISLKRRESMTSATFRDTNEDEIYAFFGILVMTAVRKDNHMS |
| Terminal | TDDLFDRSLSMVYVSVMSRDRFDFLIRCLRMDDKSIRPTLRENDVFTPVRKIWD |
| | LFIHQCIQNYTPGAHLTIDEQLLGFRGRCPFRVYIPNKPSKYGIKILMMCDSGTK |
| | YMINGMPYLGRGTQTNGVPLGEYYVKELSKPVHGSCRNITCDNWFTSIPLAKN |
| | LLQEPYKLTIVGTVASNAREIPEVLKNSRSRPVGTSMFCFDGPLTLVSYKPKPAK |
| | MVYLLSSCDEDASINESTGKPQMVMYYNQTKGGVDTLNQMCSVMTCSRKTN |
| | RWPMALLYGMINIACINSFIIYSHNVSSKGEKVQSRKKFMRNLYMSLTSSFMRK |
| | RLEAPTLKRYLRDNISNILPKEVPGTSDDSTEEPVMKKRTYCTYCPSKIRRKANA |
| | SCKKCKKVICREHNIDMCQSCF (SEQ ID NO: 89). |
| PBx Delta | RTIRGKNKHCWSTSKSTRRSRVSALNIVRSQRGPTRMCRNIYDPLLCFKLFFTDE |
| 87 N- | IISEIVKWTNAEISLKRRESMTSATFRDTNEDEIYAFFGILVMTAVRKDNHMSTD |
| Terminal | DLFDRSLSMVYVSVMSRDRFDFLIRCLRMDDKSIRPTLRENDVFTPVRKIWDLFI |
| | HQCIQNYTPGAHLTIDEQLLGFRGRCPFRVYIPNKPSKYGIKILMMCDSGTKYMI |
| | NGMPYLGRGTQTNGVPLGEYYVKELSKPVHGSCRNITCDNWFTSIPLAKNLLQ |
| | EPYKLTIVGTVASNAREIPEVLKNSRSRPVGTSMFCFDGPLTLVSYKPKPAKMV |
| | YLLSSCDEDASINESTGKPQMVMYYNQTKGGVDTLNQMCSVMTCSRKTNRWP |
| | MALLYGMINIACINSFIIYSHNVSSKGEKVQSRKKFMRNLYMSLTSSFMRKRLE |
| | APTLKRYLRDNISNILPKEVPGTSDDSTEEPVMKKRTYCTYCPSKIRRKANASCK |
| | KCKKVICREHNIDMCQSCF (SEQ ID NO: 90) |
| PBx Delta | TIRGKNKHCWSTSKSTRRSRVSALNIVRSQRGPTRMCRNIYDPLLCFKLFFTDEII |
| 88 N- | SEIVKWTNAEISLKRRESMTSATFRDTNEDEIYAFFGILVMTAVRKDNHMSTDD |
| Terminal | LFDRSLSMVYVSVMSRDRFDFLIRCLRMDDKSIRPTLRENDVFTPVRKIWDLFIH |
| | QCIQNYTPGAHLTIDEQLLGFRGRCPFRVYIPNKPSKYGIKILMMCDSGTKYMIN |
| | GMPYLGRGTQTNGVPLGEYYVKELSKPVHGSCRNITCDNWFTSIPLAKNLLQE |
| | PYKLTIVGTVASNAREIPEVLKNSRSRPVGTSMFCFDGPLTLVSYKPKPAKMVY |
| | LLSSCDEDASINESTGKPQMVMYYNQTKGGVDTLNQMCSVMTCSRKTNRWP |
| | MALLYGMINIACINSFIIYSHNVSSKGEKVQSRKKFMRNLYMSLTSSFMRKRLE |
| | APTLKRYLRDNISNILPKEVPGTSDDSTEEPVMKKRTYCTYCPSKIRRKANASCK |
| | KCKKVICREHNIDMCQSCF (SEQ ID NO: 91) |
| PBx Delta | IRGKNKHCWSTSKSTRRSRVSALNIVRSQRGPTRMCRNIYDPLLCFKLFFTDEIIS |
| 89 N- | EIVKWTNAEISLKRRESMTSATFRDTNEDEIYAFFGILVMTAVRKDNHMSTDDL |
| Terminal | FDRSLSMVYVSVMSRDRFDFLIRCLRMDDKSIRPTLRENDVFTPVRKIWDLFIH |
| | QCIQNYTPGAHLTIDEQLLGFRGRCPFRVYIPNKPSKYGIKILMMCDSGTKYMIN |
| | GMPYLGRGTQTNGVPLGEYYVKELSKPVHGSCRNITCDNWFTSIPLAKNLLQE |
| | PYKLTIVGTVASNAREIPEVLKNSRSRPVGTSMFCFDGPLTLVSYKPKPAKMVY |
| | LLSSCDEDASINESTGKPQMVMYYNQTKGGVDTLNQMCSVMTCSRKTNRWP |
| | MALLYGMINIACINSFIIYSHNVSSKGEKVQSRKKFMRNLYMSLTSSFMRKRLE |
| | APTLKRYLRDNISNILPKEVPGTSDDSTEEPVMKKRTYCTYCPSKIRRKANASCK |
| | KCKKVICREHNIDMCQSCF (SEQ ID NO: 92) |
| PBx Delta | RGKNKHCWSTSKSTRRSRVSALNIVRSQRGPTRMCRNIYDPLLCFKLFFTDEIIS |
| 90 N- | EIVKWTNAEISLKRRESMTSATFRDTNEDEIYAFFGILVMTAVRKDNHMSTDDL |
| Terminal | FDRSLSMVYVSVMSRDRFDFLIRCLRMDDKSIRPTLRENDVFTPVRKIWDLFIH |
| | QCIQNYTPGAHLTIDEQLLGFRGRCPFRVYIPNKPSKYGIKILMMCDSGTKYMIN |
| | GMPYLGRGTQTNGVPLGEYYVKELSKPVHGSCRNITCDNWFTSIPLAKNLLQE |
| | PYKLTIVGTVASNAREIPEVLKNSRSRPVGTSMFCFDGPLTLVSYKPKPAKMVY |
| | LLSSCDEDASINESTGKPQMVMYYNQTKGGVDTLNQMCSVMTCSRKTNRWP |
| | MALLYGMINIACINSFIIYSHNVSSKGEKVQSRKKFMRNLYMSLTSSFMRKRLE |
| | APTLKRYLRDNISNILPKEVPGTSDDSTEEPVMKKRTYCTYCPSKIRRKANASCK |
| | KCKKVICREHNIDMCQSCF (SEQ ID NO: 93) |
| PBx Delta | GKNKHCWSTSKSTRRSRVSALNIVRSQRGPTRMCRNIYDPLLCFKLFFTDEIISEI |
| 91 N- | VKWTNAEISLKRRESMTSATFRDTNEDEIYAFFGILVMTAVRKDNHMSTDDLF |
| Terminal | DRSLSMVYVSVMSRDRFDFLIRCLRMDDKSIRPTLRENDVFTPVRKIWDLFIHQ |
| | CIQNYTPGAHLTIDEQLLGFRGRCPFRVYIPNKPSKYGIKILMMCDSGTKYMING |
| | MPYLGRGTQTNGVPLGEYYVKELSKPVHGSCRNITCDNWFTSIPLAKNLLQEP |
| | YKLTIVGTVASNAREIPEVLKNSRSRPVGTSMFCFDGPLTLVSYKPKPAKMVYL |
| | LSSCDEDASINESTGKPQMVMYYNQTKGGVDTLNQMCSVMTCSRKTNRWPM |
| | ALLYGMINIACINSFIIYSHNVSSKGEKVQSRKKFMRNLYMSLTSSFMRKRLEAP |
| | TLKRYLRDNISNILPKEVPGTSDDSTEEPVMKKRTYCTYCPSKIRRKANASCKK |
| | CKKVICREHNIDMCQSCF (SEQ ID NO: 94) |
| PBx Delta | KNKHCWSTSKSTRRSRVSALNIVRSQRGPTRMCRNIYDPLLCFKLFFTDEIISEIV |
| 92 N- | KWTNAEISLKRRESMTSATFRDTNEDEIYAFFGILVMTAVRKDNHMSTDDLFD |
| Terminal | RSLSMVYVSVMSRDRFDFLIRCLRMDDKSIRPTLRENDVFTPVRKIWDLFIHQCI |
| | QNYTPGAHLTIDEQLLGFRGRCPFRVYIPNKPSKYGIKILMMCDSGTKYMINGM |
| | PYLGRGTQTNGVPLGEYYVKELSKPVHGSCRNITCDNWFTSIPLAKNLLQEPYK |
| | LTIVGTVASNAREIPEVLKNSRSRPVGTSMFCFDGPLTLVSYKPKPAKMVYLLSS |
| | CDEDASINESTGKPQMVMYYNQTKGGVDTLNQMCSVMTCSRKTNRWPMALL |
| | YGMINIACINSFIIYSHNVSSKGEKVQSRKKFMRNLYMSLTSSFMRKRLEAPTLK |
| | RYLRDNISNILPKEVPGTSDDSTEEPVMKKRTYCTYCPSKIRRKANASCKKCKK |
| | VICREHNIDMCQSCF (SEQ ID NO: 95) |
| PBx Delta | NKHCWSTSKSTRRSRVSALNIVRSQRGPTRMCRNIYDPLLCFKLFFTDEIISEIVK |
| 93 N- | WTNAEISLKRRESMTSATFRDTNEDEIYAFFGILVMTAVRKDNHMSTDDLFDRS |
| Terminal | LSMVYVSVMSRDRFDFLIRCLRMDDKSIRPTLRENDVFTPVRKIWDLFIHQCIQ |
| | NYTPGAHLTIDEQLLGFRGRCPFRVYIPNKPSKYGIKILMMCDSGTKYMINGMP |
| | YLGRGTQTNGVPLGEYYVKELSKPVHGSCRNITCDNWFTSIPLAKNLLQEPYKL |
| | TIVGTVASNAREIPEVLKNSRSRPVGTSMFCFDGPLTLVSYKPKPAKMVYLLSS |
| | CDEDASINESTGKPQMVMYYNQTKGGVDTLNQMCSVMTCSRKTNRWPMALL |
| | YGMINIACINSFIIYSHNVSSKGEKVQSRKKFMRNLYMSLTSSFMRKRLEAPTLK |
| | RYLRDNISNILPKEVPGTSDDSTEEPVMKKRTYCTYCPSKIRRKANASCKKCKK |
| | VICREHNIDMCQSCF (SEQ ID NO: 96) |
| PBx Delta | KHCWSTSKSTRRSRVSALNIVRSQRGPTRMCRNIYDPLLCFKLFFTDEIISEIVK |
| 94 N- | WTNAEISLKRRESMTSATFRDTNEDEIYAFFGILVMTAVRKDNHMSTDDLFDRS |
| Terminal | LSMVYVSVMSRDRFDFLIRCLRMDDKSIRPTLRENDVFTPVRKIWDLFIHQCIQ |
| | NYTPGAHLTIDEQLLGFRGRCPFRVYIPNKPSKYGIKILMMCDSGTKYMINGMP |
| | YLGRGTQTNGVPLGEYYVKELSKPVHGSCRNITCDNWFTSIPLAKNLLQEPYKL |
| | TIVGTVASNAREIPEVLKNSRSRPVGTSMFCFDGPLTLVSYKPKPAKMVYLLSS |
| | CDEDASINESTGKPQMVMYYNQTKGGVDTLNQMCSVMTCSRKTNRWPMALL |
| | YGMINIACINSFIIYSHNVSSKGEKVQSRKKFMRNLYMSLTSSFMRKRLEAPTLK |
| | RYLRDNISNILPKEVPGTSDDSTEEPVMKKRTYCTYCPSKIRRKANASCKKCKK |
| | VICREHNIDMCQSCF (SEQ ID NO: 97) |
| PBx Delta | HCWSTSKSTRRSRVSALNIVRSQRGPTRMCRNIYDPLLCFKLFFTDEIISEIVKWT |
| 95 N- | NAEISLKRRESMTSATFRDTNEDEIYAFFGILVMTAVRKDNHMSTDDLFDRSLS |
| Terminal | MVYVSVMSRDRFDFLIRCLRMDDKSIRPTLRENDVFTPVRKIWDLFIHQCIQNY |
| | TPGAHLTIDEQLLGFRGRCPFRVYIPNKPSKYGIKILMMCDSGTKYMINGMPYL |
| | GRGTQTNGVPLGEYYVKELSKPVHGSCRNITCDNWFTSIPLAKNLLQEPYKLTI |
| | VGTVASNAREIPEVLKNSRSRPVGTSMFCFDGPLTLVSYKPKPAKMVYLLSSCD |
| | EDASINESTGKPQMVMYYNQTKGGVDTLNQMCSVMTCSRKTNRWPMALLYG |
| | MINIACINSFIIYSHNVSSKGEKVQSRKKFMRNLYMSLTSSFMRKRLEAPTLKRY |
| | LRDNISNILPKEVPGTSDDSTEEPVMKKRTYCTYCPSKIRRKANASCKKCKKVIC |
| | REHNIDMCQSCF (SEQ ID NO: 98) |
| PBx Delta | CWSTSKSTRRSRVSALNIVRSQRGPTRMCRNIYDPLLCFKLFFTDEIISEIVKWTN |
| 96 N- | AEISLKRRESMTSATFRDTNEDEIYAFFGILVMTAVRKDNHMSTDDLFDRSLSM |
| Terminal | VYVSVMSRDRFDFLIRCLRMDDKSIRPTLRENDVFTPVRKIWDLFIHQCIQNYTP |
| | GAHLTIDEQLLGFRGRCPFRVYIPNKPSKYGIKILMMCDSGTKYMINGMPYLGR |
| | GTQTNGVPLGEYYVKELSKPVHGSCRNITCDNWFTSIPLAKNLLQEPYKLTIVG |
| | TVASNAREIPEVLKNSRSRPVGTSMFCFDGPLTLVSYKPKPAKMVYLLSSCDED |
| | ASINESTGKPQMVMYYNQTKGGVDTLNQMCSVMTCSRKTNRWPMALLYGMI |
| | NIACINSFIIYSHNVSSKGEKVQSRKKFMRNLYMSLTSSFMRKRLEAPTLKRYLR |
| | DNISNILPKEVPGTSDDSTEEPVMKKRTYCTYCPSKIRRKANASCKKCKKVICRE |
| | HNIDMCQSCF (SEQ ID NO: 99) |
| PBx Delta | WSTSKSTRRSRVSALNIVRSQRGPTRMCRNIYDPLLCFKLFFTDEIISEIVKWTNA |
| 97 N- | EISLKRRESMTSATFRDTNEDEIYAFFGILVMTAVRKDNHMSTDDLFDRSLSMV |
| Terminal | YVSVMSRDRFDFLIRCLRMDDKSIRPTLRENDVFTPVRKIWDLFIHQCIQNYTPG |
| | AHLTIDEQLLGFRGRCPFRVYIPNKPSKYGIKILMMCDSGTKYMINGMPYLGRG |
| | TQTNGVPLGEYYVKELSKPVHGSCRNITCDNWFTSIPLAKNLLQEPYKLTIVGT |
| | VASNAREIPEVLKNSRSRPVGTSMFCFDGPLTLVSYKPKPAKMVYLLSSCDEDA |
| | SINESTGKPQMVMYYNQTKGGVDTLNQMCSVMTCSRKTNRWPMALLYGMINI |
| | ACINSFIIYSHNVSSKGEKVQSRKKFMRNLYMSLTSSFMRKRLEAPTLKRYLRD |
| | NISNILPKEVPGTSDDSTEEPVMKKRTYCTYCPSKIRRKANASCKKCKKVICREH |
| | NIDMCQSCF (SEQ ID NO: 100) |
| PBx Delta | STSKSTRRSRVSALNIVRSQRGPTRMCRNIYDPLLCFKLFFTDEIISEIVKWTNAEI |
| 98 N- | SLKRRESMTSATFRDTNEDEIYAFFGILVMTAVRKDNHMSTDDLFDRSLSMVY |
| Terminal | VSVMSRDRFDFLIRCLRMDDKSIRPTLRENDVFTPVRKIWDLFIHQCIQNYTPGA |
| | HLTIDEQLLGFRGRCPFRVYIPNKPSKYGIKILMMCDSGTKYMINGMPYLGRGT |
| | QTNGVPLGEYYVKELSKPVHGSCRNITCDNWFTSIPLAKNLLQEPYKLTIVGTV |
| | ASNAREIPEVLKNSRSRPVGTSMFCFDGPLTLVSYKPKPAKMVYLLSSCDEDASI |
| | NESTGKPQMVMYYNQTKGGVDTLNQMCSVMTCSRKTNRWPMALLYGMINIA |
| | CINSFIIYSHNVSSKGEKVQSRKKFMRNLYMSLTSSFMRKRLEAPTLKRYLRDNI |
| | SNILPKEVPGTSDDSTEEPVMKKRTYCTYCPSKIRRKANASCKKCKKVICREHNI |
| | DMCQSCF (SEQ ID NO: 101) |
| PBx Delta | TSKSTRRSRVSALNIVRSQRGPTRMCRNIYDPLLCFKLFFTDEIISEIVKWTNAEIS |
| 99 N- | LKRRESMTSATFRDTNEDEIYAFFGILVMTAVRKDNHMSTDDLFDRSLSMVYV |
| Terminal | SVMSRDRFDFLIRCLRMDDKSIRPTLRENDVFTPVRKIWDLFIHQCIQNYTPGAH |
| | LTIDEQLLGFRGRCPFRVYIPNKPSKYGIKILMMCDSGTKYMINGMPYLGRGTQ |
| | TNGVPLGEYYVKELSKPVHGSCRNITCDNWFTSIPLAKNLLQEPYKLTIVGTVA |
| | SNAREIPEVLKNSRSRPVGTSMFCFDGPLTLVSYKPKPAKMVYLLSSCDEDASIN |
| | ESTGKPQMVMYYNQTKGGVDTLNQMCSVMTCSRKTNRWPMALLYGMINIAC |
| | INSFIIYSHNVSSKGEKVQSRKKFMRNLYMSLTSSFMRKRLEAPTLKRYLRDNIS |
| | NILPKEVPGTSDDSTEEPVMKKRTYCTYCPSKIRRKANASCKKCKKVICREHNI |
| | DMCQSCF (SEQ ID NO: 102) |
| PBx Delta | SKSTRRSRVSALNIVRSQRGPTRMCRNIYDPLLCFKLFFTDEIISEIVKWTNAEISL |
| 100 N- | KRRESMTSATFRDTNEDEIYAFFGILVMTAVRKDNHMSTDDLFDRSLSMVYVS |
| Terminal | VMSRDRFDFLIRCLRMDDKSIRPTLRENDVFTPVRKIWDLFIHQCIQNYTPGAHL |
| | TIDEQLLGFRGRCPFRVYIPNKPSKYGIKILMMCDSGTKYMINGMPYLGRGTQT |
| | NGVPLGEYYVKELSKPVHGSCRNITCDNWFTSIPLAKNLLQEPYKLTIVGTVAS |
| | NAREIPEVLKNSRSRPVGTSMFCFDGPLTLVSYKPKPAKMVYLLSSCDEDASIN |
| | ESTGKPQMVMYYNQTKGGVDTLNQMCSVMTCSRKTNRWPMALLYGMINIAC |
| | INSFIIYSHNVSSKGEKVQSRKKFMRNLYMSLTSSFMRKRLEAPTLKRYLRDNIS |
| | NILPKEVPGTSDDSTEEPVMKKRTYCTYCPSKIRRKANASCKKCKKVICREHNI |
| | DMCQSCF (SEQ ID NO: 103) |
| PBx Delta | KSTRRSRVSALNIVRSQRGPTRMCRNIYDPLLCFKLFFTDEIISEIVKWTNAEISL |
| 101 N- | KRRESMTSATFRDTNEDEIYAFFGILVMTAVRKDNHMSTDDLFDRSLSMVYVS |
| Terminal | VMSRDRFDFLIRCLRMDDKSIRPTLRENDVFTPVRKIWDLFIHQCIQNYTPGAHL |
| | TIDEQLLGFRGRCPFRVYIPNKPSKYGIKILMMCDSGTKYMINGMPYLGRGTQT |
| | NGVPLGEYYVKELSKPVHGSCRNITCDNWFTSIPLAKNLLQEPYKLTIVGTVAS |
| | NAREIPEVLKNSRSRPVGTSMFCFDGPLTLVSYKPKPAKMVYLLSSCDEDASIN |
| | ESTGKPQMVMYYNQTKGGVDTLNQMCSVMTCSRKTNRWPMALLYGMINIAC |
| | INSFIIYSHNVSSKGEKVQSRKKFMRNLYMSLTSSFMRKRLEAPTLKRYLRDNIS |
| | NILPKEVPGTSDDSTEEPVMKKRTYCTYCPSKIRRKANASCKKCKKVICREHNI |
| | DMCQSCF (SEQ ID NO: 104) |
| PBx Delta | STRRSRVSALNIVRSQRGPTRMCRNIYDPLLCFKLFFTDEIISEIVKWTNAEISLK |
| 102 N- | RRESMTSATFRDTNEDEIYAFFGILVMTAVRKDNHMSTDDLFDRSLSMVYVSV |
| Terminal | MSRDRFDFLIRCLRMDDKSIRPTLRENDVFTPVRKIWDLFIHQCIQNYTPGAHLT |
| | IDEQLLGFRGRCPFRVYIPNKPSKYGIKILMMCDSGTKYMINGMPYLGRGTQTN |
| | GVPLGEYYVKELSKPVHGSCRNITCDNWFTSIPLAKNLLQEPYKLTIVGTVASN |
| | AREIPEVLKNSRSRPVGTSMFCFDGPLTLVSYKPKPAKMVYLLSSCDEDASINES |
| | TGKPQMVMYYNQTKGGVDTLNQMCSVMTCSRKTNRWPMALLYGMINIACIN |
| | SFIIYSHNVSSKGEKVQSRKKFMRNLYMSLTSSFMRKRLEAPTLKRYLRDNISNI |
| | LPKEVPGTSDDSTEEPVMKKRTYCTYCPSKIRRKANASCKKCKKVICREHNIDM |
| | CQSCF (SEQ ID NO: 105) |
| PBx Delta | TRRSRVSALNIVRSQRGPTRMCRNIYDPLLCFKLFFTDEIISEIVKWTNAEISLKR |
| 103 N- | RESMTSATFRDTNEDEIYAFFGILVMTAVRKDNHMSTDDLFDRSLSMVYVSVM |
| Terminal | SRDRFDFLIRCLRMDDKSIRPTLRENDVFTPVRKIWDLFIHQCIQNYTPGAHLTID |
| | EQLLGFRGRCPFRVYIPNKPSKYGIKILMMCDSGTKYMINGMPYLGRGTQTNG |
| | VPLGEYYVKELSKPVHGSCRNITCDNWFTSIPLAKNLLQEPYKLTIVGTVASNA |
| | REIPEVLKNSRSRPVGTSMFCFDGPLTLVSYKPKPAKMVYLLSSCDEDASINEST |
| | GKPQMVMYYNQTKGGVDTLNQMCSVMTCSRKTNRWPMALLYGMINIACINS |
| | FIIYSHNVSSKGEKVQSRKKFMRNLYMSLTSSFMRKRLEAPTLKRYLRDNISNIL |
| | PKEVPGTSDDSTEEPVMKKRTYCTYCPSKIRRKANASCKKCKKVICREHNIDMC |
| | QSCF (SEQ ID NO: 106) |

### Fusion Proteins Comprising Transposase Domains

Also provided herein are fusion proteins comprising one or more transposase domains described herein.

In some embodiments, provided herein is a fusion protein comprising an SPB or PBx domain and a DNA targeting domain. DNA targeting domains are described further below. In some embodiments, provided herein is a fusion protein comprising an SPB or PBx domain, a DNA targeting domain and a protein stabilization domain (PSD). PSDs are described further below.

In some embodiments, a fusion protein provided herein comprises, in N-terminal to C-terminal order, a PSD, a DNA targeting domain, and a transposase domain comprising an N-terminal deletion.

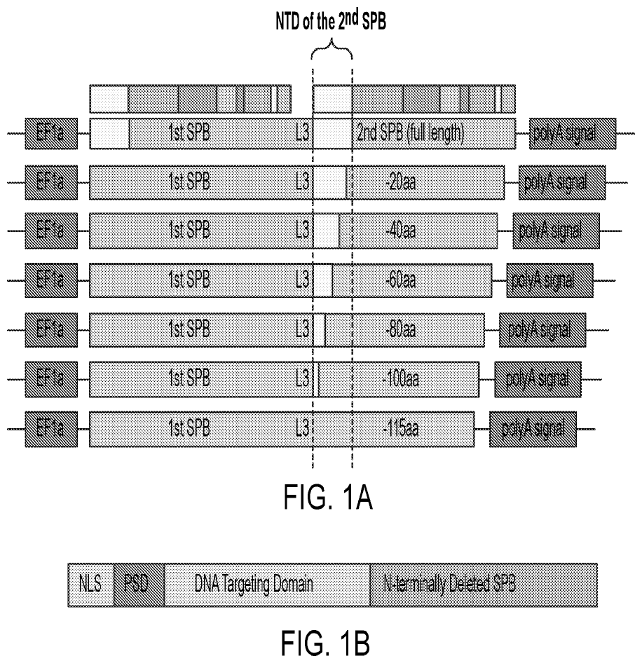

In some embodiments, the fusion protein comprises two transposase domains, e.g. SPBs or PBxs. In some embodiments, provided herein are fusion proteins comprising a first transposase domain and a second transposase domain, wherein the first transposase domain is a full-length transposase domain (e.g., the SPB set forth in SEQ ID NO: 1 or 55, or the PBx set forth in SEQ ID NO: 56, with numbering beginning at the 12^{th} residue of SEQ ID NO: 1 and at the 5^{th} residue of SEQ ID NO: 55 and 56, or the PBx set forth in SEQ ID NO: 544), and wherein the second transposase domain is the same as the first transposase domain except that the second transposase domain comprises an N-terminal deletion. In certain aspects, both the first and second transposase domains are piggyBac transposase domains. In certain aspects, the first transposase domain is a hyperactive piggyBac transposase domain. In certain aspects, the second transposase domain comprises an N-terminal deletion and is a hyperactive piggyBac transposase domain. In certain aspects, the second transposase domain comprises an N-terminal deletion and is a PBx transposase domain. In certain aspects, the second transposase domain comprises an N-terminal deletion and is an SPB. In certain aspects, both the first and second transposases domain are hyperactive piggyBac transposase domains. In some embodiments, the first and/or the second transposase domains are PBx transposase domain. A schematic showing exemplary fusion protein constructs is shown in FIG. 1A.

In some embodiments, the first transposases domain of the fusion protein is a full-length transposase domain and the second transposase domain of the fusion protein is the same as the first transposase domain except that the second transposase domain comprises an N-terminal deletion of about 20 amino acids, of about 40 amino acids, of about 60 amino acids, of about 80 amino acids, of about 81 amino acids, of about 82 amino acids, of about 83 amino acids, of about 84 amino acids, of about 85 amino acids, of about 86 amino acids, of about 87 amino acids, of about 88 amino acids, or about 89 amino acids, of about 90 amino acids, of about 91 amino acids, or about 92 amino acids, of about 93 amino acids, of about 94 amino acids, of about 95 amino acids, of about 96 amino acids, of about 97 amino acids, of about 98 amino acids, of about 99 amino acids, of about 100 amino acids, about 101 amino acids, about 102, amino acids, about 103 amino acids, or of about 115 amino acids. In some embodiments, the first transposases domain of the fusion protein is a full-length transposase domain and the second transposase domain of the fusion protein is the same as the first transposase domain except that the second transposase domain comprises an N-terminal deletion of about 15-25 amino acids, about 25-35 amino acids, about 35-45 amino acids, about 45-55 amino acids, about 55-65 amino acids, about 65-75 amino acids, about 75-85 amino acids, about 85-95 amino acids, about 95-105 amino acids, or about 105-120 amino acids. In certain aspects, the first full-length transposase domain further comprises an in-frame nuclear localization sequence (NLS). In certain aspects, the in-frame NLS is located upstream (i.e., N-terminal) of the nucleotide sequence encoding the first transposase domain. In some embodiments, the NLS comprises or consists of the sequence of SEQ ID NO: 15.

In some embodiments, the first transposases domain of the fusion protein is a full-length transposase domain and the second transposase domain of the fusion protein is the same as the first transposase domain except that the second transposase domain comprises a deletion of amino acids 1-20 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-40 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-60 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-80 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-81 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-82 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-83 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-84 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-85 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-86 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-87 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-88 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-89 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-90 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-91 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-92 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-93 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-94 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-95 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-96 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-97 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-98 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-99 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-100 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-101 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-102 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-103 of the N-terminus. In some embodiments, the first transposases domain of the fusion protein is a full-length transposase domain and the second transposase domain of the fusion protein is the same as the first transposase domain except that the second transposase domain comprises a deletion of amino acids 1-115 of the N-terminus.

In certain aspects, the amino terminus of the second transposase domain of the fusion protein is fused to the C-terminus of the first transposase domain via linker sequence. In some embodiments, the linker is 10-15 amino acids in length. In some embodiments, the linker is 13 amino acids in length. In some embodiments, the linker comprises, consists of, or consists essentially of the amino acid sequence ARLAKLGGGAPAVGGGPKAADKGLP (SEQ ID NO: 16).

In certain aspects, provided herein is a fusion protein, comprising in the N-terminal to C-terminal direction: an in-frame NLS, a first hyperactive piggyBac full length transposase domain, a linker, and a second transposase domain comprising an N-terminal deletion. Exemplary sequences of such fusion proteins are set forth in SEQ ID NOs: 8-14, however, it will be apparent to a person of skill in the art that any of the transposase domain set forth in SEQ ID NOs: 1-7, 55, 56, 58, 59, 65-67, 80-106, or 544 can be freely combined, in any order and in any orientation, in the context of a fusion protein provided herein.

An exemplary sequence of a fusion protein comprising full-length transposase domains is set forth in SEQ ID NO: 8. In some embodiments, a fusion protein provided herein comprises a sequence that is at least 75%, at least 80%, at least 85%, at least 90%, at least 95%, at least 98%, or at least 99% identical to the sequence set forth in SEQ ID NO: 8.

In some embodiments, a fusion protein provided herein comprises two transposase domains, each of which comprises an N-terminal deletion as compared to a wildtype transposase domain (e.g., the SPB transposase domain set forth in SEQ ID NO: 1 or 55, with numbering beginning at the 12^{th} residue of SEQ ID NO: 1 and at the 5^{th} residue of SEQ ID NO: 55, or the PBx transposase domain set forth in SEQ ID NO: 544). The two transposase domains may have the same sequence, or they may have different sequences. For example, each of the two transposase domains comprising an N-terminal deletion may comprise any one of SEQ ID NOs: 2-7, or a sequence that is at least 75%, at least 80%, at least 85%, at least 90%, at least 95%, at least 98%, or at least 99% identical to a sequence set forth in any one of SEQ ID NOs: 2-7. In some embodiments, each of the two transposase domains comprising an N-terminal deletion comprises any one of SEQ ID NOs: 86-106, or a sequence that is at least 75%, at least 80%, at least 85%, at least 90%, at least 95%, at least 98%, or at least 99% identical to a sequence set forth in any one of SEQ ID NOs: 86-106.

In certain embodiments, a fusion protein provided herein comprises a first full-length transposases domain (e.g., the SPB set forth in SEQ ID NO: 1 or 55, with numbering beginning at the 12^{th} residue of SEQ ID NO: 1 and at the 5^{th} residue of SEQ ID NO: 55 or the PBx set forth in SEQ ID NO: 544) and a second transposases domain, wherein the first transposase domain and the second transposase domain are the same, except that the second transposase domain comprises an N-terminal deletion (e.g., a transposase domain comprising the sequence set forth in any one of SEQ ID NOs: 2-7, or a transposase domain comprising a sequence that is at least 75%, at least 80%, at least 85%, at least 90%, at least 95%, at least 98%, or at least 99% identical to a sequence set forth in any one of SEQ ID NOs: 2-7; or a transposase domain comprising the sequence set forth in any one of SEQ ID NOs: 86-106, or a transposase domain comprising a sequence that is at least 75%, at least 80%, at least 85%, at least 90%, at least 95%, at least 98%, or at least 99% identical to a sequence set forth in any one of SEQ ID NOs: 86-106).

In some embodiments, the fusion protein comprises a first full-length transposase domain and a second transposase domain, wherein the first transposase domain and the second transposase domain are the same, except that the second transposase domain comprises an N-terminal deletion of about 20 amino acids. In some embodiments, the fusion protein comprises an amino acid sequence that is at least 75%, at least 85%, at least 90%, at least 95%, at least 98%, or at least 99% identical to the amino acid sequence set forth in SEQ ID NO: 9. In some embodiments, the fusion protein comprises the amino acid sequence set forth in SEQ ID NO: 9.

In some embodiments, the fusion protein comprises a first full-length transposase domain and a second transposase domain, wherein the first transposase domain and the second transposase domain are the same, except that the second transposase domain comprises an N-terminal deletion of about 40 amino acids. In some embodiments, the fusion protein comprises an amino acid sequence that is at least 75%, at least 85%, at least 90%, at least 95%, at least 98%, or at least 99% identical to the amino acid sequence set forth in SEQ ID NO: 10. In some embodiments, the fusion protein comprises the amino acid sequence set forth in SEQ ID NO: 10.

In some embodiments, the fusion protein comprises a first full-length transposase domain and a second transposase domain, wherein the first transposase domain and the second transposase domain are the same, except that the second transposase domain comprises an N-terminal deletion of about 60 amino acids. In some embodiments, the fusion protein comprises an amino acid sequence that is at least 75%, at least 85%, at least 90%, at least 95%, at least 98%, or at least 99% identical to the amino acid sequence set forth in SEQ ID NO: 11. In some embodiments, the fusion protein comprises the amino acid sequence set forth in SEQ ID NO: 11.

In some embodiments, the fusion protein comprises a first full-length transposase domain and a second transposase domain, wherein the first transposase domain and the second transposase domain are the same, except that the second transposase domain comprises an N-terminal deletion of about 80 amino acids. In some embodiments, the fusion protein comprises an amino acid sequence that is at least 75%, at least 85%, at least 90%, at least 95%, at least 98%, or at least 99% identical to the amino acid sequence set forth in SEQ ID NO: 12. In some embodiments, the fusion protein comprises the amino acid sequence set forth in SEQ ID NO: 12.

In some embodiments, the fusion protein comprises a first full-length transposase domain and a second transposase domain, wherein the first transposase domain and the second transposase domain are the same, except that the second transposase domain comprises an N-terminal deletion of about 100 amino acids. In some embodiments, the fusion protein comprises an amino acid sequence that is at least 75%, at least 85%, at least 90%, at least 95%, at least 98%, or at least 99% identical to the amino acid sequence set forth in SEQ ID NO: 13. In some embodiments, the fusion protein comprises the amino acid sequence set forth in SEQ ID NO: 13.

In some embodiments, the fusion protein comprises a first full-length transposase domain and a second transposase domain, wherein the first transposase domain and the second transposase domain are the same, except that the second transposase domain comprises an N-terminal deletion of about 115 amino acids. In some embodiments, the fusion protein comprises an amino acid sequence that is at least 75%, at least 85%, at least 90%, at least 95%, at least 98%, or at least 99% identical to the amino acid sequence set forth in SEQ ID NO: 14. In some embodiments, the fusion protein comprises the amino acid sequence set forth in SEQ ID NO: 14.

### DNA Targeting Domains

The transposase domains and fusion proteins provided herein may further comprise one or more DNA targeting domains. A DNA-targeting domain may be attached to the C-terminus or the N-terminus of the transposase domain or the fusion protein. In preferred embodiments, the DNA-targeting domain is attached to the N-terminus of the transposase domain, e.g., a transposase domain comprising an N-terminal deletion. Without wishing to be bound by theory, it is believed that addition a DNA targeting domain to a transposase domain improves site-specific transposase activity by targeting the transposase fused to the DNA targeting domain to the targeted site. In some embodiments, the insertion of a DNA targeting domain improves site-specific transposase activity by at least 2-fold, at least 3-fold, at least 4-fold, or at least 5-fold compared to the same transposase domain not comprising a DNA targeting domain.

Any DNA targeting domain known in the art may be used in the context of the transposase domains, fusion proteins, and tandem dimer transposases described herein, including, without limitation, CRISPR, Zinc Finger Motifs, TALE, and transcription factors. In some embodiments, the DNA targeting domain comprises three Zinc Finger Motifs. In some embodiments, the three Zinc Finger Motifs are flanked by GGGGS linkers. In some embodiments, the three Zinc Finger Motifs flanked by GGGGS linkers cumulatively comprise the sequence set forth in SEQ ID NO: 57:

(SEQ ID NO: 57)
GGGGSERPYACPVESCDRRFSRSDELTRHIRIHTGQKPFQCRICMRNFS
RSDHLTTHIRTHTGEKPFACDICGRKFARSDERKRHTKIHLRQKDGGGG
S

or a sequence having at least 75%, at least 80%, at least 85%, at least 90%, at least 95%, at least 98%, or at least 99% identity thereto.

In a specific embodiment, provided herein is a fusion protein comprising a transposase domain comprises an N-terminal deletion, an NLS, and three Zinc Finger Motifs. In some embodiments, the NLS comprises or consists of the sequence set forth in SEQ ID NO: 15.

In some aspects, the DNA targeting domain is a TAL array. TALEs (Transcription activator-like effectors) from Xanthomonas typically contain a 288 amino acid N-terminus followed by an array of a variable number of ˜34 amino acid repeats followed by a 278 amino acid C-terminus (SEQ ID NO: 77); however, truncated versions have been described in the literature (e.g., see Miller et al., Nat Biotechnol 29, 143-148 (2011). TALs fused to a FokI nuclease (called TALENs) most often contain truncations of the N and C terminus. For example, the first 152 amino acids of the N-terminus is often removed (called Delta 152; SEQ ID No 73) and the C-terminus is often truncated leaving 63 amino acids (called +63; SEQ ID NO: 76).

TALs contain arrays of 34 amino acids repeated a variable number of times. Two amino acids at position 12 and 13 are varied and determine which nucleotide the TAL repeat will recognize. This feature allows a TAL array to be programed to bind a specific DNA sequence. The amino acids NG recognize T, NI recognize A, NN recognize G or A, HD recognize C, NK recognize G, NS recognize A, C, G or T. Other amino acids within the 34 residue repeat may also be varied. For example position 11 is often changed to an N for repeats that recognize G. Also, positions 4 and 32 are often varied to reduce the repetitiveness of the array but not to determine the binding specificity. The number of 34 amino acid repeats in an array determines the length of the DNA sequence recognized (one protein repeat binds one DNA bp). Furthermore, the last bp is recognized by a “half array” that is 20 amino acids rather than 34.

In addition, the N-terminal domain of TALs (e.g., SEQ ID NO: 73) recognizes and requires a T that is located immediately 5′ of the target DNA sequence. Mutations of TAL N-terminal domains have been described in the literature that no longer require a 5′ T (Lamb et al., Nucleic Acids Res. 2013 November; 41(21):9779-85. doi: 10.1093/nar/gkt754. Epub 2013 Aug. 26. PMID: 23980031; PMCID: PMC3834825.) For example, the NT-G mutant requires a 5′G instead of a 5′T (SEQ ID NO: 74) while the NT-PN mutant does not require any specific 5′ nucleotide (SEQ ID NO: 75). These mutated N-terminal domain sequences may be used to provide additional sequence options that may be targeted using TAL Arrays.

Each TAL array comprises nine 34 amino acid repeats followed by the 20 amino acid “half” repeat and were synthesized with flanking BsmBI type IIS restriction sites. In one embodiment, individual TAL modules containing 34 amino acid or 20 amino acid “half” repeats may be designed and synthesized flanked by BsmBI type IIS restriction sites. The entire TAL module set contains 4 modules capable of recognizing either A, C, G, T for each of 10 bp positions (40 modules/10 bp target), and one TAL half repeat module. Exemplary TAL modules are set forth in SEQ ID NOs: 107-110, wherein X is any amino acid:

TAL Module Version 1:
(SEQ ID NO: 107)
LTPDQVVAIAXXXGGKQALETVQRLLPVLCQDHG
TAL Module Version 2:
(SEQ ID NO: 108)
LTPEQVVAIAXXXGGKQALETVQRLLPVLCQAHG
TAL Module Version 3”
(SEQ ID NO: 109)
LTPDQVVAIAXXXGGKQALETVQRLLPVLCQAHG
TAL Module Version 4:
(SEQ ID NO: 110)
LTPAQVVAIAXXXGGKQALETVQRLLPVLCQDHG.

An exemplary TAL Half Module is set forth in SEQ ID NO: 111, wherein X is any amino acid: LTPEQVVAIAXXXGGRPALE.

Pairs of TAL arrays targeting sequences in the desired gene may be designed and the corresponding modules selected and pooled together using “Golden Gate Assembly,” to assemble in frame each TAL-Array. The DNA sequence encoding TAL Arrays generated herein may be further codon optimized using GeneArt algorithms (Thermo Fisher).

When designing left and right TAL Arrays comprising a N-terminal domain recognizing a T and a TAL C-terminal domain to be fused to an N-terminal deleted transposase sequence (i.e., TAL-ssSPB or TAL-PBx; described below), one TAL Array recognizes a sequence 5′ of the TTAA and the other TAL Array recognizes a sequence 3′ of the TTAA. Since the sequence 5′ of TTAA is most often different from the sequence 3′ of TTAA in genomic DNA targets, TAL-ssSPB will most often be used as a heterodimer consisting of two different TAL domains that recognize two different DNA sequences. Additionally, the sequence recognized by the TAL Array is not directly adjacent to the TTAA. Instead, it is separated from the TTAA by a spacer of a given bp length, e.g., spacers of 12 bp, 13 bp or 14 bp.

A TAL array may target any DNA sequence (e.g., genomic DNA sequence) of interest. It will be apparent to a person of skill in the art that any left TAL array for a given target can be combined with any right TAL array for the same target.

In some embodiments, a TAL array targets green fluorescent protein (GFP). Illustrative sequences of left TAL arrays targeting GFP are set forth in SEQ ID NOs: 113 and 115. Illustrative sequences of right TAL arrays targeting GFP are set forth in SEQ ID NOs: 114 and 116. In some embodiments, the left TAL array targeting GFP binds to a nucleic acid molecule comprising the sequence set forth in SEQ ID NO: 240 or 242, In some embodiments, the right TAL array targeting GFP binds to a nucleic acid molecule comprising the sequence set forth in SEQ ID NO: 241 or 243.

In some embodiments, a TAL array targets ZFN268. An illustrative sequence of a TAL array targeting ZFN268, which serves as the left and the right array, is set forth in SEQ ID NO: 112. In some embodiments, the TAL array targeting ZFN268 binds to a nucleic acid molecule comprising the sequence set forth in SEQ ID NO: 239.

In some embodiments, a TAL array targets phenylalanine hydroxylase (PAH). Illustrative sequences of left TAL arrays targeting PAH are set forth in SEQ ID NOs: 117, 119, 121, 123, 125, and 127. Illustrative sequences of right TAL arrays targeting PAH are set forth in SEQ ID NOs: 118, 120, 122, 124, 126, and 128. In some embodiments, the left TAL array targeting PAH binds to a nucleic acid molecule comprising the sequence set forth in SEQ ID NO: 244, 246, 248, 250, 252, or 254. In some embodiments, the right TAL array targeting PAH binds to a nucleic acid molecule comprising the sequence set forth in SEQ ID NO: 245, 247, 249, 251, 253, or 255. Illustrative genomic target sites for PAH are set forth in SEQ ID NOs: 360-365.

In some embodiments, a TAL array targets a LINE1 repeat element. Illustrative sequences of left TAL arrays targeting a LINE1 repeat element are set forth in SEQ ID NOs: 129, 131, 134, 136, 137, 139, and 141. Illustrative sequences of right TAL arrays targeting LINE1 are set forth in SEQ ID NOs: 130, 132, 133, 135, 138, 140, 142, and 143. In some embodiments, the left TAL array targeting a LINE1 repeat element binds to a nucleic acid molecule comprising the sequence set forth in SEQ ID NO: 256, 258, 261, 263, 264, 266, or 268. In some embodiments, the right TAL array targeting a LINE1 repeat element binds to a nucleic acid molecule comprising the sequence set forth in SEQ ID NO: 257, 259, 260, 262, 265, 267, 269 or 270. Illustrative genomic target sites for a LINE1 elements are set forth in SEQ ID NOs: 366-374.

In some embodiments, a TAL array targets beta-2-microglobulin gene (B2M). Illustrative sequences of left TAL arrays targeting B2M are set forth in SEQ ID NOs: 144, 146, 148, 150, 152, 154, 156, 518 and 520. Illustrative sequences of right TAL arrays targeting B2M are set forth in SEQ ID NOs 145, 147, 149, 151, 153, 155, 157, 519, and 521. In some embodiments, the left TAL array targeting B2M binds to a nucleic acid molecule comprising the sequence set forth in SEQ ID NO: 271, 273, 275, 277, 279, 281, 283, 514, or 516. In some embodiments, the right TAL array targeting B2M binds to a nucleic acid molecule comprising the sequence set forth in SEQ ID NO: 272, 274, 276, 278, 280, 282, 284, 515, or 517. Illustrative genomic target sites for B2M are set forth in SEQ ID NOs: 375-381.

The DNA targeting domain may be fused or linked to the N-terminus of a transposase domain comprising an N-terminal deletion. For example, the DNA targeting domain may be inserted into a transposase domain at a suitable position in the N-terminal region of the transposase domain.

The DNA targeting domain may be inserted into the N-terminus of a transposase domain. In some embodiments, the DNA targeting domain is inserted between the 82^{nd} and 83^{rd} amino acid of SEQ ID NO: 55 or 56 (with numbering beginning from the 5^{th} amino acid) or SEQ ID NO: 544. In some embodiments, the DNA targeting domain is inserted between the 83^{rd} and 84^{th} amino acid of SEQ ID NO: 55 or 56 (with numbering beginning from the 5^{th} amino acid) or SEQ ID NO: 544. In some embodiments, the DNA targeting domain is inserted between the 84^{th} and 85^{th} amino acid of SEQ ID NO: 55 or 56 (with numbering beginning from the 5^{th} amino acid) or SEQ ID NO: 544. In some embodiments, the DNA targeting domain is inserted between the 85^{th} and 86^{th} amino acid of SEQ ID NO: 55 or 56 (with numbering beginning from the 5^{th} amino acid) or SEQ ID NO: 544. In some embodiments, the DNA targeting domain is inserted between the 86^{th} and 87^{th} amino acid of SEQ ID NO: 55 or 56 (with numbering beginning from the 5^{th} amino acid) or SEQ ID NO: 544. In some embodiments, the DNA targeting domain is inserted between the 87^{th} and 88^{th} amino acid of SEQ ID NO: 55 or 56 (with numbering beginning from the 5^{th} amino acid) or SEQ ID NO: 544. In some embodiments, the DNA targeting domain is inserted between the 88^{th} and 89^{th} amino acid of SEQ ID NO: 55 or 56 (with numbering beginning from the 5^{th} amino acid) or SEQ ID NO: 544. In some embodiments, the DNA targeting domain is inserted between the 89^{th} and 90^{th} amino acid of SEQ ID NO: 55 or 56 (with numbering beginning from the 5^{th} amino acid) or SEQ ID NO: 544. In some embodiments, the DNA targeting domain is inserted between the 90^{th} and 91^{st} amino acid of SEQ ID NO: 55 or 56 (with numbering beginning from the 5^{th} amino acid) or SEQ ID NO: 544. In some embodiments, the DNA targeting domain is inserted between the 91^{st} and 92^{nd} amino acid of SEQ ID NO: 55 or 56 (with numbering beginning from the 5^{th} amino acid) or SEQ ID NO: 544. In some embodiments, the DNA targeting domain is inserted between the 92^{nd} and 93^{rd} amino acid of SEQ ID NO: 55 or 56 (with numbering beginning from the 5^{th} amino acid) or SEQ ID NO: 544. In some embodiments, the DNA targeting domain is inserted between the 93^{rd} and 94^{th} amino acid of SEQ ID NO: 55 or 56 (with numbering beginning from the 5^{th} amino acid) or SEQ ID NO: 544. In some embodiments, the DNA targeting domain is inserted between the 94^{th} and 95^{th} amino acid of SEQ ID NO: 55 or 56 (with numbering beginning from the 5^{th} amino acid) or SEQ ID NO: 544. In some embodiments, the DNA targeting domain is inserted between the 95^{th} and 96^{th} amino acid of SEQ ID NO: 55 or 56 (with numbering beginning from the 5^{th} amino acid) or SEQ ID NO: 544. In some embodiments, the DNA targeting domain is inserted between the 96^{th} and 97^{th} amino acid of SEQ ID NO: 55 or 56 (with numbering beginning from the 5^{th} amino acid) or SEQ ID NO: 544. In some embodiments, the DNA targeting domain is inserted between the 97^{th} and 98^{th} amino acid of SEQ ID NO: 55 or 56 (with numbering beginning from the 5^{th} amino acid) or SEQ ID NO: 544. In some embodiments, the DNA targeting domain is inserted between the 98^{th} and 99^{th} amino acid of SEQ ID NO: 55 or 56 (with numbering beginning from the 5^{th} amino acid) or SEQ ID NO: 544. In some embodiments, the DNA targeting domain is inserted between the 99^{th} and 100^{th} amino acid of SEQ ID NO: 55 or 56 (with numbering beginning from the 5^{th} amino acid) or SEQ ID NO: 544. In some embodiments, the DNA targeting domain is inserted between the 100^{th} and 101^{st} amino acid of SEQ ID NO: 55 or 56 (with numbering beginning from the 5^{th} amino acid) or SEQ ID NO: 544. In some embodiments, the DNA targeting domain is inserted between the 101^{st} and 102^{nd} amino acid of SEQ ID NO: 55 or 56 (with numbering beginning from the 5^{th} amino acid) or SEQ ID NO: 544. In some embodiments, the DNA targeting domain is inserted between the 102^{nd} and 103^{rd} amino acid of SEQ ID NO: 55 or 56 (with numbering beginning from the 5^{th} amino acid) or SEQ ID NO: 544. In some embodiments, the DNA targeting domain is inserted between the 103^{rd} and 104^{th} amino acid of SEQ ID NO: 55 or 56 (with numbering beginning from the 5^{th} amino acid) or SEQ ID NO: 544. In some embodiments, the DNA targeting domain is inserted between the 104 and 105^{th} amino acid of SEQ ID NO: 55 or 56 (with numbering beginning from the 5^{th} amino acid) or SEQ ID NO: 544. In some embodiments, the DNA targeting domain comprises the sequence of SEQ ID NO: 57 or a sequence having at least 75%, at least 80%, at least 85%, at least 90%, at least 95%, at least 98%, or at least 99% identity thereto. The transposase domain may further comprise an NLS, for example, and NLS of SEQ ID NO: 15.

In some embodiments, the DNA targeting domain replaces the 83^{rd} amino acid of SEQ ID NO: 55 or 56 (with numbering beginning from the 5^{th} amino acid) or of SEQ ID NO: 544. In some embodiments, the DNA targeting domain replaces the 84^{th} amino acid of SEQ ID NO: 55 or 56 (with numbering beginning from the 5^{th} amino acid) or of SEQ ID NO: 544. In some embodiments, the DNA targeting domain replaces the 85^{th} amino acid of SEQ ID NO: 55 or 56 (with numbering beginning from the 5^{th} amino acid) or of SEQ ID NO: 544. In some embodiments, the DNA targeting domain replaces the 86^{th} amino acid of SEQ ID NO: 55 or 56 (with numbering beginning from the 5^{th} amino acid) or of SEQ ID NO: 544. In some embodiments, the DNA targeting domain replaces the 87^{th} amino acid of SEQ ID NO: 55 or 56 (with numbering beginning from the 5^{th} amino acid) or of SEQ ID NO: 544. In some embodiments, the DNA targeting domain replaces the 88^{th} amino acid of SEQ ID NO: 55 or 56 (with numbering beginning from the 5^{th} amino acid) or of SEQ ID NO: 544. In some embodiments, the DNA targeting domain replaces the 89^{th} amino acid of SEQ ID NO: 55 or 56 (with numbering beginning from the 5^{th} amino acid) or of SEQ ID NO: 544. In some embodiments, the DNA targeting domain replaces the 90^{th} amino acid of SEQ ID NO: 55 or 56 (with numbering beginning from the 5^{th} amino acid) or of SEQ ID NO: 544. In some embodiments, the DNA targeting domain replaces the 91^{st} amino acid of SEQ ID NO: 55 or 56 (with numbering beginning from the 5^{th} amino acid) or of SEQ ID NO: 544. In some embodiments, the DNA targeting domain replaces the 92^{nd} amino acid of SEQ ID NO: 55 or 56 (with numbering beginning from the 5^{th} amino acid) or of SEQ ID NO: 544. In some embodiments, the DNA targeting domain replaces the 93^{rd} amino acid of SEQ ID NO: 55 or 56 (with numbering beginning from the 5^{th} amino acid) or of SEQ ID NO: 544. In some embodiments, the DNA targeting domain replaces the 94^{th} amino acid of SEQ ID NO: 55 or 56 (with numbering beginning from the 5^{th} amino acid) or of SEQ ID NO: 544. In some embodiments, the DNA targeting domain replaces the 95^{th} amino acid of SEQ ID NO: 55 or 56 (with numbering beginning from the 5^{th} amino acid) or of SEQ ID NO: 544. In some embodiments, the DNA targeting domain replaces the 96^{th} amino acid of SEQ ID NO: 55 or 56 (with numbering beginning from the 5^{th} amino acid) or of SEQ ID NO: 544. In some embodiments, the DNA targeting domain replaces the 97^{th} amino acid of SEQ ID NO: 55 or 56 (with numbering beginning from the 5^{th} amino acid) or of SEQ ID NO: 544. In some embodiments, the DNA targeting domain replaces the 98^{th} amino acid of SEQ ID NO: 55 or 56 (with numbering beginning from the 5^{th} amino acid) or of SEQ ID NO: 544. In some embodiments, the DNA targeting domain replaces the 99^{th} amino acid of SEQ ID NO: 55 or 56 (with numbering beginning from the 5^{th} amino acid) or of SEQ ID NO: 544. In some embodiments, the DNA targeting domain replaces the 100^{th} amino acid of SEQ ID NO: 55 or 56 (with numbering beginning from the 5^{th} amino acid) or of SEQ ID NO: 544. In some embodiments, the DNA targeting domain replaces the 101^{st} amino acid of SEQ ID NO: 55 or 56 (with numbering beginning from the 5^{th} amino acid) or of SEQ ID NO: 544. In some embodiments, the DNA targeting domain replaces the 102^{nd} amino acid of SEQ ID NO: 55 or 56 (with numbering beginning from the 5^{th} amino acid) or of SEQ ID NO: 544. In some embodiments, the DNA targeting domain replaces the 103^{rd} amino acid of SEQ ID NO: 55 or 56 (with numbering beginning from the 5^{th} amino acid) or of SEQ ID NO: 544. In some embodiments, the DNA targeting domain replaces the 104^{th} amino acid of SEQ ID NO: 55 or 56 (with numbering beginning from the 5^{th} amino acid) or of SEQ ID NO: 544. In some embodiments, the DNA targeting domain replaces the 105^{th} amino acid of SEQ ID NO: 55 or 56 (with numbering beginning from the 5^{th} amino acid) or of SEQ ID NO: 544. In some embodiments, the DNA targeting domain comprises the sequence of SEQ ID NO: 57 or a sequence having at least 75%, at least 80%, at least 85%, at least 90%, at least 95%, at least 98%, or at least 99% identity thereto. The transposase domain may further comprise an NLS, for example, and NLS of SEQ ID NO: 15.

An exemplary sequence of a fusion protein comprising a transposase domain comprising an N-terminal deletion of 93 amino acids, an NLS, and three Zinc Finger Motifs flanked by GGGGS linkers is show in SEQ ID NO: 58, where the NLS is shown in italics, the sequence comprising the three Zinc Finger Motifs and GGGGS linkers is underlined, and the transposase domain comprising an N-terminal deletion of 93 amino acid is shown in bold:

(SEQ ID NO: 58)
MAPKKKRKVGGGGSERPYACPVESCDRRFSRSDELTRHIRIHTGQKPFQ
CRICMRNFSRSDHLTTHIRTHTGEKPFACDICGRKFARSDERKRHTKIH
LRQKDGGGGSNKHCWSTSKSTRRSRVSALNIVRSQRGPTRMCRNIYDPL
LCFKLFFTDEIISEIVKWTNAEISLKRRESMTSATFRDTNEDEIYAFFG
ILVMTAVRKDNHMSTDDLFDRSLSMVYVSVMSRDRFDFLIRCLRMDDKS
IRPTLRENDVFTPVRKIWDLFIHQCIQNYTPGAHLTIDEQLLGFRGRCP
FRVYIPNKPSKYGIKILMMCDSGTKYMINGMPYLGRGTQTNGVPLGEYY
VKELSKPVHGSCRNITCDNWFTSIPLAKNLLQEPYKLTIVGTVASNARE
IPEVLKNSRSRPVGTSMFCFDGPLTLVSYKPKPAKMVYLLSSCDEDASI
NESTGKPQMVMYYNQTKGGVDTLNQMCSVMTCSRKTNRWPMALLYGMIN
IACINSFIIYSHNVSSKGEKVQSRKKFMRNLYMSLTSSFMRKRLEAPTL
KRYLRDNISNILPKEVPGTSDDSTEEPVMKKRTYCTYCPSKIRRKANAS
CKKCKKVICREHNIDMCQSCF

An exemplary sequence of a fusion protein comprising an integration deficient transposase domain comprising an N-terminal deletion of 93 amino acids, an NLS, and three Zinc Finger Motifs flanked by GGGGS linkers is set forth in SEQ ID NO: 59, where the NLS is shown in italics, the sequence comprising the three Zinc Finger Motifs and GGGGS linkers is underlined, and the transposase domain comprising an N-terminal deletion of 93 amino acid is shown in bold:

(SEQ ID NO: 59)
MAPKKKRKVGGGGSERPYACPVESCDRRFSRSDELTRHIRIHTGQKPFQ
CRICMRNFSRSDHLTTHIRTHTGEKPFACDICGRKFARSDERKRHTKIH
LRQKDGGGGSNKHCWSTSKSTRRSRVSALNIVRSQRGPTRMCRNIYDPL
LCFKLFFTDEIISEIVKWTNAEISLKRRESMTSATFRDTNEDEIYAFFG
ILVMTAVRKDNHMSTDDLFDRSLSMVYVSVMSRDRFDFLIRCLRMDDKS
IRPTLRENDVFTPVRKIWDLFIHQCIQNYTPGAHLTIDEQLLGFRGRCP
FRVYIPNKPSKYGIKILMMCDSGTKYMINGMPYLGRGTQTNGVPLGEYY
VKELSKPVHGSCRNITCDNWFTSIPLAKNLLQEPYKLTIVGTVASNARE
IPEVLKNSRSRPVGTSMFCFDGPLTLVSYKPKPAKMVYLLSSCDEDASI
NESTGKPQMVMYYNQTKGGVDTLNQMCSVMTCSRKTNRWPMALLYGMIN
IACINSFIIYSHNVSSKGEKVQSRKKFMRNLYMSLTSSFMRKRLEAPTL
KRYLRDNISNILPKEVPGTSDDSTEEPVMKKRTYCTYCPSKIRRKANAS
CKKCKKVICREHNIDMCQSCF.

### Protein Stabilization Domains

In some embodiments, a fusion protein provided herein may further comprise a protein stabilization domain (PSD). The PSD is preferably attached to the N-terminus of the DNA targeting domain, if present. Without wishing to be bound by theory, it is believed that the addition of a PSD can enhance protein stability or enhanced stability of the transposase tetramer—DNA complex.

The PSD may be of approximately the same size as the N-terminal deletion in the transposase domain. For example, in some embodiments, the N-terminal deletion of transposase domain comprises amino acids 1-93, and the PSD comprises 92 amino acids.

In some embodiments, the PSD comprises amino acids 1-90 of SEQ ID NO: 55 (with numbering beginning at residue 5 of SEQ ID NO: 55). In some embodiments, the PSD comprises amino acids 1-90 of SEQ ID NO: 56 (with numbering beginning at residue 5 of SEQ ID NO: 56). In some embodiments, the PSD comprises amino acids 1-91 of SEQ ID NO: 55 (with numbering beginning at residue 5 of SEQ ID NO: 55). In some embodiments, the PSD comprises amino acids 1-91 of SEQ ID NO: 56 (with numbering beginning at residue 5 of SEQ ID NO: 56). In some embodiments, the PSD comprises amino acids 1-92 of SEQ ID NO: 55 (with numbering beginning at residue 5 of SEQ ID NO: 55). In some embodiments, the PSD comprises amino acids 1-92 of SEQ ID NO: 56 (with numbering beginning at residue 5 of SEQ ID NO: 56). In some embodiments, the PSD comprises amino acids 1-93 of SEQ ID NO: 55 (with numbering beginning at residue 5 of SEQ ID NO: 55). In some embodiments, the PSD comprises amino acids 1-93 of SEQ ID NO: 56 (with numbering beginning at residue 5 of SEQ ID NO: 56). In some embodiments, the PSD comprises amino acids 1-94 of SEQ ID NO: 55 (with numbering beginning at residue 5 of SEQ ID NO: 55). In some embodiments, the PSD comprises amino acids 1-94 of SEQ ID NO: 56 (with numbering beginning at residue 5 of SEQ ID NO: 56). In some embodiments, the PSD comprises amino acids 1-95 of SEQ ID NO: 55 (with numbering beginning at residue 5 of SEQ ID NO: 55). In some embodiments, the PSD comprises amino acids 1-95 of SEQ ID NO: 56 (with numbering beginning at residue 5 of SEQ ID NO: 56). In some embodiments, the PSD comprises amino acids 1-96 of SEQ ID NO: 55 (with numbering beginning at residue 5 of SEQ ID NO: 55). In some embodiments, the PSD comprises amino acids 1-96 of SEQ ID NO: 56 (with numbering beginning at residue 5 of SEQ ID NO: 56). In some embodiments, the PSD comprises amino acids 1-97 of SEQ ID NO: 55 (with numbering beginning at residue 5 of SEQ ID NO: 55). In some embodiments, the PSD comprises amino acids 1-97 of SEQ ID NO: 56 (with numbering beginning at residue 5 of SEQ ID NO: 56). In some embodiments, the PSD comprises amino acids 1-98 of SEQ ID NO: 55 (with numbering beginning at residue 5 of SEQ ID NO: 55). In some embodiments, the PSD comprises amino acids 1-98 of SEQ ID NO: 56 (with numbering beginning at residue 5 of SEQ ID NO: 56). In some embodiments, the PSD comprises amino acids 1-99 of SEQ ID NO: 55 (with numbering beginning at residue 5 of SEQ ID NO: 55). In some embodiments, the PSD comprises amino acids 1-99 of SEQ ID NO: 56 (with numbering beginning at residue 5 of SEQ ID NO: 56). In some embodiments, the PSD comprises amino acids 1-100 of SEQ ID NO: 55 (with numbering beginning at residue 5 of SEQ ID NO: 55). In some embodiments, the PSD comprises amino acids 1-100 of SEQ ID NO: 56 (with numbering beginning at residue 5 of SEQ ID NO: 56).

In some embodiments, the PSD comprises the sequence

(SEQ ID NO: 68)
GSSLDDEHILSALLQSDDELVGEDSDSEVSDHVSEDDVQSDTEEAFIDE
VHEVQPTSSGSEILDEQNVIEQPGSSLASNRILTLPQRTIRG.

Thus, provided herein are fusion proteins comprising, in N-terminal to C-terminal order: a nuclear localization signal (NLS), PSD, a DNA targeting domain, and a transposase domain comprising an N-terminal deletion as compared to the sequence set forth in SEQ ID NO: 55 or 56 (with numbering beginning at residue 5 of SEQ ID NO: 55 or 56).

Exemplary sequences of fusion proteins comprising a PSD, an NLS, a DNA targeting domain and a transposase domain comprising an N-terminal deletion are shown in SEQ ID NOs: 67 (PBx transposase domain) and 69 (SPB transposase domain) with the NLS (here: PKKKRKV) shown in italics, the NTD shown in bold and underlined, the DNA targeting domain (here: three Zinc Finger Motifs flanked by GGGGS linkers) underlined, and the N-terminally deleted transposase domain (here: PBx) shown in bold:

(SEQ ID NO: 67)
MAPKKKRKVGGGGSSLDDEHILSALLQSDDELVGEDSDSEVSDHVSEDDVQSDTEE
AFIDEVHEVQPTSSGSEILDEQNVIEQPGSSLASNRILTLPQRTIRGGGGGSERPYACP
VESCDRRFSRSDELTRHIRIHTGQKPFQCRICMRNFSRSDHLTTHIRTHTGEKPFACDIC
GRKFARSDERKRHTKIHLRQKDGGGGSNKHCWSTSKSTRRSRVSALNIVRSQRGP
TRMCRNIYDPLLCFKLFFTDEIISEIVKWTNAEISLKRRESMTSATFRDTNEDEIY
AFFGILVMTAVRKDNHMSTDDLFDRSLSMVYVSVMSRDRFDFLIRCLRMDDKSI
RPTLRENDVFTPVRKIWDLFIHQCIQNYTPGAHLTIDEQLLGFRGRCPFRVYIPN
KPSKYGIKILMMCDSGTKYMINGMPYLGRGTQTNGVPLGEYYVKELSKPVHGS
CRNITCDNWFTSIPLAKNLLQEPYKLTIVGTVASNAREIPEVLKNSRSRPVGTSMF
CFDGPLTLVSYKPKPAKMVYLLSSCDEDASINESTGKPQMVMYYNQTKGGVDT
LNQMCSVMTCSRKTNRWPMALLYGMINIACINSFIIYSHNVSSKGEKVQSRKKF
MRNLYMSLTSSFMRKRLEAPTLKRYLRDNISNILPKEVPGTSDDSTEEPVMKKR
TYCTYCPSKIRRKANASCKKCKKVICREHNIDMCQSCF.
(SEQ ID NO: 69)
MAPKKKRKVGGGGSSLDDEHILSALLQSDDELVGEDSDSEVSDHVSEDDV
QSDTEEAFIDEVHEVQPTSSGSEILDEQNVIEQPGSSLASNRILTLPQRTIRGGGGGSE
RPYACPVESCDRRFSRSDELTRHIRIHTGQKPFQCRICMRNFSRSDHLTTHIRTHTGEKPF
ACDICGRKFARSDERKRHTKIHLRQKDGGGGSNKHCWSTSKSTRRSRVSALNIVRSQ
RGPTRMCRNIYDPLLCFKLFFTDEIISEIVKWTNAEISLKRRESMTSATFRDTNED
EIYAFFGILVMTAVRKDNHMSTDDLFDRSLSMVYVSVMSRDRFDFLIRCLRMDD
KSIRPTLRENDVFTPVRKIWDLFIHQCIQNYTPGAHLTIDEQLLGFRGRCPFRVY
IPNKPSKYGIKILMMCDSGTKYMINGMPYLGRGTQTNGVPLGEYYVKELSKPV
HGSCRNITCDNWFTSIPLAKNLLQEPYKLTIVGTVRSNKREIPEVLKNSRSRPVG
TSMFCFDGPLTLVSYKPKPAKMVYLLSSCDEDASINESTGKPQMVMYYNQTKG
GVDTLDQMCSVMTCSRKTNRWPMALLYGMINIACINSFIIYSHNVSSKGEKVQS
RKKFMRNLYMSLTSSFMRKRLEAPTLKRYLRDNISNILPKEVPGTSDDSTEEPV
MKKRTYCTYCPSKIRRKANASCKKCKKVICREHNIDMCQSCF.

### Nuclear Localization Signals

In some embodiments, the transposase domains and fusion proteins provided herein may comprise an in-frame nuclear localization sequence (NLS). Examples of transposases fused to a nuclear localization signal are disclosed in U.S. Pat. Nos. 6,218,185; 6,962,810, 8,399,643 and WO 2019/173636. In some embodiments, the NLS comprises the sequence of PKKKRKV (SEQ ID NO: 15). In certain aspects, the in-frame NLS is located upstream (N-terminal) of the transposase domain comprising an N-terminal deletion.

In general, the NLS is preferably located at the N-terminal end of a fusion protein. In some embodiments, the NLS is fused or linked to the N-terminus of a transposase domain. In some embodiments, the NLS is fused or linked to the N-terminus of a DNA targeting domain. In some embodiments, the NLS is fused or linked to the N-terminus of a PSD.

In certain aspects, the in-frame NLS is fused directly to the amino terminus of the transposase domain comprising an N-terminal deletion. In some embodiments, the NLS is attached to the N-terminus of a transposase domain comprising an N-terminal deletion via a linker (e.g., a GGGGS linker or a GGS linker).

In some embodiments, an initiator methionine is introduced before the NLS. In some embodiments, additional alanine residues are introduced before and/or after the NLS to ensure in-frame translation. As such, the numbering of the residues in SEQ ID NO: 1 begins at the 12^{th} residue of SEQ ID NO: 1 for the purpose of identifying deleted and mutated residues. In SEQ ID NOs: 55 and 56, which are the sequence of SPB and PBx, respectively, which do not comprise an NLS, the numbering of residues begins at the 5^{th} residue for the purpose of identifying deleted and mutated residues. In SEQ ID NO: 544, the numbering begins at the first residue for the purpose of identifying deleted and mutated residues.

In some embodiments, a fusion protein comprises an NLS and a transposase domain comprising an N-terminal deletion of 20 amino acids. In some embodiments, the fusion protein comprises an amino acid sequence that is at least 75%, at least 80%, at least 85%, at least 90%, at least 95%, at least 98% or at least 99% identical to the amino acid sequence set forth in SEQ ID NO: 2. In some embodiments, the fusion protein comprises the amino acid sequence set forth in SEQ ID NO: 2.

In some embodiments, a fusion protein comprises an NLS and a transposase domain comprising an N-terminal deletion of 40 amino acids. In some embodiments, the fusion protein comprises an amino acid sequence that is at least 75%, at least 80%, at least 85%, at least 90%, at least 95%, at least 98% or at least 99% identical to the amino acid sequence set forth in SEQ ID NO: 3. In some embodiments, the fusion protein comprises the amino acid sequence set forth in SEQ ID NO: 3.

In some embodiments, a fusion protein comprises an NLS and a transposase domain comprising an N-terminal deletion of 60 amino acids. In some embodiments, the fusion protein comprises an amino acid sequence that is at least 75%, at least 80%, at least 85%, at least 90%, at least 95%, at least 98% or at least 99% identical to the amino acid sequence set forth in SEQ ID NO: 4. In some embodiments, the fusion protein comprises the amino acid sequence set forth in SEQ ID NO: 4.

In some embodiments, a fusion protein comprises an NLS and a transposase domain comprising es an N-terminal deletion of 80 amino acids. In some embodiments, the fusion protein comprises an amino acid sequence that is at least 75%, at least 80%, at least 85%, at least 90%, at least 95%, at least 98% or at least 99% identical to the amino acid sequence set forth in SEQ ID NO: 5. In some embodiments, the fusion protein comprises the amino acid sequence set forth in SEQ ID NO: 5.

In some embodiments, a fusion protein comprises an NLS and a transposase domain comprising an N-terminal deletion of 100 amino acids. In some embodiments, the fusion protein comprises an amino acid sequence that is at least 75%, at least 80%, at least 85%, at least 90%, at least 95%, at least 98% or at least 99% identical to the amino acid sequence set forth in SEQ ID NO: 6. In some embodiments, the fusion protein comprises the amino acid sequence set forth in SEQ ID NO: 6.

In some embodiments, a fusion protein comprises an NLS and a transposase domain comprising an N-terminal deletion of 115 amino acids. In some embodiments, the fusion protein comprises an amino acid sequence that is at least 75%, at least 80%, at least 85%, at least 90%, at least 95%, at least 98% or at least 99% identical to the amino acid sequence set forth in SEQ ID NO: 7. In some embodiments, the fusion protein comprises the amino acid sequence set forth in SEQ ID NO: 7.

In some embodiments, a fusion protein comprises an NLS and a transposase domain comprising an N-terminal deletion of 93 amino acids. In some embodiments, the fusion protein comprises an amino acid sequence that is at least 75%, at least 80%, at least 85%, at least 90%, at least 95%, at least 98% or at least 99% identical to the amino acid sequence set forth in SEQ ID NO: 65. In some embodiments, the fusion protein comprises the amino acid sequence set forth in SEQ ID NO: 65.

### Obligate Heterodimers and Tandem Dimers

In another aspect, provided herein are tandem dimer transposases comprising two fusion proteins, each fusion protein comprising a first and a second transposase domain and one or both fusion proteins further comprising a DNA targeting domain. In some embodiments, both fusion proteins comprise a DNA targeting domain. In some embodiments, both fusion proteins comprise DNA targeting domains and the DNA targeting domains target DNA sequences that are adjacent to the DNA sequence which is the insertion site targeted by the transposase. In some embodiments, only one of the two fusion proteins in the tandem dimer transposase comprises a DNA targeting domain. A DNA-targeting domain may be attached to the C-terminus or the N-terminus of the fusion protein.

Thus, in some embodiments, provided herein is a complex comprising (a) a first fusion protein comprising a first transposase domain, a linker, a second transposase domain, and a first DNA targeting domain, wherein (i) the first and second transposase domain are the same; or (ii) the first and second transposase domain are the same, except that the second transposase domain comprises an N-terminal deletion; and (b) a second fusion protein comprising a first transposase domain, a linker, a second transposase domain, and a second DNA targeting domain, wherein (i) the first and second transposase domain are the same; or (ii) the first and second transposase domain are the same, except that the second transposase domain comprises an N-terminal deletion; wherein the first DNA targeting domain and the second DNA targeting domain are different; wherein the transposase domains of the first fusion protein and the transpose domains of the second fusion protein have opposing charge that permits the two fusion proteins to form a complex.

In some embodiments, provided herein is a complex comprising (a) a first fusion protein comprising, in N-terminal to C-terminal order: a first NLS, a first DNA targeting domain, a first transposase domain comprising an N-terminal deletion, a linker, and a second transposase domain; and (b) a second fusion protein comprising in N-terminal to C-terminal order: a second NLS, a second DNA targeting domain, a third transposase domain comprising an N-terminal deletion, a linker, and a fourth transposase domain; wherein the transposase domains of the first fusion protein and the transpose domains of the second fusion protein have opposing charge that permits the two fusion proteins to form a complex. In some embodiments, the first, second, third, and/or fourth transposase domains are SPB domains. In some embodiments, the first, second, third, and/or fourth transposase domains are PBx transposase domains. In some embodiments, the first and/or third transposase domain comprises an N-terminal deletion of 83, 84, 85, 86, 87, 88, 89, 90, 91, 21, 93, 94, 95, 96, 97, 98, 99, 100, 101, 102, or 103 amino acids. In some embodiments, the first and third transposase domains comprise the sequence of SEQ ID NO: 65 or 66. In some embodiments the second and fourth transposase domains comprise the sequence of SEQ ID NO: 55, 56, or 544. In some embodiment, the first and/or second DNA targeting domain comprises three Zinc Fingers Motifs. In some embodiments, the first and/or second DNA targeting domain comprises the sequence of SEQ ID NO: 57. In some embodiment, the first and/or second DNA targeting domain comprises TAL motifs.

In some embodiments, provided herein is a complex comprising (a) a first fusion protein comprising, in N-terminal to C-terminal order: a first NLS, a first PSD, a first DNA targeting domain, a first transposase domain comprising an N-terminal deletion, a linker, and a second transposase domain; and (b) a second fusion protein comprising in N-terminal to C-terminal order: a second NLS, a second PSD, a second DNA targeting domain, a third transposase domain comprising an N-terminal deletion, a linker, and a fourth transposase domain; wherein the transposase domains of the first fusion protein and the transpose domains of the second fusion protein have opposing charge that permits the two fusion proteins to form a complex. In some embodiments, the first, second, third, and/or fourth transposase domains are SPB domains. In some embodiments, the first, second, third, and/or fourth transposase domains are PBx transposase domains. In some embodiments, the first and third transposase domains comprise the sequence of SEQ ID NO: 65 or 66. In some embodiments the second and fourth transposase domains comprise the sequence of SEQ ID NO: 55, 56, or 544. In some embodiments, the first and/or second PSD comprises the sequence of SEQ ID NO: 68. In some embodiment, the first and/or second DNA targeting domain comprises three Zinc Fingers Motifs. In some embodiments, the first and/or second DNA targeting domain comprises the sequence of SEQ ID NO: 57.

In some embodiments, provided herein is a complex comprising (a) a first fusion protein comprising, in N-terminal to C-terminal order: a first NLS, a first transposase domain comprising the sequence of SEQ ID NO: 55, 56, or 544, a linker, a second transposase domain; and (b) a second fusion protein comprising in N-terminal to C-terminal order: a second NLS, a third transposase domain comprising the sequence of SEQ ID NO: 55, 56, or 544, a linker, and a fourth transposase domain; wherein the first and the third transposase domain comprise a DNA targeting domain, and wherein the transposase domains of the first fusion protein and the transpose domains of the second fusion protein have opposing charge that permits the two fusion proteins to form a complex. In some embodiments, the second and/or fourth transposase domains are SPB domains. In some embodiments, the, second and/or fourth transposase domains are PBx transposase domains. In some embodiments the second and fourth transposase domains comprise the sequence of SEQ ID NO: 55, 56, or 544. In some embodiments, the first and/or second PSD comprises the sequence of SEQ ID NO: 68. In some embodiment, the first and/or second DNA targeting domain comprises three Zinc Fingers Motifs. In some embodiments, the first and/or second DNA targeting domain comprises the sequence of SEQ ID NO: 57. In some embodiments the first DNA targeting domain replaces the 83^{rd}, 84^{th}, 85^{th}, 86^{th}, 87^{th}, 88^{th}, 89^{th}, 90^{th}, 91^{st}, 92^{nd}, 93^{rd}, 94^{th}, 95^{th}, 96^{th}, 97^{th}, 98^{th}, 99^{th}, 100^{th}, 101^{st}, 102^{nd}, or 103^{rd} residue of the first transposase domain, with numbering beginning at residue 5 of SEQ ID NO: 55 or 56. In some embodiments, the second DNA targeting domain replaces the 83^{rd}, 84^{th}, 85^{th}, 86^{th}, 87^{th}, 88^{th}, 89^{th}, 90^{th}, 91^{st}, 92^{nd}, 93^{rd}, 94^{th}, 95^{th}, 96^{th}, 97^{th}, 98^{th}, 99^{th}, 100^{th}, 101^{st}, 102^{nd}, or 103^{rd} residue of the third transposase domain, with numbering beginning at residue 5 of SEQ ID NO: 55 or 56.

In some embodiments, provided herein is a complex comprising (a) a first fusion protein comprising a first transposase domain, a linker, a second transposase domain, and a first DNA targeting domain, wherein the first and/or the second transposase domain of the first fusion protein comprise the same amino acid sequence set forth in any one of SEQ ID NOs: 31-43; and (b) a second fusion protein comprising a first transposase domain, a linker, a second transposase domain, and a second DNA targeting domain, wherein the first and/or the second transposase domain of the second fusion protein comprise the same amino acid sequence set forth in any one of SEQ ID NOs: 44-53.

In another aspect, provided herein are fusion proteins comprising a first transposase domain and a second transposase domain that can form obligate heterodimers with another fusion protein comprising a first transposase domain and a second transposase domain. Without wishing to be bound by theory, it is believed that two such fusion protein assemble into a tandem dimer structure held together through a combination of charge interactions, hydrogen bonds, pi-cation pairs, and hydrophobic interactions. Such a tandem dimer structure is referred to herein as a “tandem dimer transposase.” Thus, each tandem dimer comprises four transposase domains. In some embodiments, two fusion proteins provided herein form a complex, said complex comprising (a) a first fusion protein comprising a first transposase domain, a linker, and a second transposase domain; and (b) a second fusion protein comprising a first transposase domain, a linker, and a second transposase domain; wherein the transposase domains of the first fusion protein and the transpose domains of the second fusion protein have opposing charge that permits the two fusion proteins to form a complex.

In some embodiments, the first fusion protein comprises a first transposase domain of SEQ ID NO: 55 and/or a second transposase domain of SEQ ID NO: 65. In some embodiments, the second fusion protein comprises a first transposase domain of SEQ ID NO: 55 and/or a second transposase domain of SEQ ID NO: 65. In some embodiments, the first fusion protein comprises a first transposase domain of SEQ ID NO: 55 and/or a second transposase domain of SEQ ID NO: 66. In some embodiments, the second fusion protein comprises a first transposase domain of SEQ ID NO: 55 and/or a second transposase domain of SEQ ID NO: 66. In some embodiments, the first fusion protein comprises a first transposase domain of SEQ ID NO: 55 and/or a second transposase domain of SEQ ID NO: 67. In some embodiments, the second fusion protein comprises a first transposase domain of SEQ ID NO: 55 and/or a second transposase domain of SEQ ID NO: 67.

In some embodiments, the first fusion protein comprises a first transposase domain of SEQ ID NO: 56 and/or a second transposase domain of SEQ ID NO: 65. In some embodiments, the second fusion protein comprises a first transposase domain of SEQ ID NO: 56 and/or a second transposase domain of SEQ ID NO: 65. In some embodiments, the first fusion protein comprises a first transposase domain of SEQ ID NO: 56 and/or a second transposase domain of SEQ ID NO: 66. In some embodiments, the second fusion protein comprises a first transposase domain of SEQ ID NO: 56 and/or a second transposase domain of SEQ ID NO: 66. In some embodiments, the first fusion protein comprises a first transposase domain of SEQ ID NO: 56 and/or a second transposase domain of SEQ ID NO: 67. In some embodiments, the second fusion protein comprises a first transposase domain of SEQ ID NO: 56 and/or a second transposase domain of SEQ ID NO: 67.

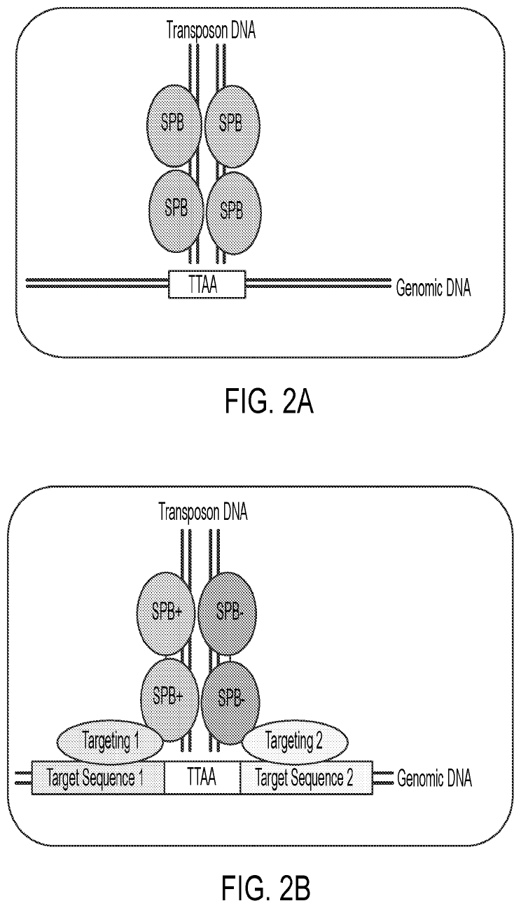

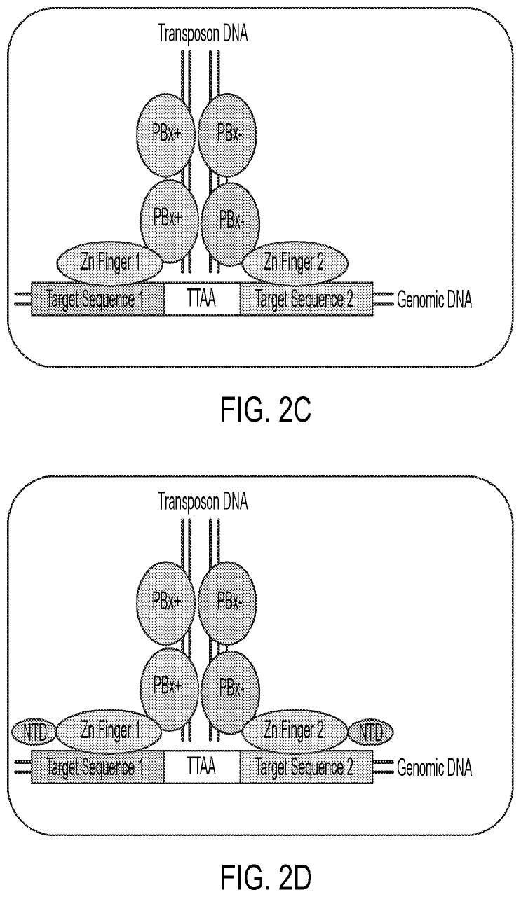

By introducing charged residues into the amino acids that contribute to the dimerization with a second fusion protein, it is possible to design pairs of fusion proteins that can only associate with each other into a tandem dimer in a predetermined configuration. By introducing mutations that only allow for one configuration of the tandem dimer, it becomes feasible to introduce DNA targeting domains into the fusion proteins, thus increasing specificity of the transposase domains. This is illustrated in FIGS. 2A and 2B for SPB and in FIGS. 2C and 2D for PBx: Introducing DNA targeting domains into fusion proteins that can dimerize in any configuration, including homodimerization, would lead to four DNA targeting domains being present in a tandem dimer transposase. However, only two DNA targeting domains would interact with the DNA, leaving the other two to potentially sterically hinder the transposase-DNA interaction. Any suitable DNA targeting domain described herein or known in the art may be used in the fusion proteins described herein.

A person of skill in the art will readily be able to determine mutations in the transposase domains that confer a positive or negative charge. In the case of a fusion protein comprising a first and second transposase domain, the crystal structure published in Chen et al. (Nat Commun 11, 3446 (2020)) may be used to identify residue pairs in the transposase domains that are in close proximity in the tandem dimer formed by two such fusion proteins. Changing the charge of such residue pairs to create a positively charged transposase domain and a negatively charged transposase domain can be accomplished using standard techniques, such as site-directed mutagenesis.

For example, one or more of M185, R189, K190, D191, H193, M194, D198, D201, S203, L204, 5205, V207, K500, R504, K575, K576, R583, N586, I587, D588, M589, C593, and/or F594 may be mutated in an SPB transposase domain (e.g., the SPB set forth in SEQ ID NO: 1 or 55, with numbering beginning at the 12^{th} residue of SEQ ID NO: 1 and at the 5^{th} residue of SEQ ID NO: 55) to generate an SPB− or an SPB+ transposase domain. Similarly, one or more of M185, R189, K190, D191, H193, M194, D198, D201, S203, L204, S205, V207, K500, R504, K575, K576, R583, N586, I587, D588, M589, C593, and/or F594 may be mutated in a PBx transposase domain (e.g., the PBx transposase domain of SEQ ID NO: 56 with numbering beginning at the 5^{th} residue of SEQ ID NO: 56, or the PBx transposase domain of SEQ ID NO: 544) to generate a PBx− or a PBx+ transposase domain.

A fusion protein described herein may comprise (i) one or two SPB+ transposase domains, or (ii) one or two SPB− transposase domains.

To accomplish formation of an obligate heterodimer, pairs of mutations may be introduced into fusion proteins or transposase domains to generate positive and negatively charged fusion proteins or transposase domains which can then interact for form a heterodimer. In some embodiments, the residue pair being mutated is one set forth in Table 2. For example, one or more of the mutations listed in the column labeled “Protein 1” may be introduced into a first SPB or PBx domain and the corresponding mutation or mutations listed in the column labeled “Protein 2” may be introduced into a second SPB or PBs domain. In some embodiments, the members of a residue pair are mutated to have opposing charges.

TABLE 2
Exemplary Residue Pairs; numbering begins at residue 5 of SEQ ID NO: 55 or 56
or residue 12 of SEQ ID NO: 1.

| Protein 1 | Protein 2 | Protein 1 | Protein 2 | Protein 1 | Protein 2 |
|---|---|---|---|---|---|
| M185 | L204 | D201 | R504 | R583 | D588 |
| R189 | R189 | S203 | R504 | N586 | D588 |
| R189 | D191 | L204 | R189 | I587 | R583 |
| R189 | M194 | L204 | L204 | I587 | I587 |
| R189 | L204 | L204 | S205 | D588 | I587 |
| K190 | K190 | L204 | R504 | D588 | D588 |
| K190 | H193 | S205 | L204 | D588 | M589 |
| K190 | M194 | V207 | S203 | M589 | M589 |
| D191 | R189 | V207 | L204 | M589 | F594 |
| H193 | K190 | K500 | D198 | C593 | M589 |
| M194 | R189 | R504 | D201 | F594 | K575 |
| M194 | K190 | K575 | F594 | F594 | K576 |
| D198 | K500 | K576 | F594 | F594 | M589 |

To introduce a positive charge, amino acids with uncharged side chains, such as methionine, or amino acids with a negatively charged side chain, such as aspartic acid, may be changed to positively charged amino acids, such as lysine or arginine. To introduce a negative charge, amino acids with positively charged side chains, such as arginine or lysine, or amino acids with hydrophobic side chains, such as leucine, may be changed to negatively charged amino acids, such as aspartic acid or glutamic acid.

In certain embodiments, one or more of the following mutations is/are introduced into one or both SPB transposase domains (e.g., the SPB set forth in SEQ ID NO: 1 or 55, with numbering beginning at the 12^{th} residue of SEQ ID NO: 1 and at the 5^{th} residue of SEQ ID NO: 55) of a fusion protein provided herein to generate an SPB+ fusion protein: M185R, M185K, D197K, D197R, D198K, D198R, D201K, and D201R. In some embodiments, an SPB+ transposase domain comprises an M185R mutation and a D198K mutation. In some embodiments, an SPB+ transposase domain comprises an M185R mutation and a D201R mutation. In some embodiments, an SPB+ transposase domain comprises a D197K mutation and a D201R mutation. In some embodiments, an SPB+ transposase domain comprises a D198K mutation and a D201R mutation. In some embodiments, an SPB+ transposase domain comprises an M185R mutation, a D198K mutation, and a D201R mutation.

In certain embodiments, one or more of the following mutations is/are introduced into one or both PBx transposase domains (e.g., the PBx transposase domain of SEQ ID NO: 56 with numbering beginning at the 5^{th} residue of SEQ ID NO: 56; or the PBx transposase domain of SEQ ID NO: 544) of a fusion protein provided herein to generate an PBx+ fusion protein: M185R, M185K, D197K, D197R, D198K, D198R, D201K, and D201R. In some embodiments, an PBx+ transposase domain comprises an M185R mutation and a D198K mutation. In some embodiments, an PBx+ transposase domain comprises an M185R mutation and a D201R mutation. In some embodiments, an PBx+ transposase domain comprises a D197K mutation and a D201R mutation. In some embodiments, an SPB+ transposase domain comprises a D198K mutation and a D201R mutation. In some embodiments, an PBx+ transposase domain comprises an M185R mutation, a D198K mutation, and a D201R mutation.

In certain embodiments, one or more of the following mutations is/are introduced into one or both SPB transposase domains (e.g., the SPB set forth in SEQ ID NO: 1 or 55, with numbering beginning at the 12^{th} residue of SEQ ID NO: 1 and at the 5^{th} residue of SEQ ID NO: 55) of a fusion protein provided herein to generate an SPB− fusion protein: L204D, L204E, K500D, K500E, R504E, and R504D. In some embodiments, an SPB− transposase domain comprises an L204E mutation and a K500D mutation. In some embodiments, an SPB− transposase domain comprises an L204E mutation and an R504D mutation. In some embodiments, an SPB− transposase domain comprises a K500 mutation and an R504D mutation. In some embodiments, an SPB− transposase domain comprises an L204E mutation, a K500D mutation, and an R504D mutation.

In certain embodiments, one or more of the following mutations is/are introduced into one or both PBx transposase (e.g., the PBx transposase domain of SEQ ID NO: 56 with numbering beginning at the 5^{th} residue of SEQ ID NO: 56 or the PBx transposase domain of SEQ ID NO: 544) of a fusion protein provided herein to generate a PBx− fusion protein: L204D, L204E, K500D, K500E, R504E, and R504D. In some embodiments, a PBx-transposase domain comprises an L204E mutation and a K500D mutation. In some embodiments, a PBx− transposase domain comprises an L204E mutation and an R504D mutation. In some embodiments, a PBx− transposase domain comprises a K500 mutation and an R504D mutation. In some embodiments, an PBx− transposase domain comprises an L204E mutation, a K500D mutation, and an R504D mutation.

Exemplary sequences of SPB+ transposase domains are set forth in SEQ ID NOs: 31-43 Exemplary sequences of SPB− transposase domains are set forth in SEQ ID NOs: 44-53. In some embodiments, a transposase domain provided herein comprises the amino acid sequence set forth in any one of SEQ ID NOs: 31-53. In some embodiments, a transposase domain provided herein comprises the amino acid sequence set forth in any one of SEQ ID NOs: 31-53 further comprising one or more conservative amino acid sequences.

In some embodiments, a fusion protein described herein comprises a first transposase domain and a second transposase domain, wherein both the first and the second transposase domain comprise an amino acid sequence set forth in any one of SEQ ID NOs: 31-43. In some embodiments, the first and the second transposase domain comprise the same sequence. In some embodiments, the first and the second transposase domain comprise different sequences. In some embodiments, both the first and the second transposase domain comprise an amino acid sequence set forth in any one of SEQ ID NOs: 31-43 further comprising one or more conservative amino acid sequences.

In some embodiments, a fusion protein described herein comprises a first transposase domain and a second transposase domain, wherein both the first and the second transposase domain comprise an amino acid sequence set forth in any one of SEQ ID NOs: 44-53. In some embodiments, the first and the second transposase domain comprise the same sequence. In some embodiments, the first and the second transposase domain comprise different sequences. In some embodiments, both the first and the second transposase domain comprise an amino acid sequence set forth in any one of SEQ ID NOs: 44-54 further comprising one or more conservative amino acid sequences.

In some embodiments, provided herein is a complex comprising (a) a first fusion protein comprising a first transposase domain, a linker, and a second transposase domain, wherein the first and/or the second transposase domain of the first fusion protein comprise the same amino acid sequence set forth in any one of SEQ ID NOs: 31-43.; and (b) a second fusion protein comprising a first transposase domain, a linker, and a second transposase domain, wherein the first and/or the second transposase domain of the second fusion protein comprise the same amino acid sequence set forth in any one of SEQ ID NOs: 44-53.

The SPB+, SPB−, PBx+, and PBx− fusion proteins and transposase domains may further comprise the N-terminal deletions of the second transposase domain described herein. Thus, in some embodiments, provided herein is an SPB+ fusion protein comprising a first and a second SPB+ transposase domain, wherein the first and the second SPB+ transposase domain are the same, except that the second transposase domain comprises an N-terminal deletion of about 20 amino acids, about 40 amino acids, about 60 amino acids, about 80 amino acids, about 100 amino acids, or about 115 amino acids. In some embodiments, the second transposase domain comprises an N-terminal deletion of 83 amino acids. In some embodiments, the second transposase domain comprises an N-terminal deletion of 90 amino acids. In some embodiments, the second transposase domain comprises an N-terminal deletion of 84 amino acids. In some embodiments, the second transposase domain comprises an N-terminal deletion of 85 amino acids. In some embodiments, the second transposase domain comprises an N-terminal deletion of 86 amino acids. In some embodiments, the second transposase domain comprises an N-terminal deletion of 87 amino acids. In some embodiments, the second transposase domain comprises an N-terminal deletion of 88 amino acids. In some embodiments, the second transposase domain comprises an N-terminal deletion of 89 amino acids. In some embodiments, the second transposase domain comprises an N-terminal deletion of 90 amino acids. In some embodiments, the second transposase domain comprises an N-terminal deletion of 91 amino acids. In some embodiments, the second transposase domain comprises an N-terminal deletion of 92 amino acids. In some embodiments, the second transposase domain comprises an N-terminal deletion of 93 amino acids. In some embodiments, the second transposase domain comprises an N-terminal deletion of 94 amino acids. In some embodiments, the second transposase domain comprises an N-terminal deletion of 95 amino acids. In some embodiments, the second transposase domain comprises an N-terminal deletion of 96 amino acids. In some embodiments, the second transposase domain comprises an N-terminal deletion of 97 amino acids. In some embodiments, the second transposase domain comprises an N-terminal deletion of 98 amino acids. In some embodiments, the second transposase domain comprises an N-terminal deletion of 99 amino acids. In some embodiments, the second transposase domain comprises an N-terminal deletion of 100 amino acids. In some embodiments, the second transposase domain comprises an N-terminal deletion of 101 amino acids. In some embodiments, the second transposase domain comprises an N-terminal deletion of 102 amino acids. In some embodiments, the second transposase domain comprises an N-terminal deletion of 103 amino acids.

In some embodiments, provided herein is an SPB− fusion protein comprising a first and a second SPB− transposase domain, wherein the first and the second SPB-transposase domain are the same, except that the second transposase domain comprises an N-terminal deletion of about 20 amino acids, about 40 amino acids, about 60 amino acids, about 80 amino acids, about 81 amino acids, about 82 amino acids, about 83 amino acids, about 84 amino acids, about 85 amino acids, about 86 amino acids, about 87 amino acids, about 88 amino acids, about 89 amino acids, about 90 amino acids, about 91 amino acids, about 92 amino acids, about 93 amino acids, about 94 amino acids, about 95 amino acids, about 96 amino acids, about 97 amino acids, about 98 amino acids, about 99 amino acids, about 100 amino acids, about 101 amino acids, about 102 amino acids, about 103 amino acids, or about 115 amino acids. In some embodiments, the second transposase domain comprises an N-terminal deletion of 83 amino acids. In some embodiments, the second transposase domain comprises an N-terminal deletion of 84 amino acids. In some embodiments, the second transposase domain comprises an N-terminal deletion of 85 amino acids. In some embodiments, the second transposase domain comprises an N-terminal deletion of 86 amino acids. In some embodiments, the second transposase domain comprises an N-terminal deletion of 87 amino acids. In some embodiments, the second transposase domain comprises an N-terminal deletion of 88 amino acids. In some embodiments, the second transposase domain comprises an N-terminal deletion of 89 amino acids. In some embodiments, the second transposase domain comprises an N-terminal deletion of 90 amino acids. In some embodiments, the second transposase domain comprises an N-terminal deletion of 91 amino acids. In some embodiments, the second transposase domain comprises an N-terminal deletion of 92 amino acids. In some embodiments, the second transposase domain comprises an N-terminal deletion of 93 amino acids. In some embodiments, the second transposase domain comprises an N-terminal deletion of 94 amino acids. In some embodiments, the second transposase domain comprises an N-terminal deletion of 95 amino acids. In some embodiments, the second transposase domain comprises an N-terminal deletion of 96 amino acids. In some embodiments, the second transposase domain comprises an N-terminal deletion of 97 amino acids. In some embodiments, the second transposase domain comprises an N-terminal deletion of 98 amino acids. In some embodiments, the second transposase domain comprises an N-terminal deletion of 99 amino acids. In some embodiments, the second transposase domain comprises an N-terminal deletion of 100 amino acids. In some embodiments, the second transposase domain comprises an N-terminal deletion of 101 amino acids. In some embodiments, the second transposase domain comprises an N-terminal deletion of 102 amino acids. In some embodiments, the second transposase domain comprises an N-terminal deletion of 103 amino acids.

In some embodiments, provided herein is a PBx+ fusion protein comprising a first and a second PBx+ transposase domain, wherein the first and the second PBx+ transposase domain are the same, except that the second transposase domain comprises an N-terminal deletion of about 20 amino acids, about 40 amino acids, about 60 amino acids, about 80 amino acids, about 100 amino acids, or about 115 amino acids. In some embodiments, the second transposase domain comprises an N-terminal deletion of 83 amino acids. In some embodiments, the second transposase domain comprises an N-terminal deletion of 84 amino acids. In some embodiments, the second transposase domain comprises an N-terminal deletion of 85 amino acids. In some embodiments, the second transposase domain comprises an N-terminal deletion of 86 amino acids. In some embodiments, the second transposase domain comprises an N-terminal deletion of 87 amino acids. In some embodiments, the second transposase domain comprises an N-terminal deletion of 88 amino acids. In some embodiments, the second transposase domain comprises an N-terminal deletion of 89 amino acids. In some embodiments, the second transposase domain comprises an N-terminal deletion of 90 amino acids. In some embodiments, the second transposase domain comprises an N-terminal deletion of 91 amino acids. In some embodiments, the second transposase domain comprises an N-terminal deletion of 92 amino acids. In some embodiments, the second transposase domain comprises an N-terminal deletion of 93 amino acids. In some embodiments, the second transposase domain comprises an N-terminal deletion of 94 amino acids. In some embodiments, the second transposase domain comprises an N-terminal deletion of 95 amino acids. In some embodiments, the second transposase domain comprises an N-terminal deletion of 96 amino acids. In some embodiments, the second transposase domain comprises an N-terminal deletion of 97 amino acids. In some embodiments, the second transposase domain comprises an N-terminal deletion of 98 amino acids. In some embodiments, the second transposase domain comprises an N-terminal deletion of 99 amino acids. In some embodiments, the second transposase domain comprises an N-terminal deletion of 100 amino acids. In some embodiments, the second transposase domain comprises an N-terminal deletion of 101 amino acids. In some embodiments, the second transposase domain comprises an N-terminal deletion of 102 amino acids. In some embodiments, the second transposase domain comprises an N-terminal deletion of 103 amino acids.

In some embodiments, provided herein is a PBx− fusion protein comprising a first and a second PBx− transposase domain, wherein the first and the second PBx− transposase domain are the same, except that the second transposase domain comprises an N-terminal deletion of about 20 amino acids, about 40 amino acids, about 60 amino acids, about 80 amino acids, about 81 amino acids, about 82 amino acids, about 83 amino acids, about 84 amino acids, about 85 amino acids, about 86 amino acids, about 87 amino acids, about 88 amino acids, about 89 amino acids, about 90 amino acids, about 91 amino acids, about 92 amino acids, about 93 amino acids, about 94 amino acids, about 95 amino acids, about 96 amino acids, about 97 amino acids, about 98 amino acids, about 99 amino acids, about 100 amino acids, about 101 amino acids, about 102 amino acids, about 103 amino acids, or about 115 amino acids. In some embodiments, the second transposase domain comprises an N-terminal deletion of 83 amino acids. In some embodiments, the second transposase domain comprises an N-terminal deletion of 84 amino acids. In some embodiments, the second transposase domain comprises an N-terminal deletion of 85 amino acids. In some embodiments, the second transposase domain comprises an N-terminal deletion of 86 amino acids. In some embodiments, the second transposase domain comprises an N-terminal deletion of 87 amino acids. In some embodiments, the second transposase domain comprises an N-terminal deletion of 88 amino acids. In some embodiments, the second transposase domain comprises an N-terminal deletion of 89 amino acids. In some embodiments, the second transposase domain comprises an N-terminal deletion of 90 amino acids. In some embodiments, the second transposase domain comprises an N-terminal deletion of 91 amino acids. In some embodiments, the second transposase domain comprises an N-terminal deletion of 92 amino acids. In some embodiments, the second transposase domain comprises an N-terminal deletion of 93 amino acids. In some embodiments, the second transposase domain comprises an N-terminal deletion of 94 amino acids. In some embodiments, the second transposase domain comprises an N-terminal deletion of 95 amino acids. In some embodiments, the second transposase domain comprises an N-terminal deletion of 96 amino acids. In some embodiments, the second transposase domain comprises an N-terminal deletion of 97 amino acids. In some embodiments, the second transposase domain comprises an N-terminal deletion of 98 amino acids. In some embodiments, the second transposase domain comprises an N-terminal deletion of 99 amino acids. In some embodiments, the second transposase domain comprises an N-terminal deletion of 100 amino acids. In some embodiments, the second transposase domain comprises an N-terminal deletion of 101 amino acids. In some embodiments, the second transposase domain comprises an N-terminal deletion of 102 amino acids. In some embodiments, the second transposase domain comprises an N-terminal deletion of 103 amino acids.

In some embodiments, provided herein is a complex comprising (a) a first fusion protein comprising a first transposase domain, a linker, and a second transposase domain, wherein (i) the first and second transposase domain are the same; or (ii) the first and second transposase domain are the same, except that the second transposase domain comprises an N-terminal deletion; and

(b) a second fusion protein comprising a first transposase domain, a linker, and a second transposase domain, wherein (i) the first and second transposase domain are the same; or (ii) the first and second transposase domain are the same, except that the second transposase domain comprises an N-terminal deletion.

The transposon domain sequences provided herein may be freely combined. Thus, in some embodiments, provided herein is a fusion protein comprising a first transposon domain and a second transposon domain, wherein the first transposon domain comprises the amino acid sequence set forth in any of SEQ ID NOs: 31-53, and the second transposon domain comprises the amino acid sequence set forth in any one of SEQ ID NOs: 1-7. In some embodiments, provided herein is a fusion protein comprising a first transposon domain and a second transposon domain, wherein the first transposon domain comprises an amino acid sequence that is at least 75%, at least 80%, at least 85%, at least 90%, at least 95%, at least 98%, or at least 99% identical to the sequence set forth in any of SEQ ID NOs: 31-53, and the second transposon domain comprises an amino acid sequence that is at least 75%, at least 80%, at least 85%, at least 90%, at least 95%, at least 98%, or at least 99% identical to the sequence set forth in any one of SEQ ID NOs: 1-7.

### Integration Cassettes

Also provided herein are integration cassettes for site-specific transposition of a DNA molecule into the genome of a cell. In some embodiments, the integration cassette for site-specific transposition of a nucleic acid into the genome of a cell comprises a nucleic acid consisting of a central transposon ITR integration site TTAA sequence flanked by an upstream Zinc Finger Motif DNA-binding domain binding site (“ZFM-DBD”) and a downstream ZFM-DBD, wherein each of the upstream and the downstream ZFM-DBD is separated from the TTAA sequence by 7 base pairs. In some embodiments, each of the at least one upstream and downstream ZFM-DBD sites is a ZFM268 binding site. In some embodiments, each of the ZFM268 binding sites comprises SEQ ID NO: 60. In some embodiments, the integration cassette comprises or consists of SEQ ID NO: 62.

Also provided here are cells comprising the integration cassette for site-specific transposition of DNA molecule stably integrated into the genome of the cell. In some embodiments, the integration cassette comprises or consists of SEQ ID NO: 62.

Also provided are methods for site-specific transposition of DNA molecule into the genome of a cell comprising a stably integrated integration cassette, comprising introducing into the cell: a) a nucleic acid encoding a fusion protein comprising a DNA binding domain and a transposase; wherein the fusion protein is expressed in the cell, and b) a DNA molecule comprising a transposon; wherein the expressed fusion protein integrates the transposon by site-specific transposition into the TTAA sequence of the stably integrated integration cassette. In some embodiments of the method, the integration cassette comprises or consists of SEQ ID NO: 62.

Also provided are methods for generating an engineered cell by site-specific transposition comprising: introducing into a cell comprising a stably integrated integration cassette: a) a nucleic acid encoding a fusion protein comprising a DNA binding domain and a transposase; wherein the fusion protein is expressed in the cell, and b) a DNA molecule comprising a transposon; wherein the expressed fusion protein integrates the transposon by site-specific transposition into the TTAA sequence of the stably integrated integration cassette thereby generating the engineered cell. In some embodiments of the method, the integration cassette comprises or consists of SEQ ID NO: 62.

### Nucleic Acids

Also provided herein are polynucleotides comprising nucleic acid sequences encoding the fusion proteins described herein. In some embodiments, the polynucleotides are isolated.

The isolated polynucleotides of the disclosure can be made using (a) recombinant methods, (b) synthetic techniques, (c) purification techniques, and/or (d) combinations thereof, as well-known in the art.

Methods of constructing nucleic acids encoding the transposase domains comprising an N-terminal deletion described herein are well known in the art or described herein, for example, PCR-based mutagenesis. Exemplary primers that may be used to construct transposase domains comprising an N-terminal deletion are shown in Table 3.

TABLE 3
Exemplary Primer Sequences
Forward Primers

| | |
|---|---|
| Delete 20 aa (#1) | GTGGGCGAAGATAGCGACAG (SEQ ID NO: 17) |
| Delete 40 aa (#2) | GATACCGAGGAAGCCTTCATC (SEQ ID NO: 18) |
| Delete 60 aa (#3) | GAGATCCTGGACGAGCAG (SEQ ID NO: 19) |
| Delete 80 aa (#4) | ATCCTGACACTGCCCCAG (SEQ ID NO: 20) |
| Delete 100 aa (#5) | AAGAGCACCAGACGGTCTAG (SEQ ID NO: 21) |
| Delete 115 aa (#6) | AGCCAGAGGGGCCCTAC (SEQ ID NO: 22) |

Reverse Primer

| | |
|---|---|
| Reverse primer (#7) | TCCGCCGCCAACTTTCC (SEQ ID NO: 23) |

The fusion of the present invention can be generated using any suitable method known in the art or described herein.

The isolated polynucleotides of this disclosure, such as RNA, cDNA, genomic DNA, or any combination thereof, can be obtained from biological sources using any number of cloning methodologies known to those of skill in the art. In some aspects, oligonucleotide probes that selectively hybridize, under stringent conditions, to the polynucleotides of the present disclosure are used to identify the desired sequence in a cDNA or genomic DNA library.

Methods of amplification of RNA or DNA are well known in the art and can be used according to the disclosure without undue experimentation, based on the teaching and guidance presented herein. Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

The polynucleotides of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Expression Vectors and Host Cells

The disclosure also relates to vectors that include polynucleotides of the disclosure, host cells that are genetically engineered with the recombinant vectors, and the production of at least one protein scaffold by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. In some embodiments, the promoter is an EF-1α promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Sh bla gene), puromycin (pac gene), hygromycin B (hygB gene), G418/Geneticin (neo gene), DHFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotes (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors will preferably but optionally include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22, CD20, CD33, CD52, or any combination thereof. Cell surface markers further include the suicide gene marker RQR8 (Philip B et al. Blood. 2014 Aug. 21; 124(8):1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, DHFR, TYMS, FRANCF, RAD51C, GCS, MDR1, ALDH1, NKX2.2, or any combination thereof.

Those of ordinary skill in the art are knowledgeable in the numerous expression systems available for expression of a nucleic acid encoding a protein of the disclosure. Alternatively, nucleic acids of the disclosure can be expressed in a host cell by turning on (by manipulation) in a host cell that contains endogenous DNA encoding a protein scaffold of the disclosure. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the protein scaffolds, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a preferred aspect, the recombinant cell is a P3X63Ab8.653 or an SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present disclosure are known and/or available, for instance, from the American Type Culture Collection Catalogue of Cell Lines and Hybridomas (www.atcc.org) or other known or commercial sources.

When eukaryotic host cells are employed, polyadenylation or transcription terminator sequences are typically incorporated into the vector. An example of a terminator sequence is the polyadenylation sequence from the bovine growth hormone gene. In some embodiments, the polyA sequence is an SV40 polyA sequence.

Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

The plasmid constructs described herein may be used to deliver nucleic acids encoding the transposase domains or fusion proteins described herein to a cell.

The transposase domains and fusion proteins described herein may also be delivered to a cell using mRNA constructs. Thus, in one embodiment, provided herein is an mRNA sequence encoding a transposase domain or a fusion protein described herein. Such mRNA sequences may be delivered to a cell using a nanoparticle, for example, a lipid nanoparticle. Examples of lipid nanoparticles are described in, e.g., International Patent Applications No. PCT/US2021/055876, No. PCT/US2022/017570, U.S. Provisional Application No. 63/397,268, U.S. Provisional Application No. 63/301,855 and U.S. Provisional Application No. 63/348,614, each of which is incorporated herein by reference in its entirety for examples of lipid nanoparticles that may be used to deliver mRNA constructs encoding the fusion proteins or transposase domains described herein. An mRNA construct may also be delivered to a cell by electroporation or nucleofection. The mRNA may be capped or oherwise modified.

### Cells and Modified Cells

The tandem dimer transposases and fusion proteins described herein may be used in conjunction with a transposon to modify cells. The transposon can be a piggyBac™ (PB) transposon. In some embodiments, when the transposon is a PB transposon, the transposase is a piggyBac™ (PB) transposase a piggyBac-like (PBL) transposase or a Super piggyBac™ (SPB) transposase. Non-limiting examples of PB transposons are described in detail in U.S. Pat. Nos. 6,218,182; 6,962,810; 8,399,643 and PCT Publication No. WO 2010/099296. The transposons can comprise a nucleic acid encoding a therapeutic protein or therapeutic agent. Examples of therapeutic proteins include those disclosed in PCT Publication No. WO 2019/173636 and PCT/US2019/049816.

Thus, provided herein are modified cells comprising one or more transposon and one or more tandem dimer transposase or fusion proteins described herein. Cells and modified cells of the disclosure can be mammalian cells. Preferably, the cells and modified cells are human cells.

A cell modified using a tandem dimer transposase described herein can be a germline cell or a somatic cell. Cells and modified cells of the disclosure can be immune cells, e.g., lymphoid progenitor cells, natural killer (NK) cells, T lymphocytes (T-cell), stem memory T cells (TSCM cells), central memory T cells (TCM), stem cell-like T cells, B lymphocytes (B-cells), antigen presenting cells (APCs), cytokine induced killer (CIK) cells, myeloid progenitor cells, neutrophils, basophils, eosinophils, monocytes, macrophages, platelets, erythrocytes, red blood cells (RBCs), megakaryocytes or osteoclasts. The modified cell can be differentiated, undifferentiated, or immortalized. The modified undifferentiated cell can be a stem cell. The modified undifferentiated cell can be an induced pluripotent stem cell. The modified cell can be a T cell, a hematopoietic stem cell, a natural killer cell, a macrophage, a dendritic cell, a monocyte, a megakaryocyte, or an osteoclast. The modified cell can be modified while the cell is quiescent, in an activated state, resting, in interphase, in prophase, in metaphase, in anaphase, or in telophase. The modified cell can be fresh, cryopreserved, bulk, sorted into sub-populations, from whole blood, from leukapheresis, or from an immortalized cell line. A detailed description for isolating cells from a leukapheresis product or blood is disclosed in in PCT Publication No. WO 2019/173636 and PCT/US2019/049816.

The methods of the disclosure can modify and/or produce a population of modified T cells, wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between of the plurality of modified T cells in the population expresses one or more cell-surface marker(s) of a stem memory T cell (TSCM) or a TscM-like cell; and wherein the one or more cell-surface marker(s) comprise CD45RA and CD62L. The cell-surface markers can comprise one or more of CD62L, CD45RA, CD28, CCR7, CD127, CD45RO, CD95, CD95 and IL-2RD. The cell-surface markers can comprise one or more of CD45RA, CD95, IL-2RD, CCR7, and CD62L.

The disclosure provides methods of expressing a CAR on the surface of a cell. The method comprises (a) obtaining a cell population; (b) contacting the cell population to a composition comprising a CAR or a sequence encoding the CAR, under conditions sufficient to transfer the CAR across a cell membrane of at least one cell in the cell population, thereby generating a modified cell population; (c) culturing the modified cell population under conditions suitable for integration of the sequence encoding the CAR; and (d) expanding and/or selecting at least one cell from the modified cell population that express the CAR on the cell surface. A more detailed description of methods for expressing a CAR on the surface of a cell is disclosed in PCT Publication No. WO 2019/049816 and PCT/US2019/049816.

The present disclosure provides a cell or a population of cells wherein the cell comprises a composition comprising (a) an inducible transgene construct, comprising a sequence encoding an inducible promoter and a sequence encoding a transgene, and (b) a receptor construct, comprising a sequence encoding a constitutive promoter and a sequence encoding an exogenous receptor, such as a CAR, wherein, upon integration of the construct of (a) and the construct of (b) into a genomic sequence of a cell, the exogenous receptor is expressed, and wherein the exogenous receptor, upon binding a ligand or antigen, transduces an intracellular signal that targets directly or indirectly the inducible promoter regulating expression of the inducible transgene (a) to modify gene expression.

The disclosure further provides a composition comprising the modified, expanded and selected cell population of the methods described herein.

The modified cells of disclosure (e.g., CAR T-cells) can be further modified to enhance their therapeutic potential. Alternatively, or in addition, the modified cells may be further modified to render them less sensitive to immunologic and/or metabolic checkpoints, for example by blocking and/or diluting specific checkpoint signals delivered to the cells (e.g., checkpoint inhibition) naturally, within the tumor immunosuppressive microenvironment.

The modified cells of disclosure (e.g., CAR T-cells) can be further modified to silence or reduce expression of (i) one or more gene(s) encoding receptor(s) of inhibitory checkpoint signals; (ii) one or more gene(s) encoding intracellular proteins involved in checkpoint signaling; (iii) one or more gene(s) encoding a transcription factor that hinders the efficacy of a therapy; (iv) one or more gene(s) encoding a cell death or cell apoptosis receptor; (v) one or more gene(s) encoding a metabolic sensing protein; (vi) one or more gene(s) encoding proteins that that confer sensitivity to a cancer therapy, including a monoclonal antibody; and/or (vii) one or more gene(s) encoding a growth advantage factor. Non-limiting examples of genes that may be modified to silence or reduce expression or to repress a function thereof include, but are not limited the exemplary inhibitory checkpoint signals, intracellular proteins, transcription factors, cell death or cell apoptosis receptors, metabolic sensing protein, proteins that that confer sensitivity to a cancer therapy and growth advantage factors that are disclosed in PCT Publication No. WO 2019/173636.

The modified cells of disclosure (e.g., CAR T-cells) can be further modified to express a modified/chimeric checkpoint receptor. The modified/chimeric checkpoint receptor can comprise a null receptor, decoy receptor or dominant negative receptor. Exemplary null, decoy, or dominant negative intracellular receptors/proteins include, but are not limited to, signaling components downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene. Non-limiting examples of cytokines, cytokine receptors, chemokines and chemokine receptors are disclosed in PCT Publication No. WO 2019/173636.

Genome modification can comprise introducing a nucleic acid sequence, transgene and/or a genomic editing construct into a cell ex vivo, in vivo, in vitro or in situ to stably integrate a nucleic acid sequence, transiently integrate a nucleic acid sequence, produce site-specific integration of a nucleic acid sequence, or produce a biased integration of a nucleic acid sequence. The nucleic acid sequence can be a transgene.

The stable chromosomal integration can be a random integration, a site-specific integration, or a biased integration. Without wishing to be bound by theory, it is believed that the addition of DNA binding domains to the tandem dimer transposases described herein improves the site-specificity of the transposases.

The site-specific integration can occur at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Non-limiting examples of potential genomic safe harbors include intronic sequences of the human albumin gene, the adeno-associated virus site 1 (AAVS1), a naturally occurring site of integration of AAV virus on chromosome 19, the site of the chemokine (C—C motif) receptor 5 (CCR5) gene and the site of the human ortholog of the mouse Rosa26 locus.

The site-specific transgene integration can occur at a site that disrupts expression of a target gene. Disruption of target gene expression can occur by site-specific integration at introns, exons, promoters, genetic elements, enhancers, suppressors, start codons, stop codons, and response elements. Non-limiting examples of target genes targeted by site-specific integration include TRAC, TRAB, PDI, any immunosuppressive gene, and genes involved in allo-rejection.

The site-specific transgene integration can occur at a site that results in enhanced expression of a target gene. Enhancement of target gene expression can occur by site-specific integration at introns, exons, promoters, genetic elements, enhancers, suppressors, start codons, stop codons, and response elements.

The site-specific transgene integration site can be a non-stable chromosomal insertion. The non-stable integration can be a transient non-chromosomal integration, a semi-stable non chromosomal integration, a semi-persistent non-chromosomal insertion, or a non-stable chromosomal insertion. The transient non-chromosomal insertion can be epi-chromosomal or cytoplasmic. In an aspect, the transient non-chromosomal insertion of a transgene does not integrate into a chromosome and the modified genetic material is not replicated during cell division.

The site-specific transgene integration site can be a modified binding site for the DNA targeting domain in a transposon domain, fusion protein, or tandem dimer described herein. For example, the TTAA target DNA integration site for SPB may modified to insert flanking DNA binding sites for the DNA targeting domain comprising three Zinc Finger Motifs (e.g., a DNA targeting domain comprising or consisting of the sequence of SEQ ID NO: 57 or a sequence having at least 75%, at least 80%, at least 85%, at least 90%, at least 95%, at least 98%, or at least 99% identity thereto). For example, it is believed that a DNA targeting domain comprising three Zinc Finger Motifs binds to the DNA sequence GCGTGGGCG (SEQ ID NO: 60). Therefore, the introduction of two copies of SEQ ID NO: 60 flanking the TTAA target integration site for SPB, is believed to improve site-specific integration of an SPB transposase domain comprising a DNA targeting domain comprising three Zinc Finger Motifs. The two copies of SEQ ID NO: 60 are in reverse (5′) and complement (3′) orientation.

In some embodiments, provided herein is a polynucleotide comprising, in 5′ to 3′ order, the reverse of the sequence of a target site for a DNA targeting domain, a first spacer, the TTAA target integration site for SPB, a second spacer, and the complement of the sequence of target site for a DNA targeting domain. In some embodiments, the first spacer and the second spacer have the same length. In some embodiments, the first and/or the second spacer are 3 bp in length. In some embodiments, the first and/or the second spacer are 4 bp in length. In some embodiments, the first and/or the second spacer are 5 bp in length. In some embodiments, the first and/or the second spacer are 6 bp in length. In some embodiments, the first and/or the second spacer are 7 bp in length. In some embodiments, the first and/or the second spacer are 8 bp in length. In some embodiments, the first and/or the second spacer are 9 bp in length. In some embodiments, the first and/or the second spacer are 10 bp in length.

Exemplary sequences of polynucleotides comprising, in 5′ to 3′ order, the reverse of the sequence of the target site for a DNA targeting domain comprising three Zinc Finger Motifs, a first spacer, the TTAA target integration site for SPB, a second spacer, and the complement of the sequence of the target site for the DNA targeting domain comprising three Zinc Finger Motifs are set forth in SEQ ID NOs: 61-64. The length of the first and second spacer in SEQ ID NOs: 61-64 is 8 bp, 7 bp, 6 bp, and 5 bp, respectively and the reverse and the complement of the target site for the DNA targeting domain is underlined and the TTAA sequence is shown in bold:

(SEQ ID NO: 61)
ACGCCCACGCTTACATCTTTAAAGATGTAAGCGTGGGCGT
(SEQ ID NO: 62)
ACGCCCACGCTACATCTTTAAAGATGTAGCGTGGGCGT
(SEQ ID NO: 63)
ACGCCCACGCTCATCTTTAAAGATGAGCGTGGGCGT
(SEQ ID NO: 64)
ACGCCCACGCTCTCTTTAAAGAGAGCGTGGGCGT

The modified target site may be introduced into a cell or a cell line to facilitate targeted genomic engineering. For example, a cell line which has been engineered to comprise a modified target site for an SPB or a PBx provided herein can be transfected with said SPB or PBx as well as a transposon comprising donor DNA such that the donor DNA is inserted at the modified target site. In some embodiments, the cell line is a T cell line. In some embodiments, the modified target sequence is introduced into a highly expressed genomic region. In a specific embodiment, provided herein is a cell line comprising stably integrated in its genomic sequence a nucleic acid sequence comprising, in 5′ to 3′ order, the reverse of the sequence of the target site for a DNA targeting domain comprising three Zinc Finger Motifs, a first spacer, the TTAA target integration site for SPB, a second spacer, and the complement of the sequence of the target site for the DNA targeting domain comprising three Zinc Finger Motifs. In some embodiments, the cell line comprises the sequence of any one of SEQ ID NOs: 61-64 stably integrated in its genome. In some embodiments, the cell is an in vitro cell, e.g., a cell in cell culture.

For DNA binding domains comprising TALENs, the target site is determined by the sequence of the TALENs. A person of skill in the art will be able to modify the TALEN sequences to achieve the desired target specificity. Methods of engineering Zinc-Finger Nucleases that bind to specific targets are described in, for example, Sander et al., Nat Methods. 2011 January; 8(1): 67-69.

The genome modification can be a non-stable chromosomal integration of a transgene. The integrated transgene can become silenced, removed, excised, or further modified.

In some embodiments, the transposase domains, fusion proteins and tandem dimer complexes provided herein have better transposase efficacy than their wildtype equivalents. Transposase activity may be measured by any suitable assay known in the art or described herein, for example, a Split GFP assay. For example, the transposase domains, fusion proteins and tandem dimer complexes provided herein may have comparable on-target genome integration activity to their wildtype counterparts, but have decreased off-target genome integration activity compared to their wildtype counterparts.

In some embodiments, a transposase domain comprising an N-terminal deletion and a DNA targeting domain provided herein has a ratio of on-target to off-target activity of at least 50-fold, at least about 100-fold, at least about 150-fold, at least about 200-fold, at least about 250-fold, at least about 300-fold, at least about 350-fold, at least about 400-fold, at least about 450-fold, at least about 500-fold, at least about 550-fold, at least about 600-fold, at least about 650-fold, at least about 700-fold, at least about 750-fold, at least about 800-fold, at least about 850-fold, at least about 900-fold, at least about 950-fold, or at least about 1000-fold compared to the wildtype transposase domain.

In some embodiments, a transposase domain comprising a DNA targeting domain inserted into the N-terminal region of the transposase domain provided herein has a ratio of on-target to off-target activity of at least 50-fold, at least about 100-fold, at least about 150-fold, at least about 200-fold, at least about 250-fold, at least about 300-fold, at least about 350-fold, at least about 400-fold, at least about 450-fold, at least about 500-fold, at least about 550-fold, at least about 600-fold, at least about 650-fold, at least about 700-fold, at least about 750-fold, at least about 800-fold, at least about 850-fold, at least about 900-fold, at least about 950-fold, or at least about 1000-fold compared to the wildtype transposase domain.

In certain embodiments, the modified cells are used therapeutically in adoptive cell therapy.

Adoptive cell compositions that are “universally” safe for administration to any patient (not just the patient from which they are derived) requires a significant reduction or elimination of alloreactivity. Towards this end, cells of the disclosure (e.g., allogenic cells) can be modified to interrupt expression or function of a T-cell Receptor (TCR) and/or a class of Major Histocompatibility Complex (MHC). The TCR mediates graft vs host (GvH) reactions whereas the MHC mediates host vs graft (HvG) reactions. In preferred aspects, any expression and/or function of the TCR is eliminated to prevent T-cell mediated GvH that could cause death to the subject. Thus, in a preferred aspect, the disclosure provides a pure TCR-negative allogeneic T-cell composition (e.g., each cell of the composition expresses at a level so low as to either be undetectable or non-existent).

Expression and/or function of MHC class I (MHC-I, specifically, HLA-A, HLA-B, and HLA-C) is reduced or eliminated to prevent HvG and, consequently, to improve engraftment of cells in a subject. Improved engraftment results in longer persistence of the cells, and, therefore, a larger therapeutic window for the subject. Specifically, expression and/or function of a structural element of MHC-I, Beta-2-Microglobulin (B2M), is reduced or eliminated. Non-limiting examples of guide RNAs (gRNAs) for targeting and deleting MHC activators are disclosed in PCT Application No. PCT/US2019/049816.

A detailed description of non-naturally occurring chimeric stimulatory receptors, genetic modifications of endogenous sequences encoding TCR-alpha (TCR-α), TCR-beta (TCR-β), and/or Beta-2-Microglobulin (β2M), and non-naturally occurring polypeptides comprising an HLA class I histocompatibility antigen, alpha chain E (HLA-E) polypeptide is disclosed in PCT Application No. PCT/US2019/049816.

Under normal conditions, full T-cell activation depends on the engagement of the TCR in conjunction with a second signal mediated by one or more co-stimulatory receptors (e.g., CD28, CD2, 4-1BBL) that boost the immune response. However, when the TCR is not present, T cell expansion is severely reduced when stimulated using standard activation/stimulation reagents, including agonist anti-CD3 mAb. Thus, the present disclosure provides a non-naturally occurring chimeric stimulatory receptor (CSR) comprising: (a) an ectodomain comprising a activation component, wherein the activation component is isolated or derived from a first protein; (b) a transmembrane domain; and (c) an endodomain comprising at least one signal transduction domain, wherein the at least one signal transduction domain is isolated or derived from a second protein; wherein the first protein and the second protein are not identical.

The activation component can comprise a portion of one or more of a component of a T-cell Receptor (TCR), a component of a TCR complex, a component of a TCR co-receptor, a component of a TCR co-stimulatory protein, a component of a TCR inhibitory protein, a cytokine receptor, and a chemokine receptor to which an agonist of the activation component binds. The activation component can comprise a CD2 extracellular domain or a portion thereof to which an agonist binds.

The signal transduction domain can comprise one or more of a component of a human signal transduction domain, T-cell Receptor (TCR), a component of a TCR complex, a component of a TCR co-receptor, a component of a TCR co-stimulatory protein, a component of a TCR inhibitory protein, a cytokine receptor, and a chemokine receptor. The signal transduction domain can comprise a CD3 protein or a portion thereof. The CD3 protein can comprise a CD3(protein or a portion thereof.

The endodomain can further comprise a cytoplasmic domain. The cytoplasmic domain can be isolated or derived from a third protein. The first protein and the third protein can be identical. The ectodomain can further comprise a signal peptide. The signal peptide can be derived from a fourth protein. The first protein and the fourth protein can be identical. The transmembrane domain can be isolated or derived from a fifth protein. The first protein and the fifth protein can be identical.

The present disclosure also provides a non-naturally occurring chimeric stimulatory receptor (CSR) wherein the ectodomain comprises a modification. The modification can comprise a mutation or a truncation of the amino acid sequence of the activation component or the first protein when compared to a wild type sequence of the activation component or the first protein. The mutation or a truncation of the amino acid sequence of the activation component can comprise a mutation or truncation of a CD2 extracellular domain or a portion thereof to which an agonist binds. The mutation or truncation of the CD2 extracellular domain can reduce or eliminate binding with naturally occurring CD58.

The present disclosure provides a nucleic acid sequence encoding any CSR disclosed herein. The present disclosure provides a transposon or a vector comprising a nucleic acid sequence encoding any CSR disclosed herein.

The present disclosure provides a cell comprising any CSR disclosed herein. The present disclosure provides a cell comprising a nucleic acid sequence encoding any CSR disclosed herein. The present disclosure provides a cell comprising a vector comprising a nucleic acid sequence encoding any CSR disclosed herein. The present disclosure provides a cell comprising a transposon comprising a nucleic acid sequence encoding any CSR disclosed herein.

The present disclosure provides a composition comprising any CSR disclosed herein. The present disclosure provides a composition comprising a nucleic acid sequence encoding any CSR disclosed herein. The present disclosure provides a composition comprising a vector comprising a nucleic acid sequence encoding any CSR disclosed herein. The present disclosure provides a composition comprising a transposon comprising a nucleic acid sequence encoding any CSR disclosed herein. The present disclosure provides a composition comprising a modified cell disclosed herein or a composition comprising a plurality of modified cells disclosed herein.

Also provided herein are methods site-specific gene integration. The transposon domains and fusion proteins provided herein may be used to deliver a transgene to a cell and integrate the transgene into a target site. The target site may be, for example, a genomic safe harbor, i.e., a genomic sites where a transgene can be integrated in a manner that ensures that the transgene functions predictably and does not cause alterations of the host genomic DNA sequence. In some embodiments, the target site is a repetitive element, such as a LINE-1 or ALU sequence. Repetitive elements do not encode gene products, making it unlikely that that an insertion leads to detrimental changes in the gene expression profile of a cell. There may be one, two or more target sites within one repetitive element. In some embodiments, the target site is located within an intron (e.g., an intro of the PAH gene).

The site-specific integration may be used in vitro or in vivo. An example of an in vivo application is gene therapy, which involves the delivery of a transgene to the genomic DNA of a cell.

### Formulations, Dosages and Modes of Administration

The present disclosure provides formulations, dosages and methods for administration of the compositions and cells described herein. In one aspect, provided herein is a pharmaceutical composition comprising a tandem dimer transposase or a fusion protein described herein and a pharmaceutically acceptable carrier. In another aspect, provided herein is a pharmaceutical composition comprising a modified cell described herein and a pharmaceutically acceptable carrier.

The disclosed compositions and pharmaceutical compositions can comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the protein scaffold, fragment or variant composition as well known in the art or as described herein.

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Non-limiting examples of carbohydrate excipients suitable for use include monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferably, the carbohydrate excipients are mannitol, trehalose, and/or raffinose.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate.

Additionally, the disclosed compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means. In preferred embodiments, a composition comprising a modified cell described herein is administered intravenously, e.g., by intravenous infusion.

A composition of the disclosure can be prepared for use for parenteral (subcutaneous, intramuscular or intravenous) or any other administration particularly in the form of liquid solutions or suspensions. For parenteral administration, a composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection or infusion can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

It can be desirable to deliver the disclosed compounds to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g., a zinc tannate salt. Additionally, the disclosed compounds or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

### Methods of Treatment

In another aspect, provided herein are methods of treating a disease or disorder in a subject, the method comprising administering to the subject a composition comprising the modified cells described herein. The terms “subject” and “patient” are used interchangeably herein. In preferred embodiments, the patient is human.

The modified cells may be allogeneic or autologous to the patient. In some preferred embodiments, the modified cell is an allogeneic cell. In some embodiments, the modified cell is an autologous T-cell or a modified autologous CAR T-cell. In some preferred embodiments, the modified cell is an allogeneic T-cell or a modified allogeneic CAR T-cell.

In some embodiments, the disease or disorder treated in accordance with the methods described herein is a cancer. In some embodiments, a method of treatment described herein may delay cancer progression and/or reduce tumor burden.

The dosage of a pharmaceutical composition to be administered to a subject can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired.

In aspects where the compositions to be administered to a subject in need thereof are modified cells as disclosed herein, between about 1×10³ and about 1×10⁴ cells; between about 1×10⁴ and about 1×10⁵ cells; between about 1×10⁵ and about 1×10⁶ cells; between about 1×10⁶ and about 1×10⁷ cells; between about 1×10⁷ and about 1×10⁸ cells; between about 1×10⁸ and about 1×10⁹ cells; between about 1×10⁹ and about 1×10¹⁰ cells, between about 1×10¹⁰ and about 1×10¹¹ cells, between about 1×10¹¹ and about 1×10¹² cells, between about 1×10¹² and about 1×10¹³ cells, between about 1×10¹³ and about 1×10¹⁴ cells, between about 1×10¹⁴ and about 1×10¹⁵ cells, between about 1×10¹⁵ and about 1×10¹⁶ cells, between about 1×10¹⁶ and about 1×10¹⁷ cells, between about 1×10¹⁷ and about 1×10¹⁸ cells, between about 1×10¹⁸ and about 1×10¹⁹ cells; or between about 1×10¹⁹ and about 1×10²⁰ cells may be administered. In some embodiments, the cells are administered at a dose of between about 5×10⁶ and about 25×10⁶ cells.

In other embodiments, the dosage of cells may depend on the body weight of the person, e.g., between about 1×10³ and about 1×10⁴ cells; between about 1×10⁴ and about 1×10⁵ cells; between about 1×10⁵ and about 1×10⁶ cells; between about 1×10⁶ and about 1×10⁷ cells; between about 1×10⁷ and about 1×10⁸ cells; between about 1×10⁸ and about 1×10⁹ cells; between about 1×10⁹ and about 1×10¹⁰ cells, between about 1×10¹⁰ and about 1×10¹¹ cells, between about 1×10¹¹ and about 1×10¹² cells, between about 1×10¹² and about 1×10¹³ cells, between about 1×10¹³ and about 1×10¹⁴ cells, between about 1×10¹⁴ and about 1×10¹⁵ cells, between about 1×10¹⁵ and about 1×10¹⁶ cells, between about 1×10¹⁶ and about 1×10¹⁷ cells, between about 1×10¹⁷ and about 1×10¹⁸ cells, between about 1×10¹⁸ and about 1×10¹⁹ cells; or between about 1×10¹⁹ and about 1×10²⁰ cells may be administered per kg body weight of the subject.

A more detailed description of pharmaceutically acceptable excipients, formulations, dosages and methods of administration of the disclosed compositions and pharmaceutical compositions is disclosed in PCT Publication No. WO 2019/049816.

The transposon domains and fusion proteins provided herein may be used to deliver a gene therapy. Gene therapy usually involves the delivery of a transgene to the genomic DNA of a cell. Usually, the transgene replaces a gene that is mutated or otherwise not expressed properly in the cell. The fusion proteins, transposase domains, and complexes described herein may be used to deliver a therapeutic transgene to a cell and integrate the transgene into a target site. In some embodiments, a method of treatment comprises introducing into the cell the fusion protein of any one of claims 1-13 and a transposon, wherein the transposon comprises, in 5′ to 3′ order: a 5′ITR, the transgene, and a 3′ ITR.

### Kits

In another aspect, provided herein is a kit comprising a cell line which has been engineered to comprise a modified target site for an SPB or a PBx provided herein within its genome, preferably in a highly expressed genomic region. The kit may further comprise a composition comprising one or more SPB or PBx transposase domains or fusion proteins described herein. In some embodiments, the cell line is a T cell line.

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof. In the context of nucleic acids, a promoter may be operatively linked to a nucleotide sequence encoding a transpose domain or fusion protein described herein, bringing the expression of the nucleotide sequence under the control of the promoter.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157: 105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within +2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Table 4, Table 5, and Table 6 below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table 4.

TABLE 4
Conservative Substitutions I

| | |
|---|---|
| Side chain characteristics | Amino Acid |

| | | |
|---|---|---|
| Aliphatic | Non-polar | G A P I L V F |
| | Polar - uncharged | C S T M N Q |
| | Polar - charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table 5.

TABLE 5
Conservative Substitutions II

| | |
|---|---|
| Side Chain Characteristic | Amino Acid |

| | | |
|---|---|---|
| Non-polar (hydrophobic) | Aliphatic: | A L I V P |
| | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table 6.

TABLE 6
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, identity between two sequences may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. In some embodiments, the sequence identify is determined over the entire length of a sequence. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

In certain embodiments, if a sequence has a certain sequence identity (e.g., 75%, 80%, 85%, 90%, 95%, 98%, or 99%) to a certain SEQ ID NO, the sequence and the sequence of the SEQ ID NO have the same length. In certain embodiments, if a sequence has a certain sequence identity (e.g., 75%, 80%, 85%, 90%, 95%, 98%, or 99%) to a certain SEQ ID NO, the sequence and the sequence of the SEQ ID NO only differ due to conservative amino acid substitutions.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### EXAMPLES

The Examples in this section are provided for illustration and are not intended to limit the invention.

### Example 1: Construction of a Set of Nested Deletions of the N-terminal Portion of the SPB Transposase Domain

A set of nested deletions of the N-terminal portion of the SPB transposase was constructed using PCR-based mutagenesis. A plasmid comprising the DNA sequence encoding wild type SPB transposase comprising an N-terminal NLS (SEQ ID NO: 24) under the control of the EF-1α promoter was used as the DNA template for PCR-based mutagenesis to generate deletions of 20, 40, 60, 80, 100 or 115 amino acids of the N-terminus of the of SPB transposase sequence. Briefly, forward primers were designed complementary to downstream sequences flanking the C-terminal deletion boundary (SEQ ID Nos. 17-22) and a reverse primer (SEQ ID NO: 23) was designed complementary to the upstream amino-terminal NLS sequence. SPB transposase encoding fragments were generated using a thermocycler and a Q5 Hotstart kit (NEB Labs) under the conditions shown in Table 7 and Table 8 and in accordance with the manufacturer's instructions.

TABLE 7
Q5 2x Master Mix

| | Volume/uL |
|---|---|
| Water | 21.5 |
| 10 uM each primer mix | 2.5 |
| DNA Sag/100 ul | 1 |
| QS hot start 2X mix | 25 |
| Total | 50 |

TABLE 8
PCR Conditions

| | | |
|---|---|---|
| Steps | Temp/° C. | Time |

| | | | | |
|---|---|---|---|---|
| Denature | 98 | C. | 1 | min |
| 24 cycles | 98 | C. | 15 | s |

| | | |
|---|---|---|
| 60, 65 | 20 | s |

| | |
|---|---|
| 72 C. (20-30 s/kb) | 2 min 30 s |

| | | | | |
|---|---|---|---|---|
| Final extension | 72 | C. | 2 | min |
| Hold | 4 | C. | | |

Crude PCR products were directly treated with the KLD enzyme kit (Grainger) following manufacture's protocol. The KLD enzyme mix contains kinase, ligase and the restriction enzyme DpnI resulting in ligated, full-length fragments suitable for direct cloning into plasmid vectors. SPB transposase fragments were sized by gel electrophoresis and those DNA fragments of desired size were cloned into plasmid vectors. Resulting plasmids were transformed into Zymo DH5a MixAndGo (T3007) competent cells following manufacturer's protocol. The nucleotide sequence of each SPB construct comprising an N-terminal deletion was confirmed by direct Sanger DNA sequencing.

### Example 2: Construction of Fusion Proteins

This example illustrates exemplary methods for constructing tandem dimer transposases of the present invention using two-fragment Gibson Assembly.

Two fragments were used for the Gibson Assembly of the tandem dimer SPB expressing plasmid (1) the plasmid backbone containing EF1α promoter, the NLS sequence, the 1st SPB transposon domain, the poly-A signal, and the essential elements for plasmid replication, etc.; (2) L3 linker plus the 2nd SPB full length transposon domain with different codon usage. This fragment is directly supplied as gene block fragment. To assemble the plasmid backbone, the wildtype SPB plasmid (SEQ ID NO: 24) is amplified using the following primers: Forward: tctagaaccggtcatggccg (SEQ ID NO: 25), reverse: GAAGCAGCTCTGGCACATG (SEQ ID NO: 26).

The Insert fragment containing the second SPB transposase domain is supplied directly as double-stranded gene block DNA fragment. The sequence of the insert fragment is set forth in SEQ ID NO: 27. The DNA sequence of the assembled product is set forth in SEQ ID NO: 30.

The amplified region of the template fragment shares a region of complementarity after the C-terminus of the SPB coding sequence with a region located upstream of the 5′ end of the second SPB coding sequence whereupon 5′ exonuclease digestion, polymerase fill ins and DNA ligation results in the fusion of the first transposase domain sequence in frame with the second transposase domain sequence comprising an intervening 13 amino acid linker to generate tdSPB.

To construct the fusion protein of the present invention comprising a deletion of a portion of the amino terminus of the SPB transposase domain, the tdSPB was used as a DNA template in PCR mutagenesis assays described in Example 1 to generate fusion proteins comprising an amino terminal deletion of 20 amino acids, 40 amino acids, 60 amino acids, 80 amino acids, 100 amino acids or 115 amino acids (SEQ ID Nos. 9-14) in only the second transposase domain. The two SPB transposase domain sequences have differing codon usage in the N-terminally deleted sequence to allow for forward primers to be designed with complementarity to the second transposase domain coding sequence. The presence of each deletion of the second transposase domain and integrity of the coding sequence of the first transposase domain was confirmed by Sanger DNA sequencing.

### Example 3: Methods for Measuring Excision Activity of SPB Transposase Domains and Fusion Proteins

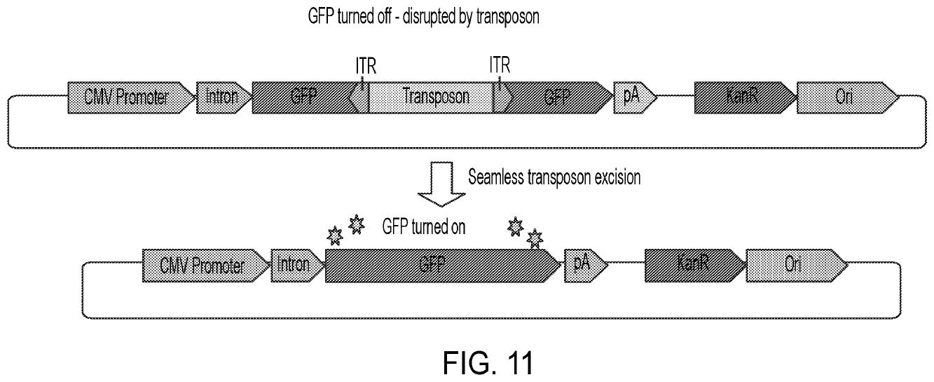

This assay is designed to measure the excision activity of transposase domains and fusion proteins comprising transposase domains. In this assay, the transpose domain or the fusion protein comprising a first and a second transposase domain are co-administered to cells together with a reporter transposon construct, in which the transposon comprises a DNA nucleotide sequence encoding a non-functional GFP in which the coding sequence has been interrupted by an intervening piece of DNA flanked by TTAA sequences and the inverse terminal repeat (ITR) sequences of the PB transposon. A schematic of the reporter (GFP Excision Only Reporter) is shown in FIG. 11. The TTAA sequences and ITRs serve as recognition sites for the SPB transposase and if the transposase domain or fusion protein possesses excision activity, the intervening DNA will be excised, restoring the intact, full-length coding sequence of the GFP gene. Thus, transposase domains and fusion proteins possessing transposase activity produce GFP positive cells in this assay that may be identified and quantified by FACS.

In a first experiment, the excision activity of SPB transposase domains harboring various sized N-terminal deletions described in Example 1 was determined. On Day 0, HEK293 cells were seeded into 48 well plates at a density of 70,000 cells/well and to each well DMEM medium supplemented with 10% FBS was added and cells were cultured at 37° C. at 5% CO2. On Day 1, the culture medium was removed by aspiration and the cells were resuspended in buffer comprising Jetprime transfection reagent (Polyplus Transfection) according to the manufacturer's instructions. SPB transposase domains and the reporter transposon construct were added at the concentrations per well shown in Table 9.

TABLE 9

| SPBase/ng | Transposon/ng | jetPrime/μL | Total Complex/μL |
|---|---|---|---|
| 10 | 240 | 0.5 | 25 |

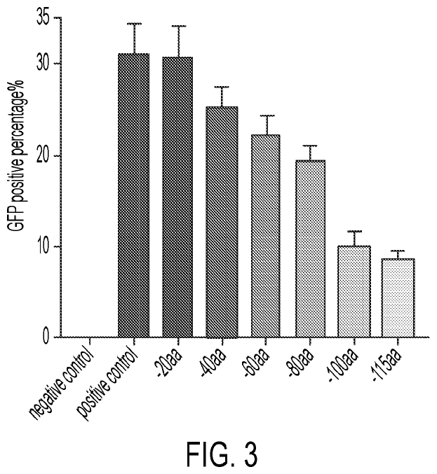

After approximately 24 hours, cells were resuspended in PBS supplemented with 5% FBS and the number of GFP expressing cells was determined using flow cytometry. The results are shown in FIG. 3.

As shown in FIG. 3, the wild type, full length SPB transposase domain generated approximately 31% GFP positive cells. The deletion of the first 20 amino acid residues of the N-terminus of the SPB transposase domain had little effect on the percentage of GFP positive cells and the deletion of 40, 60 or even 80 amino acids of the N-terminus of the SPB transposase domain reduced the percentage of GFP positive cells by only 25-50% of wild type activity. The deletion of 100 or 115 amino acid residues had a further reduction on SPB transposase activity, but SPB transposase domains harboring the deletion of 115 amino acids (˜1/3 of SB transposase coding sequence) still retain 25% of wild type activity.

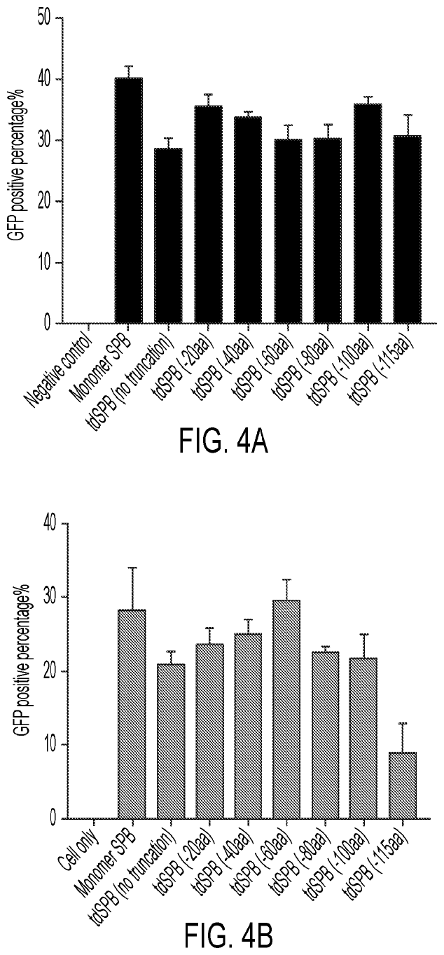

In a second experiment, HEK 293 were seeded on Day 0 and the cells were transfected as described above in the first experiment except that the reporter transposon construct was co-administered with one of the fusion proteins comprising one of the N-terminally deleted transposase domains prepared in Example 2 at the same concentrations and under the same conditions, and the number of GFP expressing cells was determined. The results are shown in FIG. 4A.

As shown in FIG. 4A, all fusion proteins (“tdSPB” in FIG. 4A) comprising a wild type SPB transposase domain linked to an N-terminally deleted SPB transposase domain retained excision activity at a level of approximately 75% of the wild type SPB transposase domain (“monomer SPB” in FIG. 4A), demonstrating that the N-terminally-deleted fusion proteins are functional at recognizing and excising DNA.

### Example 4: Methods for Measuring Integration Activity of SPB Transposase Domains and Fusion Proteins

This assay is designed to measure the integration activity of fusion proteins comprising two SPB transposase domains. In this assay, the fusion proteins are co-administered to cells together with a reporter transposon construct, in which the transposon comprises a DNA nucleotide sequence encoding GFP in which the coding sequence is flanked by TTAA sequences and the ITR sequences of the PB transposon. The TTAA and ITR sequences serve as recognition sites for the SPB transposase domains and if the fusion protein possesses integration activity, the DNA encoding GFP is integrated into genomic DNA, whereupon it is expressed and produces GFP positive cells that may be identified and quantified by FACS.

The integration activity of the fusion proteins comprising one wildtype transposase domain and one N-terminally deleted transposase domain was determined. On Day 0, HEK293 cells were seeded into 48 well plates at a density of 70,000 cells/well in and to each well DMEM medium supplemented with 10% FBS was added and cells were cultured at 37° C. at 5% CO2. On Day 1, the culture medium was removed by aspiration and the cells were resuspended in Jetprime buffer comprising the transfection reagent (Polyplus Transfection) according to the manufacturer's instructions and the fusion proteins comprising SPB transposase domains and the reporter transposon constructs were added at the concentrations per well shown in Table 10.

TABLE 10

| SPBase/ng | Transposon/ng | jetPrime/μL | Total Complex/μL |
|---|---|---|---|
| 10 | 240 | 0.5 | 25 |

After approximately 24 hours (Day 2), the culture medium was removed, the cells were resuspended in fresh DMEM culture medium supplemented with 1% FBS and incubated for an additional three days. On Day 6, the culture medium was again removed and the cells were resuspended in fresh DMEM culture medium supplemented with 1% FBS and incubated for an additional two days. On Day 8, the cells were resuspended in PBS supplemented with 5% FBS and the number of GFP expressing cells was determined using flow cytometry. The results are shown in FIG. 4B.

As shown in FIG. 4B, a fusion protein (“tdSPB” in FIG. 4B) comprising a wild type SPB transposase domain fused to second wild type SPB transposase domain through a linker reduces the integration activity by about 33% compared to a wildtype SPB transposase domain alone (“monomer SPB” in FIG. 4B). Fusion proteins comprising one wildtype transposase domain and one N-terminally deleted transposase domain harboring deletions of as large as 100 amino acids of the N-terminus of the second SPB transposase domain exhibit activity as good or better than the fusion protein comprising two wildtype SPB transposase domains. The deletion of 60 amino acids off the N-terminus of the second transposase domain, however, increased integration activity to levels equivalent to the wild type SPB transposase domain alone, and approximately 33% above that of the fusion protein comprising two wildtype SPB transposase domains.

### Example 5: Rational Design of SPB Heterodimers

The SPB dimer is believed to be held together through a combination of salt bridges, hydrogen bonds, pi-cation pairs, and hydrophobic interactions. The residues involved in these interactions in the SPB dimer can be identified by looking at the published structures of piggyBac (PB) Transposases (see, e.g., Structural basis of seamless excision and specific targeting by piggyBac transposase. Chen Q, Luo W, Veach R A, Hickman A B, Wilson M H, Dyda F. Nat Commun (2020) 11 p. 3446). Two structures, 6×67 and 6×68, which have been deposited in NCBI, were analyzed using the “Interaction Analysis” tool in NCBI's protein structure 3D viewer to find amino acids likely involved in dimerization between two PB transposase monomers. The default settings were used, which searched for potential hydrogen bonds of 3.8 Å or less, salt bridges of 6 Å or less, pi-cation pairs of 6 Å or less and other contacts of 4 Å or less. The residue pairs show in Table 2 were identified. These residues are found within the “DNA binding and dimerization domain (DDBD)” (residues 118-263, 458-535) or within the “Cysteine rich C-terminal domain (CRD)” (residues 554-594). Although each and all of these residues, as well as surrounding residues, could theoretically be mutated in the SPB or PBx transposase monomers to create obligate heterodimers, the residues in the DDBD were investigated first, since the structure of the SPB dimer is more symmetrical around the DDBD than it is around the CRD. For example, within the DDBD, D198 of monomer 1 interacts with K500 of monomer 2 and K500 of monomer 1 interacts with D198 of monomer 2. However, within the CRD, R583 of monomer 1 interacts with D588 of monomer 2 but D588 of monomer 1 does not interact with R583 of monomer 2.

Initial studies were focused on two salt bridges which are likely involved in holding together the PB dimer, namely those between D198 and K500 and between D201 and R504. By swapping the negatively charged residues (D) for positively charged residues (K,R) in one SPB transposase domain and swapping the positively charged residues for negatively charged residues in the second SPB transposase domain, two new types of SPB mutants—SPB+ and SPB−—were created. It was expected that SPB+ would repel SPB+, and likewise, SPB− would repel SPB−. As opposite charges attract, SPB+ was expected to heterodimerize with SPB−.

Subsequently, uncharged residues were also mutated to charged residues to create additional charge at the dimerization interface. For example, M185 of one PB transposase monomer is located within close proximity of L204 of the second PB transposase monomer. To add positive charge to monomer 1, a M185K mutation was introduced, and to add negative charge to monomer 2, a L204E mutation was introduced.

The individual point mutations making up the different versions of SPB+ could be combined in all possible combinations to create additional SPB+ mutants. The same is true of the SPB− mutations. The SPB+ and SPB− mutant monomers can be used as the transposase domains of the fusion proteins described herein.

### Example 6: Testing SPB Heterodimers

The SPB+ or SPB− transposase domain mutants described in Example 5 were cloned into an expression vector driven by the EF1a promoter. In particular, the SPB mutants comprising SEQ ID NOs 31, 32, 33, 34, 35, 36, 37, 38, 39, 44, 45, 46, 47, 48, 49 or 50 were tested. The nucleotide sequence of the expression vector is set forth in SEQ ID NO: 54.

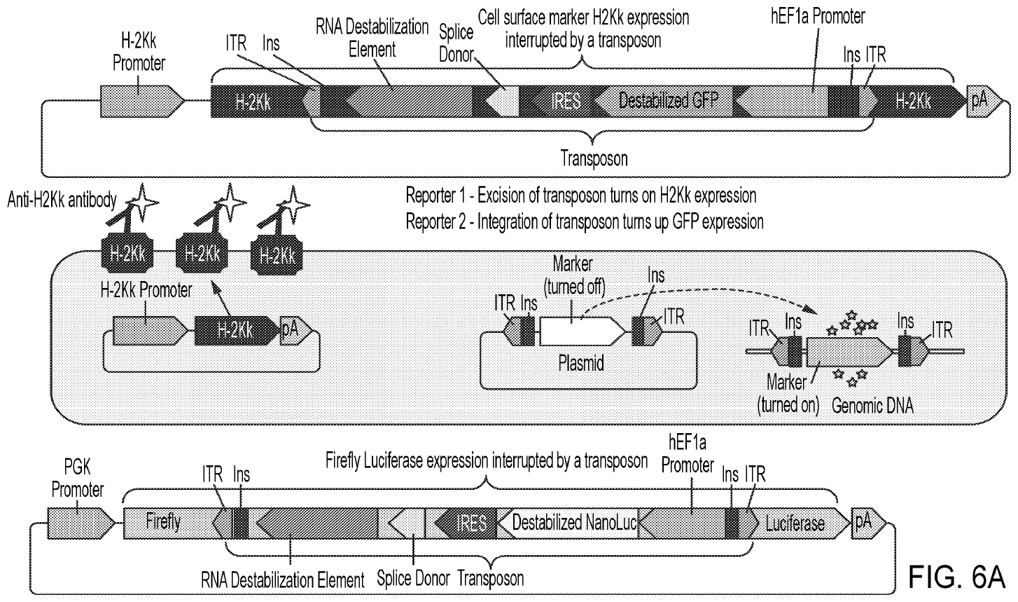

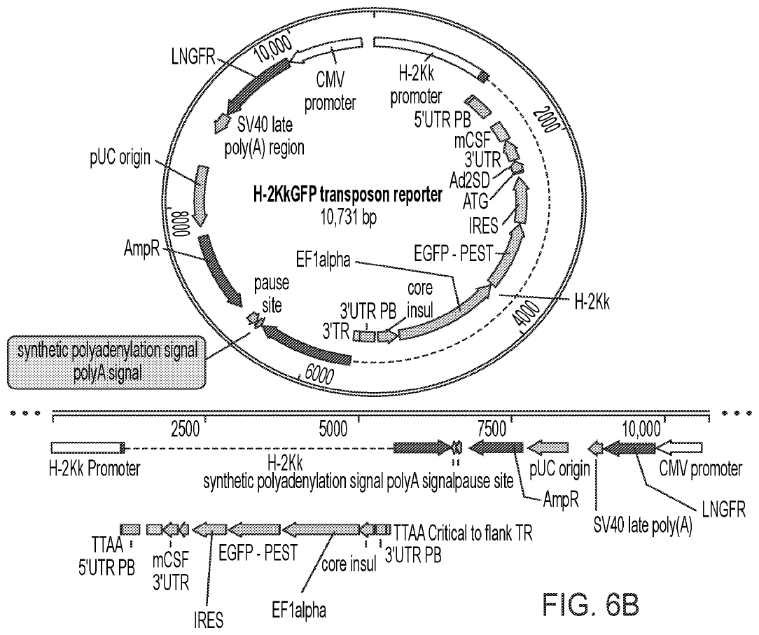

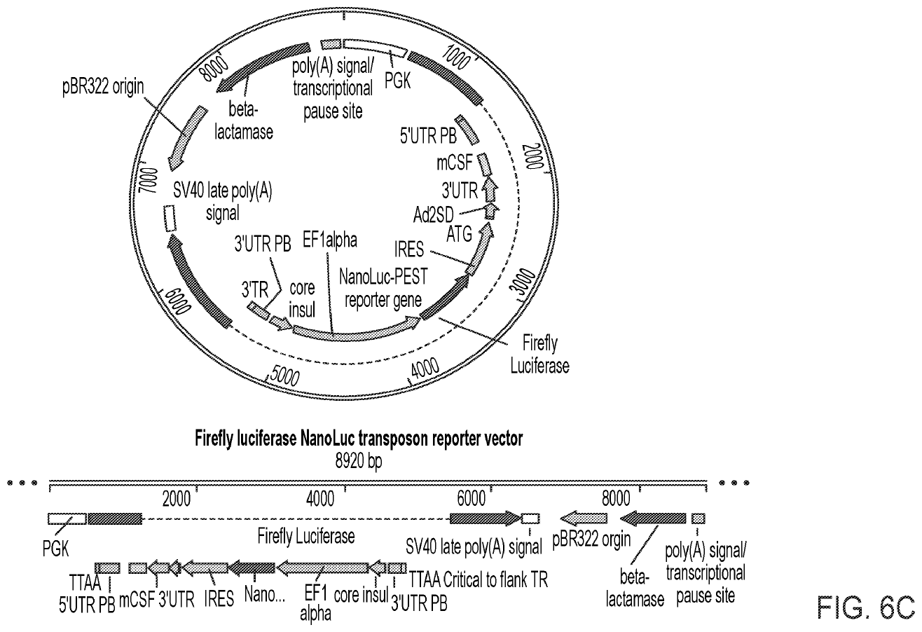

Each mutant was then nucleofected into K562 cells either alone (to form a homodimer, e.g., two SPB+ mutants) or with its respective heterodimer counterpart (e.g., an SPB+ mutant and the corresponding SPB− mutant). To assay for transposition activity, the cells were co-transfected with a dual excision/integration luciferase reporter vector. The vector was designed such that a firefly luciferase open reading frame is disrupted by a SPB transposon. Initially, firefly luciferase is not expressed, but SPB-mediated excision of the transposon and seamless repair results in expression. The transposon itself expresses a destabilized Nanoluc luciferase mRNA. Nanoluc expression from the episomal vector is unstable as the mRNA lacks a polyA tail and contains 3′ destabilization element. Integration of the transposon into genomic DNA allows the mRNA to pick up a polyA and splice out the destabilization element using a splice donor sequence on the transposon, leading to luciferase expression. The reporter vector is illustrated in the bottom panel of FIG. 6A.

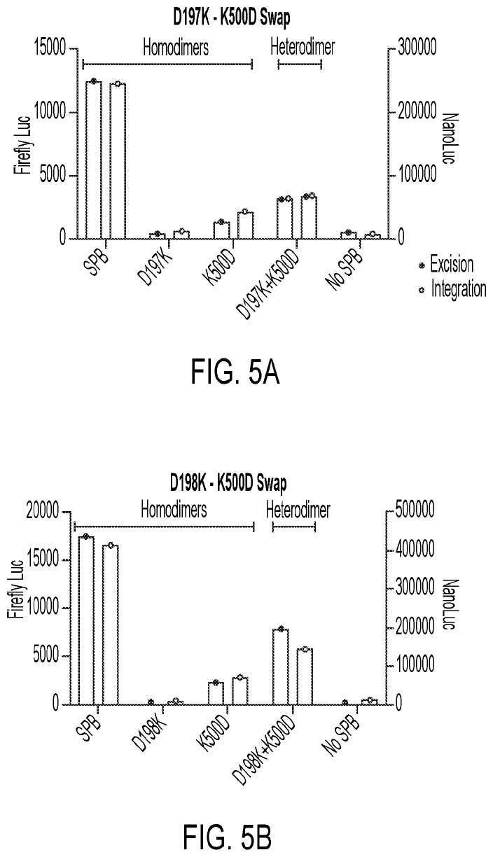

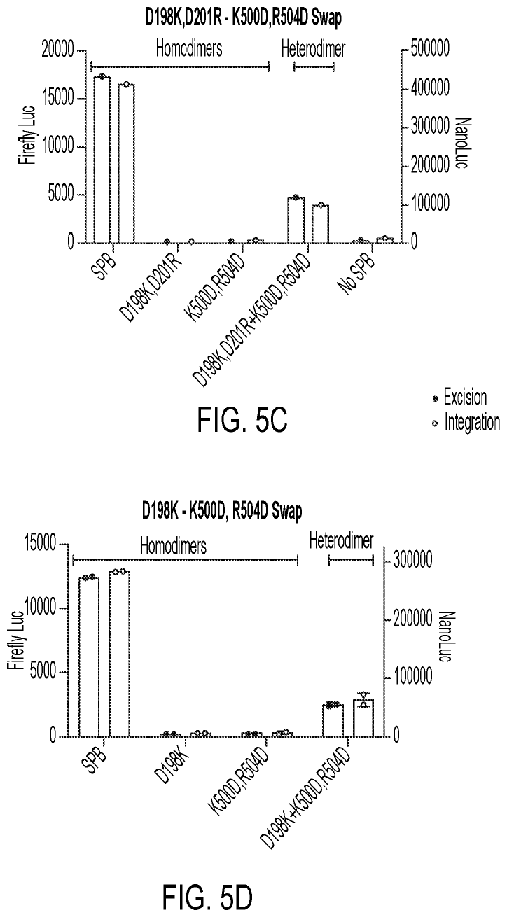

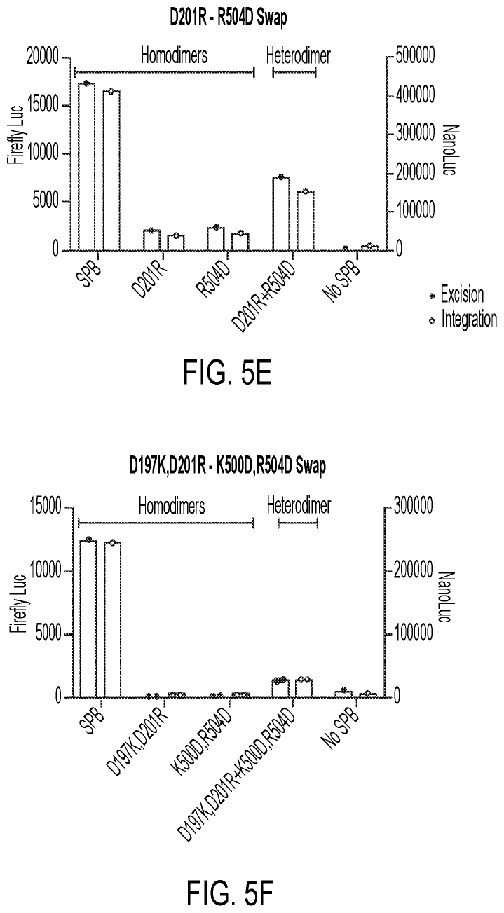

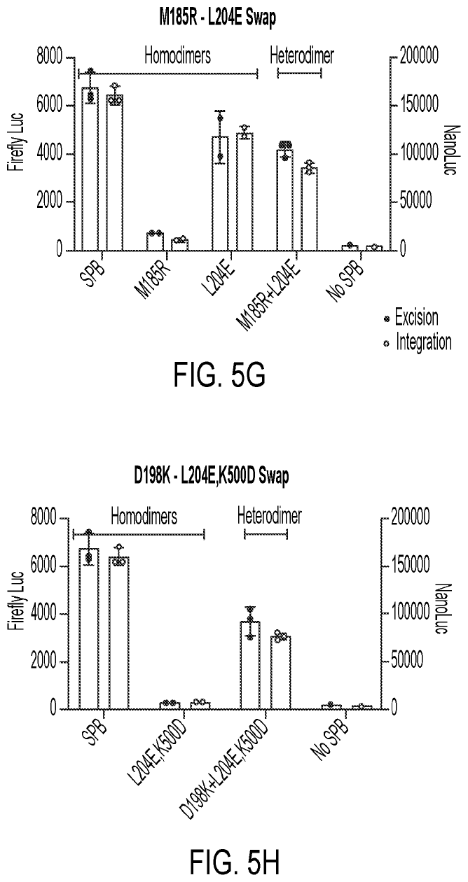

K562 cells were nucleofected using 20 μl of SF buffer and program FF-120. Each reaction contained 50 ng of the dual luciferase reporter and 500 ng of a SPB-expressing plasmid. For testing the SPB homodimers, 500 ng of the SPB-expressing plasmid was used. For testing SPB as heterodimers, 250 ng of each SPB expressing plasmid was used. One day post transfection, luciferase signal was measure using Promega's dual luciferase reagents and a plate reader. Results are shown in FIGS. 5A-5H. Several constructs showed little to no activity as homodimers but did show activity has heterodimers. Heterodimer activity reached 25-50% of the activity of wildtype SPB. The best transposase activity was observed with the following combinations: SPB+ D198K and SPB− K500D, R504D; SPB+ D198K and SPB-L204E, K500D; and SPB+ D198K, D201R and SPB− K500D, R504D.

### Example 7: Construction of Amino-Terminal Deletions of Super PiggyBac Transposases

Plasmids comprising a nucleotide sequence encoding a full-length, wild type Super PiggyBac transposase (SPB; SEQ ID NO: 55) or a nucleotide sequence encoding an integration-deficient variant of Super PiggyBac transposase comprising amino acid substitutions at positions R372A, K375A and D450N (PBx; SEQ ID NO: 56) were used as templates for PCR mutagenesis to generate N-terminal deletion transposase variants lacking the N-terminal 93 amino acids (SPBA1-93 and PBxΔ1-93, respectively).

Briefly, forward and reverse primers were designed to amplify a portion of the SPB and PBx coding sequences corresponding to amino acids 94-594. The resulting DNA fragments encoding SPBA1-93 or PBxA1-93 were used together with a purchased gBlock gene fragment to construct DNA binding domain—transposase fusion proteins via a state-of-the-art 2-fragment Gibson Assembly.

### Example 8: Construction of Transposases Comprising DNA Binding Domains

DNA-binding domain-comprising transposases were generated by fusing in-frame three zinc finger DNA binding motifs (ZF268) to the N-terminus (amino acid 94) of SPBA1-93 and PBxA1-93. Briefly, a gBlock DNA fragment encoding the ZF268 zinc finger protein binding motifs flanked by GGGGS linkers (SEQ ID NO: 57) was assembled with the DNA fragments encoding SPBA1-93 or PBxA1-93 from Example 7 and cloned into an expression vector comprising an in-frame initiator methionine and alanine codons followed by an SV40 nuclear localization sequence (NLS).

The expression plasmids for ZFM-SPB (SPB comprising a 93 amino acid N-terminal deletion and a DNA targeting domain comprising three Zing Finger Motifs ZF268) or ZFM-PBx (PBx comprising a 93 amino acid N-terminal deletion and a DNA targeting domain comprising three Zing Finger Motifs ZF268) were assembled using Gibson assembly. The reaction was carried out under isothermal conditions using three enzymatic activities: a 5′ exonuclease generates long overhangs, a polymerase fills in the gaps of the annealed single strand regions, and a DNA ligase seals the nicks of the annealed and filled-in gaps to assemble DNA fragments in the correct order.

The resulting expression plasmids encode the full-length DNA-binding domain-comprising transposases ZFM-SPB (SEQ ID NO: 58) and ZFM-PBx (SEQ ID NO: 59) comprising an N-terminal NLS. The expression of ZFM-SPB and ZFM-PBx is under the control of the EF1a promoter, and each coding sequence is followed by a C-terminal polyadenylation signal.

### Example 9: Design of Targeted Integration Sequences Flanking TTAA Integration Site

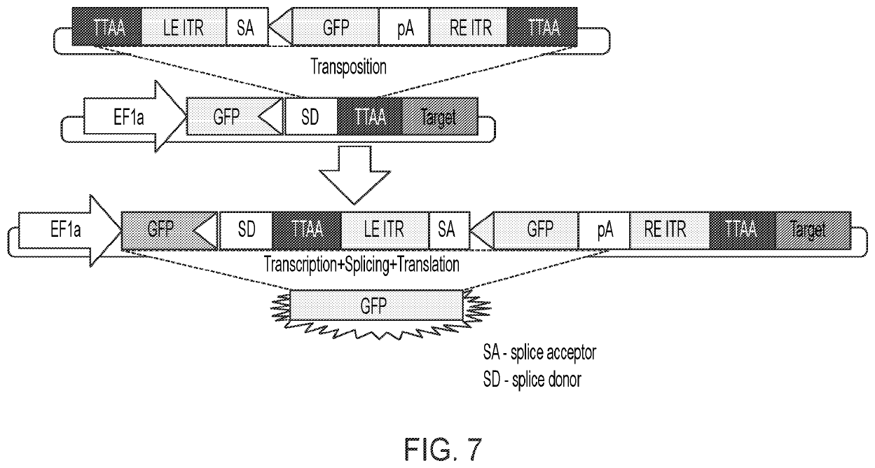

The TTAA target DNA integration site for SPB was modified to insert flanking DNA binding sites for the zinc finger protein ZF268. ZF268 binds to the 9-nucleotide DNA sequence GCGTGGGCG (SEQ ID NO: 60). A series of four constructs was prepared in which the distance between the TTAA site and the ZF268 binding sites was varied by 8, 7, 6 or 5 bp (SEQ ID NOS 61-64, respectively). The four constructs were individually cloned into the SplitGFP site-specific integration reporter plasmid to determine the relative differences in linker length on transposase-based integration. A schematic of the SplitGFP reporter plasmid is shown in FIG. 7.

### Example 10: Effect of Linker Length Between TTAA Integration Site and Flanking DNA binding Domain Sites on Integration and Excision Activity

The four targeted TTAA integration site constructs comprising various linker lengths generated in Example 9 were tested for transposase integration and excision activity. The reporter systems used to test for integration or excision are shown in FIGS. 6A-6C (dual excision/integration reporter) and FIG. 7 (SplitGFP Splicing Site Specific Reporter). FIG. 6A shows a schematic of the assays and FIGS. 6B and 6C show vector maps of the plasmids used.

### Integration Activity

The integration activity of the DNA-binding domain-comprising transposases was measured using a site-specific TTAA integration GFP reporter plasmid. If the DNA-binding domain-comprising transposases retain integration activity, then integration of a transposon into the site-specific TTAA integration site by a functional transposase restores a full-length GFP coding sequence resulting in expression of GFP from which positive GFP cells may be identified and quantified. Results are shown as percent positive GFP cells per cell population.

On Day 0, 60,000 HEK293 cells were seeded into 48 well plates. On Day 1, 25 ng plasmid encoding for transposase (e.g., wt-SPB, ZFM-SPB, or ZFM-PBx), 112.5 ng transposon donor plasmid, and 112.5 ng site-specific integration reporter plasmid comprising one of the differing linker lengths were delivered into specified wells of the 48-well plate and cells were co-transfected using jetPrime reagent (Polyplus) in accordance with the manufacturer's instructions. On Day 4, transfected cells were analyzed by flow cytometry to determine the percentage of GFP positive cells.

### Excision Activity

The excision activity of the DNA-binding domain-comprising transposases was measured using a transposon donor plasmid comprising the nucleotide sequence encoding the H2Kk gene containing an integrated transposon which interrupts the H2Kk coding sequence inactivating expression of a functional H2Kk protein. If the DNA-binding domain-comprising transposases retain excision activity, then the expressed fusion protein excises the integrated transposon restoring a full-length H2Kk coding sequence. H2Kk is a cell-surface protein, and its expression may be detected on the cell surface using a fluorescent anti-H2Kk antibody.

On Day 0, 60,000 HEK293 cells were seeded into 48 well plates. On Day 1, 25 ng plasmid encoding for transposase (e.g., wildtype-SPB, ZFM-SPB, or ZFM-PBx) and 112.5 ng transposon donor plasmid were delivered into each well of the 48-well plate and cells were co-transfected using jetPrime reagent (Polyplus) in accordance with the manufacturer's instructions. On Day 2, the cells were treated with a fluorescent anti-H2Kk antibody and analyzed by flow cytometry to determine the percentage of H2Kk positive cells.

### Results

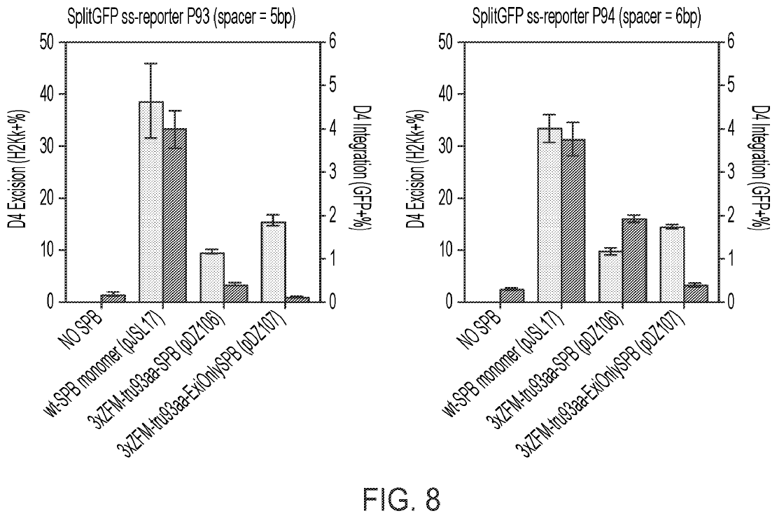

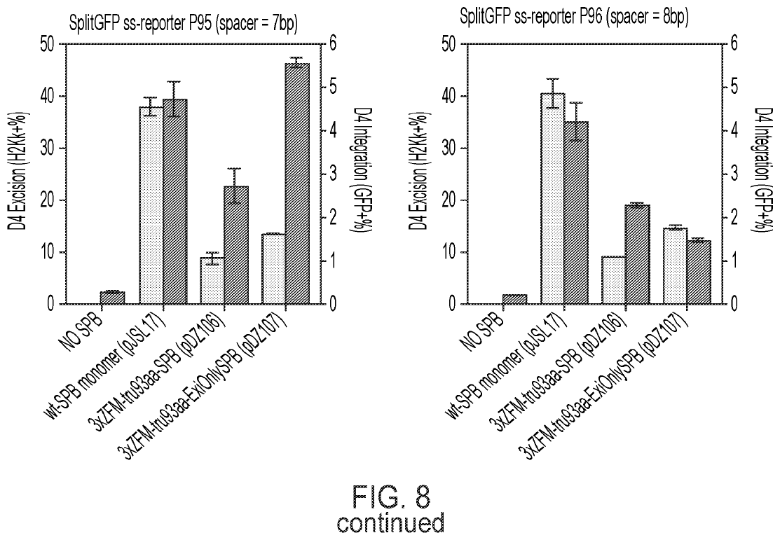

As shown in FIG. 8, wild type SPB, which lacks DNA binding domains, exhibited high levels of integration and excision activity irrespective of linker length, while ZMF-SPB demonstrated reduced but similar excision activity for all linker lengths compared to wild type SPB, and showed reduced but varied levels of integration activity compared to wild type SPB, with the highest level of integration activity detected with a 7 bp linker (˜50% WT SPB) and next highest level detected with an 8 bp linker.

ZFM-PBx demonstrated reduced but similar excision activity for all linker lengths compared to wild type SPB but slightly greater levels than ZFM-SPB. In contrast, however, ZFM-PBx showed widely varied levels of integration activity compared to wild type SPB and ZFM-SPB. ZFM-PBx exhibited reduced integration activity with linker lengths of 5, 6 and 8 compared to ZFM-SPB, and greatly reduced compared to wildtype SPB. For targeted TTAA integration sites comprising a linker length of 7 bp, ZFM-PBx exhibited integration levels that exceeded wild type SPB and were nearly double that of ZFM-SPB. The combined integration activity results suggest that a 7 bp linker between the TTAA integration site and flanking DNA binding sites is optimal for integration activity of the DNA-binding domain-comprising transposases described in example 8.

### Example 11: Random Genomic Integration Activity for Wild Type SPB, ZFM-SPB and ZFM-PBx

To determine excision activity and random genomic integration activity of the wild type SPB, ZFM-SPB and ZFM-PBx, a transposon containing a EF1a promoter and a full-length GFP coding sequence was used. Once the transposon is excised from the donor plasmid by the transposase (for example, the wild type SPB, ZFM-SPB or ZFM-PBx), integration takes place at random genomic TTAA sites. The random genomic integration activity is presented as the percentage of GFP positive cells.

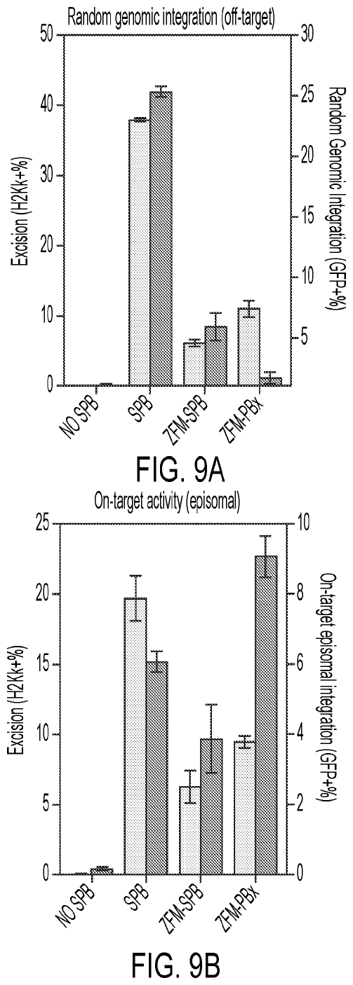

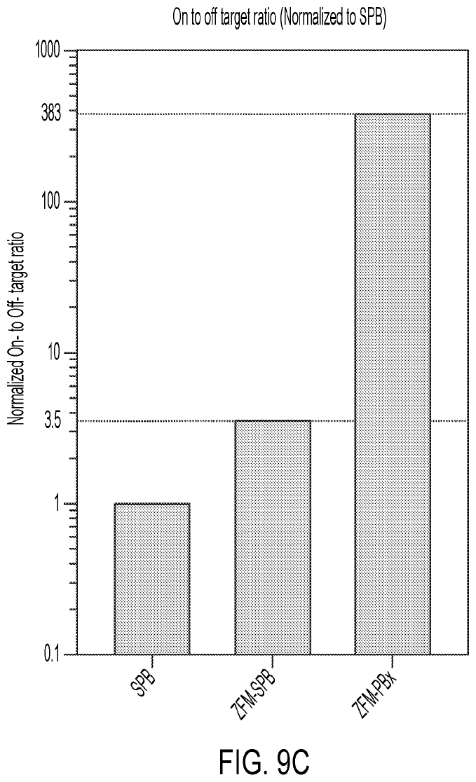

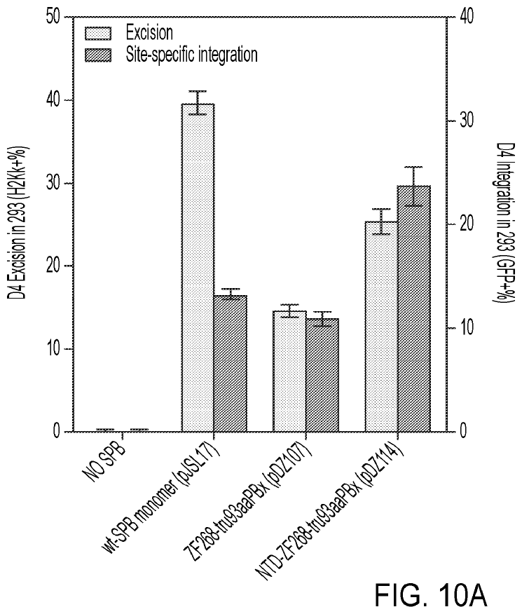

As shown in FIG. 9A, wild type SPB exhibits the highest level of random, off target genomic integration activity. In comparison, the ZFM-SPB showed reduced excision activity as well as random genomic integration activity. The reduced overall activity of ZFM-SPB is likely due to the truncated N-terminal of SPB. Notably, the excision activity of ZFM-PBx was significantly higher than the ZFM-SPB. This is likely because ZFM-PBx contains a D450N mutation, which is known to boost excision activity of piggyBac transposase. Importantly, the random genomic integration activity of ZFM-PBx was dramatically reduced, likely because the fusion protein is based on the integration deficient PBx. This elimination of random genomic integration is believed to be key to achieve a greater on-to-off integration ratio for ZFM-PBx.

### Example 12: Ratio of On Target to Off Target Integration Activity for Wild Type SPB, ZFM-SPB and ZFM-PBx

The SplitGFP site-specific episomal reporter plasmid comprising the TTAA integration site flanked by ZF268 binding sites with the optimal 7 bp linkers was used as a reporter to test the on-target episomal integration using wild type SPB, ZFM-SPB and ZFM-PBx transposases. Transposon integration at the site-specific TTAA target site restores functional GFP activity. Site-specific integration activity for wild type SPB, ZFM-SPB and ZFM-PBx was determined as described in Example 10 and is shown in FIG. 9B.

The ratio of on target to off target integration for ZFM-SPB and ZFM-PBx was calculated by dividing the on-target integration activity by the corresponding random genomic integration activity. Then the on target to off target integration ratio of ZFM-SPB and ZFM-PBx is normalized to the wild type SPB.

The results are shown in FIG. 9C. As shown in FIG. 9C, the ratio of on target to off target activity of ZFM-SPB is 3.5-fold compared to the wild type SPB. This result suggests that the zinc-finger binding motif indeed prioritized integration at the on-target TTAA site. However, this 3.5-fold enhancement is only a moderate improvement because even with a zinc-finger binding motif, the ZFM-SPB retains the ability to integrate randomly onto the genomic TTAA sites. In contrast, the ratio of on-target to off-target activity of ZFM-PBx was 383-fold compared to wild type SPB and over 100-fold greater than ZFM-SPB demonstrating enhanced on target and decreased off target, site-specific transposition.

### Example 13: Off and On Target Activity of ZFM-PBx with Intact N-Terminus

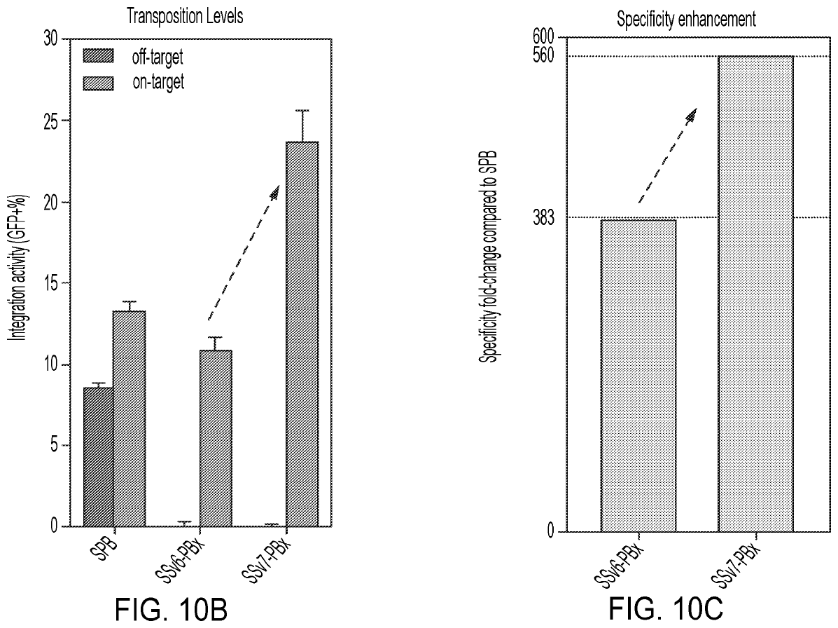

Excision activity and random genomic integration activity of SPB, ZFM-PBx and ZFM-PBx with a PSD (NTD-ZFM-PBx, SEQ ID NO: 67) were measured as described in Example 10 above. Results are shown in FIG. 10A. Both excision activity and integration activity were increased with NTD-ZFM-PBx compared to ZFM-PBx. FIG. 10B shows that on-target activity was increased in NTD-ZFM-PBx, while both ZFM-PBx and NTD-ZFM-PBx showed decreased off-target activity compared to SPB. FIG. 10C shows that the specificity of ND-ZFM-PBx relative to SPB is increased compared to the specificity of ZFM-PBx relative to SPB.

### Example 14: Design & Construction of TAL Arrays Targeting Specific Genes

This Example illustrates the design and construction of TAL Array compositions targeting exemplary genes that may be used to in methods to validate the target specificity of TAL Arrays.

Using the design criteria described herein or as set forth below, TAL Arrays were constructed targeting the following genes: GFP, zinc finger 268 (ZFN268), phenylalanine hydroxylase (PAH), beta-2-microglobulin (B2M) and LINE1 repeat elements.

### A. GFP

For proof-of-concept, TAL Array pairs comprising N-terminal domain recognizing a T were designed targeting specific, 10 bp right and 10 bp left pair sequences in the GFP coding region previously described (see e.g., Reyon et al., Nat Biotechnol. 2012 May; 30(5):460-5. doi: 10.1038/nbt.2170. PMID: 22484455; PMCID: PMC355894)7. In one instance, the left and right TAL Array pairs were designed to target TGCCACCTACG (SEQ ID NO: 240) and TGCAGATGAAC (SEQ ID NO: 241), respectively, generating GFP1 Left TAL Array (SEQ ID No 113) and GFP1 Right TAL Array (SEQ ID NO: 114).

A second set of TAL Array pairs comprising a N-terminal domain recognizing a T targeting GFP were designed to target the 10 bp GFP sequences TGGCCCACCCT (SEQ ID NO: 242) and TGCACGCCGTA (SEQ ID NO: 243), generating GFP2 Left TAL Array (SEQ ID No 115) and GFP2 Right TAL Array (SEQ ID NO: 116).

B. Zinc finger 268

A TAL Array comprising a N-terminal domain recognizing a T was designed targeting a specific, 10 bp sequence of a ZFM268 target site. The TAL Array was designed to target the zinc finger 268 sequence TACGCCCACGC (SEQ ID NO: 239) generating the ZFM268 TAL Array (SEQ ID NO: 112).

### C. PAH

TAL Array pairs comprising a N-terminal domain recognizing a T were designed targeting six, specific, 10 bp right and left pair sequences of the PAH gene, specifically present in introns 1 and 2 of the PAH gene. The TTAA sites are located 24 bp downstream of a T nucleotide and 24 bp upstream of an A nucleotide allowing for a 10 bp TAL recognition target site and a 13 bp spacer on either side of the TTAA. The left and right target sequences used to generate TAL Arrays that target the PAH gene are shown in Table 11.

TABLE 11
Illustrative TAL Arrays Targeting PAH

| PAH PAIR # | LEFT TARGET SEQUENCE | RIGHT TARGET SEQUENCE |
|---|---|---|
| 1 | TGAGATGATGT (SEQ ID NO: 244) | TCTCTTGTAAG (SEQ ID NO: 245) |
| 2 | TTCAGTTTGTT (SEQ ID NO: 246) | TCTTTTAGGAG (SEQ ID NO: 247) |
| 3 | TGCTTCATAGG (SEQ ID NO: 248) | TTTAGATCACA (SEQ ID NO: 249) |
| 4 | TATGATCCTAA (SEQ ID NO: 250) | TGATTGCTAAG (SEQ ID NO: 251) |
| 5 | TTCTAGGAAAC (SEQ ID NO: 252) | TTTTGTTTCCT (SEQ ID NO: 253) |
| 6 | TTGGCAGCCAC (SEQ ID NO: 254) | TGCCACTATAA (SEQ ID NO: 255) |

The six left and right pair combinations were used to design and construct PAH Left TAL Arrays 1-6 (SEQ ID Nos 117, 119, 121, 123, 125 & 127, respectively) and PAH Right TAL Arrays 1-6 (SEQ ID Nos 118, 120, 122, 124, 126, & 128, respectively).

### D. B2M

TAL Array pairs comprising a N-terminal domain recognizing a T were designed targeting seven, specific, 10 bp right and left pair sequences of the B2M gene. The left and right TAL Array target sequences used to design TAL Arrays targeting the B2M gene are shown in Table 12.

TABLE 12
Illustrative TAL Arrays Targeting B2M

| B2M PAIR # | LEFT TARGET SEQUENCE | RIGHT TARGET SEQUENCE |
|---|---|---|
| 1 | TGATACAAAGC (SEQ ID NO: 271) | TGACATGTGAT (SEQ ID NO: 272) |
| 2 | TGAAGAAACTA (SEQ ID NO: 273) | TTATCCCCTGT (SEQ ID NO: 274) |
| 3 | TGGCTGTAATT (SEQ ID NO: 275) | TCACGCAGAAG (SEQ ID NO: 276) |
| 4 | TCTGTGCTCTG (SEQ ID NO: 277) | TGAGCTTCTAA (SEQ ID NO: 278) |
| 5 | TTGATGGGGCT (SEQ ID NO: 279) | TATCTCTCTAG (SEQ ID NO: 280) |
| 6 | TTTTATCGGGT (SEQ ID NO: 281) | TGCATACAAGA (SEQ ID NO: 282) |
| 7 | TTGAGAGCCTC (SEQ ID NO: 283) | TCACTGGAGAT (SEQ ID NO: 284) |
| 8 | TTTGTTCCCAT (SEQ ID NO: 514) | TAACGGGTAGT (SEQ ID NO: 515) |
| 9 | TTGCTGGTTAT (SEQ ID NO: 516) | TTTAAATATCA (SEQ ID NO: 517) |

Individual TAL modules containing 34 amino acid or 20 amino acid “half” repeats were synthesized flanked by BsmBI type IIS restriction sites. The entire module set contains 4 modules capable of recognizing either A, C, G, T for each of 10 bp positions within a target sequence (40 modules/10 bp target). Pairs of TAL arrays targeting sequences in the B2M gene were designed and the corresponding modules were selected and pooled together using “Golden Gate Assembly,” to assemble in frame to create each B2M TAL-Arrays. All coding sequences used were codon optimized for human expression.

The nine left and right pair combinations were used to design and construct B2M Left TAL Arrays 1-7 (SEQ ID Nos 144, 146, 148, 150, 152, 154, 156, 518, and 520 respectively) and B2M Right TAL Arrays 1-7 (SEQ ID Nos 145, 147, 149, 151, 153, 155, 157, 519, and 521, respectively).

### E. LINE1 Repeat Elements

TAL Array pairs comprising a N-terminal domain recognizing a T were designed targeting six, specific, 10 bp right and left pair sequences of the LINE-1 repeat elements. Some of the LINE1 pairs had more than one left or right target sequence designed against the same location.

The left and right target sequences used to design TAL Array pairs targeting LINE1 repeat elements are shown in Table 13.

TABLE 13
Ilustrative TAL Arrays Targeting LINE1

| LRE PAIR # | LEFT TARGET SEQUENCE | RIGHT TARGET SEQUENCE |
|---|---|---|
| 1 | TATAAATGGAC (SEQ ID NO: 256) | TCCAACTTGCC (SEQ ID NO: 257) |
| 2 | TCCTAGTCTCT (SEQ ID NO: 258) | TGTCTCTTTTG (SEQ ID NO: 259) |
| | | TTGTCTCTTTT (SEQ ID NO: 260) |
| 3 | TGCAATCAAAC (SEQ ID NO: 261) | TTGAGCGGCTT (SEQ ID NO: 262) |
| 4 | TTCACAGAATT (SEQ ID NO: 263) | TCTTTTTTGGT (SEQ ID NO: 265) |
| | TCACAGAATTG (SEQ ID NO: 264) | |
| 5 | TACAAAAATCA (SEQ ID NO: 266) | TTTTAGGTTTA (SEQ ID NO: 267) |
| 6 | TCAATTCAAGA (SEQ ID NO: 268) | TTTTATGGTTT (SEQ ID NO: 269) |
| | | TTTTTATGGTT (SEQ ID NO: 270) |

Individual TAL modules containing 34 amino acid or 20 amino acid “half” repeats were synthesized flanked by BsmBI type IIS restriction sites. The entire module set contains 4 modules capable of recognizing either A, C, G, T for each of 10 bp positions (40 modules/10 bp target). Pairs of TAL arrays targeting sequences in the LINE1 repeats were designed and the corresponding modules were selected and pooled together using “Golden Gate Assembly,” to assemble in frame each LRE TAL-Arrays. All coding sequences used were codon optimized for human expression.

The nine left and right pair target sequences were used to design and construct LINE1 repeat element (LRE) Left TAL Arrays LREL1, LREL2, LREL3, LRE4L1, LRE4L2, LREL5, and LREL6 (SEQ ID Nos 129, 131, 134, 136, 137, 139 & 141, respectively) and LINE1 repeat elements right TAL Arrays LRE1, LRE2R1+, LRE2R2+, LRER3, LRER4, LRER5, LRE6R1+ and LRE6R2+(SEQ ID Nos, 130, 132, 133, 135, 138, 140, 142 & 143 respectively).

### Example 15: General Methods for Design & Construction of TAL-FokI Fusions (aka TALENs)

This Example illustrates exemplary general methods for the design and construction of TALENs that may be used in methods to validate TAL Array target specificity.

The target site specificity of TAL Arrays, e.g., TAL Arrays constructed in Example 14, was determined, in part, by construction of TAL-FokI fusion proteins (TALENs) that were used in subsequent assays to measure TAL-specific endonuclease activity at designed target site locations.

An TALEN expression plasmid was designed and synthesized that contains from the 5′ to 3′ direction: a CMV promoter, a T7 promoter, a Kozak sequence, a 3× Flag tag (SEQ ID NO: 70), an SV40 NLS (SEQ ID NO: 71), the Delta 152 TAL N-terminal domain (SEQ ID NO: 73), two BsmBI type IIS restriction enzyme sites for the insertion of a left TAL Array or a right TAL Array, the +63 TAL C-terminal domain (SEQ ID NO: 76), a GS linker, a FokI nuclease domain (SEQ ID NO: 79), and a bGH poly adenylation sequence.

Cloning of BsmBI-flanked left or right TAL Arrays into the BsmBI sites of the expression plasmid results in-frame fusion of the TAL Array and the FokI coding sequence via a linker generating full-length TALENs. All coding sequences used were codon optimized for human expression using GeneArt algorithms (Thermo Fisher).

### Example 16: Construction of TAL-FokI Fusions (TALENs) Targeting Specific Genes

This Example illustrates the construction of TALENs comprising the TAL Arrays designed and constructed in Example 14.

Expression vectors comprising TALENs comprising each of the TAL Arrays comprising a N-terminal domain recognizing a T constructed in Example 14 were prepared as generally set forth in Example 15.

### A. GFP

The DNA sequence encoding the GFP1 left TAL or right TAL Arrays, or the GFP2 left TAL or right TAL Arrays of Example 14A containing flanking BsmBI ends were individually cloned into the BsmBI type IIS restriction enzyme sites of the TALEN expression vector generating GFP1 TALENS (SEQ ID Nos. 159 & 160) and GFP2 TALENs (SEQ ID Nos. 161 & 162).

### B. ZFN268

The DNA sequence encoding the ZFN268 TAL Array of Example 14B containing flanking BsmBI ends were cloned into the BsmBI type IIS restriction enzyme sites of the TALEN expression vector to generate ZFN268 TALEN (SEQ ID NO: 158).

### C. PAH

The DNA sequence encoding the PAH Pair Nos 1-6 left or right TAL Arrays of Example 14C containing flanking BsmBI ends were individually cloned into the BsmBI type IIS restriction enzyme sites of the TALEN expression vector generating 12 PAH left and right TALENs (SEQ ID Nos. 163, 165, 167, 169, 171 & 173) and (SEQ ID Nos. 164, 166, 168, 170, 172 & 174), respectively.

### D. LINE1 Repeat Elements

The DNA sequence encoding the LINE1 repeat elements (LRE) Pair Nos 1-6 left or right TAL Arrays of Example 14E containing flanking BsmBI ends were individually cloned into the BsmBI type IIS restriction enzyme sites of the TALEN expression vector of generating 16 LRE left and right TALENs LRE1L, 2L, 3L, 4L1, 4L2, 5L, and 6L (SEQ ID Nos. 175, 177, 180, 182, 183, 185 & 187) and LRE1R, 2R1+, 2R2+, 3R, 4R, 5R, 6 R1+ and 6R2+(SEQ ID Nos. 176, 178, 179, 181, 184, 186, 188 & 189), respectively.

### Example 17: Methods for Analyzing TAL Array Target Site Specificity Using TALENs in a Single Strand Annealing (SSA) Assay

This Examples illustrates an exemplary assay for determining site-specific cleavage of target sites by TALENs comprising TAL Arrays of the presentation invention.

The sequence-specificity of TALENs (including those constructed in Example 16) comprising TAL Arrays, e.g., TAL Arrays constructed in Example 14, was determined, in part, by using a single strand annealing (SSA) assay.

A SSA luciferase reporter plasmid was designed and synthesized as previously described (e.g., see Juillerat A, et al., Comprehensive analysis of the specificity of transcription activator-like effector nucleases. Nucleic Acids Res. 2014 April; 42(8):5390-402. doi: 10.1093/nar/gku155. Epub 2014 Feb. 24. PMID: 24569350; PMCID: PMC4005648). The plasmid contains in a 5′ to 3′ direction: a CMV promoter, a Kozak sequence, the first N-terminal segment of the Firefly luciferase coding sequence (SEQ ID NO: 237), two stop codons, two BsaI type IIS restriction sites, the second C-terminal segment of the Firefly luciferase coding sequence (SEQ ID NO: 238) and an SV40 poly adenylation sequence. The two segments of Firefly luciferase coding sequence contain 628 bp of overlapping sequence. If the target site for a TALEN is cloned at the BsaI sites and the reporter construction is cut, it can be repaired in cells by single strand annealing leading to a full-length Firefly luciferase coding sequence and expression of Firefly luciferase (SEQ ID NO: 236) indicating that the TALEN site-specifically recognizes its target site.

Complementary oligos were synthesized containing the target site for each TAL Array downstream of a T followed by a 16 bp spacer followed by the reverse complement of the TAL target site followed by an A. Additionally, complementary oligos containing the target site for a left TAL Array followed by a 16 bp spacer followed by the reverse complement of the target site for a right TAL Array followed by an A were synthesized. The complementary oligos contained 4 bp overhangs compatible with the overhangs created in the SSA reporter following digestion with BsaI. The oligos were annealed and ligated into the digested vector to create an SSA reporter compatible with each TALEN.

### GFP

For instance, GFP1 reporter plasmids comprising two left TAL Array target sequences (SEQ ID NO: 287), two right TAL Array target sequences (SEQ ID NO: 288), one left and one right TAL Array (SEQ ID NO: 286), and GFP2 reporter plasmids comprising two left TAL Array target sequences (SEQ ID NO: 290), two right TAL Array target sequences (SEQ ID NO: 291), one left and on right TAL Array (SEQ ID NO: 289). Furthermore, a ZFN268 TAL Array target site (SEQ ID NO: 285) was prepared as a second target. All of these constructs were used in subsequent SSA assays.

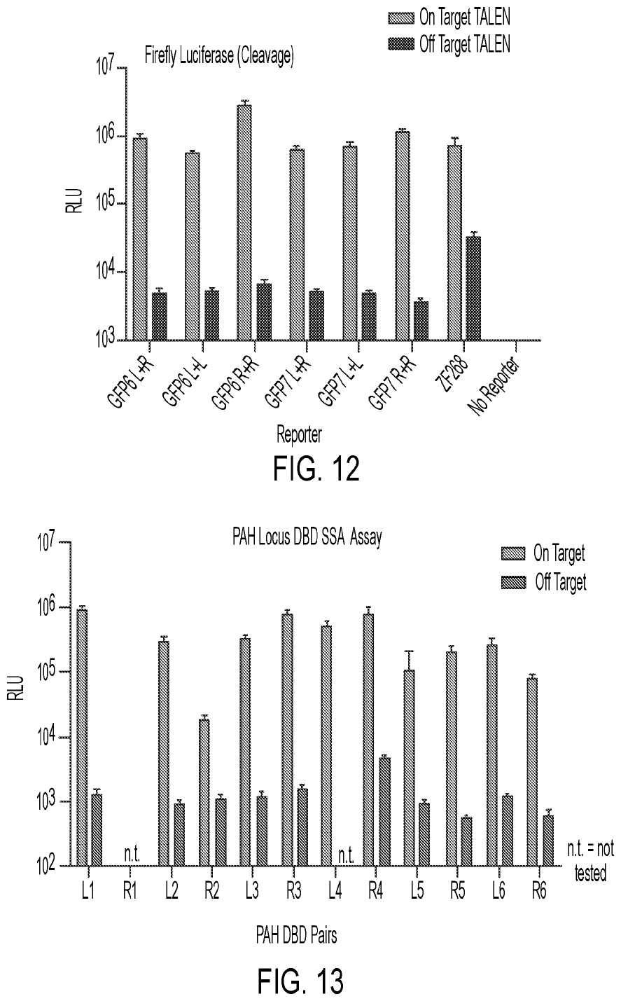

The cleavage activity of the six GFP TALENS (GFP1 & GFP2) and the ZFM268 TALEN constructed in Example 16 was determined. A transfection mixture containing 45 ng of the left TALEN, 45 ng of the right TALEN, 10 ng of the corresponding reporter and 0.3 μl of Transit-2020 transfection reagent in a total volume of 20 μl of Serum Free OptiMem medium were assembled. As a negative control, each TALEN pair was also co-transfected with a reporter lacking the correct target site sequence. 60,000 HEK293T cells in 180 μl of DMEM medium supplemented with 10% FBS were added and the transfection mixture was plated in 96 well plates and incubated for one day at 37° C. at 5% CO2. The following day, a lysis buffer was added to the cells and the lysate was transferred to a white 96 well plate. A buffer containing substrate for Firefly luciferase was mixed with the cells and luciferase luminescence was detected using a plate reader. The results are shown in Table 14 and FIG. 12.

TABLE 14

Luminescence

| Reporter | On-Target TALENs | Off-Target TALENs |
|---|---|---|
| GFP1 L + R | 992215 | 5120 |
| GFP1 L + L | 576598 | 5575 |
| GFP1 R + R | 2955917 | 7187 |
| GFP2 L + R | 722351 | 5475 |
| GFP2 L + L | 738908 | 5093 |
| GFP2 R + R | 1279891 | 3937 |
| ZFN268 | 847643 | 33555 |
| No Reporter | 335 | 91 |

As shown in Table 14 luciferase was readily detected at levels orders of magnitude higher when the corresponding TALEN and reporter pair was cotransfected together than in the negative controls demonstrating onsite target activity of each TALEN construct.

### PAH

In another experiment, SSA reporter plasmids targeting PAH were designed and constructed for each constructed PAH TALEN in Example 16C: PAH1-6 Left TALEN (SEQ ID Nos. 163, 165, 167, 169, 171 & 173) and PAH1-6 Right TALEN (SEQ ID Nos. 164, 166, 168, 170, 172 & 174).

The SSA assay was performed using methods described above. Briefly, two copies of each PAH target site separated by a 16 bp spacer, PAH1 Left and Right (SEQ ID Nos. 292 & 293); PAH2 Left and Right (SEQ ID Nos. 294 & 295); PAH3 Left and Right (SEQ ID Nos. 296 & 297); PAH4 Left and Right (SEQ ID Nos. 298 & 299); PAH5 Left and Right (SEQ ID Nos. 300 & 301); and PAH6 Left and Right (SEQ ID Nos. 302 & 303) were cloned into the SSA reporter plasmid.

Each TALEN was co-transfected with its corresponding reporter or a reporter containing a non-target sequence and luciferase was measured the following day. The results are show in Table 15 and FIG. 13.

TABLE 15
Luminescence

| PAH | On-Target | Off-Target |
|---|---|---|
| TALEN | Reporter | Reporter |
| L1 | 949448 | 1253 |
| R1 | | |
| L2 | 301341 | 935 |
| R2 | 18694 | 1158 |
| L3 | 333157 | 1229 |
| R3 | 783785 | 1617 |
| L4 | 513293 | |
| R4 | 819902 | 4796 |
| L5 | 107539 | 922 |
| R5 | 202932 | 570 |
| L6 | 258454 | 1276 |
| R6 | 79699 | 627 |

### LINE-1 Repeat Elements

In another experiment, SSA reporter plasmids with two copies of each LINE1 target site separated by a 16 bp spacer (SEQ ID Nos. 304-318) targeting LINE1 Repeat Elements were designed and constructed for each constructed LINE1 TALEN in Example 16D: TALENs LRE1L, 2L, 3L, 4L1, 4L2, 5L, and 6L (SEQ ID Nos. 175, 177, 180, 182, 183, 185 & 187) and LRE1R, 2R1+, 2R2+, 3R, 4R, 5R, 6 R1+ and 6R2+(SEQ ID Nos. 176, 178, 179, 181, 184, 186, 188 & 189), respectively. Results are shown in Table 16.

TABLE 16

| | On-target | | | Off-target | | |
|---|---|---|---|---|---|---|
| | Replicate 1 | Replicate 2 | Average | Replicate 1 | Replicate 2 | Average |
| LINE-L1 | 909906 | 969818 | 939862 | 8271 | 8139 | 8205 |
| LINE-R1 | 1080209 | 1014380 | 1047295 | 6751 | 6297 | 6524 |
| LINE-R2.1 | 2878385 | 2711672 | 2795029 | 18834 | 17107 | 17971 |
| LINE-R2.2 | 1032426 | 1040898 | 1036662 | 5562 | 5048 | 5305 |
| LINE-L2 | 1511468 | 1452962 | 1482215 | 6880 | 6333 | 6607 |
| LINE-L3 | 919092 | 922022 | 920557 | 5364 | 4265 | 4815 |
| LINE-R3 | 894269 | 879554 | 886912 | 6011 | 6509 | 6260 |
| LINE-L4.1 | 549160 | 596327 | 572744 | not tested | | |
| LINE_R4 | 467252 | 467172 | 467212 | 12820 | 12345 | 12583 |
| LINE_L4.2 | 744872 | 827243 | 786058 | 12210 | 10940 | 11575 |
| LINE_L5 | 42147 | 39382 | 40765 | 5568 | 4579 | 5074 |
| LINE-R5 | 249997 | 252029 | 251013 | 8989 | 8177 | 8583 |
| LINE-R6.1 | 145949 | 130527 | 138238 | 15065 | 14921 | 14993 |
| LINE-L6 | 588448 | 569939 | 579194 | 37224 | 32600 | 34912 |
| LINE-R6.2 | 9836 | 9357 | 9597 | 25882 | 22347 | 24115 |

As shown in Table 16, most TALENs tested resulted in luciferase signal greater than an order of magnitude higher when using the on-target reporter vs the off-target reporter. The SSA assay demonstrates that the newly designed TALs are capable of recognizing their intended target sequence allowing for a fused FokI nuclease to cut adjacent DNA, resulting in single strand annealing and luciferase expression.

### Example 18: Construction and Analysis of TAL Array—piggyBac Transposase (ss-SPB) Compositions (TAL-PBxs) Designed for Site-Specific Transposition at Specific Genes

This Example illustrates the construction of TAL Array—Super piggyBac transposase fusion protein compositions (TAL-ssSPB) that are useful in methods for achieving site-specific transposition at a specific target locus.

Analogous to the ZFM268-PBx constructs described in Examples 14 and 16 above, TAL-PBx fusion constructs were prepared. An expression plasmid was synthesized that contains from 5′ to 3′ direction: a CMV promoter, a T7 promoter, a Kozak sequence, a 3× Flag tag (SEQ ID NO: 70), an SV40 NLS (SEQ ID NO: 71), the Delta 152 TAL N-terminal domain (SEQ ID NO: 73), two BsmBI type IIS restriction enzyme sites, the +63 TAL C-terminal domain (SEQ ID NO: 76), a GGGS linker, delta 1-93 PBx (comprising a N-terminal 93 amino acid deletion and mutations at R372A, K375A, D450N in the Super piggyBac transposase codon sequence; SEQ ID NO: 66), and a bGH poly adenylation sequence.

Cloning of a BsmBI-flanked left or right TAL Array into the BsmBI sites of the expression plasmid results in-frame fusion of the TAL Array and the PBx coding sequence via a linker sequence generating full-length TAL-PBx constructs. All coding sequences used were codon optimized for human expression using GeneArt algorithms (Thermo Fisher).

### A. GFP1 & 2 TAL-PBx & ZFM 268 TAL-PBx

The two pairs of TAL arrays targeting sequences in the GFP coding sequence in Example 14A as well as a TAL array targeting a ten base pair sequence (ACGCCCACGC downstream of a T; SEQ ID NO: 239) that contains the reverse complement of the ZFM 268 target site in Example 14B were designed. Each TAL Array containing nine 34 amino acid repeats followed by the 20 amino acid “half” repeat were synthesized flanked by BsmBI type IIS restriction sites. This allowed for cloning of each TAL array in-frame with the rest of the open reading frame in the expression plasmid to generating GFP1 Left TAL-PBx (SEQ ID NO: 191), GFP1 Right TAL-PBx (SEQ ID NO: 192), GFP2 Left TAL-PBx (SEQ ID NO: 193), GFP2 Right TAL-PBx (SEQ ID NO: 194) and ZFM 268 TAL-PBx (SEQ ID NO: 190). All coding sequences used were codon optimized for human expression.

The GFP TAL-PBx and ZFM 268 TAL-PBx constructs were used in Example 19 to determine optimal spacer distance between TTAA integration site and positioning of left and right TAL target sequence for TAL-PBx constructs.

### B. PAH1-6 Left & Right TAL-PBx

The PAH locus was chosen as a target for site-specific transposition into genomic DNA. Within the first two introns, six TTAA sites were selected that fit the motif described herein. TAL arrays targeting these sequences were synthesized in Example 14C and cloned into TAL-ssSPB expression vectors using methods described in the Examples 17, thereby generating PAH 1-6 Left TAL-PBx (SEQ ID Nos. 195, 197, 199, 201, 203 & 205, respectively) and PAH 1-6 Right TAL-PBx sequences (SEQ ID Nos. 196, 198, 200, 202, 204 & 206, respectively).

### C. B2M Left & Right TAL-PBx

The nine TAL Arrays designed and constructed in Example 14D flanked with BsmBI ends were cloned into the BsmBI restriction sites of the expression plasmid described above to generate eighteen B2M1-9 TAL-PBx constructs: B2M1-9 Left TAL-PBx (SEQ ID Nos. 222, 224, 226, 228, 230, 232, 234, 522, and 524 respectively) and B2M1-9 Right TAL-PBx (SEQ ID Nos. 223, 225, 227, 229, 231, 233, 235, 523, and 525 respectively).

### D. LINE1 Repeat Elements Left & Right TAL-PBx

LINE1 repeat elements occur thousands of times throughout the human genome making them potential attractive targets for optimizing the chance of a site-specific transposition event at a target sequence thereby leading to increased number of transposed cells.

The fifteen TAL Arrays designed and constructed in Example 14E flanked with BsmBI ends were cloned into the BsmBI restriction sites of the expression plasmid described above to generate fifteen LRE1-6 TAL-PBx constructs: LRE1L, LRE2L, LRE3L, LRE4.1L, LRE4.2L, LRE5L, and LRE6L Left TAL-PBxs (SEQ ID Nos. 207, 209, 212, 214, 215, 217 & 219, respectively) and LRE1R, LRE2.1R, LRE2.2R, LRE3R, LRE4R, LRE5R, LRE6.1R and LRE6.2R Right TAL-PBxs (SEQ ID Nos. 208, 210, 211, 213, 216, 218, 220 & 221, respectively).

### Example 19: Determination of Optimal Spacer Length Between TTAA Integration Site and Left and Right TAL Target Sequences Using an Episomal Split GFP Splicing Reporter System

This Example illustrates exemplary compositions and methods for preparing optimal target sites for site-specific transposition using TAL Array—SPB transposase fusion proteins.

An episomal split GFP splicing reporter system was employed to evaluate differing spacer length on site-specific transposition efficiency. The reporter system consists of two plasmids. The first plasmid, “the reporter,” was constructed containing from 5′ to 3′ direction: an EF1a promoter (SEQ ID NO: 325), a Kozak sequence, the first portion of a GFP open reading frame (SEQ ID NO: 326), a splice donor (SEQ ID NO: 327), and two BsaI type IIS restriction enzyme sites. The BsaI sites allow for cloning a target TTAA sequence flanked by spacers of variable length flanked by target recognition sequences for TAL arrays. The second plasmid, “the donor,” was constructed containing from 5′ to 3′ direction: a TTAA sequence, the 35 bp PiggyBac minimal 5′ ITR (SEQ ID NO: 319), a splice acceptor site (SEQ ID NO: 321), the second portion of a GFP open reading frame (SEQ ID NO: 322), a synthetic poly adenylation sequence (SEQ ID NO: 323), the 63 bp PiggyBac minimal 3′ ITR (SEQ ID NO: 320), and a TTAA sequence.

Complementary oligos were synthesized containing the target site for the GFP1 Right TAL downstream of a T followed by a 6 bp spacer followed by TTAA followed by a 6 bp spacer, followed by the reverse complement of the TAL target site followed by an A (SEQ ID NO: 330). The complementary oligos contained 4 bp overhangs compatible with the overhangs created in the split GFP splicing reporter following digestion with BsaI. The oligos were annealed and ligated into the digested vector to create a reporter compatible with the GFP1 Right TAL-PBx. Similar oligos where synthesized replacing the two 6 bp spacers with spacers of 7 bp (SEQ ID NO: 331), 8 bp (SEQ ID NO: 332), 9 bp (SEQ ID NO: 333), 10 bp (SEQ ID NO: 334), 11 bp (SEQ ID NO: 335), 12 bp (SEQ ID NO: 336), 13 bp (SEQ ID NO: 337), 14 bp (SEQ ID NO: 338), and 15 bp (SEQ ID NO: 339) in length. These were cloned in the same fashion to create reporters with spacers of variable lengths.

Each reporter plasmid and the donor plasmid were cotransfected into HEK293T cells with the GFP1 Right TAL-PBx expression plasmid. As a negative control, the ZFM268 TAL-PBx expression plasmid, which does not recognize the GFP1 target sequence, was transfected in place of the GFP1 Right TAL-PBx expression plasmid. Transfection mixtures containing 26 ng of the TAL-ssSPB expression vector, 170 ng of the reporter plasmid, 117 ng of donor plasmid and 0.78 ul of Transit-2020 transfection reagent in a total volume of 26 μl of Serum Free OptiMem medium were assembled. 95,000 HEK293T cells in 250 ul of DMEM medium supplemented with 10% FBS were added and the transfection mixture was plated in 48 well plates and incubated for four days at 37° C. at 5% CO2, splitting the cells 1:3 at day two.

When the reporter and donor plasmids are co-transfected into cells along with TAL-PBx, TAL-PBx catalyzes the excision of the transposon from the donor plasmid and its site-specific integration into the TTAA target site of the reporter plasmid. Following site-specific transposition, transcription, splicing, and translation, a reconstituted GFP coding sequence is produced (DNA, SEQ ID NO: 328; Amino acid; SEQ ID NO: 329) and fluorescence can be detected. The percentage of on-target site-specific transposition positive cells for the various spacer length constructs were determined by FACS analysis and the results are shown in Table 17.

TABLE 17
% GFP+ Cells

| Spacer Length | GFP1 RIGHT TAL-PBx | ZFM268 TAL-PBx |
|---|---|---|
| 6 | 3.0 | 3.6 |
| 7 | 3.0 | 3.1 |
| 8 | 2.9 | 2.4 |
| 9 | 3.6 | 2.7 |
| 10 | 3.2 | 2.6 |
| 11 | 2.7 | 2.5 |
| 12 | 10.4 | 2.9 |
| 13 | 15.3 | 3.2 |
| 14 | 9.0 | 3.2 |
| 15 | 4.5 | 3.0 |

As shown in FIG. 13, the GFP1 Right TAL-PBx catalyzed site-specific transposition leading to GFP signal above background levels with target sites containing 12 bp, 13 bp, and 14 bp spacers separating the TTAA integration site from the TAL binding sites. The negative control ZFM268 TAL-PBx resulted in no GFP signal above background using the GFP1 Right specific reporters.

To determine if the optimal spacer length is consistent from one TAL-ssSPB to the next, similar reporters were constructed with TAL target sites for the GFP1 Left, GFP2 Right, GFP2 Left, and ZFM268 TAL-PBxs as described above. These constructs were tested using a narrower set of spacer lengths of 11 bp, 12 bp, 13 bp, 14 bp, 15 bp constructs for GFP1 Left (SEQ ID Nos. 345-349), GFP2 Left (SEQ ID Nos. 350-354), GFP2 Right (SEQ ID Nos. 355-359) and ZFM268 (SEQ ID NOs: 340-344).

Each reporter plasmid and the donor plasmid were cotransfected into HEK293T cells with the corresponding TAL-ssSPB expression plasmid. 120,000 HEK293T cells were plated in 24 well plates in 500 ul of DMEM medium supplemented with 10% FBS. The following day, a transfection mixture containing 50 ng of the TAL-ssSPB expression vector, 225 ng of the reporter plasmid, 225 ng of donor plasmid and 1 μl of JetPrime transfection reagent in a total volume of 50 μl of JetPrime buffer were assembled. The mixture was added to the HEK293T cells and the cells were incubated for four days at 37° C. at 5% CO2, splitting the cells 1:6 at day one. The percentage of on-target site-specific transposition positive cells for the various spacer length constructs were determined by FACS analysis and the results are shown in Table 18.

TABLE 18
Linker Length

| Construct | 11 | 12 | 13 | 14 | 15 |
|---|---|---|---|---|---|
| GFP1 Right | 3.4 | 13.2 | 15.3 | 8.1 | 5.4 |
| GFP1 Left | 4.6 | 13.7 | 17.0 | 8.6 | 5.1 |
| GFP2 Right | 6.6 | 16.9 | 15.8 | 11.4 | ND |
| GFP2 Left | ND | 22.4 | 23.5 | 12.4 | 5.2 |
| ZFN268 | ND | 21.8 | 21.2 | 11.6 | ND |

As shown in Table 18, the 12 bp and 13 bp spacers were optimal resulting in the highest GFP expression from site-specific transposition of the donor transposon into the reporter plasmid in the cell population for all TAL-PBx constructs and targets tested.

In another experiment, the donor plasmid target integration site comprising optimal 13 bp spacers was modified to mutate the flanking 5′ and 3′ nucleotide immediately adjacent to the TTAA integration sequence to a T and an A, respectively, to generate a TTTAAA integration site flanked by 12 bp spacers between the two TAL target sequences: GFP1 Right (SEQ ID NO: 382); GFP2 Left (SEQ ID NO: 383); GFP2 Right (SEQ ID NO: 384); GFP2 Left (SEQ ID NO: 385) and ZFM268 (SEQ ID NO: 386). The modified TTTAAA (13 bp v2) and TTAA (13 bp) donor plasmids were compared using the episomal split GFP splicing reporter system using GFP1 Left TAL-PBx, GFP1 Right TAL-PBx, GFP2 Left TAL-PBx, GFP2 Right TAL-PBx, ZFM268 TAL-PBx expression plasmids described in Example 18A.

Briefly, each reporter plasmid and the donor plasmid were cotransfected into HEK293T cells with the corresponding TAL-PBx expression plasmid. Approximately 120,000 HEK293T cells were plated in 24 well plates in 500p of DMEM medium supplemented with 10% FBS. The following day, a transfection mixture containing 50 ng of the GFP1 TAL-PBx or ZFM268 TAL-PBx expression vector, 225 ng of the reporter plasmid, 225 ng of donor plasmid and 1 μl of JetPrime transfection reagent in a total volume of 50 μl of JetPrime buffer were assembled. This mixture was added to the HEK293T cells and they were incubated for four days at 37° C. at 5% CO2, splitting the cells 1:6 at day one. The percentage of GFP positive cells was determined for each TTAA or TTTAAA integration site construct and the results are shown in Table 19.

TABLE 19

| | 13 bp | | | 13 bp v2 | | |
|---|---|---|---|---|---|---|
| | Duplicate 1 | Duplicate 2 | Average | Duplicate 1 | Duplicate 2 | Average |
| ZFM268 | 17.9 | 15.6 | 16.75 | 33 | 29.5 | 31.25 |
| GFP1-L | 18.9 | 14.3 | 16.6 | 36.6 | 35.5 | 36.05 |
| GFP1-R | 15.3 | 13.2 | 14.25 | 33 | 31.9 | 32.45 |
| GFP2-L | 25.7 | 26.2 | 25.95 | 43.9 | 43.3 | 43.6 |
| GFP2-R | 16.6 | 16.1 | 16.35 | 35.3 | 35.1 | 35.2 |

As shown in Table 19, the modification of the TTAA integration site to TTTAAA resulted in approximately a 2-fold increase in the number of GFP expressing cells within the transposed cell population for each GFP TAL-PBx as well as ZFM268 TAL-PBx.

### Example 20: TAL-PBx Targeted Site-specific Transposition at Specific Gene Loci

This Example illustrates that the TAL-ssSPB (TAL-PBx) compositions of the present invention are capable of site-specific transposition of a transposon at specific episomal and genomic loci.

### A. PAH Episomal and Genomic Target Site-Specific Transposition

i. Episomal

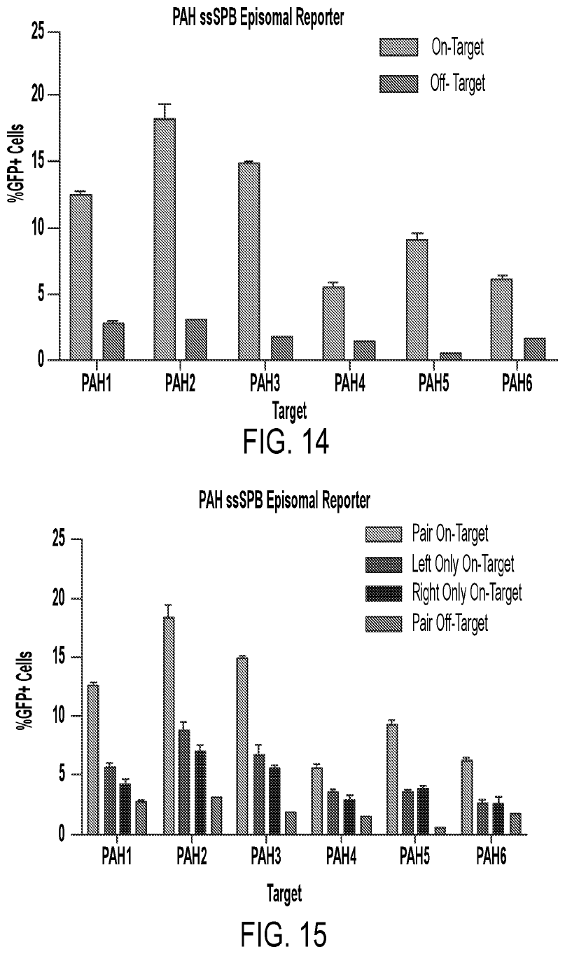

Episomal split GFP splicing reporter constructs were designed and cloned as described above. Six PAH target sequences naturally found in genomic DNA (SEQ ID Nos. 360-365) were cloned into the episomal reporter plasmid. These plasmids were cotransfected with the TAL recognition sequence, an optimal length 13 bp spacer, TTAA, a second optimal length 13 bp spacer, the reverse complement of a TAL recognition sequence, and an A. TAL Arrays were designed and constructed to create heterodimeric pairs of TAL-ssSPBs (i.e., one left and one right TAL Array—PBx). The PAH1-6-TAL-PBx construct pairs were assayed as described above and the results are shown in Table 20 and FIG. 14.

TABLE 20

| | % GFP | |
|---|---|---|
| Target | Pair On-Target | Pair Off-Target |
| PAH1 | 12.6 | 2.8 |
| PAH2 | 18.4 | 3.2 |
| PAH3 | 14.9 | 1.8 |
| PAH4 | 5.6 | 1.5 |
| PAH5 | 9.2 | 0.6 |
| PAH6 | 6.2 | 1.7 |

As shown in Table 20 the split GFP splicing reporter assay demonstrates that the newly constructed PAH TAL-PBxs are capable of performing site-specific transposition into the target sequences that are naturally found in genomic DNA.

In another experiment, the reporter plasmids also were co-transfected with either the PAH left or right TAL-PBx constructs (i.e., homodimers) and assayed as described above. The results are shown in Table 21 and FIG. 15.

TABLE 21
% GFP

| | Pair On- | Left Only On- | Right Only On- | Pair Off- |
|---|---|---|---|---|
| Target | Target | Target | Target | Target |
| PAH1 | 12.6 | 5.7 | 4.2 | 2.8 |
| PAH2 | 18.4 | 8.8 | 7.0 | 3.2 |
| PAH3 | 14.9 | 6.8 | 5.7 | 1.8 |
| PAH4 | 5.6 | 3.6 | 2.9 | 1.5 |
| PAH5 | 9.2 | 3.6 | 3.9 | 0.6 |
| PAH6 | 6.2 | 2.7 | 2.5 | 1.7 |

As shown in Table 21 the PAH TAL-PBx homodimers capable of recognizing only the left or right target sequence of integration sites comprising a both left and right target sequence still resulted in site-specific transposition at the target site compared to off target controls, albeit at lower levels than the corresponding heterodimer pairs.

ii. Genomic Site-Specific Transposition

After confirming the newly designed PAH TALs were functional and recognize its target sequence, the PAH TAL-PBx constructs were used to catalyze site-specific transposition into endogenous genomic DNA. Briefly, 120,000 HEK293T cells were plated in 24 well plates in 500 ul of DMEM medium supplemented with 10% FBS. The following day, a transfection mixture containing 25 ng of the PAH left TAL-PBx expression vector, 25 ng of the PAH right TAL-PBx expression vector, 450 ng of a PiggyBac transposon donor plasmid, and 1 μl of JetPrime transfection reagent in a total volume of 50 μl of JetPrime buffer were assembled. The mixture was added to the HEK293T cells and they were incubated for four days at 37° C. at 5% CO2, splitting the cells 1:6 at day one.

The transposon donor plasmid contained a PiggyBac transposon containing from 5′ to 3′ direction: TTAA, a 309 bp fragment containing the Piggybac 5′ ITR (SEQ ID NO: 319) and part of the UTR, a “cargo” consisting of multiple restriction enzyme recognition sites, a 238 bp fragment containing the Piggybac 3′ ITR (SEQ ID NO: 320) and part of the UTR, and TTAA. As controls, transfections were also performed using Super PiggyBac transposase (SPB; SEQ ID NO: 80) or no transposase in place of PAH TAL-PBx to assess random integration or no integration of the transposon from the donor plasmid.

To assess site-specific integration of the transposon donor into the PAH locus, genomic DNA was extracted from the transfected cells and analyzed by digital droplet PCR (ddPCR) using a probe-based detection scheme. One primer that binds within the transposon was paired with a primer that binds PAH genomic DNA near the TTAA integration site. Therefore, an amplicon should only be generated following site-specific transposition into the PAH locus. Since integration is not directional, two assays were designed for each PAH target to detect integration of the transposon in forward and reverse direction.

Amplicons corresponding to forward and/or reverse transposon integration were detected from genomic DNA isolated with cells transfected with PAH1 TAL-PBx, PAH2 TAL-PBx and PAH3 TAL-PBx constructs providing direct evidence of genomic integration at the PAH locus. A reduced number of amplicons were detected using SPB transposase, likely resulting from low level random integration events, whereas no amplicons were detected in the absence of transposase suggesting site-specific transposition at the PAH1, PAH2 and PAH3 target sequences only in the presence of TAL-PBx constructs.

### B. LINE1 Repeat Element Episomal Target Site-specific Transposition

Nine different LINE1 repeat element genomic sequences derived from the LINE1 Ta1d Consensus Sequence (SEQ ID NOs: 366-374) were selected as target sequences for episomal site-specific transposition using LRE1-6 TAL-PBx construct pairs.

Episomal split GFP splicing reporter constructs were designed and cloned as described above for each constructed LRE1-6 Left & Right TAL-PBx in Example 18D: LRE1-6 TAL-PBx constructs: LRE1L, LRE2L, LRE3L, LRE4.1L, LRE4.2L, LRE5L, and LRE6L Left TAL-PBxs (SEQ ID Nos. 207, 209, 212, 214, 215, 217 & 219, respectively) and LRE1R, LRE2.1R, LRE2.2R, LRE3R, LRE4R, LRE5R, LRE6.1R and LRE6.2R Right TAL-PBxs (SEQ ID Nos. 208, 210, 211, 213, 216, 218, 220 & 221).

The Episomal split GFP splicing assay was performed as described above. Briefly, each LINE1 genomic target site (SEQ ID Nos. 366-374) was cloned into a reporter.

Each TAL-PBx construct was co-transfected with its corresponding reporter or a reporter containing a non-target sequence and GFP was measured the following day. The results are show in Table 22.

TABLE 22

| | On-target | | | Off-target | | |
|---|---|---|---|---|---|---|
| | Replicate 1 | Replicate 2 | Average | Replicate 1 | Replicate 2 | Average |
| LINE L1/R1 | 14.3 | 18.1 | 16.2 | 3.2 | 3.8 | 3.5 |
| LINE L2/R2.1 | 15.9 | 18.8 | 17.4 | 3.1 | 3.4 | 3.2 |
| LINE L2/R2.2 | 16.9 | 16.4 | 16.7 | 3.1 | 3.4 | 3.2 |
| LINE L3/R3 | 9.0 | 8.3 | 8.6 | 3.5 | 3.2 | 3.4 |
| LINE L4.1/R4 | 16.9 | 17.0 | 17.0 | 4.0 | 3.8 | 3.9 |
| LINE L4.2/R4 | 19.1 | 17.5 | 18.3 | 4.1 | 3.7 | 3.9 |
| LINE L5/R5 | 7.7 | 7.6 | 7.7 | 3.0 | 2.9 | 2.9 |
| LINE L6/R6.1 | 15.5 | 15.5 | 15.5 | 4.1 | 4.5 | 4.3 |
| LINE L6/R6.2 | 16.0 | 14.6 | 15.3 | 3.0 | 3.1 | 3.0 |

ii. Genomic Site-Specific Transposition

After confirming the newly designed LINE1 TALs were functional and recognize their target sequence, the LINE1 TAL-PBx constructs were used to catalyze site-specific transposition into endogenous genomic DNA. Briefly, 120,000 HEK293T cells were plated in 24 well plates in 500 ul of DMEM medium supplemented with 10% FBS. The following day, a transfection mixture containing 25 ng of the LINE1 left TAL-PBx expression vector, 25 ng of the LINE1 right TAL-PBx expression vector, 225 ng of a PiggyBac transposon donor plasmid, and 1 μl of JetPrime transfection reagent in a total volume of 50 μl of JetPrime buffer were assembled. The mixture was added to the HEK293T cells and they were incubated for three days at 37° C. at 5% CO2, splitting the cells 1:6 at day one.

The transposon donor nanoplasmid contained a PiggyBac transposon containing from 5′ to 3′ direction: TTAA, a 309 bp fragment containing the Piggybac 5′ ITR and part of the UTR, a “cargo” consisting of an EF1a promoter, a puromycin resistance gene, a 2A peptide, and a GFP reporter, followed by a 238 bp fragment containing the Piggybac 3′ ITR and part of the UTR, and TTAA. As controls, transfections were also performed using PBx transposase (SEQ ID NO: 56) or no transposase in place of LINE1 TAL-PBx to assess random integration or no integration of the transposon from the donor plasmid.

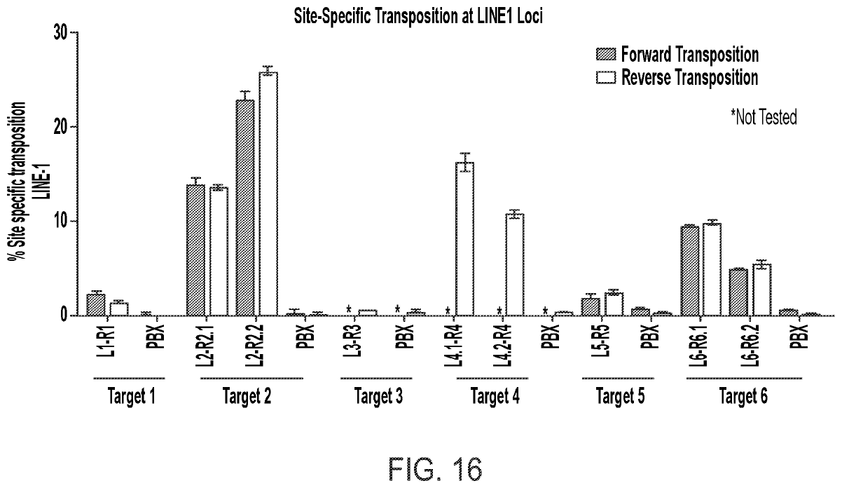

To assess site-specific integration of the transposon donor into the LINE1 loci, genomic DNA was extracted from the transfected cells three days post transfections and analyzed by digital droplet PCR (ddPCR) using a probe-based detection scheme. One primer that binds within the transposon was paired with a primer that binds LINE1 genomic DNA near the TTAA integration site. Therefore, an amplicon should only be generated following site-specific transposition into a LINE1 locus. Since integration is not directional, two assays were designed for each LINE1 target to detect integration of the transposon in forward and reverse direction. The results are shown in FIG. 16 and Table 23.

TABLE 23

| | | Forward | Reverse |
|---|---|---|---|
| Target | Transposase | Integration % | Integration % |
| 1 | LINE L1/R1 | 2.3 | 1.4 |
| | PBX | 0.2 | 0.0 |
| 2 | LINE L2/R2.1 | 13.7 | 13.6 |
| | LINE L2/R2.2 | 22.9 | 25.9 |
| | PBX | 0.3 | 0.1 |
| 3 | LINE L3/R3 | Not tested | 0.7 |
| | PBX | Not tested | 0.4 |
| 4 | LINE L4.1/R4 | Not tested | 16.2 |
| | LINE L4.2/R4 | Not tested | 10.8 |
| | PBX | Not tested | 0.3 |
| 5 | LINE L5/R5 | 1.8 | 2.4 |
| | PBX | 0.7 | 0.3 |
| 6 | LINE L6/R6.1 | 9.5 | 9.9 |
| | LINE L6/R6.2 | 4.9 | 5.4 |
| | PBX | 0.5 | 0.2 |

As shown in FIG. 16 and Table 23, amplicons corresponding to forward and/or reverse transposon integration were detected from genomic DNA isolated with cells transfected with LINE1 TAL-PBx constructs providing direct evidence of genomic integration at LINE1 loci. Higher levels of transposition were detected for targets 2, 4, and 6 than for targets 1, 3, and 5. Amplicons were not detected at high levels in the absence of TAL-PBx constructs suggesting site-specific transposition at the LINE1 target sequences only in the presence of TAL-PBx constructs. An additional primer set detecting a reference single copy gene was used to determine the number of genomes represented per ddPCR reaction. This allowed for quantification of the percent of genomes containing an edited LINE1 locus (on average).

The target sites with the most robust integration, targets 2, 4, and 6, all contain a TTTAAA integration site as shown in FIG. 16. These data are in agreement with the data shown in Example 19 and Table 19 demonstrating TAL-PBx fusion compositions preference for TTTAAA integration sites over TTAA integration sites.

### C. B2M Episomal and Genomic Target Site-Specific Transposition

i. Episomal

Genomic sequences derived from the first intron of the B2M gene (SEQ ID Nos. 375-381) were selected as target sequences for episomal site-specific transposition using B2M 1-7 TAL-PBx construct pairs (SEQ ID Nos. 222-235). The B2M genomic sequences (SEQ ID Nos. 375-381) were cloned into the episomal split GFP reporter vector and the episomal split GFP splicing assay was performed as described above. Briefly, each B2M TAL-PBx pair was co-transfected with its corresponding reporter and GFP was measure four days post transfection. The results are shown in Table 24.

TABLE 24

| | Site-Specific Transposition (% GFP+) | | | | | |
|---|---|---|---|---|---|---|
| | On-target | | | Off-target | | |
| | Replicate | Replicate | | Replicate | Replicate | |
| | 1 | 2 | Average | 1 | 2 | Average |
| B2M L1/R1 | 16.3 | 14.9 | 15.6 | 9.4 | 9.6 | 9.5 |
| B2M L2/R2 | 18.6 | 19.4 | 19.0 | 10.2 | 10.5 | 10.4 |
| B2M L3/R3 | 13.1 | 13.7 | 13.4 | 8.5 | 9.3 | 8.9 |
| B2M L4/R4 | 55.8 | 63.3 | 59.6 | 8.6 | 7.5 | 8.0 |
| B2M L5/R5 | 40.8 | 61.5 | 51.2 | 6.4 | 7.1 | 6.8 |
| B2M L6/R6 | 39.3 | 43.5 | 41.4 | 5.6 | 5.7 | 5.6 |
| B2M L7/R7 | 33.8 | 33.5 | 33.7 | 7.6 | 6.9 | 7.3 |

As shown in Table 24, four of the seven B2M TAL-PBx pairs (pairs 4, 5, 6, and 7) catalyzed site-specific transposition at an appreciable frequency.

ii. Genomic Site-Specific Transposition

After confirming the newly designed B2M TALs were functional and recognize their target sequence, the active B2M TAL-PBx constructs were used to catalyze site-specific transposition into endogenous genomic DNA. Briefly, 120,000 HEK293T cells were plated in 24 well plates in 500 ul of DMEM medium supplemented with 10% FBS. The following day, a transfection mixture containing 25 ng of the B2M left TAL-PBx expression vector, 25 ng of the B2M right TAL-PBx expression vector, 225 ng of a PiggyBac transposon donor plasmid, and 1 μl of JetPrime transfection reagent in a total volume of 50 μl of JetPrime buffer were assembled. The mixture was added to the HEK293T cells and they were incubated for five days at 37° C. at 5% CO2, splitting the cells 1:8 at day one.

The transposon donor nanoplasmid contained a PiggyBac transposon containing from 5′ to 3′ direction: TTAA, a 309 bp fragment containing the Piggybac 5′ ITR (SEQ ID NO: 319) and part of the UTR, a “cargo” consisting of an EF1a promoter, a puromycin resistance gene, a 2A peptide, and a GFP reporter, followed by a 238 bp fragment containing the Piggybac 3′ ITR (SEQ ID NO: 320) and part of the UTR, and TTAA. As controls, transfections were also performed using PBx transposase (SEQ ID NO: 56) or no transposase in place of B2M TAL-PBx to assess random integration or no integration of the transposon from the donor plasmid.

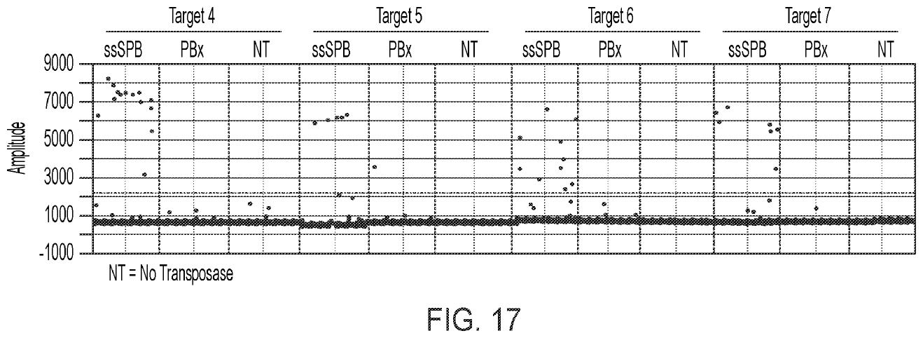

To assess site-specific integration of the transposon donor into the B2M locus, genomic DNA was extracted from the transfected cells five days post transfections and analyzed by digital droplet PCR (ddPCR) using a probe-based detection scheme. One primer that binds within the transposon was paired with a primer that binds B2M genomic DNA near the TTAA integration site. Therefore, an amplicon should only be generated following site-specific transposition into a B2M locus. The results are shown in FIG. 17.

As shown in FIG. 17, amplicons corresponding to transposon integration were detected from genomic DNA isolated with cells transfected with B2M TAL-PBx constructs providing direct evidence of genomic integration at the B2M locus. Amplicons were not detected at high levels in the absence of TAL-PBx constructs suggesting site-specific transposition at the B2M target sequences only in the presence of TAL-PBx constructs.

### Example 21: Construction of PBx Fusion Proteins

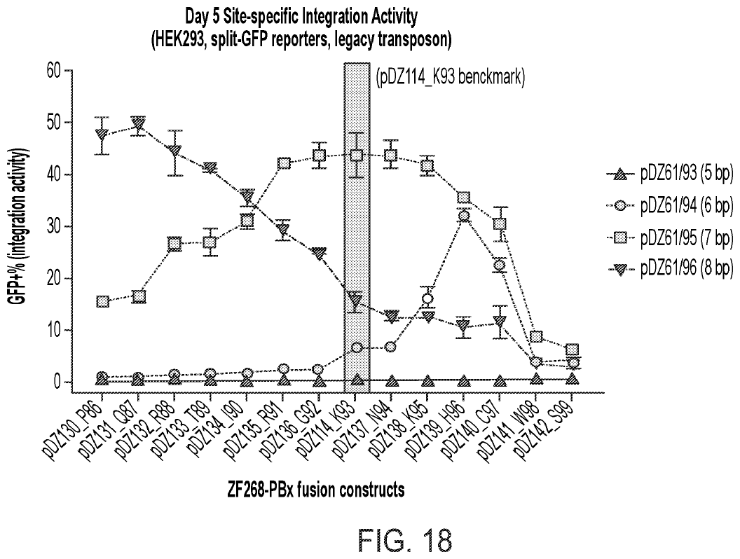

Zinc finger domains flanked by GGGGS linkers at both N- and C-terminals (SEQ ID NO: 57) were inserted into SV40 NLS PBx, replacing one of various positions between P86 and S99 (the ZF-ssSPB fusion points shown in Table 25). Thus, the constructs retained the N-terminus of PBx upstream of the zinc finger domain. The sequences of the constructs are set forth in SEQ ID NOs: 67 and 387-399. These sequences were used to assess integration activity using the split-GFP reporter shown in FIG. 7 using the targets shown in SEQ ID NOs: 61-64. Results are shown in FIG. 18 and Table 25.

TABLE 25

| Fusion point | 5 bp | | | 6 bp | | |
|---|---|---|---|---|---|---|
| PBx | Replicate 1 | Replicate 2 | Average | Replicate 1 | Replicate 2 | Average |
| P86 | 0.33 | 0.46 | 0.395 | 1.47 | 1.19 | 1.33 |
| Q87 | 0.36 | 0.37 | 0.365 | 1.26 | 0.96 | 1.11 |
| R88 | 0.63 | 0.39 | 0.51 | 1.63 | 1.49 | 1.56 |
| T89 | 0.42 | 0.45 | 0.435 | 1.96 | 1.46 | 1.71 |
| I90 | 0.53 | 0.18 | 0.355 | 1.99 | 1.58 | 1.785 |
| R91 | 0.7 | 0.46 | 0.58 | 2.79 | 2.5 | 2.645 |
| G92 | 0.36 | 0.47 | 0.415 | 2.81 | 2.42 | 2.615 |
| K93 | 0.58 | 0.57 | 0.575 | 6.89 | 7.04 | 6.965 |
| N94 | 0.59 | 0.46 | 0.525 | 6.79 | 6.63 | 6.71 |
| K95 | 0.5 | 0.49 | 0.495 | 15.1 | 17.8 | 16.45 |
| H96 | 0.41 | 0.4 | 0.405 | 31.3 | 33.1 | 32.2 |
| C97 | 0.52 | 0.52 | 0.52 | 21.6 | 23.7 | 22.65 |
| W98 | 0.73 | 0.58 | 0.655 | 4.61 | 3.69 | 4.15 |
| S99 | 0.8 | 0.5 | 0.65 | 4.75 | 4.15 | 4.45 |
| Fusion Point | 7 bp | | | 8 bp | | |
| PBx | Replicate 1 | Replicate 2 | Average | Replicate 1 | Replicate 2 | Average |
| P86 | 15.6 | 16 | 15.8 | 50 | 44.9 | 47.45 |
| Q87 | 17.7 | 16 | 16.85 | 50.6 | 48 | 49.3 |
| R88 | 27.8 | 25.9 | 26.85 | 47.3 | 41.1 | 44.2 |
| T89 | 25.3 | 28.9 | 27.1 | 41.3 | 40.8 | 41.05 |
| I90 | 32 | 29.9 | 30.95 | 36.8 | 34.4 | 35.6 |
| R91 | 41.6 | 42.9 | 42.25 | 30.7 | 27.9 | 29.3 |
| G92 | 45.4 | 42 | 43.7 | 24.8 | 24.6 | 24.7 |
| K93 | 46.9 | 40.9 | 43.9 | 17 | 14.3 | 15.65 |
| N94 | 42 | 45.8 | 43.9 | 12.2 | 12.9 | 12.55 |
| K95 | 43.1 | 40.5 | 41.8 | 12.4 | 12.8 | 12.6 |
| H96 | 35.8 | 35.4 | 35.6 | 9.09 | 12.2 | 10.645 |
| C97 | 32.9 | 28.3 | 30.6 | 13.9 | 9.4 | 11.65 |
| W98 | 9.44 | 7.78 | 8.61 | 3.6 | 3.3 | 3.45 |
| S99 | 6.5 | 6.12 | 6.31 | 3.23 | 2.75 | 2.99 |

### Example 22: Construction of TALENS and TAL-PBx Fusions Recognizing Alternative Nucleotides Other than Thymidine 5′ of Target Binding Site

### A. TALENs

Wild type TAL sequences that most efficiently recognize target sequences immediately 3′ of a T were mutated to recognize a 5′G instead of a 5′T (NT-G Mutant; SEQ ID NO: 74) or a mutant that does not require any specific 5′ nucleotide (NT-ON; SEQ ID NO: 75). These mutations were introduced into the GFP1 Right TALEN (SEQ ID NO: 160; Example 16) by mutating the amino acid sequence QW located at positions 119-120 to the amino acid sequence SR to generate the NT-G variant or by replacing the amino acid sequence QWS at positions 119-121 with YH to generate the NT-ON variant to create GFP1 Right TALEN NT-G (SEQ ID NO: 401) and GFP1 Right TALEN NT-ON (SEQ ID NO: 402).

The TALEN NT-G and NT-ON designs were tested using the single strand annealing reporter (Example 17). The target site corresponding to the GFP1 Right TALEN (SEQ ID NO: 288) was modified to replace T 5′ of the target sites with either an A, a C, or a G to create SEQ ID NOs: 403-405). A transfection mixture containing 90 ng of each TALEN, 10 ng of the corresponding reporter and 1.5p of Transit-2020 transfection reagent in a total volume of 20 μl of Serum Free OptiMem medium were assembled. A TALEN or a reporter were transfected alone as negative controls. An aliquot of 30,000 HEK293T cells in 180 μl of DMEM medium supplemented with 10% FBS was added and the transfection mixture was plated in 96 well plates and incubated for one day at 37° C. at 5% CO2. The following day, a lysis buffer was added to the cells and the lysate was transferred to a white 96 well plate. A buffer containing substrate for Firefly luciferase was mixed with the cells and luciferase luminescence was detected using a plate reader. The results are shown in Table 26.

TABLE 26

Reporter Luciferase (RLU)

| TALEN | 5′A | 5′C | 5′G | 5′T |
|---|---|---|---|---|
| WT | 517595 | 418674 | 294260 | 1594204 |
| NT-G | 1136491 | 692819 | 1067635 | 1214379 |
| NT-βN | 1560024 | 1116975 | 1209825 | 1445861 |

As shown in Table 25, while the WT TALEN led to the highest cleavage of targets comprising a 5′T, the NT-G and NT-PN versions also were capable of similar cleavage at targets comprising 5′—A, C, G, or T.

### B. TAL-PBx Fusions

The NT-G and NT-PN mutations were introduced into the GFP1 Right TAL-PBx fusion (SEQ ID NO: 192; Example 18) to create GFP1 Right NT-G TAL-PBx fusion (SEQ ID NO: 406) and GFP1 Right NT-PN TAL-PBx fusion (SEQ ID NO: 407). The new TAL-PBx fusion designs were tested using the episomal split GFP splicing reporter system (Example 19). The GFP1 Right target site with 13 bp spacers (SEQ ID No: 337) was modified to replace the T 5′ of the target sites with either an A, a C, or a G to create SEQ ID NOs: 408-410.

The activity of the new mutant TAL-PBx fusions was determined using their respective episomal split GFP splicing reporters. Briefly, each reporter plasmid and the donor plasmid were cotransfected into HEK293T cells with the corresponding TAL-PBx expression plasmid. Approximately 120,000 HEK293T cells were plated in 24 well plates in 500p of DMEM medium supplemented with 10% FBS. The following day, a transfection mixture containing 50 ng of the TAL-PBx expression vector, 225 ng of the reporter plasmid, 225 ng of donor plasmid and 1 μl of JetPrime transfection reagent in a total volume of 50p of JetPrime buffer were assembled. This mixture was added to the HEK293T cells and they were incubated for four days at 37° C. at 5% CO2, splitting the cells 1:6 at day one. The percentage of GFP positive cells was determined for each sample. The results are shown in Table 27.

TABLE 27

Integration (% GFP+)

| TAL-PBx | 5′A | 5′C | 5′G | 5′T |
|---|---|---|---|---|
| WT | 11.3 | 11.5 | 11.0 | 30.9 |
| NT-G | 21.5 | 18.5 | 20.4 | 27.6 |
| NT-βN | 23.7 | 22.3 | 20.4 | 30.7 |

As shown in Table 26, the WT TAL-PBx fusion exhibited the highest percentage of integration at targets with a 5′T, similar to the corresponding TALEN version, while the mutated NT-G at targets with a 5′G and NT-PN at targets with 5′—A, C, G, or T were capable of similar integration demonstrating that these alternative targets sites may be effectively targeted and modified using the TALEN and TAL-PBx fusion compositions of the present disclosure.

### Example 23: Construction of TAL-PBx Fusions Comprising Varying Sized Deletions of the N-Terminus of PBx

The first exemplary TAL-PBx fusion was constructed using a 93 amino acid N-terminal deletion of PBx (SEQ ID NO: 66; Example 7). To further explore the position of the deletion site, ten amino acids of PBx sequence were added back in one amino acid increments to create PBx Delta 83—PBx Delta 92 (SEQ ID NO: 86-95). Additionally, ten amino acids were further deleted in one amino acid increments to create PBx Delta 94—PBx Delta 103 (SEQ ID NO: 97-106). These twenty new truncated PBx sequences were used to replace PBx Delta 93 in GFP1 Right TAL-PBx (SEQ ID NO 192) to create GFP1 Right Tal-PBx Delta 83-92 (SEQ ID NOs. 450-459) and GFP1 Right Tal-PBx Delta 94-103 (SEQ ID NOs. 460-469).

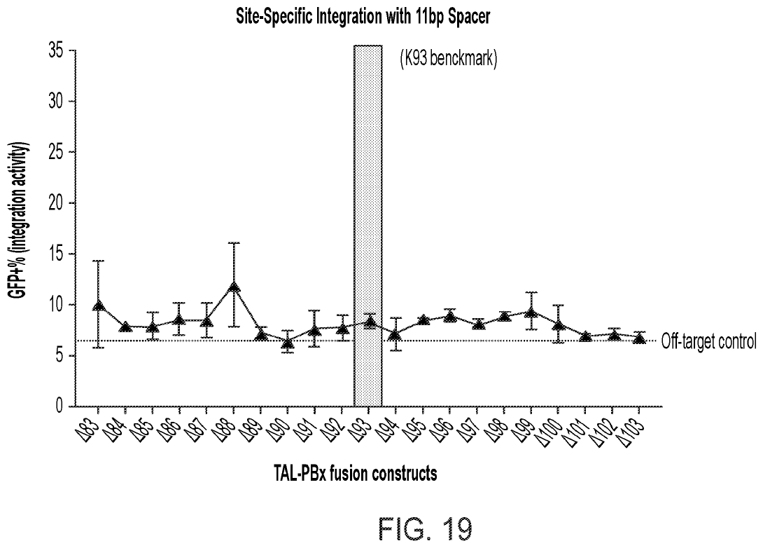

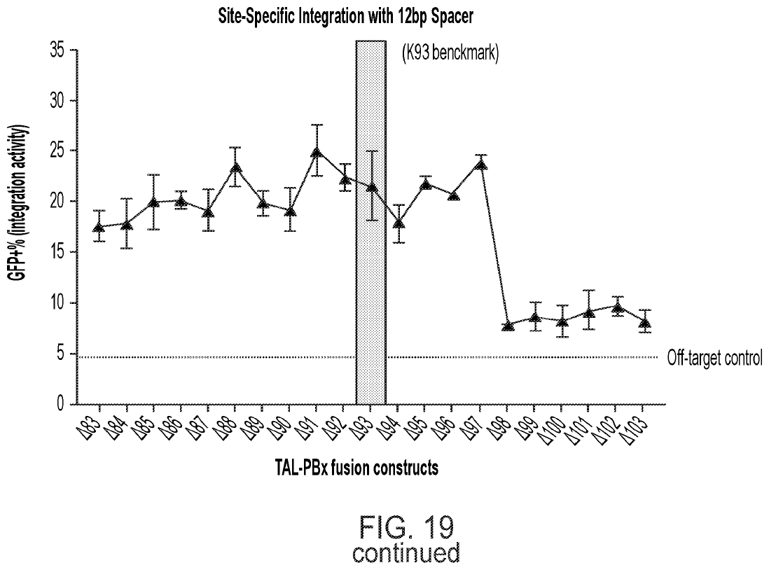

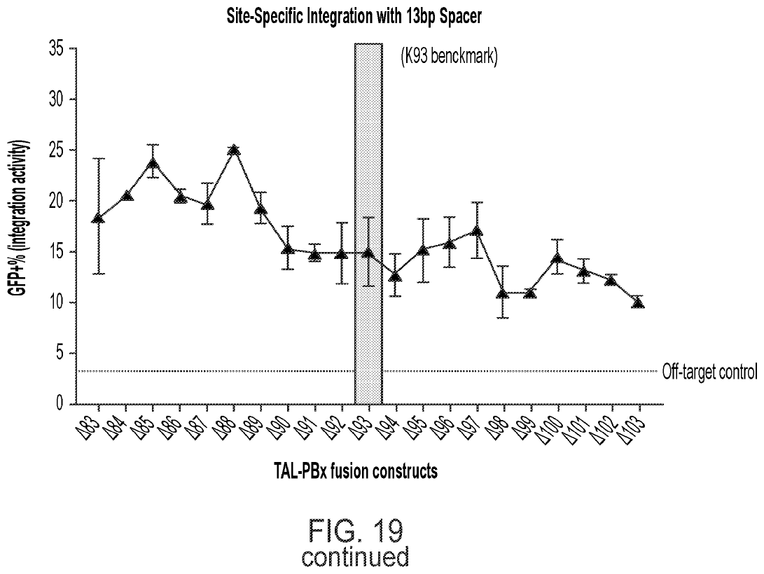

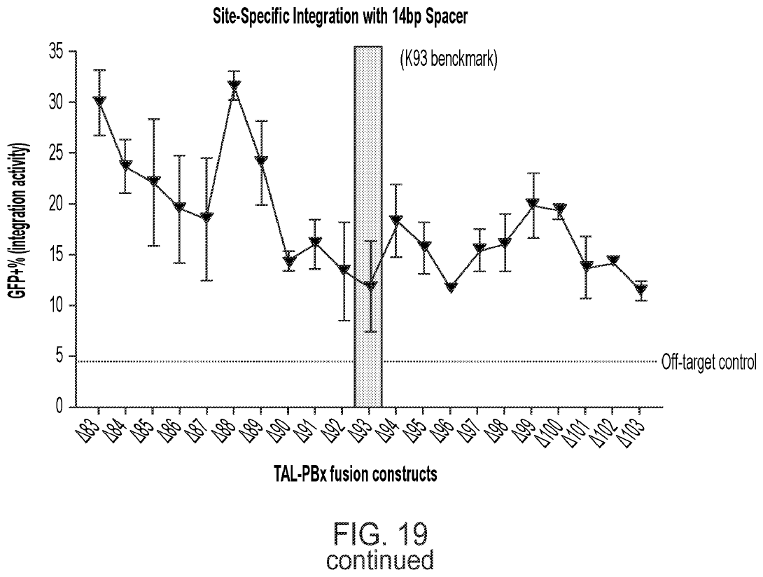

The new mutant GFP1 Right TAL-PBx fusions were tested using their respective episomal split GFP splicing reporters as described in Example 19. Briefly, a site-specific reporter plasmid and the donor plasmid were cotransfected into HEK293T cells with the corresponding GFP1 Right TAL-PBx expression plasmid. As a benchmark control, the original GFP1 Right TAL-PBx fusion with the 93 amino acid truncation of PBx was transfected (SEQ ID NO: 192). As a negative control, a non-targeting (GFP1 Left TAL-PBx) was transfected (SEQ ID NO: 191). The reporter plasmid contained two target GFP1 right target sites (downstream of a 5′T) flanking 13 bp spacers with a TTAA insertion site in the middle (SEQ ID NO: 470). The experiment was repeated using reporters with spacers containing 11 bp spacers (SEQ ID NO: 335), 12 bp spacers (SEQ ID NO: 336), and 14 bp spacers (SEQ ID NO: 338). To perform the transfections, approximately 120,000 HEK293T cells were plated in 24 well plates in 500 μl of DMEM medium supplemented with 10% FBS. The following day, a transfection mixture containing 50 ng of the TAL-PBx expression vector, 225 ng of the reporter plasmid, 225 ng of donor plasmid and 1 μl of JetPrime transfection reagent in a total volume of 50 μl of JetPrime buffer were assembled. This mixture was added to the HEK293T cells and they were incubated for four days at 37° C. at 5% CO2, splitting the cells 1:6 at day one. The percentage of GFP positive cells was determined for each sample four days post transfection. The results are shown in FIG. 19 and Table 28.

TABLE 28
Site Specific Integration (% GFP+)

| | |
|---|---|
| PBx | Target Spacer Length |

| Truncation | 11 bp | 12 bp | 13 bp | 14 bp |
|---|---|---|---|---|
| Δ83 | 10.0 | 17.4 | 18.4 | 30.0 |
| Δ84 | 7.9 | 17.7 | 20.6 | 23.7 |
| Δ85 | 7.8 | 19.8 | 23.9 | 22.1 |
| Δ86 | 8.6 | 19.9 | 20.5 | 19.6 |
| Δ87 | 8.5 | 18.9 | 19.7 | 18.5 |
| Δ88 | 11.9 | 23.2 | 25.1 | 31.6 |
| Δ89 | 7.2 | 19.7 | 19.2 | 24.0 |
| Δ90 | 6.4 | 19.0 | 15.3 | 14.3 |
| Δ91 | 7.6 | 24.8 | 14.8 | 16.1 |
| Δ92 | 7.8 | 22.2 | 14.8 | 13.4 |
| Δ93 (Benchmark) | 8.4 | 21.4 | 15.0 | 11.7 |
| Δ94 | 7.1 | 17.7 | 12.7 | 18.3 |
| Δ95 | 8.5 | 21.7 | 15.2 | 15.7 |
| Δ96 | 9.0 | 20.5 | 15.9 | 11.5 |
| Δ97 | 8.2 | 23.7 | 17.2 | 15.5 |
| Δ98 | 8.9 | 7.7 | 11.0 | 16.1 |
| Δ99 | 9.4 | 8.6 | 10.9 | 19.8 |
| Δ100 | 8.1 | 8.2 | 14.5 | 19.3 |
| Δ101 | 6.9 | 9.2 | 13.1 | 13.8 |
| Δ102 | 7.2 | 9.6 | 12.2 | 14.3 |
| Δ103 | 6.8 | 8.1 | 10.1 | 11.4 |
| Off-Target | 6.4 | 4.6 | 3.1 | 4.6 |

As shown in FIG. 19 and Table 27, all of the new constructs were capable of catalyzing site-specific transposition above background levels with the 12 bp, 13 bp, and 14 bp spacer targets at various levels with some TAL-PBx constructs outperforming the benchmark. The broad activity across a wide range of deletions and various spacer lengths allows for the flexible design of TAL-PBx fusion constructs that are capable of targeting a diverse set of genomic targets of various spacing and TAL-PBx design.

### Example 24: Construction of TAL-PBx Fusions Comprising Varying Sized Deletions of TAL C-terminal Domain

Naturally occurring TALs comprise a 278 amino acid C-terminal domain (SEQ ID NO: 77). The first exemplary TAL-PBx fusion constructed contained a truncated C-terminal domain that retains 63 amino acids (SEQ ID NO: 76). To explore the role of the size of the C-terminal domain, alternative truncations of the TAL C-terminal domain were designed. Truncated TAL C-terminal domains retaining 13, 23, 33, 43, 53, or 73 amino acids were constructed (SEQ ID NOs. 471-476). These C-terminal domain deletions were used to replace the 63 amino acid TAL C-terminal domain in GFP1 Right TAL-PBx (SEQ ID NO: 192) to create GFP1 Right TAL-PBx+13 (SEQ ID NO: 477), GFP1 Right TAL-PBx+23 (SEQ ID NO: 478), GFP1 Right TAL-PBx+33 (SEQ ID NO: 479), GFP1 Right TAL-PBx+43 (SEQ ID NO: 480), GFP1 Right TAL-PBx+53 (SEQ ID NO: 481), and GFP1 Right TAL-PBx+73 (SEQ ID NO: 482).

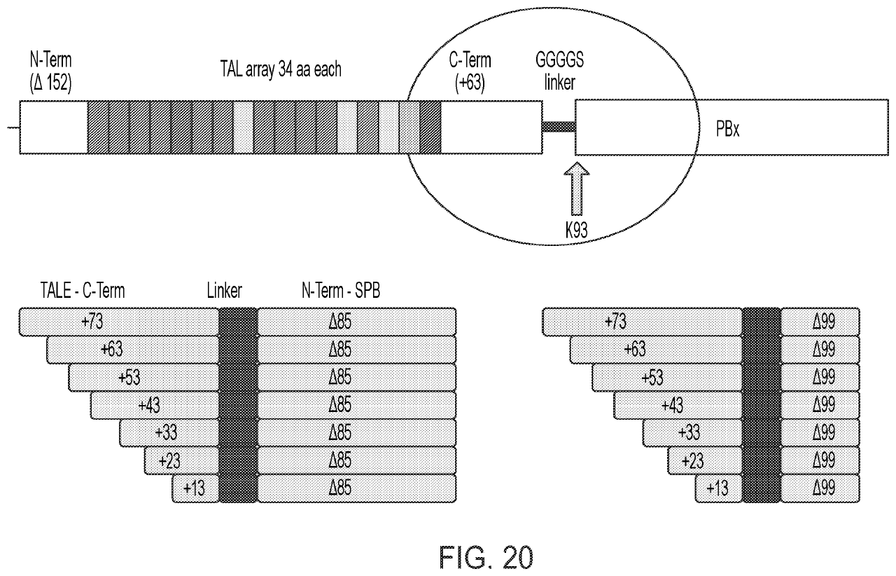

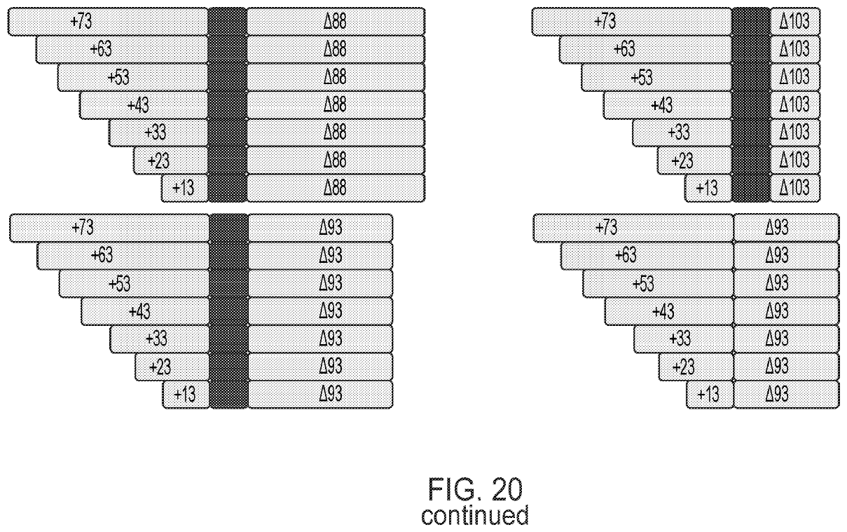

To test the effect of the GGGGS linker sequence positioned between the TAL and PBx sequences, a second set of constructs comprising the 13, 23, 33, 43, 53, 63, and 73 amino acid C-terminal domain of the TAL were created that lacked the GGGGS linker to create GFP1 Right TAL-PBx+13-GGGGS linker (SEQ ID NO: 483), GFP1 Right TAL-PBx+23-GGGGS (SEQ ID NO: 484), GFP1 Right TAL-PBx+33-GGGGS (SEQ ID NO: 485), GFP1 Right TAL-PBx+43-GGGGS (SEQ ID NO: 486), GFP1 Right TAL-PBx+53-GGGGS (SEQ ID NO: 487), GFP1 Right TAL-PBx+63-GGGGS (SEQ ID NO: 488), and GFP1 Right TAL-PBx+73-GGGGS (SEQ ID NO: 489). Furthermore, the array of truncated TAL C-terminal domains was used in combination with several of the alternative PBx N-terminal variants constructed in Example 23. The 63 amino acid TAL C-terminal domain in GFP1 Right TAL-PBx Delta 85 (SEQ ID NO: 452) was replaced with the alternative TAL C-terminal domain truncations to create GFP1 Right TAL-PBx Delta 85+13 (SEQ ID NO: 490), GFP1 Right TAL-PBx Delta 85+23 (SEQ ID NO: 491), GFP1 Right TAL-PBx Delta 85+33 (SEQ ID NO: 492), GFP1 Right TAL-PBx Delta 85+43 (SEQ ID NO: 493), GFP1 Right TAL-PBx Delta 85+53 (SEQ ID NO: 494), GFP1 Right TAL-PBx Delta 85+73 (SEQ ID NO: 495). The 63 amino acid TAL C-terminal domain in GFP1 Right TAL-PBx Delta 88 (SEQ ID NO: 455) was replaced with the alternative TAL C-terminal domain truncations to create GFP1 Right TAL-PBx Delta 88+13 (SEQ ID NO: 496), GFP1 Right TAL-PBx Delta 88+23 (SEQ ID NO: 497), GFP1 Right TAL-PBx Delta 88+33 (SEQ ID NO: 498), GFP1 Right TAL-PBx Delta 88+43 (SEQ ID NO: 499), GFP1 Right TAL-PBx Delta 88+53 (SEQ ID NO: 500), GFP1 Right TAL-PBx Delta 88+73 (SEQ ID NO: 501). The 63 amino acid TAL C-terminal domain in GFP1 Right TAL-PBx Delta 99 (SEQ ID NO: 465) was replaced with the alternative TAL C-terminal domain truncations to create GFP1 Right TAL-PBx Delta 99+13 (SEQ ID NO: 502), GFP1 Right TAL-PBx Delta 99+23 (SEQ ID NO: 503), GFP1 Right TAL-PBx Delta 99+33 (SEQ ID NO: 504), GFP1 Right TAL-PBx Delta 99+43 (SEQ ID NO: 505), GFP1 Right TAL-PBx Delta 99+53 (SEQ ID NO: 506), GFP1 Right TAL-PBx Delta 99+73 (SEQ ID NO: 507). The 63 amino acid TAL C-terminal domain in GFP1 Right TAL-PBx Delta 103 (SEQ ID NO: 469) was replaced with the alternative TAL C-terminal domain truncations to create GFP1 Right TAL-PBx Delta 103+13 (SEQ ID NO: 508), GFP1 Right TAL-PBx Delta 103+23 (SEQ ID NO: 509), GFP1 Right TAL-PBx Delta 103+33 (SEQ ID NO: 510), GFP1 Right TAL-PBx Delta 103+43 (SEQ ID NO: 511), GFP1 Right TAL-PBx Delta 103+53 (SEQ ID NO: 512), GFP1 Right TAL-PBx Delta 103+73 (SEQ ID NO: 513). These constructs are shown graphically in FIG. 20.

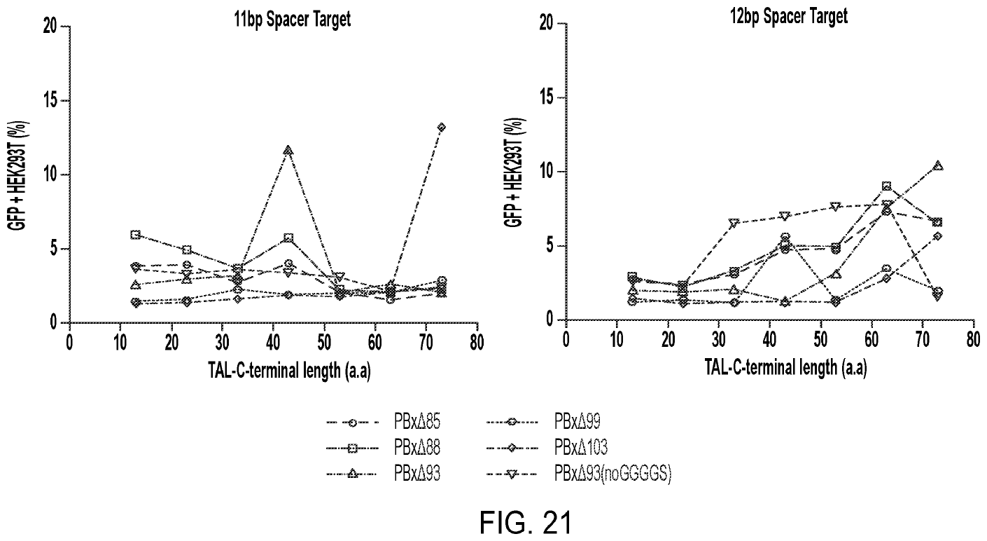

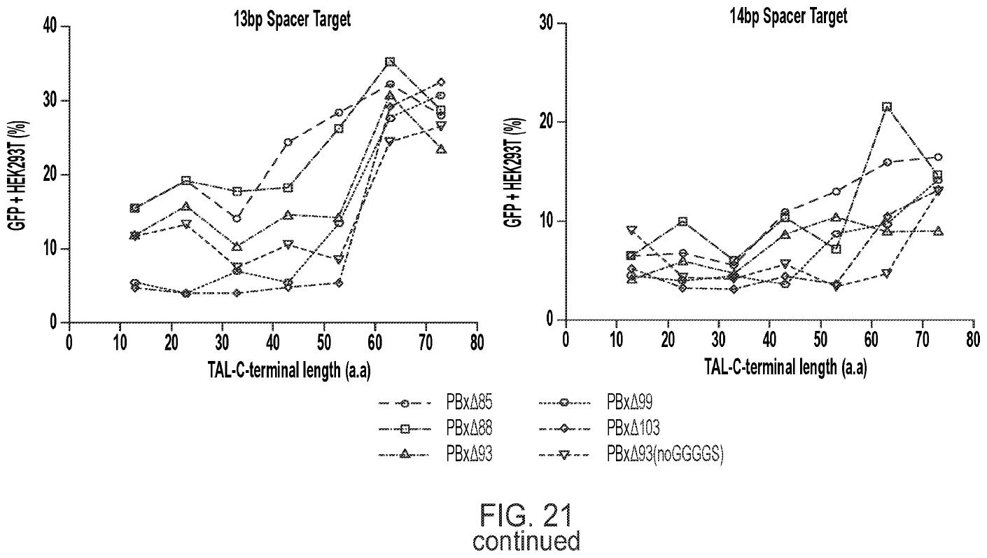

The site-specific integration (percent GFP positive cells) was determined for each construct and the results are shown in FIG. 21 and Table 29.

TABLE 29
Site-Specific Integration (% GFP+)

| | | | | | | |
|---|---|---|---|---|---|---|
| TAL C-Term | PBxΔ85 | PBxΔ88 | PBxΔ93 | PBxΔ99 | PBxΔ103 | PBxΔ93 (no GGGGS) |

11 bp Spacer Target

| | | | | | | |
|---|---|---|---|---|---|---|
| +13 | 3.84 | 5.96 | 2.61 | 1.45 | 1.3 | 3.65 |
| +23 | 3.92 | 4.93 | 2.95 | 1.54 | 1.42 | 3.3 |
| +33 | 2.8 | 3.67 | 3.16 | 2.24 | 1.63 | 3.62 |
| +43 | 4.02 | 5.72 | 11.7 | 1.87 | 1.87 | 3.39 |
| +53 | 2.05 | 2.29 | 1.98 | 2.01 | 1.79 | 3.11 |
| +63 | 1.57 | 2.05 | 2.65 | 2.1 | 2.09 | 2.15 |
| +73 | 2 | 2.29 | 2.05 | 2.9 | 13.2 | 2.46 |

12 bp Spacer Target

| | | | | | | |
|---|---|---|---|---|---|---|
| +13 | 2.75 | 2.93 | 2.02 | 1.22 | 1.44 | 2.54 |
| +23 | 2.41 | 2.14 | 1.89 | 1.36 | 1.09 | 2.38 |
| +33 | 3.07 | 3.31 | 2.04 | 1.18 | 1.18 | 6.54 |
| +43 | 4.75 | 5.04 | 1.23 | 5.62 | 1.23 | 6.99 |
| +53 | 4.71 | 5 | 3.13 | 1.35 | 1.19 | 7.66 |
| +63 | 7.32 | 9 | 7.54 | 3.47 | 2.8 | 7.78 |
| +73 | 6.7 | 6.59 | 10.44 | 1.98 | 5.67 | 1.57 |

13 bp Spacer Target

| | | | | | | |
|---|---|---|---|---|---|---|
| +13 | 15.7 | 15.5 | 11.9 | 5.5 | 4.8 | 11.7 |
| +23 | 19.3 | 19.2 | 15.8 | 4.04 | 4.12 | 13.3 |
| +33 | 14.1 | 17.8 | 10.4 | 7.13 | 4.13 | 7.61 |
| +43 | 24.4 | 18.3 | 14.6 | 5.47 | 4.92 | 10.6 |
| +53 | 28.4 | 26.3 | 14.3 | 13.5 | 5.4 | 8.61 |
| +63 | 32.3 | 35.3 | 30.8 | 27.6 | 29.2 | 24.5 |
| +73 | 28 | 28.8 | 23.5 | 30.7 | 32.5 | 26.6 |

14 bp Spacer Target

| | | | | | | |
|---|---|---|---|---|---|---|
| +13 | 6.55 | 6.48 | 4.17 | 4.52 | 5.25 | 9.091 |
| +23 | 6.81 | 10 | 6.02 | 4 | 3.32 | 4.38 |
| +33 | 5.61 | 6.08 | 4.81 | 4.43 | 3.16 | 4.12 |
| +43 | 10.9 | 10.4 | 8.73 | 3.64 | 4.46 | 5.67 |
| +53 | 13 | 7.23 | 10.4 | 8.73 | 3.68 | 3.35 |
| +63 | 16 | 21.6 | 9.01 | 9.7 | 10.5 | 4.72 |
| +73 | 16.5 | 14.7 | 9.1 | 14.2 | 13.2 | 13 |

As shown in in FIG. 21 and Table 29, the 88 and 89 amino acid N-terminal truncations of PBx often outperformed the 93, 99, and 103 amino acid truncations. Additionally, the 73, 63, 53, and 43 amino acid length TAL C-terminal domains often outperformed the 33, 23, and 13 amino acid TAL C-terminal domains. Various combinations are superior to the benchmark for different target spacer lengths allowing for flexibility in the design of TAL-PBx fusion constructs for targeting diverse genomic loci.

### Example 25: Site-saturated Mutagenesis of PBx R372A and K372A Mutations and Relative Integration-Excision Activities

Mutations R372A and K375A in the integration domain of PiggyBac transposase amino acid sequence renders the transposase integration deficient, while retaining the excision function. It has been proposed that converting the positively charged lysine and arginine residues to the neutrally charged alanine reduces the transposases affinity for the negatively charged DNA backbone adjacent to its TTAA integration site.

As a strategy for increasing site-specific transposition, additional mutations in these “PBx” positions 372 and 375 were explored as a way of titrating PBx transposase affinity for DNA. Site-saturation mutagenesis (or SSM) is a technique of mutating an amino acid at a given position to all other 19 amino acids. SSM was performed at position 372 in the context of TAL-PBx fusions containing the K375A mutation. Additionally, SSM was performed at position 375 in the context of TAL-PBx fusions containing the R372A mutation. Specifically, SSM was performed on the GFP1 Right TAL-PBx fusion (SEQ ID NO: 192). In the context of this TAL-PBx fusion, PBx positions 372 and 375 correspond to positions 849 and 852 of TAL-PBx. The SSM resulted in 19 position 372 mutants (SEQ ID NOs. 411-429) and 19 position 375 mutants (SEQ ID NOs. 430-448).

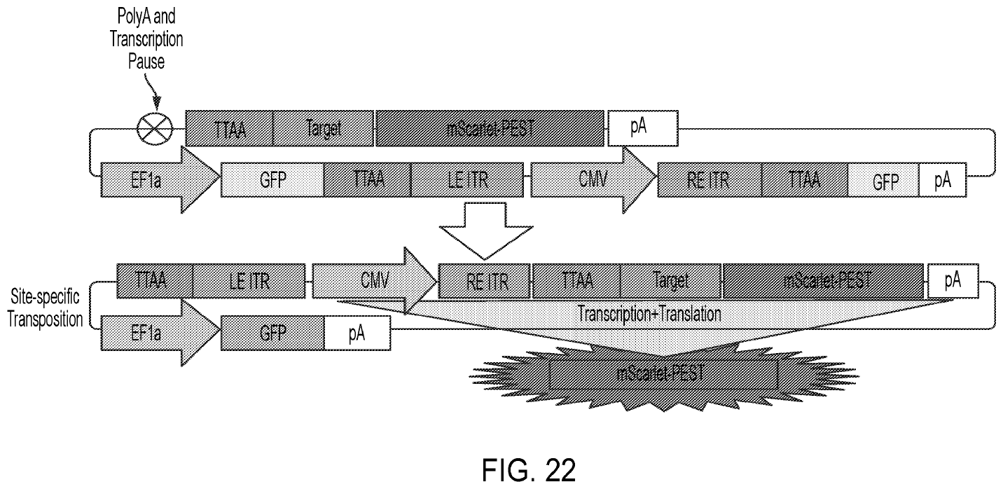

An “all-in-one site-specific excision/integration episomal reporter” system was developed to test the new mutants' ability to catalyze site-specific transposition (FIG. 22). This episomal reporter system comprises a plasmid containing a transposon donor along with a transposon integration site all on the same plasmid. The transposon consists of, in 5′ to 3′ direction: a TTAA sequence, the 35 bp PiggyBac minimal 5′ ITR (SEQ ID NO: 319), a CMV promoter, the 63 bp PiggyBac minimal 3′ ITR (SEQ ID NO: 320), and a TTAA sequence. The transposon in this plasmid disrupts the open reading frame of a GFP preceded by an EF1a promoter and followed by poly-adenylation signal sequence. The vector also contains, in the opposite orientation, a polyA and transcription pause site, a TTAA integration site adjacent to GFP1 right target sequences and 13 bp spacers, followed by a PEST destabilized mScarlet reporter and a poly-adenylation signal sequence. This “all-in-one site-specific excision/integration episomal reporter” (SEQ ID NO: 449), when transfected into cells alone, should express no GFP and no or little mScarlet. Upon transposon excision (catalyzed by SPB, PBx, or ssSPB) GFP should be expressed. Upon site-specific integration of the CMV promoter containing transposon into its target site upstream of mScarlet, mScarlet should be expressed at above background levels (FIG. 22).

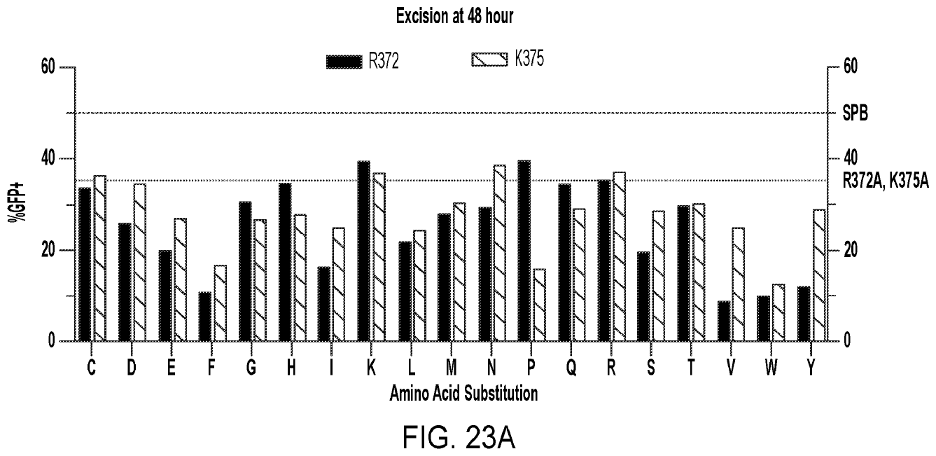

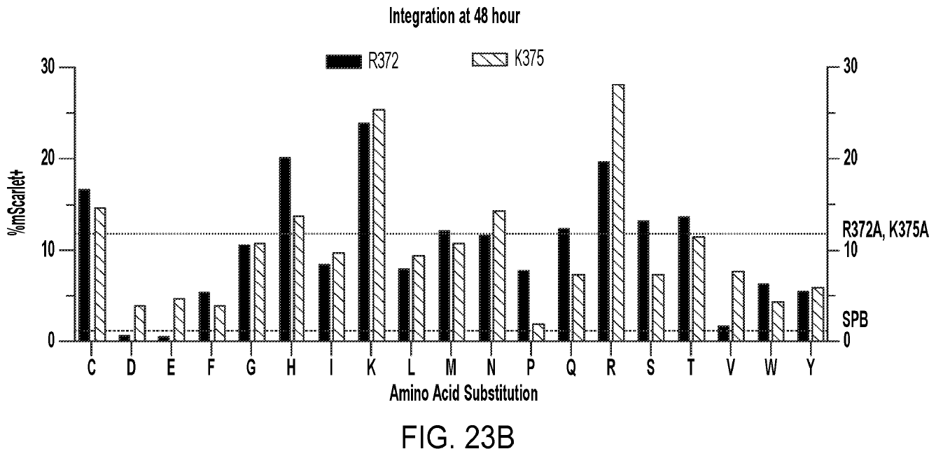

Each of the TAL-PBx SSM mutant expression vectors were co-transfected into HEK293T along with the all-in-one site-specific excision/integration episomal reporter. Briefly, a transfection mix containing 50 ng of a mutant TAL-PBx, 50 ng of the reporter plasmid, 0.3 μl of Transit2020 transfection reagent, in a total volume of 20 μl of serum free OptiMEM medium was assembled. To this, approximately 60,000 HEK293T cells in 180 μl of DMEM medium supplemented with 10% FBS were added, then 80 μl of this transfection mixture was plated in duplicate in clear bottom 96 well plates and incubated at 37° C. at 5% CO2. As controls, the original R372A, K375A TAL-PBx as well as SPB were transfected in place of the SSM mutant TAL-PBx's. GFP and mScarlet fluorescence were detected using an Incucyte live cell analysis instrument. The percent fluorescent cells for each of the excision (GFP) and site-specific integration (mScarlet) reporters is displayed in FIG. 23 and Table 30.

TABLE 30

| | |
|---|---|
| Excision | Integration |
| (% GFP+) | (% mScarlet+) |

| Mutation | R372 | K375 | R372 | K375 |
|---|---|---|---|---|
| C | 33.4 | 36.0 | 16.7 | 14.6 |
| D | 25.7 | 34.3 | 0.6 | 3.8 |
| E | 19.7 | 26.7 | 0.5 | 4.6 |
| F | 10.6 | 16.5 | 5.3 | 3.7 |
| G | 30.4 | 26.4 | 10.5 | 10.6 |
| H | 34.4 | 27.7 | 20.1 | 13.7 |
| I | 15.9 | 24.6 | 8.4 | 9.6 |
| K | 39.2 | 36.8 | 23.8 | 25.3 |
| L | 21.7 | 24.2 | 7.9 | 9.4 |
| M | 27.7 | 30.0 | 12.0 | 10.6 |
| N | 29.0 | 38.7 | 11.5 | 14.2 |
| P | 39.6 | 15.6 | 7.7 | 1.8 |
| Q | 34.5 | 29.0 | 12.2 | 7.2 |
| R | 35.1 | 37.0 | 19.6 | 28.0 |
| S | 19.4 | 28.3 | 13.1 | 7.2 |
| T | 29.7 | 30.2 | 13.5 | 11.5 |
| V | 8.6 | 24.7 | 1.6 | 7.6 |
| W | 9.8 | 12.3 | 6.2 | 4.2 |
| Y | 11.9 | 28.7 | 5.4 | 5.8 |
| R372A, K375A | 35.2 | 35.2 | 11.7 | 11.7 |
| SPB | 50.2 | 50.2 | 1.2 | 1.2 |

As shown in FIGS. 23 A & B and Table 29, several of the SSM mutants resulted in similar or higher site-specific integration than the benchmark R372A, K375A TAL-PBx fusion demonstrating that the integration/excision activity of the PBx sequence may be titrated depending on the amino acid positions at positions 372 and 375.

### Example 26: Identification of TTAA Genomic Sites Suitable for Site-Specific Integration and Design of Zinc Finger Motif—PBx Fusions Targeting Specific TTAA Genomic Locations

As shown in Example 9, zinc finger motif PBx (ZFM-PBx) fusion protein requires precise spacing (6 bp, 7 bp or 8 bp) between the zinc finger binding site and the TTAA integration site for efficient site-specific integration. ZFM-PBx fusions also require two zinc finger binding sites flanking the target TTAA integration site to promote a greater activity. A custom software program, which considers the published CoDA zinc finger library as well as the spacing requirements between the zinc finger motif binding site and TTAA, was developed to select zinc finger targetable TTAAs along the genome. Three TTAA target sites on the human genome were selected (SEQ ID NOs. 526-528). To target these three sites, a total number of six zinc finger PBx fusions were generated. (Table 31).

TABLE 31

| Site ID | ZFM-PBx Left | ZFM-PBx Right |
|---|---|---|
| chr17-1 | Chr17-1L ZF-PBx | Chr17-1R ZF-PBx |
| | (SEQ ID NO: 529) | (SEQ ID NO: 530) |
| chr21-1 | Chr21-1L ZF-PBx | Chr21-1R ZF-PBx |
| | (SEQ ID NO: 531) | (SEQ ID NO: 532) |
| chr21-2 | Chr21-2L ZF-PBx | Chr21-2R ZF-PBx |
| | (SEQ ID NO: 533) | (SEQ ID NO: 534) |

As shown in Table 31 two sites are located at chromosome 21 (referred as chr21-1, chr21-2) (SEQ ID Nos. 526-527) and one site is located at chromosome 17 (referred as chr17-1) (SEQ ID NO: 528). A total number of 6 ZFM-PBx fusions were generated by Gibson Assembly to target these 3 endogenous sites.

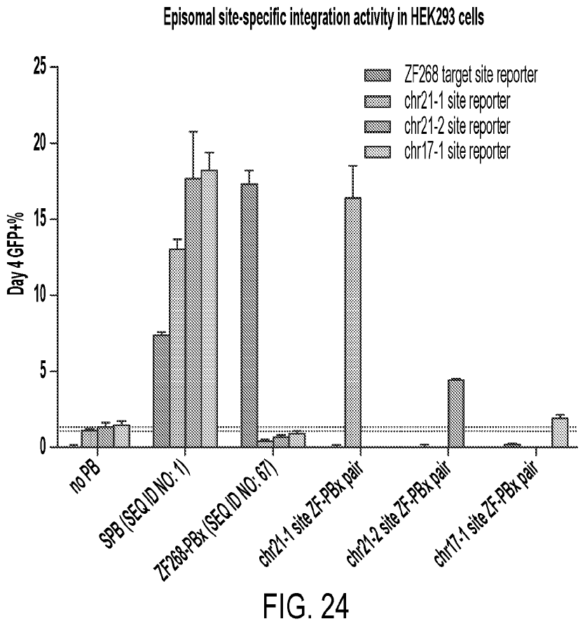

To determine whether the newly generated ZFM-PBx fusions are functional and can perform site-specific integration, the episomal site-specific integration assay was conducted using the split-GFP reporter system. Flow cytometry was performed to obtain GFP+ percentage as a measurement of site-specific integration activity following transfection of the ZFM-PBx fusions, the corresponding episomal synthetic reporter and the split-GFP transposon. The results are shown in FIG. 24 and Table 32.

TABLE 32

| | ZFM | | | |
|---|---|---|---|---|
| | target | chr21-1 | chr21-2 | chr17-1 |
| | site | site | site | site |
| no PB | 0.1025% | 1.09% | 1.345% | 1.445% |
| SPB (SEQ ID NO: 1) | 7.355% | 13.1% | 17.75% | 18.25% |
| ZF268-PBx (SEQ ID | 17.3% | 0.4% | 0.71% | 0.895% |
| NO: 67) | | | | |
| chr21-1 site ZF-PBx pair | 0.09% | 16.4% | | |
| chr21-2 site ZF-PBx pair | 0.0995% | | 4.415% | |
| chr17-1 site ZF-PBx pair | 0.16% | | | 1.905% |

As shown in FIG. 24 and Table 31, SPB showed integration activity at all 4 episomal targets, because of its random integration nature. As expected, the ZFM-PBx fusion only shows integration activity at its target site (ZF268 target site) not the other 3 sites, demonstrating site-specific integration of ZFM-PBx. Notably, the new ZFM-PBx pair (SEQ ID NOs. 531-532) which targets the chr21-1 site showed good site-specific integration activity as compared to the previous benchmark ZFM-PBx. The chr21-2 ZFM-PBx pair (SEQ ID NOs. 533-534) showed moderate activity, whereas the chr17-1 ZFM-PBx pair (SEQ ID NOs. 529-530) showed minimal activity. In summary, these data demonstrate that the zinc finger motif PBx fusion strategy can be applied to different endogenous TTAA sites with good activity and specificity.

### Example 27: Construction of Zinc Finger Motif—Tandem PBx Fusion Constructs (ZFM-tdPBx) and Relative Integration—Excision Activities

A ZFM tandem PBx fusion (ZFM-tdPBx) was constructed by ligating a second PBx sequence to the C-terminal of the ZFM-PBx fusion (SEQ ID NO: 67) via a L3 linker sequence (SEQ ID NO: 16). The 2^{nd} PBx sequence comprises a 10 amino acid deletion at its N-terminal to promote greater activity of the tandem dimer. The resulting final ZFM-tdPBx construct (SEQ ID NO: 535) was obtained with the following elements in order: NLS+92aa N terminal domain of the 1^{st} PBx+ZF268 DNA binding domain+rest sequence of the 1^{st} PBx+L3 linker+the 2^{nd} PBx comprising a 10 amino acid N terminal truncation.

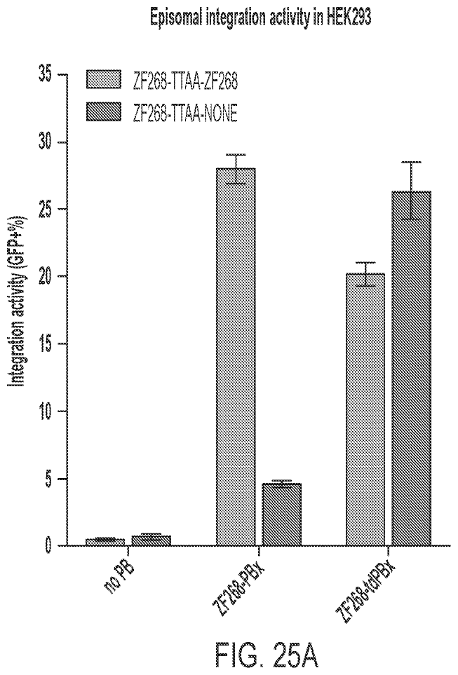

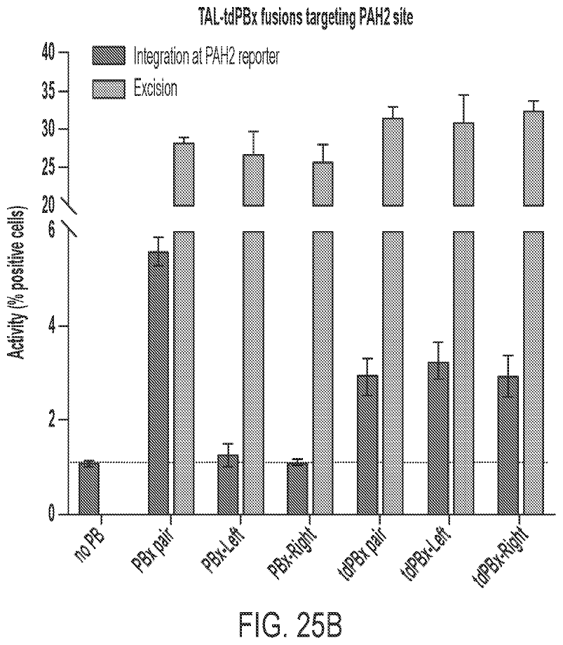

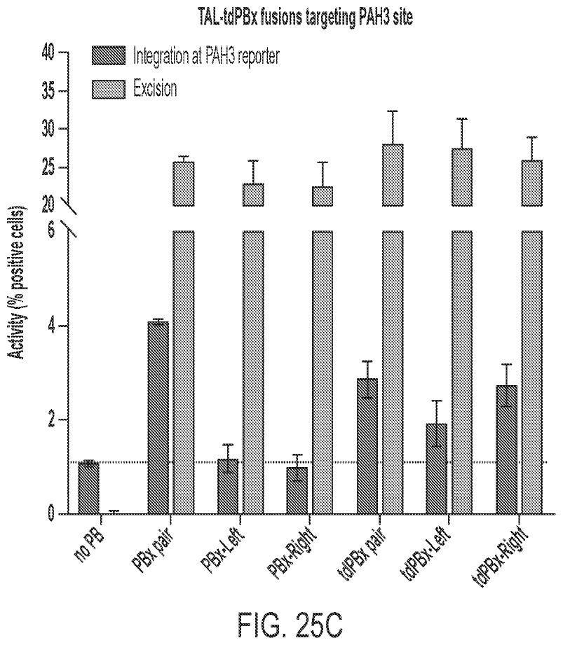

The activity of ZFM-tdPBx fusion was tested together with the ZFM-PBx monomer fusion against two targets in the episomal site-specific integration assay: the first target has two ZF268 binding domain flanking TTAA (ZF268-TTAA-ZF268, SEQ ID NO: 62); the second target only has a single ZF268 binding domain next to the TTAA (ZF268-TTAA-NONE, SEQ ID NO: 545). Both targets comprise the ideal 7 bp spacing between the zinc finger binding site and the TTAA integration site. The excision activities (percentage H2Kk+) and integration activities (percentage GFP+) were determined at Day 4 (72 hours after transfection). The results are shown in FIG. 25A and Table 33.

TABLE 33

| | ZF268-TTAA- | ZF268-TTAA- | Excision activity |
|---|---|---|---|
| | ZF268 | NONE | (% H2Kk+) |
| No PB | 0.47% | 0.68% | 0.05% |
| ZF268-PBx | 27.95% | 4.61% | 31.5% |
| ZF268-tdPBx | 20.15% | 26.35% | 39.1% |

As shown in FIG. 25A and Table 31, the monomeric PBx fusion, ZFM-PBx had greatly reduced activity towards the ZF268-TTAA-NONE target compared to the double sided ZF268-TTAA-ZF268 target, demonstrating that ZF268 fusion with monomer PBx requires two DNA binding sites flanking the target TTAA site for efficient site-specific integration. However, ZF268-tdPBx fusion has uncompromised activity (26.35%) towards the single-sided target, ZF268-TTAA-NONE, suggesting that ZFM-tdPBx only requires one DBD binding site flanking the TTAA to be functional. Notably, ZFM-tdPBx favored the single-sided TTAA target versus the double-sided TTAA target. One possibility is the tandem dimer PBx adopts a side-by-side orientation where the 2^{nd} PBx folds down and sits alongside of the 1^{st} PBx (other than head-to-tail), stabilizing the transposase-transposon complex. As a result, the 2^{nd} PBx did not require a 2^{nd} DNA binding domain at the other side of the TTAA integration site, promoting a single DNA binding domain mediated site-specific integration. Also, ZFM-tdPBx fusion exhibited higher excision activity compared to the monomeric ZFM-PBx fusion (Table 32).

### Example 28: Construction of TAL—Tandem PBx Fusion Constructs (TAL-tdPBx) and Relative Integration—Excision Activities

TAL-tdPBx fusions targeting the PAH2 and PAH3 sites were generated using a similar design described in Example 27, and the excision and integration activities of the PAH TAL-tdPBx fusions (SEQ ID NOs. 536-539) were compared to their corresponding monomeric TAL-PBx fusions. The results are shown for PAH2 and PAH3 constructs in FIGS. 25B & 25C and in Table 34 and Table 35, respectively.

TABLE 34

| | Excision | Integration (episomal) |
|---|---|---|
| no PB | 0.0335% | 1.11% |
| PBx pair | 28.35% | 5.58% |
| PBx-Left | 26.9% | 1.29% |
| PBx-Right | 26% | 1.145% |
| tdPBx pair | 31.7% | 2.945% |
| tdPBx-Left | 31.15% | 3.275% |
| tdPBx-Right | 32.6% | 2.945% |

TABLE 35

| | Excision | Integration (episomal) |
|---|---|---|
| no PB | 0.025% | 1.075% |
| PBx pair | 25.7% | 4.065% |
| PBx-Left | 22.65% | 1.185% |
| PBx-Right | 22.35% | 0.995% |
| tdPBx pair | 28.1% | 2.86% |
| tdPBx-Left | 27.4% | 1.91% |
| tdPBx-Right | 25.8% | 2.73% |

As shown in FIGS. 25B and 25C and Tables 33 and 34, both PAH TAL-tdPBx constructs only required a single DBD binding site flanking the TTAA target whereas the monomeric PAH TAL-PBx constructs worked as a pair and require two DBD binding sites flanking the TTAA target. Although the excision activities of TAL-tdPBx fusions were slightly higher than TAL-PBx fusions, the integration activities were slightly lower than monomer PBx fusions in episomal assays. These results demonstrate that TAL-tandem PBx fusions may be constructed that are active even at TTAA sites comprising on a single DBD site.

### Example 29: Construction of TAL-PBx Fusions Targeting Chromosome 17 Recognizing One 5′T and one 5′non-T Base

A second genomic location at chromosome 17 was specifically targeted to demonstrate the programmability and versatility of the TAL-PBx site-specific integration system. In his example, another target at chromosome 17 was chosen (referred as chr17-TAL). This genomic location on chromosome 17 shares several advantageous features of this target site: i. The genomic sequence at this site repeats multiple times within a small section of chromosome 17; and ii. This site has sequence composition which allows for more efficient site-specific integration by the TAL-PBx fusion protein.

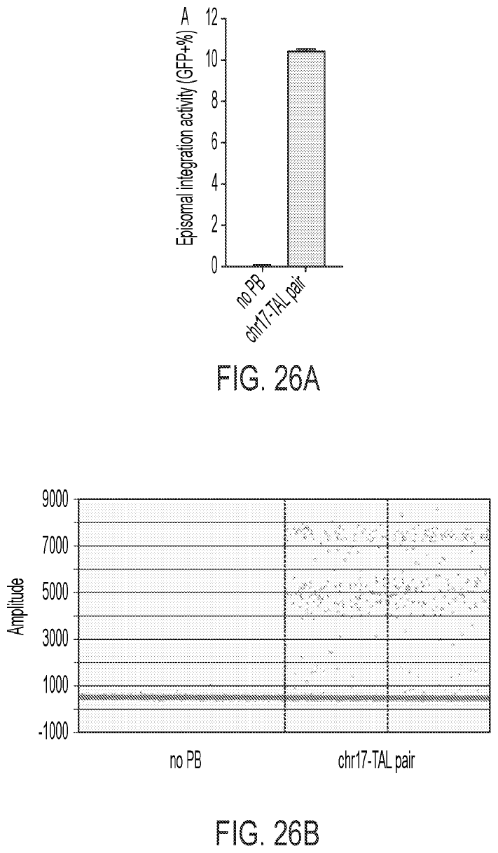

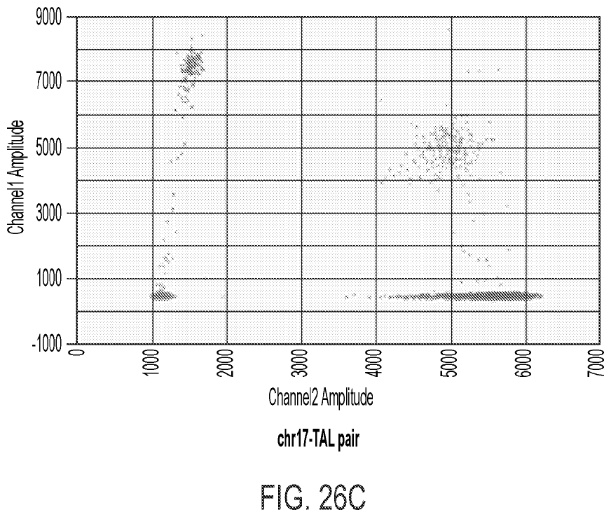

TAL binding sites 13 base pairs away from the target TTAA site, Chr17 Target L1 (SEQ ID NO:540) and Chr17 Target R1 (SEQ ID NO:541) were selected as DNA binding sites for efficient site-specific integration. A TAL-PBx pair (SEQ ID NOs. 542-543) were constructed targeting these two genomic sites. On the left side of the TTAA, the TAL binding site does not have a “T” base at its 5′-terminus and, therefore, a NT-PN variant TAL was employed to expand the programmability of the TAL design. On the right side of the TTAA, a traditional TAL design strategy was utilized given the presence of a 5′-terminal “T”. An episomal reporter plasmid containing the chr17-TAL target sequence was constructed as described herein to validate the TAL-PBx pair. The episomal integration activity (percentage of GFP+ cells) was determined and the results are shown in FIG. 26A. As shown in FIG. 26A, the chr17-TAL pair showed good site-specific integration activity of greater than 10% in this episomal assay.

The next experiment was designed to determine whether the chr17-TAL-PBx pair was able to site-specifically integrate a transposon at its genome target. The chr17-TAL pair and the transposon DNA were introduced into cells via transient transfection. Three days after transfection, genomic DNA was harvested and ddPCR was performed to quantify site-specific integration activity at the chr17-TAL site. As shown in FIG. 26B and FIG. 26C, site-specific integration was detected at the chr17 genomic site shown as positive clusters of droplets demonstrating the ability of TAL-PBx constructs of the present disclosure to site-specifically transpose a DNA molecule at a specific target site.

What is claimed is:

1. A fusion protein comprising, in N-terminal to C-terminal order: a DNA targeting domain and a first transposase domain comprising the sequence set forth in SEQ ID NO: 544, wherein the first transposase domain comprises a deletion of the 83-103 most N-terminal amino acids of SEQ ID NO: 544.

2. The fusion protein of claim 1, wherein the DNA targeting domain comprises three Zinc Finger Motifs.

3. The fusion protein of claim 1, wherein the DNA targeting domain comprises one or more TAL domains.

4. The method of claim 3, wherein the TAL domain comprises the sequence set forth in any one of SEQ ID NOs: 107-110.

5. The fusion protein of any one of claims 1-4, wherein the DNA targeting domain binds to a nucleic acid sequence encoding GFP, zinc finger 268 (ZFM268), phenylalanine hydroxylase (PAH), beta-2-microglobulin (B2M) or a LINE1 repeat element.

6. The fusion protein of any one of claims 1-5, wherein the first transposase domain and the DNA targeting domain are connected by a linker.

7. The fusion protein of claim 6, wherein the linker comprises the sequence GGGGS.

8. The fusion protein of any one of claims 1-7, wherein the first transposase domain comprises an N-terminal deletion of amino acids 1-83, 1-84, 1-85, 186, 1-87, 1-88, 1-89, 1-90, 1-91, 1-92, 1-93, 1-94, 1-95, 1-96, 1-97, 1-98, 1-99, 1-100, 1-101, 1-102 or 1-103.

9. The fusion protein of any one of claims 1-8, wherein the transposase domain comprises the sequence set forth in any one of SEQ ID NOs: 86-106.

10. The fusion protein of any one of claims 1-9, wherein the first transposase domain comprises (a) at least one mutation selected from the group consisting of M185R, M185K, D197K, D197R, D198K, D198R, D201K, and D201R; or (b) at least one mutation selected from the group consisting of L204D, L204E, K500D, K500E, R504E, and R504D.

11. The fusion protein of any one of claim 1-10, further comprising a second transposase domain C-terminal to the first transposase domain, wherein the second transposase domain comprises the sequence set forth in SEQ ID NO: 544.

12. The fusion protein of claim 11, wherein the second transposase domain comprises a deletion of N-terminal amino acids 1-83, 1-84, 1-85, 186, 1-87, 1-88, 1-89, 1-90, 1-91, 1-92, 1-93, 1-94, 1-95, 1-96, 1-97, 1-98, 1-99, 1-100, 1-101, 1-102 or 1-103 of SEQ ID NO: 544.

13. The fusion protein of claim 11 or 12, wherein the second transposase domain comprises (a) at least one mutation selected from the group consisting of M185R, M185K, D197K, D197R, D198K, D198R, D201K, and D201R; or (b) at least one mutation selected from the group consisting of L204D, L204E, K500D, K500E, R504E, and R504D.

14. A polynucleotide comprising a nucleic acid sequence encoding the fusion protein of any one of claims 1-13.

15. A vector comprising the polynucleotide of claim 14.

16. A method of integrating a transgene into a genomic target site of a cell, the method comprising introducing into the cell the fusion protein of any one of claims 1-13 and a transposon, wherein the transposon comprises, in 5′ to 3′ order: a 5′ITR, the transgene, and a 3′ ITR.

17. The method of claim 16, wherein the transposon further comprises an exogenous promoter between the 5′ ITR and the transgene.

18. The method of claim 16 or 17, wherein the transgene encodes a detectable marker.

19. The method of claim 18, wherein the detectable marker is GFP.

20. The method of claim 16 or 17, wherein the transgene is a gene that is not expressed by the cell prior to the introduction of the fusion protein and the transposon.

21. The method of any one of claims 16-20, wherein the genomic target site is located on chromosome 17 or 21.

22. The method of any one of claims 16-20, wherein the genomic target site is located in the B2M gene.

23. The method of any one of claims 16-20, wherein the genomic target site is located in a repetitive element.

24. The method of claim 23, wherein the repetitive element is a LINE element.

25. The method of any one of claims 16-20, wherein the genomic target site is located in an intron of a gene.

26. The method of claim 25, wherein the genomic target site is located in the intron of the PAH gene.

27. The method of any one of claims 16-26, wherein the cell is in vivo.

28. A method of modifying the genome of a cell, the method comprising: providing the cell with the fusion protein of any one of claims 1-13, wherein the cell comprises a modified binding site comprising, in 5′ to 3′ order, the reverse of the sequence of a target site for the DNA targeting domain, a first spacer, a TTAA target integration site for SPB, a second spacer, and the complement of the sequence of the target site for the DNA targeting domain.

29. An integration cassette for site-specific transposition of a nucleic acid into the genome of a cell comprising a nucleic acid comprising or consisting of a central transposon ITR integration site TTAA sequence flanked by at least one upstream Zinc Finger Motif DNA-binding domain binding site (“ZFM-DBD”) and at least one downstream ZFM-DBD, wherein each of the upstream and the downstream ZFM-DBD is separated from the TTAA sequence by 7 base pairs.

30. An integration cassette for site-specific transposition of a nucleic acid into the genome of a cell comprising or consisting of a nucleic acid comprising or consisting of a central transposon ITR integration site TTAA sequence flanked by an upstream TAL array target sequence and a downstream TAL array target sequence, wherein each of the upstream and the downstream TAL array target sequences is separated from the TTAA sequence by 12-14 base pairs.

31. An integration cassette for site-specific transposition of a nucleic acid into the genome of a cell comprising a nucleic acid comprising a central transposon ITR integration site TTTAAA sequence flanked by an upstream TAL array target sequence and a downstream TAL array target sequence, wherein each of the upstream and the downstream TAL array target sequences is separated from the TTTAAA sequence by 12 base pairs.

32. The integration cassette of claims 30 or 31, wherein each of the at least one upstream and downstream TAL array target site sequences are the same.

33. The integration cassette of claims 30 or 31, wherein each of the at least one upstream and downstream TAL array target site sequences are different.

34. The integration cassette of any of claims 30-33, wherein each of the at least one upstream and downstream TAL Array target sites target a 10 bp sequence of beta-2-microglobulin gene (“B2M”), phenylalanine hydroxylase gene (“PAH”) or a LINE1 repeat element.

35. The integration cassette of claim 32, wherein the at least one upstream TAL array target sequence and the at least one downstream TAL array target sequence bind to a nucleic acid comprising the sequence GCGTGGGCG.

36. A cell, comprising the integration cassette of any one of claims 29-35 stably integrated into the genome of the cell.

37. A method for site-specific transposition of a DNA molecule into the genome of a cell, comprising introducing into the cell of claim 36:
a) a nucleic acid encoding a fusion protein comprising a DNA binding domain and a transposase; wherein the fusion protein is expressed in the cell; and
b) a DNA molecule comprising a transposon; wherein the expressed fusion protein integrates the transposon by site-specific transposition into the TTAA sequence of the stably integrated integration cassette.

38. A method for generating an engineered cell by site-specific transposition, comprising introducing into the cell of claim 36:
a) a nucleic acid encoding a fusion protein comprising a DNA binding domain and a transposase; wherein the fusion protein is expressed in the cell; and
b) a DNA molecule comprising a transposon; wherein the expressed fusion protein integrates the transposon by site-specific transposition into the TTAA sequence of the stably integrated integration cassette thereby generating the engineered cell.
